(19) United States
(12) Patent Application Publication
(10) Pub. No.: US 2014/0058106 A1
(43) Pub. Date: Feb. 27, 2014

(54) SYSTEM FOR FLUORINATING ORGANIC COMPOUNDS

(71) Applicant: President and Fellows of Harvard College, Cambridge, MA (US)

(72) Inventors: Tobias Ritter, Cambridge, MA (US); Takeru Furuya, Cambridge, MA (US); Hanns M. Kaiser, Hamburg (DE)

(21) Appl. No.: 13/953,449

(22) Filed: Jul. 29, 2013

Related U.S. Application Data
(63) Continuation of application No. 12/865,703, filed on Nov. 16, 2010, now abandoned.
(60) Provisional application No. 61/063,096, filed on Jan. 31, 2008.
(60) Provisional application No. 61/050,446, filed on May 5, 2008.
(60) Provisional application No. 61/075,463, filed on Jun. 25, 2008.

(51) Int. Cl. B01J 31/22; C07C 29/62; C07C 45/63; C07D 307/82; C07C 37/62; C07C 41/22; C07D 209/08; C07C 17/093; C07C 231/12

(52) U.S. Cl. CPC B01J 31/2295; C07C 17/093; C07C 29/62; C07C 45/63; C07C 231/12; C07C 37/62; C07C 41/22; C07D 209/08; C07D 307/82
USPC 546/4; 546/10; 546/12; 548/109; 570/141; 568/812; 568/436; 564/183; 568/775; 568/663; 548/469; 549/471

(57) ABSTRACT

Described herein are fluorinated organic compounds and methods of making fluorinated organic compounds, for example, using palladium complexes. Also described herein are compositions and kits containing compounds and palladium complexes described herein.

### RELATED APPLICATIONS

The present application is a Continuation of U.S. application Ser. No. 12/865,703, which is a 371 U.S. National Phase of International Application No. PCT/US2009/032855, filed Feb. 2, 2009, published as International Publication No. WO 2009/100014 on Aug. 13, 2009, claims priority under 35 U.S.C. §119(e) to U.S. provisional applications, U.S. Ser. No. 61/075,463, filed Jun. 25, 2008, U.S. Ser. No. 61/050,446, filed May 5, 2008, and U.S. Ser. No. 61/063,096, filed Jan. 31, 2008, each of which is incorporated herein by reference.

### BACKGROUND OF THE INVENTION

The regioselective fluorination of organic compounds is an important challenge in the synthesis of pharmaceuticals and agrochemicals (see, for example, Muller et al., Science 2007, 317, 1881-1886; Park et al., Annual Review of Pharmacology and Toxicology 2001, 41, 443-470; Bohm et al., ChemBioChem 2004, 5, 637-643; and Jeschke, P. ChemBioChem 2004, 5, 570-589).

Syntheses of simple fluoroarenes currently rely on the pyrolysis of diazonium tetrafluoroborates (Balz, G.; Schiemann, G. Ber. Deut. Chem. Ges. 1927, 60, 1186-1190), direct fluorination using highly reactive, elemental fluorine (Sandford, G. J. Fluorine Chem. 2007, 128, 90-104), or nucleophilic aromatic substitution reactions of electron-poor aromatic systems by displacement of other halogens or nitro groups (Sun et al., Angew. Chem., Int. Ed. 2006, 45, 2720-2725; Adams et al., Chem. Soc. Rev. 1999, 28, 225-231). The reductive elimination of arylfluorides from palladium(II) fluoride complexes is an attractive potential alternative that has been investigated by Grushin (Grushin, Chem.-Eur. J. 2002, 8, 1006-1014) over the past decade and more recently by Yandulov. A single substrate—p-fluoronitrobenzene—has been prepared successfully in 10% yield in the Yandulov study from a stoichiometric palladium fluoride complex (Yandulov et al., J. Am. Chem. Soc. 2007, 129, 1342-1358). Directed electrophilic fluorination of phenylpyridine derivatives and related structures using catalytic palladium(II) acetate and N-fluoropyridinium salts has been reported by Sanford in 2006 (Hull et al., J. Am. Chem. Soc. 2006, 128, 7134-7135). Taking advantage of the directing effect of a pyridine substituent, proximal carbon-hydrogen bonds can be fluorinated using microwave irradiation at high temperatures (100-150° C., 1-4 h, 33-75% yield). However, the fact that there is an absence in the literature of any general, functional-group-tolerant fluorination reaction methodology reflects the difficulty of forming carbon-fluorine bonds.

The use of ¹⁸F-labeled organic compounds for positron-emission tomography (PET) requires the controlled, efficient introduction of fluorine into functionalized molecules (see, for example, Couturier et al., Eur. J. Nucl. Med. Mol. Imaging. 2004, 31, 1182-1206; Lasne et al., “Chemistry of beta(+)-emitting compounds based on fluorine-18” In Contrast Agents II, 2002; Vol. 222, pp 201-258; and Phelps, Proc. Natl. Acad. Sci. U.S.A. 2000, 97, 9226-9233). PET has been used to measure presynaptic accumulation of ¹⁸F-fluorodopa tracer in the dopaminergic regions of the brain (see, for example, Ernst et al., “Presynaptic Dopaminergic Deficits in Lesch-Nyhan Disease” New England Journal of Medicine (1996) 334:1568-1572), but fluorination of other organic compounds has been difficult due to lack of an appropriate fluorination method.

### SUMMARY OF THE INVENTION

Described herein are palladium complexes, as well as methods of using palladium complexes to fluorinate organic compounds. Also described herein are compositions and kits containing the compounds described herein.

In one aspect, the invention features a palladium complex of formula (I),

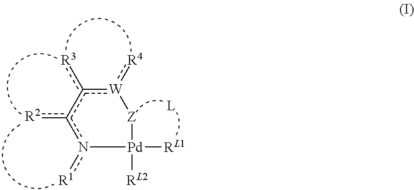

wherein:

Pd has a valency of +2;

R^{L1} and R^{L2} are, independently, optionally substituted aliphatic, optionally substituted heteroaliphatic, optionally substituted aryl, optionally substituted heteroaryl, halogen, —OR^{a}, —SR^{b}, —N(R^{c})₂, —N(R^{c})₃, or —P(R^{x})₃;

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{al}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group [symbol] C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group;

wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

wherein each instance of R^{x} is, independently, hydrogen, an optionally substituted aliphatic, optionally substituted heteroaliphatic, optionally substituted aryl, or optionally substituted heteroaryl group;

when W is —C— or —C(R^{d})— then:
(i) Z is a bond, —O—, —S—, —C(R^{d})₂—, —C(R^{d})═C(R^{d})—, —C(R^{d})═N—, or —N(R^{e})—;

or
(ii) Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring;

or

when W is —N— or —N(R^{e})— then Z is a bond, —C(R^{d})₂—, —C(R^{d})═C(R^{d})—, or —C(R^{d})═N—,

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

R² and R³ are optionally joined to form an optionally substituted 5- to 7-membered heteroaryl, aryl, heterocyclic or carbocyclic ring;

R³ and R⁴ are optionally joined to form an optionally substituted 5- to 7-membered heteroaryl, aryl, heterocyclic or carbocyclic ring;

wherein each of the curved dotted lines [symbol] independently represents optional joining of an optionally substituted 5- to 7-membered ring;

wherein [symbol] represents a single or double bond; and

wherein at least one of R^{L1} and R^{L2} comprises a negatively charged moiety, or the complex further comprises a negatively charged counterion X⁻.

In some embodiments, the palladium complex is of the formula:

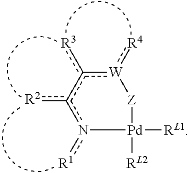

In some embodiments, the palladium complex is of the formula:

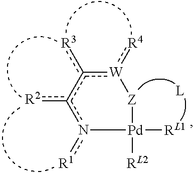

wherein Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring; and

wherein curved solid lines [symbol] represent joining of the 5- to 7-membered palladacycle.

In some embodiments, W is —C—.

In some embodiments, Z is —N(R^{e})—. In some embodiments, R^{e} is —S(O)₂R^{e1}. In some embodiments, R^{e1} is optionally substituted aryl. In some embodiments, R^{e} is:

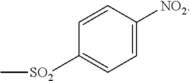

In some embodiments, R¹ and R² are joined to form an optionally substituted 6-membered heteroaryl ring. In some embodiments, R³ and R⁴ are joined to form an optionally substituted 6-membered aryl ring.

In some embodiments, R^{L1} comprises a 6-membered ring. In some embodiments, R^{L1} is —N(R^{c})₂. In some embodiments, the two R^{c} groups of —N(R^{c})₂ are joined to form an optionally substituted heteroaryl ring, e.g., pyridyl. In some embodiments, R^{L2} is —N(R^{c})₂. In some embodiments, the two R^{c} groups of —N(R^{c})₂ are joined to form the group ≡≡C(R^{c1}). In some embodiments, R^{L2} is acetonitrile. In some embodiments, R^{L2} is —OR^{a}. In some embodiments, R^{L2} is acetate. In some embodiments, R^{L2} is halogen (e.g., fluoro or chloro).

In some embodiments, Z is —N— joined via a linker group -L- to the group R^{L1} to form a 5- to 7-membered palladacycle, wherein -L- is —S(O)₂— and wherein —N(R)₂ is a group wherein two R^{c} groups are joined to form an optionally substituted heteroaryl ring. In some embodiments, Z, L and R^{L1} provide a group of the formulae:

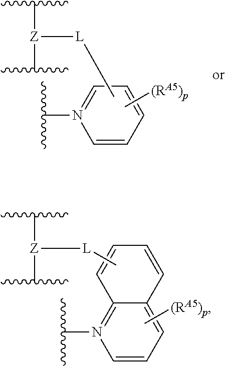

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is an integer between 0 to 5, inclusive.

In some embodiments, the palladium complex is:

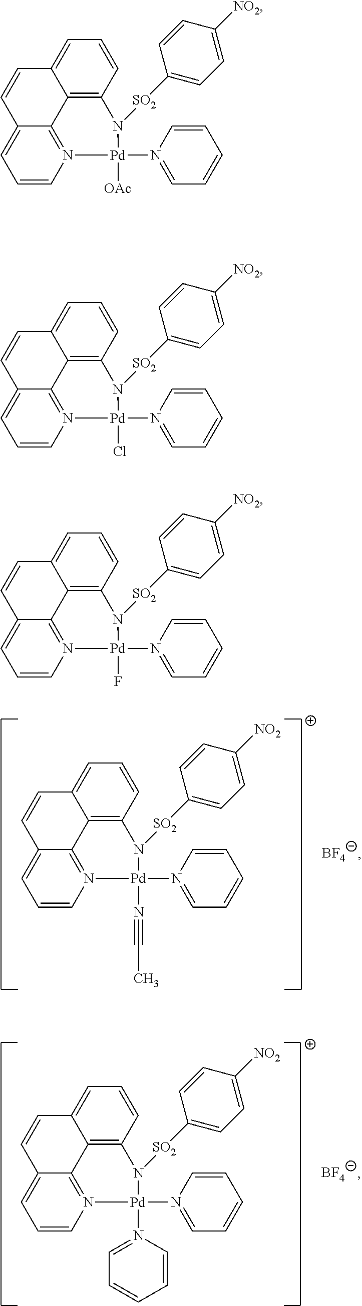

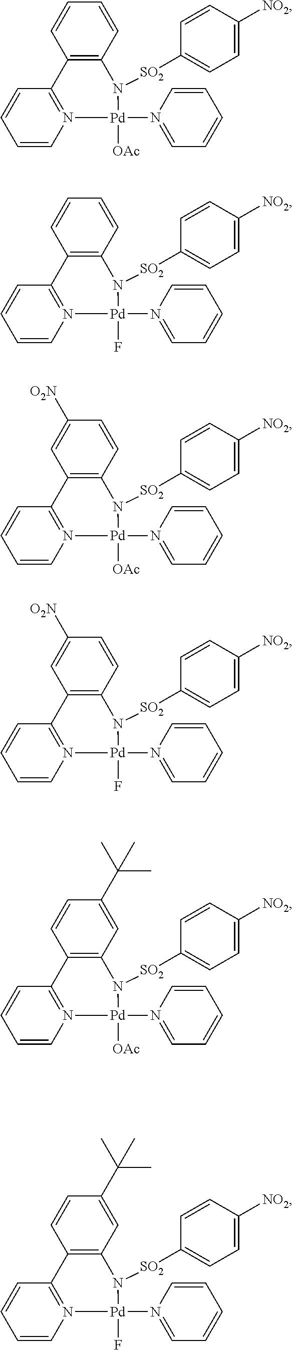

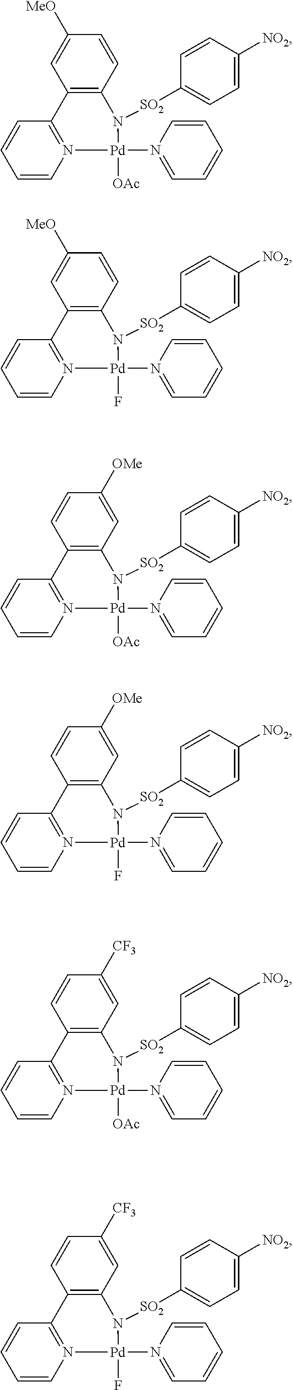

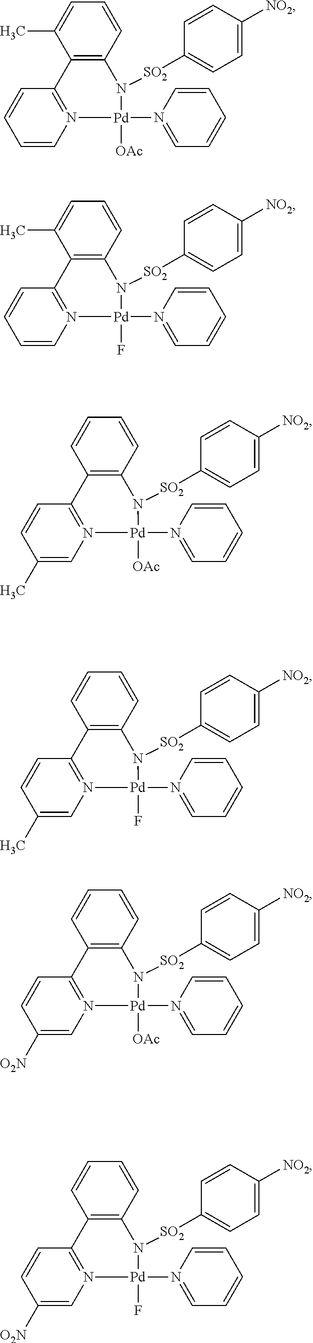

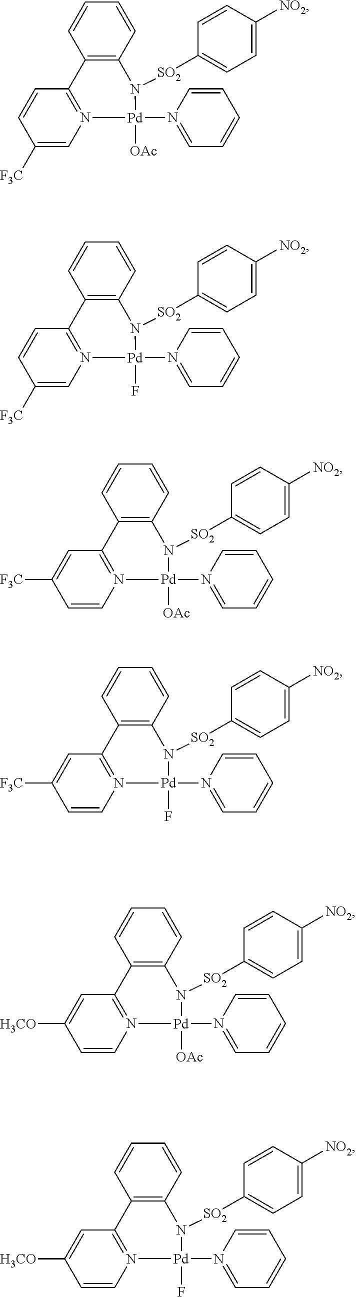

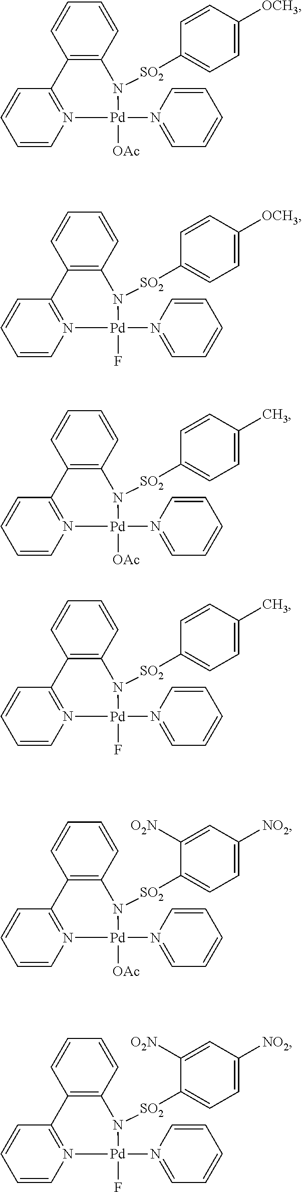

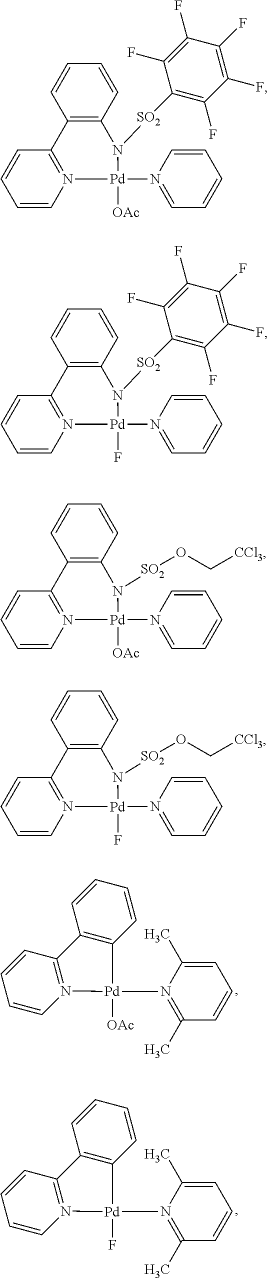

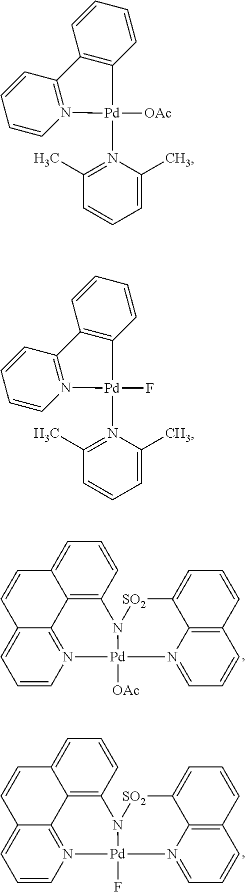

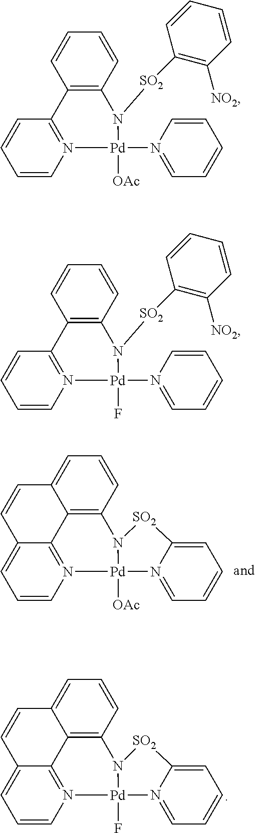

In some embodiments, the palladium complex is crystalline.

In one aspect, the invention features a method of fluorinating an organic compound, the method comprising mixing a palladium complex of formula (I), with a fluorinating agent and an organic compound, wherein the organic compound comprises a boron, organostannane or silane substituent, under conditions sufficient to fluorinate the organic compound, thereby providing a fluorinated organic compound.

In some embodiments, the organic compound comprises an aryl group.

In some embodiments, the organic compound comprises a boron substituent, e.g., a group of the formulae:

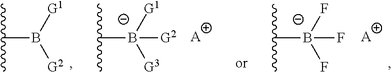

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

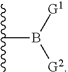

In some embodiments, G¹ and G² are both —OH.

In some embodiments, the method further comprises reacting a halogen-containing precursor of the organic compound with a boron-containing reagent to provide the organic compound comprising a boron substituent.

In some embodiments, the organic compound comprises an organostannane substituent, e.g., a trialkylstannane, e.g., trimethylstannane or tributylstannane. In some embodiments, the method further comprises reacting a precursor of the organostannane comprising a halogen substituent, with a tin-containing reagent to provide the organostannane. In some embodiments, the method further comprises reacting a precursor of the organostannane comprising a Grignard substituent, with a tin-containing reagent to provide the organostannane. In some embodiments, the method further comprises reacting a precursor of the organostannane comprising a trifluoromethanesulfonyl substituent, with a tin-containing reagent to provide the organostannane.

In some embodiments, the organic compound comprises a silane substituent. In some embodiments, the silane substituent has the formula —Si(OG⁴)₃. In some embodiments, G⁴ is an alkyl group, e.g., methyl or ethyl.

In some embodiments, the boron, organostannane or silane substituent is replaced by a fluorine substituent regiospecifically.

In some embodiments, the fluorinating agent comprises ¹⁸F or ¹⁹F. In some embodiments, the fluorinating agent provides a source of F⁺. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile. In some embodiments, the solvent is a mixture of methanol and benzene.

In some embodiments, the method further comprises a reagent.

In some embodiments, the reagent is a base, e.g., an inorganic base, e.g., K₂CO₃. In some embodiments, the method further comprises an inert atmosphere. In some embodiments, the reaction is performed under anhydrous conditions. In some embodiments, the reaction comprises a source of energy. In some embodiments, the reaction comprises heat.

In some embodiments, the palladium complex of formula (I) is combined with the organic compound comprising a boron, organostannane or silane substituent, prior to the addition of the fluorinating agent.

In some embodiments, the method proceeds via an intermediate palladium complex of formula (II):

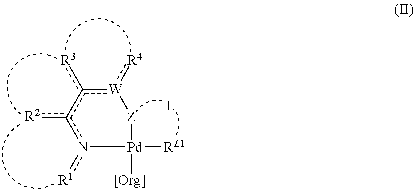

wherein:

Pd has a valency of +2;

the substituents R¹, R², R³, R⁴, W, Z, L and R^{L1} are as defined above; and

[Org] is an organic compound coordinated to Pd via a carbon atom.

In some embodiments, the intermediate palladium complex is isolated.

In some embodiments, the fluorinated organic compound is an imaging agent, e.g., a PET imaging agent or an MRI imaging agent. In some embodiments, the fluorinated organic compound may be used as a probe, e.g., a biological NMR probe. In some embodiments, the fluorinated organic compound is a pharmaceutically acceptable compound.

In one aspect, the invention features a method of making a palladium complex of formula (II), the method comprising mixing a palladium complex of formula (I) with an organic compound comprising a boron, organostannane or silane substituent, under conditions sufficient for transmetalation, to provide the palladium complex of formula (II).

In some embodiments, the organic compound comprises an aryl group.

In some embodiments, the organic compound comprises a boron substituent, e.g., a group of the formulae:

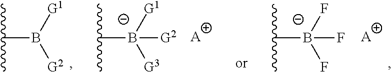

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

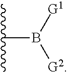

In some embodiments, G¹ and G² are both —OH.

In some embodiments, the method further comprises reacting a halogen-containing precursor of the organic compound with a boron-containing reagent to provide the organic compound comprising a boron substituent.

In some embodiments, the organic compound comprises an organostannane substituent. In some embodiments, the organostannane substituent is a trialkylstannane, e.g., a trimethylstannane or tributylstannane. In some embodiments, the method further comprises reacting a precursor of the organostannane comprising a halogen substituent, with a tin-containing reagent to provide the organostannane. In some embodiments, the method further comprises reacting a precursor of the organostannane comprising a Grignard substituent, with a tin-containing reagent to provide the organostannane. In some embodiments, the method further comprises reacting a precursor of the organostannane comprising a trifluoromethanesulfonyl substituent, with a tin-containing reagent to provide the organostannane.

In some embodiments, the organic compound comprises a silane substituent. In some embodiments, the silane substituent has the formula —Si(OG⁴)₃. In some embodiments, G⁴ is an alkyl group, e.g., methyl or ethyl.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile. In some embodiments, the solvent is a mixture of methanol and benzene.

In some embodiments, the method further comprises a reagent.

In some embodiments, the reagent is a base. In some embodiments, the base is an inorganic base, e.g., K₂CO₃.

In some embodiments, the method further comprises an inert atmosphere. In some embodiments, the reaction is performed under anhydrous conditions. In some embodiments, the reaction comprises a source of energy. In some embodiments, the reaction comprises heat.

In one aspect, the invention features a method of making a fluorinated Pd(IV) complex, the method comprising reacting a palladium complex of formula (I) with a fluorinating agent, to provide the fluorinated Pd(IV) complex.

In some embodiments, the fluorinating agent comprises ¹⁸F or ¹⁹F. In some embodiments, the fluorinating agent provides a source of F⁺. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile.

In some embodiments, the method further comprises an inert atmosphere. In some embodiments, the reaction is performed under anhydrous conditions. In some embodiments, the reaction comprises a source of energy. In some embodiments, the reaction comprises heat.

In one aspect, the invention features a method of storing a palladium complex of formula (I), the method comprising maintaining the palladium complex in a sealed container for at least about 12 hours.

In some embodiments, the sealed container is a vial. In some embodiments, the sealed container is an ampule. In some embodiments, the sealed container is substantially free of dioxygen. In some embodiments, the sealed container contains an inert gas.

In one aspect, the invention features a composition comprising a palladium complex of formula (I) and an additional component.

In some embodiments, the component is a reagent. In some embodiments, the reagent is a fluorinating agent. In some embodiments, the fluorinating agent comprises ¹⁸F or ¹⁹F. In some embodiments, the fluorinating agent provides a source of F⁺. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the reagent is an organic compound comprising an aryl group. In some embodiments, the reagent is an organic compound comprising a boron substituent. In some embodiments, the boron substituent is a group of the formulae:

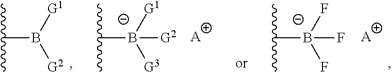

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

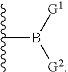

In some embodiments, G¹ and G² are both —OH.

In some embodiments, the reagent is an organic compound comprising an organostannane substituent. In some embodiments, the organostannane substituent is a trialkylstannane, e.g., trimethylstannane or tributylstannane. In some embodiments, the composition further comprises reacting a precursor of the organostannane comprising a halogen substituent, with a tin-containing reagent to provide the organostannane. In some embodiments, the composition further comprises reacting a precursor of the organostannane comprising a Grignard substituent, with a tin-containing reagent to provide the organostannane. In some embodiments, the composition further comprises reacting a precursor of the organostannane comprising a trifluoromethanesulfonyl substituent, with a tin-containing reagent to provide the organostannane.

In some embodiments, the reagent is an organic compound comprising a silane substituent. In some embodiments, the silane substituent has the formula —Si(OG⁴)₃. In some embodiments, G⁴ is an alkyl group, e.g., methyl or ethyl.

In some embodiments, the composition comprises a plurality of reagents.

In some embodiments, the component is a solvent. In some embodiments, the solvent is a polar aprotic solvent, e.g., acetonitrile or acetone. In some embodiments, the solvent comprises a mixture of solvents. In some embodiments, the solvent is a mixture of acetone and acetonitrile. In some embodiments, the solvent is a mixture of methanol and benzene.

In some embodiments, the component is a reagent. In some embodiments, the reagent is a base. In some embodiments, the base is an inorganic base, e.g., K₂CO₃.

In one aspect, the invention features a kit comprising a palladium complex of formula (I) and a container.

In some embodiments, the container is a vial. In some embodiments, the container is a sealed ampule. In some embodiments, the container is substantially free of dioxygen. In some embodiments, the container contains an inert gas. In some embodiments, the kit further comprises instructions for use of the palladium complex.

In some embodiments, the kit further comprises a reagent.

In some embodiments, the reagent is a fluorinating agent. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the reagent is an organ ic compound comprising an aryl group. In some embodiments, the reagent is an organic compound comprising a boron substituent. In some embodiments, the boron substituent is a group of the formulae:

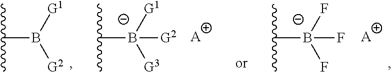

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

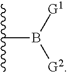

In some embodiments, G¹ and G² are both —OH.

In some embodiments, the reagent is an organic compound comprising an organostannane substituent. In some embodiments, the organostannane substituent is a trialkylstannane, e.g., trimethylstannane or tributylstannane.

In some embodiments, the reagent is an organic compound comprising a silane substituent. In some embodiments, the silane substituent has the formula —Si(OG⁴)₃. In some embodiments, G⁴ is an alkyl group, e.g., methyl or ethyl.

In one aspect, the invention features a palladium complex of formula (II),

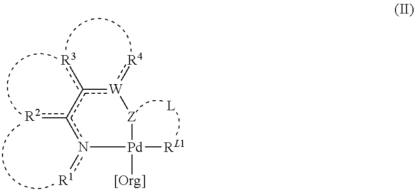

wherein:

Pd has a valency of +2;

[Org] is an organic compound coordinated to Pd via a carbon atom;

R^{L1} is an optionally substituted aliphatic, optionally substituted heteroaliphatic, optionally substituted aryl, optionally substituted heteroaryl, halogen, —OR^{a}, —SR^{b}, —N(R^{c})₂, —N(R^{c})₃, or —P(R^{x})₃;

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group [symbol] C(R^{c1}), wherein R^{c1} is an optionally substituted aliphatic, optionally substituted R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

wherein each instance of R^{x} is, independently, hydrogen, an optionally substituted aliphatic, optionally substituted heteroaliphatic, optionally substituted aryl, or optionally substituted heteroaryl group;

when W is —C— or —C(R^{d})— then:
(i) Z is a bond, —O—, —S—, —C(R^{d})₂—, —C(R^{d})═C(R^{d})—, —C(R^{d})═N—, or —N(R^{e})—;

or
(ii) Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring;

or

when W is —N— or —N(R^{e})— then Z is a bond, —C(R^{d})₂—, —C(R^{d})═C(R^{d})—, or —C(R^{d})═N—,

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

R² and R³ are optionally joined to form an optionally substituted 5- to 7-membered heteroaryl, aryl, heterocyclic or carbocyclic ring;

R³ and R⁴ are optionally joined to form an optionally substituted 5- to 7-membered heteroaryl, aryl, heterocyclic or carbocyclic ring,

wherein each curved dotted line [symbol] independently represents optional joining of an optionally substituted 5- to 7-membered ring, and

wherein [symbol] represents a single or double bond.

In some embodiments, the palladium complex is of the formula:

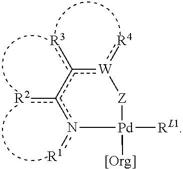

In some embodiments, the palladium complex is of the formula:

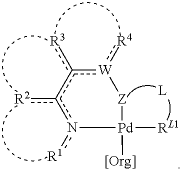

wherein Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring; and

wherein curved solid lines [symbol] represent joining of the 5- to 7-membered palladacycle.

In some embodiments, W is —C—. In some embodiments, Z is —N(R^{e})—. In some embodiments, R^{e} is —S(O)₂R^{e1}. In some embodiments, R^{e1} is optionally substituted aryl. In some embodiments, R^{e} is:

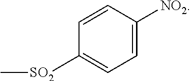

In some embodiments, R¹ and R² are joined to form an optionally substituted 6-membered heteroaryl ring. In some embodiments, R³ and R⁴ are joined to form an optionally substituted 6-membered aryl ring.

In some embodiments, R^{L1} comprises a 6-membered ring. In some embodiments, R^{L1} is —N(R^{c})₂. In some embodiments, the two R^{c} groups of —N(R^{c})₂ are joined to form an optionally substituted heteroaryl ring, e.g., pyridyl.

In some embodiments, Z is —N— joined via a linker group -L- to the group R^{L1} to form a 5- to 7-membered palladacycle, wherein -L- is —S(O)₂— and wherein-N(R)₂ is a group wherein two R^{c} groups are joined to form an optionally substituted heteroaryl ring. In some embodiments, Z, L and R^{L1} provide a group of the formulae:

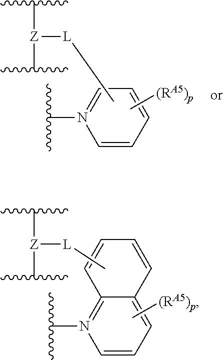

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is an integer between 0 to 5, inclusive.

In some embodiments, [Org] comprises an aryl group.

In some embodiments, the palladium complex is crystalline.

In one aspect, the invention features a method of fluorinating an organic compound, the method comprising mixing a palladium complex of formula (II), wherein [Org] is the organic compound to be fluorinated, with a fluorinating agent under conditions sufficient to fluorinate the organic compound, thereby providing a fluorinated organic compound.

In some embodiments, the organic compound comprises an aryl group.

In some embodiments, the organic compound is fluorinated regiospecifically.

In some embodiments, the fluorinating agent comprises ¹⁸F or ¹⁹F. In some embodiments, the fluorinating agent provides a source of F⁺. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile.

In some embodiments, the method further comprises an inert atmosphere. In some embodiments, the reaction is performed under anhydrous conditions. In some embodiments, the reaction comprises a source of energy. In some embodiments, the reaction comprises heat.

In some embodiments, the method proceeds via an intermediate palladium complex of formula (III):

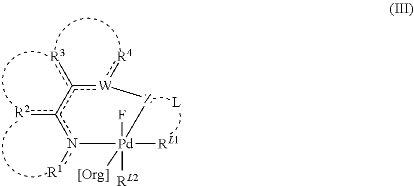

wherein:

Pd has a valency of +4;

the substituents R¹, R², R³, R⁴, W, Z, L and R^{L1} are as defined above; and

[Org] is an organic compound coordinated to Pd via a carbon atom.

In some embodiments, the intermediate palladium complex is isolated.

In some embodiments, the fluorinated organic compound is an imaging agent, e.g., a PET imaging agent or an MRI imaging agent. In some embodiments, the fluorinated organic compound may be used as a probe, e.g., a biological NMR probe. In some embodiments, the fluorinated organic compound is a pharmaceutically acceptable compound.

In one aspect, the invention features a method of making a fluorinated Pd(IV) complex, the method comprising reacting a palladium complex of formula (II) with a fluorinating agent to provide the fluorinated Pd(IV) complex.

In some embodiments, the fluorinating agent comprises ¹⁸F or ¹⁹F. In some embodiments, the fluorinating agent provides a source of F⁺. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile.

In some embodiments, the method further comprises an inert atmosphere. In some embodiments, the reaction is performed under anhydrous conditions. In some embodiments, the reaction comprises a source of energy. In some embodiments, the reaction comprises heat.

In one aspect, the invention features a method of storing a palladium complex of formula (II), the method comprising maintaining the palladium complex in a sealed container for at least about 12 hours.

In some embodiments, the sealed container is a vial. In some embodiments, the sealed container is an ampule. In some embodiments, the sealed container is substantially free of dioxygen. In some embodiments, the sealed container contains an inert gas.

In one aspect, the invention features a composition comprising a palladium complex of formula (II) and an additional component.

In some embodiments, the component is a reagent. In some embodiments, the reagent is a fluorinating agent. In some embodiments, the fluorinating agent comprises ¹⁸F or ¹⁹F. In some embodiments, the fluorinating agent provides a source of F⁺. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide

(e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the composition comprises a plurality of reagents.

In some embodiments, the component is a solvent. In some embodiments, the solvent is a polar aprotic solvent, e.g., acetonitrile or acetone. In some embodiments, the solvent comprises a mixture of solvents. In some embodiments, the solvent is a mixture of acetone and acetonitrile.

In one aspect, the invention features a kit comprising a palladium complex of formula (II) and a container.

In some embodiments, the container is a vial. In some embodiments, the container is a sealed ampule. In some embodiments, the container is substantially free of dioxygen. In some embodiments, the container contains an inert gas. In some embodiments, the kit further comprises instructions for use of the palladium complex.

In some embodiments, the kit further comprises a reagent. In some embodiments, the reagent is a fluorinating agent. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In one aspect, the invention features a palladium complex of formula (III),

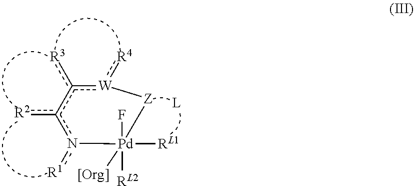

wherein:

Pd has a valency of +4;

[Org] is an organic compound coordinated to Pd via a carbon atom;

R^{L1} and R^{L2} are, independently, optionally substituted aliphatic, optionally substituted heteroaliphatic, optionally substituted aryl, optionally substituted heteroaryl, halogen, —OR^{a}, —SR^{b}, —N(R^{c})₂, —N(R^{c})₃, or —P(R^{x})₃;

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a2})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group [symbol] (R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

wherein each instance of R^{x} is, independently, hydrogen, an optionally substituted aliphatic, optionally substituted heteroaliphatic, optionally substituted aryl, or optionally substituted heteroaryl group;

when W is —C— or —C(R^{d})— then:
(i) Z is a bond, —O—, —S—, —C(R^{d})₂—, —C(R^{d})═C(R^{d})—, —C(R^{d})═N—, or —N(R^{e})—;

or
(ii) Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring;

or

when W is —N— or —N(R^{e})— then Z is a bond, —C(R^{d})₂—, —C(R^{d})═C(R^{d})—, or —C(R^{d})═N—,

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

R² and R³ are optionally joined to form an optionally substituted 5- to 7-membered heteroaryl, aryl, heterocyclic or carbocyclic ring;

R³ and R⁴ are optionally joined to form an optionally substituted 5- to 7-membered heteroaryl, aryl, heterocyclic or carbocyclic ring,

wherein each of the curved dotted lines [symbol] independently represents optional joining of an optionally substituted 5- to 7-membered ring, and

wherein [symbol] represents a single or double bond;

wherein at least one of R^{L1} and R^{L2} comprises a negatively charged moiety, or the complex further comprises a negatively charged counterion X⁻; and

F comprises ¹⁸F or ¹⁹F.

In some embodiments, the palladium complex is of the formula:

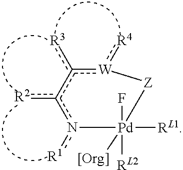

In some embodiments, the palladium complex is of the formula:

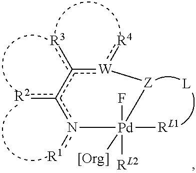

wherein Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring; and

wherein curved solid lines [symbol] represent joining of the 5- to 7-membered palladacycle.

In some embodiments, W is —C—. In some embodiments, Z is —N(R^{e})—. In some embodiments, R^{e} is —S(O)₂R^{e1}. In some embodiments, R^{e1} is optionally substituted aryl. In some embodiments, R^{e} is:

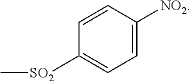

In some embodiments, R¹ and R² are joined to form an optionally substituted 6-membered heteroaryl ring. In some embodiments, R³ and R⁴ are joined to form an optionally substituted 6-membered aryl ring.

In some embodiments, R^{L1} comprises a 6-membered ring. In some embodiments, RL¹ is —N(R^{c})₂. In some embodiments, the two R^{c} groups of —N(R^{c})₂ are joined to form an optionally substituted heteroaryl ring, e.g., pyridyl. In some embodiments, R^{L2} is —N(R^{c})₂. In some embodiments, the two R^{c} groups of —N(R^{c})₂ are joined to form the group ≡≡C(R^{c1}). In some embodiments, R^{L2} is acetonitrile. In some embodiments, R^{L2} is —OR^{a}. In some embodiments, R^{L2} is acetate.

In some embodiments, Z is —N— joined via a linker group -L- to the group R^{L1} to form a 5- to 7-membered palladacycle, wherein -L- is —S(O)₂— and wherein —N(R)₂ is a group wherein two R^{c} groups are joined to form an optionally substituted heteroaryl. In some embodiments, Z, L and R^{L1} provide a group of the formulae:

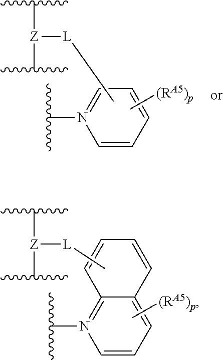

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is an integer between 0 to 5, inclusive.

In some embodiments, the palladium complex is crystalline.

In one aspect, the invention features a method of fluorinating an organic compound, the method comprising subjecting a complex of formula (III), wherein [Org] is the organic compound to be fluorinated, to conditions sufficient to cause reductive elimination, thereby fluorinating the organic compound to provide the fluorinated organic compound.

In some embodiments, the fluorinated organic compound comprises ¹⁸F or ¹⁹F. In some embodiments, the fluorinated organic compound comprises an aryl group.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile.

In some embodiments, the method further comprises an inert atmosphere. In some embodiments, the reaction is performed under anhydrous conditions. In some embodiments, the reaction comprises a source of energy. In some embodiments, the reaction comprises heat.

In some embodiments, the fluorinated organic compound is an imaging agent, e.g., a PET imaging agent or an MRI imaging agent. In some embodiments, the fluorinated organic compound may be used as a probe, e.g., a biological NMR probe. In some embodiments, the fluorinated organic compound is a pharmaceutically acceptable compound.

In one aspect, the invention features a method of storing a palladium complex of formula (III), the method comprising maintaining the palladium complex in a sealed container for at least 12 hours.

In some embodiments, the sealed container is a vial. In some embodiments, the sealed container is an ampule. In some embodiments, the sealed container is substantially free of dioxygen. In some embodiments, the sealed container contains an inert gas.

In one aspect, the invention features a composition comprising a palladium complex of formula (III) and an additional component.

In some embodiments, the component is a reagent. In some embodiments, the composition comprises a plurality of reagents. In some embodiments, the component is a solvent. In some embodiments, the solvent is a polar aprotic solvent, e.g., acetonitrile or acetone. In some embodiments, the solvent comprises a mixture of solvents. In some embodiments, the solvent is a mixture of acetone and acetonitrile.

In one aspect, the invention features a kit comprising a palladium complex of formula (III) and a container.

In some embodiments, the container is a vial. In some embodiments, the container is a sealed ampule. In some embodiments, the container is substantially free of dioxygen. In some embodiments, the container contains an inert gas. In some embodiments, the kit further comprises instructions for use of the palladium complex. In some embodiments, the kit further comprises a reagent.

In one aspect, the invention features a palladium complex of formula (IV),

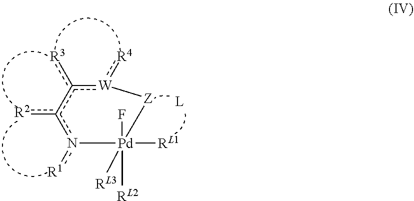

wherein:

Pd has a valency of +4;

F comprises ¹⁸F or ¹⁹F;

R^{L1}, R^{L2} and R^{L3} are, independently, optionally substituted aliphatic, optionally substituted heteroaliphatic, optionally substituted aryl, optionally substituted heteroaryl, halogen, —OR^{a}, —SR^{b}, —N(R^{c})₂, —N(R^{c})₃, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group [symbol] C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

wherein each instance of R^{x} is, independently, hydrogen, an optionally substituted aliphatic, optionally substituted heteroaliphatic, optionally substituted aryl, or optionally substituted heteroaryl group;

when W is —C— or —C(R^{d})— then:
(i) Z is a bond, —O—, —S—, —C(R^{d})₂—, —C(R^{d})═C(R^{d})—, —C(R^{d})═N—, or —N(R^{e})—;

or
(ii) Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring;

or

when W is —N— or —N(R^{e})— then Z is a bond, —C(R^{d})₂—, —C(R^{d})═C(R^{d})—, or —C(R^{d})═N—,

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

R² and R³ are optionally joined to form an optionally substituted 5- to 7-membered heteroaryl, aryl, heterocyclic or carbocyclic ring;

R³ and R⁴ are optionally joined to form an optionally substituted 5- to 7-membered heteroaryl, aryl, heterocyclic or carbocyclic ring,

wherein each of the curved dotted lines [symbol] independently represents optional joining of an optionally substituted 5- to 7-membered ring;

wherein [symbol] represents a single or double bond; and

wherein at least two of R^{L1}, R^{L2} and R^{L3} comprise a negatively charged moieties, or the complex further comprises a one or more negatively charged counterions X⁻.

In some embodiments, the palladium complex is of the formula:

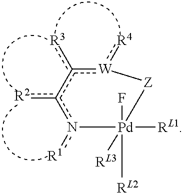

In some embodiments, the palladium complex is of the formula:

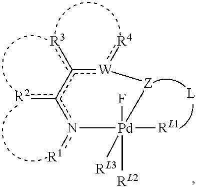

wherein Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring; and

wherein curved solid lines [symbol] represent joining of the 5- to 7-membered palladacycle.

In some embodiments, W is —C—. In some embodiments, Z is —N(R^{e})—. In some embodiments, R^{e} is —S(O)₂R^{e1}. In some embodiments, R^{e1} is optionally substituted aryl. In some embodiments, R^{e} is:

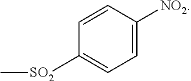

In some embodiments, R¹ and R² are joined to form an optionally substituted 6-membered heteroaryl ring. In some embodiments, R³ and R⁴ are joined to form an optionally substituted 6-membered aryl ring.

In some embodiments, R^{L1} comprises a 6-membered ring. In some embodiments, R^{L1} is —N(R^{c})₂. In some embodiments, the two R^{c} groups of —N(R^{c})₂ are joined to form an optionally substituted heteroaryl ring, e.g., pyridyl.

In some embodiments, R^{L2} is —N(R^{c})₂. In some embodiments, the two R^{c} groups of —N(R^{c})₂ are joined to form the group ≡≡C(R^{c1}). In some embodiments, R^{L2} is acetonitrile. In some embodiments, R^{L2} is —OR^{a}. In some embodiments, R^{L2} is acetate.

In some embodiments, R^{L3} is —N(R^{c})₂. In some embodiments, the two R^{c} groups of —N(R^{c})₂ are joined to form the group ≡≡C(R^{c1}). In some embodiments, R^{L3} is acetonitrile. In some embodiments, the two R^{c} groups of —N(R^{c})₂ are joined to form an optionally substituted heteroaryl ring, e.g., pyridyl. In some embodiments, R^{L3} is halogen, e.g., fluorine. In some embodiments, R^{L3} is —P(R^{x})₃. In some embodiments, R^{L3} is optionally substituted heteroaryl. In some embodiments, R^{L3} is an N-heterocyclic carbene.

In some embodiments, Z is —N— joined via a linker group -L- to the group R^{L1} to form a 5- to 7-membered palladacycle, wherein -L- is —S(O)₂— and wherein-N(R)₂ is a group wherein two R^{c} groups are joined to form an optionally substituted heteroaryl ring. In some embodiments, Z, L and R^{L1} provide a group of the formulae:

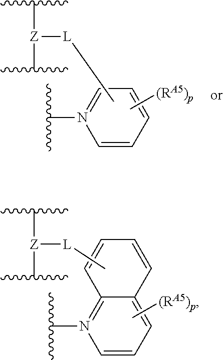

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is an integer between 0 to 5, inclusive.

In some embodiments, the palladium complex is crystalline.

In one aspect, the invention features a method of fluorinating an organic compound, the method comprising mixing a palladium complex of formula (IV), with an organic compound, wherein the organic compound comprises a boron, organostannane or silane substituent, under conditions sufficient to fluorinate the organic compound, thereby providing a fluorinated organic compound.

In some embodiments, the fluorinated organic compound comprises ¹⁸F or ¹⁹F. In some embodiments, the organic compound comprises an aryl group.

In some embodiments, the organic compound comprises a boron substituent. In some embodiments, the boron substituent is a group of the formulae:

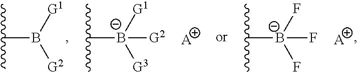

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

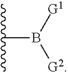

In some embodiments, G¹ and G² are both —OH.

In some embodiments, the method further comprises reacting a halogen-containing precursor of the organic compound with a boron-containing reagent to provide the organic compound comprising a boron substituent.

In some embodiments, the organic compound comprises an organostannane substituent. In some embodiments, the organostannane substituent is a trialkylstannane, e.g, trimethylstannane or tributylstannane. In some embodiments, the method further comprises reacting a precursor of the organostannane comprising a halogen substituent, with a tin-containing reagent to provide the organostannane. In some embodiments, the method further comprises reacting a precursor of the organostannane comprising a Grignard substituent, with a tin-containing reagent to provide the organostannane. In some embodiments, the method further comprises reacting a precursor of the organostannane comprising a trifluoromethanesulfonyl substituent, with a tin-containing reagent to provide the organostannane.

In some embodiments, the organic compound comprises a silane substituent. In some embodiments, the silane substituent has the formula —Si(OG⁴)₃. In some embodiments, G⁴ is an alkyl group, e.g., methyl or ethyl.

In some embodiments, the boron, organostannane or silane substituent is replaced by a fluorine substituent regiospecifically.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile. In some embodiments, the solvent is a mixture of methanol and benzene.

In some embodiments, the method further comprises a reagent.

In some embodiments, the reagent is a base. In some embodiments, the base is an inorganic base, e.g., K₂CO₃.

In some embodiments, the method further comprises an inert atmosphere. In some embodiments, the reaction is performed under anhydrous conditions. In some embodiments, the reaction comprises a source of energy. In some embodiments, the reaction comprises heat.

In some embodiments, the fluorinated organic compound is an imaging agent, e.g., a PET imaging agent or an MRI imaging agent. In some embodiments, the fluorinated organic compound may be used as a probe, e.g., a biological NMR probe. In some embodiments, the fluorinated organic compound is a pharmaceutically acceptable compound.

In one aspect, the invention features a method of storing a palladium complex of formula (IV), the method comprising maintaining the palladium complex in a sealed container for at least about 12 hours.

In some embodiments, the sealed container is a vial. In some embodiments, the sealed container is an ampule. In some embodiments, the sealed container is substantially free of dioxygen. In some embodiments, the sealed container contains an inert gas.

In one aspect, the invention features a composition comprising a palladium complex of formula (IV) and an additional component.

In some embodiments, the component is a reagent.

In some embodiments, the reagent is an organic compound comprising an aryl group.

In some embodiments, the reagent is an organic compound comprising a boron substituent. In some embodiments, the boron substituent is a group of the formulae:

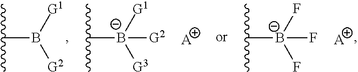

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

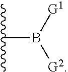

In some embodiments, G¹ and G² are both —OH.

In some embodiments, the reagent is an organic compound comprising an organostannane substituent. In some embodiments, the organostannane substituent is a trialkylstannane, e.g., trimethylstannane or tributylstannane. In some embodiments, the composition further comprises reacting a precursor of the organostannane comprising a halogen substituent, with a tin-containing reagent to provide the organostannane. In some embodiments, the composition further comprises reacting a precursor of the organostannane comprising a Grignard substituent, with a tin-containing reagent to provide the organostannane. In some embodiments, the composition further comprises reacting a precursor of the organostannane comprising a trifluoromethanesulfonyl substituent, with a tin-containing reagent to provide the organostannane.

In some embodiments, the reagent is an organic compound comprising a silane substituent. In some embodiments, the silane substituent has the formula —Si(OG⁴)₃. In some embodiments, G⁴ is an alkyl group, e.g., methyl or ethyl.

In some embodiments, the composition comprises a plurality of reagents.

In some embodiments, the component is a solvent. In some embodiments, the solvent is a polar aprotic solvent, e.g., acetonitrile or acetone. In some embodiments, the solvent comprises a mixture of solvents. In some embodiments, the solvent is a mixture of acetone and acetonitrile. In some embodiments, the solvent is a mixture of methanol and benzene.

In some embodiments, the reagent is a base. In some embodiments, the base is an inorganic base, e.g., K₂CO₃.

In one aspect, the invention features a kit comprising a palladium complex of formula (IV) and a container.

In some embodiments, the container is a vial. In some embodiments, the container is a sealed ampule. In some embodiments, the container is substantially free of dioxygen. In some embodiments, the container contains an inert gas.

In some embodiments, the kit further comprises instructions for use of the palladium complex.

In some embodiments, the kit further comprises a reagent.

In some embodiments, the reagent is an organic compound comprising an aryl group.

In some embodiments, the reagent is an organic compound comprising a boron substituent. In some embodiments, the boron substituent is a group of the formulae:

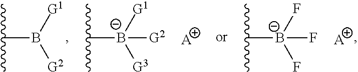

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

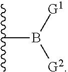

In some embodiments, G¹ and G² are both —OH.

In some embodiments, the reagent is an organic compound comprising an organostannane substituent. In some embodiments, the organostannane substituent is a trialkylstannane, e.g., trimethylstannane or tributylstannane.

In some embodiments, the reagent is an organic compound comprising a silane substituent. In some embodiments, the silane substituent has the formula —Si(OG⁴)₃. In some embodiments, G⁴ is an alkyl group, e.g., methyl or ethyl.

In one aspect, the invention features a method of fluorinating an organic compound, the method comprising mixing a palladium(II) complex with a fluorinating agent and an organic compound, wherein the organic compound comprises a boron, organostannane or silane substituent, under conditions sufficient to fluorinate the organic compound, thereby providing a fluorinated organic compound.

In some embodiments, the organic compound comprises an aryl group.

In some embodiments, the organic compound comprises a boron substituent. In some embodiments, the boron substituent is a group of the formulae:

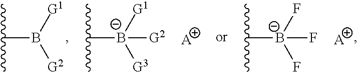

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

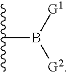

In some embodiments, G¹ and G² are both —OH.

In some embodiments, the method further comprises reacting a halogen-containing precursor of the organic compound with a boron-containing reagent to provide the organic compound comprising a boron substituent.

In some embodiments, the organic compound comprises an organostannane substituent. In some embodiments, the organostannane substituent is a trialkylstannane, e.g., trimethylstannane or tributylstannane. In some embodiments, the method further comprises reacting a precursor of the organostannane comprising a halogen substituent, with a tin-containing reagent to provide the organostannane. In some embodiments, the method further comprises reacting a precursor of the organostannane comprising a Grignard substituent, with a tin-containing reagent to provide the organostannane. In some embodiments, the method further comprises reacting a precursor of the organostannane comprising a trifluoromethanesulfonyl substituent, with a tin-containing reagent to provide the organostannane.

In some embodiments, the organic compound comprises a silane substituent. In some embodiments, the silane substituent has the formula —Si(OG⁴)₃. In some embodiments, G⁴ is an alkyl group, e.g., methyl or ethyl.

In some embodiments, the boron, organostannane or silane substituent is replaced by a fluorine substituent regiospecifically.

In some embodiments, the fluorinating agent comprises ¹⁸F or ¹⁹F. In some embodiments, the fluorinating agent provides a source of F⁺. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile. In some embodiments, the solvent is a mixture of methanol and benzene.

In some embodiments, the method further comprises a reagent.

In some embodiments, the reagent is a base. In some embodiments, the base is an inorganic base, e.g., K₂CO₃.

In some embodiments, the method further comprises an inert atmosphere. In some embodiments, the reaction is performed under anhydrous conditions. In some embodiments, the reaction comprises a source of energy. In some embodiments, the reaction comprises heat.

In some embodiments, the palladium complex is combined with the organic compound comprising a boron, organostannane or silane substituent, prior to the addition of the fluorinating agent.

In some embodiments, the method proceeds via an intermediate palladium complex. In some embodiments, the intermediate palladium complex is isolated.

In some embodiments, the fluorinated organic compound is an imaging agent, e.g., a PET imaging agent or an MRI imaging agent. In some embodiments, the fluorinated organic compound may be used as a probe, e.g., a biological NMR probe. In some embodiments, the fluorinated organic compound is a pharmaceutically acceptable compound.

In one aspect, the invention features a method of fluorinating an organic compound, the method comprising mixing a organopalladium(II) complex, wherein the organic ligand bound to palladium(II) is the organic compound to be fluorinated, with a fluorinating agent under conditions sufficient to fluorinate the organic compound, thereby providing a fluorinated organic compound.

In some embodiments, the organic compound comprises an aryl group.

In some embodiments, the organic compound is fluorinated regiospecifically.

In some embodiments, the fluorinating agent comprises ¹⁸F or ¹⁹F. In some embodiments, the fluorinating agent provides a source of F⁺. In some embodiments, the fluorinating agent is selected from the group consisting of N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In some embodiments, the fluorinating agent is N-chloromethyl-N′-fluorotriethylenediammonium bis(tetrafluoroborate) (SELECTFLUOR®). In some embodiments, the fluorinating agent is XeF₂.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile. In some embodiments, the solvent is a mixture of methanol and benzene.

In some embodiments, the method further comprises a reagent.

In some embodiments, the reagent is a base. In some embodiments, the base is an inorganic base, e.g., K₂CO₃.

In some embodiments, the method further comprises an inert atmosphere. In some embodiments, the reaction is performed under anhydrous conditions. In some embodiments, the reaction comprises a source of energy. In some embodiments, the reaction comprises heat.

In some embodiments, the fluorinated organic compound is an imaging agent, e.g., a PET imaging agent or an MRI imaging agent. In some embodiments, the fluorinated organic compound may be used as a probe, e.g., a biological NMR probe. In some embodiments, the fluorinated organic compound is a pharmaceutically acceptable compound.

In one aspect, the invention features a method of making a fluorinated organic compound, the method comprising subjecting a an organopalladium(IV) fluoride complex, wherein the organic ligand bound to palladium(IV) is the organic compound to be fluorinated, to conditions sufficient to cause reductive elimination, thereby providing a fluorinated organic compound.

In some embodiments, the organic ligand bound to palladium(IV) comprises an aryl group.

In some embodiments, the organic compound is fluorinated regiospecifically.

In some embodiments, the organopalladium(IV) fluoride complex comprises ¹⁸F or ¹⁹F.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile.

In some embodiments, the method further comprises an inert atmosphere. In some embodiments, the reaction is performed under anhydrous conditions. In some embodiments, the reaction comprises a source of energy. In some embodiments, the reaction comprises heat.

In some embodiments, the fluorinated organic compound is an imaging agent, e.g., a PET imaging agent or an MRI imaging agent. In some embodiments, the fluorinated organic compound may be used as a probe, e.g., a biological NMR probe. In some embodiments, the fluorinated organic compound is a pharmaceutically acceptable compound.

In one aspect, the invention features a method of fluorinating an organic compound, the method comprising mixing a palladium(IV) fluoride complex with an organic compound comprising a boron, organostannane or silane substituent, under conditions sufficient to fluorinate the organic compound, thereby providing a fluorinated organic compound.

In some embodiments, the palladium(IV) fluoride complex comprises ¹⁸F or ¹⁹F.

In some embodiments, the organic compound comprises an aryl group.

In some embodiments, the organic compound comprises a boron substituent. In some embodiments, the boron substituent is a group of the formulae:

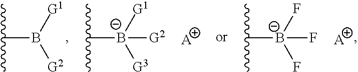

wherein G¹, G² and G³ are, independently, —OH, —OR^{G}, or —R^{G};

each R^{G} is, independently, optionally substituted aliphatic, optionally substituted heteroaliphatic, optionally substituted aryl, or optionally substituted heteroaryl,

or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and

wherein A^{⊕} is a metal cation or ammonium.

In some embodiments, the boron substituent is a group of the formula:

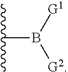

In some embodiments, G¹ and G² are both —OH.

In some embodiments, the method further comprises reacting a halogen-containing precursor of the organic compound with a boron-containing reagent to provide the organic compound comprising a boron substituent.

In some embodiments, the organic compound comprises an organostannane substituent. In some embodiments, the organostannane substituent is a trialkylstannane, e.g., trimethylstannane or tributylstannane. In some embodiments, the method further comprises reacting a precursor of the organostannane comprising a halogen substituent, with a tin-containing reagent to provide the organostannane. In some embodiments, the method further comprises reacting a precursor of the organostannane comprising a Grignard substituent, with a tin-containing reagent to provide the organostannane. In some embodiments, the method further comprises reacting a precursor of the organostannane comprising a trifluoromethanesulfonyl substituent, with a tin-containing reagent to provide the organostannane.

In some embodiments, the organic compound comprises a silane substituent. In some embodiments, the silane substituent has the formula —Si(OG⁴)₃. In some embodiments, G⁴ is an alkyl group, e.g., methyl or ethyl.

In some embodiments, the boron, organostannane or silane substituent is replaced by a fluorine substituent regiospecifically.

In some embodiments, the method further comprises a solvent.

In some embodiments, the solvent is a polar aprotic solvent, e.g., acetonitrile or acetone. In some embodiments, the solvent comprises a mixture of solvents. In some embodiments, the solvent is a mixture of acetone and acetonitrile. In some embodiments, the solvent is a mixture of methanol and benzene.

In some embodiments, the method further comprises a reagent.

In some embodiments, the reagent is a base. In some embodiments, the base is an inorganic base, e.g., K₂CO₃.

In some embodiments, the method further comprises an inert atmosphere. In some embodiments, the reaction is performed under anhydrous conditions. In some embodiments, the reaction comprises a source of energy. In some embodiments, the reaction comprises heat.

In some embodiments, the fluorinated organic compound is an imaging agent, e.g., a PET imaging agent or an MRI imaging agent. In some embodiments, the fluorinated organic compound may be used as a probe, e.g., a biological NMR probe. In some embodiments, the fluorinated organic compound is a pharmaceutically acceptable compound.

In one aspect, the invention features a palladium complex described herein (e.g., a palladium complex of formula (I), (II), (III), or (IV)), wherein the complex is attached to a solid support.

In one aspect, a compound described herein may be prepared by a method described herein; exemplary methods include those methods using a Pd complex and methods using electrophilic fluorination of a lithium-containing precursor.

### BRIEF DESCRIPTION OF THE DRAWINGS

FIGS. 1A-1E. FIG. 1A: ORTEP diagram of (Acetato){benzo[h]quinolin-10-yl(4-nitrophenylsulfonyl)amide}(pyridine) palladium(II) at 193 Kelvin (complex 1). The X-ray crystal structure of complex 1 with hydrogens and with the atom labeling scheme employed. The non-hydrogen atoms are depicted with 50% probability ellipsoids. FIG. 1B: A unit cell diagram for complex 1 viewed down the crystallographic a-axis. The non-hydrogen atoms are depicted with 50% probability ellipsoids. FIG. 1C: A unit cell diagram for complex 1 viewed down the crystallographic b-axis. The non-hydrogen atoms are depicted with 50% probability ellipsoids. FIG. 1D: A unit cell diagram for complex 1 viewed down the crystallographic c-axis. The non-hydrogen atoms are depicted with 50% probability ellipsoids. FIG. 1E: Photograph of complex 1 crystal loaded to a loop.

FIGS. 2A-2E. FIG. 2A: ORTEP diagram of (Phenyl){benzo[h]quinolin-10-yl(4-nitrophenylsulfonyl)amide}(pyridine) palladium(II) at 193 K (complex 4a). The x-ray structure of complex 4a with hydrogens and with the atom labeling scheme employed. The non-hydrogen atoms are depicted with 50% probability ellipsoids. FIG. 2B: A unit cell diagram for complex 4a viewed down the crystallographic a-axis. The non-hydrogen atoms are depicted with 50% probability ellipsoids. FIG. 2C: A unit cell diagram for complex 4a viewed down the crystallographic b-axis. The non-hydrogen atoms are depicted with 50% probability ellipsoids. FIG. 2D: A unit cell diagram for complex 4a viewed down the crystallographic c-axis. The non-hydrogen atoms are depicted with 50% probability ellipsoids. FIG. 2E: Photograph of complex 4a crystal loaded to a loop.

FIG. 3. ORTEP drawing of the palladium(IV)difluoride 11 with 50% probability ellipsoids (hydrogen atoms and solvent omitted for clarity). Selected bond lengths [Å] and angles [°]: Pd—F(1) 2.040 (3), Pd—F(2) 1.955 (3), Pd—C(35) 2.008 (5), Pd—N(13) 2.019 (4), Pd—N(1) 2.027 (5), Pd—N(26) 2.012 (5), F(1)-Pd—F(2) 88.27 (13), F(2)-Pd—N(13) 173.48 (15).

FIG. 4. The structure of the difluoro palladium(IV) complex 11 with hydrogens and with selected atom labels. The nonhydrogen atoms are depicted with 50% probability ellipsoids.

FIG. 5. A unit cell diagram for the difluoro palladium(IV) complex 11 viewed down the crystallographic a-axis. Hydrogen atoms have been removed for clarity.

FIG. 6. A unit cell diagram for the difluoro palladium(IV) complex 11 viewed down the crystallographic b-axis. Hydrogen atoms have been removed for clarity.

FIG. 7. A unit cell diagram for the difluoro palladium(IV) complex 11 viewed down the crystallographic c-axis. Hydrogen atoms have been removed for clarity.

FIG. 8. Photograph of a crystal of difluoro palladium(IV) complex 11 loaded on a loop.

FIG. 9. Another view of a crystal of difluoro palladium(IV) complex 11 loaded on a loop.

FIG. 10. The structure of the palladium(II) fluoride complex 13 with cocrystallized dichloromethane solvent molecule, with hydrogens and with selected atom labels. The nonhydrogen atoms are depicted with 50% probability ellipsoids.

FIG. 11. Photograph of a crystal of the palladium(II) fluoride complex 13 loaded on a loop.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds of the present invention can comprise one or more asymmetric centers, and thus can exist in various isomeric forms, e.g., stereoisomers and/or diastereomers. Thus, compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

As used herein a “bond” refers to a single bond.

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-10 carbon atoms. In certain embodiments, aliphatic groups contain 1-8 carbon atoms, 1-7 carbon atoms, 1-6 carbon atoms, 1-5 carbon atoms, 1-4 carbon atoms, 1-3 carbon atoms, or 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “unsaturated”, as used herein, means that a moiety has one or more double or triple bonds.

The terms “carbocyclyl” and “carbocyclic” refer to a saturated or partially unsaturated cyclic aliphatic monocyclic or bicyclic ring systems, as described herein, having from 3 to 10 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In certain embodiments, the cycloalkyl has 3-6 carbons. The terms “cycloaliphatic”, “carbocycle” or “carbocyclic” also include aliphatic rings that are fused to one or more aromatic or nonaromatic rings, such as decahydronaphthyl or tetrahydronaphthyl, where the radical or point of attachment is on the aliphatic ring.

The term “alkyl,” as used herein, refers to saturated, straight- or branched-chain hydrocarbon radicals derived from an aliphatic moiety containing between one and six carbon atoms by removal of a single hydrogen atom. In certain embodiments, the alkyl group employed in the invention contains 1-10 carbon atoms. In certain embodiments, the alkyl group employed contains 1-8 carbon atoms, 1-7 carbon atoms, 1-6 carbon atoms, 1-5 carbon atoms, 1-4 carbon atoms, 1-3 carbon atoms, or 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. In certain embodiments, the alkenyl group employed in the invention contains 2-10 carbon atoms. In certain embodiments, the alkenyl group employed in the invention contains 2-8 carbon atoms, 2-7 carbon atoms, 2-6 carbon atoms, 2-5 carbon atoms, 2-4 carbon atoms, 2-3 carbon atoms or 2 carbon atoms. Alkenyl groups include, for example, ethenyl, propenyl, butenyl, 1-methyl-2-buten-1-yl, and the like.

The term “alkynyl,” as used herein, refers to a monovalent group derived from a straight- or branched-chain aliphatic moiety having at least one carbon-carbon triple bond by the removal of a single hydrogen atom. In certain embodiments, the alkynyl group employed in the invention contains 2-10 carbon atoms. In certain embodiments, the alkynyl group employed in the invention contains 2-8 carbon atoms, 2-7 carbon atoms, 2-6 carbon atoms, 2-5 carbon atoms, 2-4 carbon atoms, 2-3 carbon atoms or 2 carbon atoms. Representative alkynyl groups include, but are not limited to, ethynyl, 2-propynyl (propargyl), 1-propynyl, and the like.

The term “aryl” refers to monocyclic, bicyclic or tricyclic aromatic ring system having a total of five to 14 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl, phenanthrenyl, phenalenyl, and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenantriidinyl, or tetrahydronaphthyl, and the like.

The term “heteroaryl” refers to a monocyclic, bicyclic or tricyclic aromatic ring system having 5 to 14 ring atoms, wherein the ring atoms include carbon atoms and from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring” any of which terms include rings that are optionally substituted.

As used herein, the terms “heterocyclyl” and “heterocyclic ring” are used interchangeably and refer to a monocyclic, bicyclic or tricyclic nonaromatic ring system that is either saturated or partially unsaturated, and having, in addition to carbon atoms, one to five heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, and “heterocyclyl ring”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R′; —(CH₂)₀₋₄OR′; —O—(CH₂)₀₋₄C(O)OR′; —(CH₂)₀₋₄CH(OR′)₂; —(CH₂)₀₋₄SR′; —(CH₂)₀₋₄Ph, which may be substituted with R′; —(CH₂)₀₋₄O(CH₂)₀₋₁Ph which may be substituted with R′; —CH═CHPh, which may be substituted with R′; —NO₂; —CN; —N₃; —(CH₂)₀₋₄N(R′)₂; —(CH₂)₀₋₄N(R′)C(O)R′; —N(R′)C(S)R′; —(CH₂)₀₋₄N(R′)C(O)NR′₂; —N(R′)C(S)NR′₂; —(CH₂)₀₋₄N(R′)C(O)OR′; —N(R′)N(R′)C(O)R′; —N(R′)N(R′)C(O)NR′₂; —N(R′)N(R′)C(O)OR′; —(CH₂)₀₋₄C(O)R^{o}; —C(S)R^{o}; —(CH₂)₀₋₄C(O)OR′; —(CH₂)₀₋₄C(O)SR′; —(CH₂)₀₋₄C(O)OSiR′₃; —(CH₂)₀₋₄OC(O)R′; —OC(O)(CH₂)₀₋₄SR—, SC(S)SR′; —(CH₂)₀₋₄SC(O)R′; —(CH₂)₀₋₄C(O)NR′₂; —C(S)NR′₂; —C(S)SR′; —SC(S)SR′, —(CH₂)₀₋₄OC(O)NR′₂; —C(O)N(OR′)R′; —C(O)C(O)R′; —C(O)CH₂C(O)R′; —C(NOR′)R′; —(CH₂)₀₋₄SSR′; —(CH₂)₀₋₄S(O)₂R′; —(CH₂)₀₋₄S(O)₂OR′; —(CH₂)₀₋₄OS(O)₂R′; —S(O)₂NR′₂; —(CH₂)₀₋₄S(O)R′; —N(R′)S(O)₂NR′₂; —N(R′)S(O)₂R′; —N(OR′)R′; —C(NH)NR′₂; —P(O)₂R′; —P(O)R′₂; —OP(O)R′₂; —OP(O)(OR′)₂; SiR′₃; —(C₁₋₄ straight or branched alkylene)O—N(R′)₂; or —(C₁₋₄ straight or branched alkylene)C(O)O—N(R′)₂, wherein each R′ may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R′, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R′ (or the ring formed by taking two independent occurrences of R′ together with their intervening atoms), are independently halogen, —(CH₂)₀₋₂R″, -(haloR″), —(CH₂)₀₋₂OH, —(CH₂)₀₋₂OR″, —(CH₂)₀₋₂CH(OR″)₂; —O(haloR″), —CN, —N₃, —(CH₂)₀₋₂C(O)R″, —(CH₂)₀₋₂C(O)OH, —(CH₂)₀₋₂C(O)OR″, —(CH₂)₀₋₂SR″, —(CH₂)₀₋₂SH, —(CH₂)₀₋₂NH₂, —(CH₂)₀₋₂NHR″, —(CH₂)₀₋₂NR″₂, —NO₂, —SiR″₃, —OSiR″₃, —C(O)SR″, —(C₁₋₄ straight or branched alkylene)C(O)OR″, or —SSR″ wherein each R″ is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R′ include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R″, -(haloR″), —OH, —OR″, —O(haloR″), —CN, —C(O)OH, —C(O)OR″, —NH₂, —NHR″, —NR′₂, or —NO₂, wherein each R″ is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R″, -(haloR″), —OH, —OR″, —O(haloR″), —CN, —C(O)OH, —C(O)OR″, —NH₂, —NHR″, —NR″₂, or —NO₂, wherein each R″ is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

A “suitable amino-protecting group,” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino-protecting groups include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

A “suitable hydroxyl protecting group” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxyl protecting groups include methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, a-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

A “pharmaceutically acceptable form thereof” includes any pharmaceutically acceptable salts, isomers, and/or polymorphs of a palladium complex, or any pharmaceutically acceptable salts, prodrugs and/or isomers of an organic compound, as described below and herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “prodrug” refers to a derivative of a parent compound that requires transformation within the body in order to release the parent compound. In certain cases, a prodrug has improved physical and/or delivery properties over the parent compound. Prodrugs are typically designed to enhance pharmaceutically and/or pharmacokinetically based properties associated with the parent compound. The advantage of a prodrug can lie in its physical properties, such as enhanced water solubility for parenteral administration at physiological pH compared to the parent compound, or it enhances absorption from the digestive tract, or it may enhance drug stability for long-term storage. The compounds of the invention readily undergo dehydration to form oligomeric anhydrides, for example, by dehydration of the boronic acid moiety to form dimers, trimers, and tetramers, and mixtures thereof. These oligomeric species hydrolyze under physiological conditions to reform the boronic acid. As such, the oligomeric anhydrides are contemplated as a “prodrug” of the compounds of the present invention, and may be used in the treatment of disorder and/or conditions a wherein the inhibition of FAAH provides a therapeutic effect.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

As used herein, “polymorph” refers to a crystalline complex or compound existing in more than one crystalline form/structure. When polymorphism exists as a result of difference in crystal packing it is called packing polymorphism. Polymorphism can also result from the existence of different conformers of the same molecule in conformational polymorphism. In pseudopolymorphism the different crystal types are the result of hydration or solvation.

As used herein “palladacycle” is a 5- to 7-membered ring comprising a palladium(II) atom as a ring member.

As used herein “coordinated” means the organic compound is covalently attached to palladium.

As used herein, “inert gas” refers to a gas that does not chemically react with the compounds, compositions or reaction mixtures described herein. Examples of inert gases are nitrogen (N₂), helium, and argon. As used herein, an “inert atmosphere” refers to an atmosphere composed primarily of an inert gas.

### DETAILED DESCRIPTION OF CERTAIN EMBODIMENTS OF THE INVENTION

The present invention provides a method for fluorinating an organic compound.

Described herein are palladium complexes, compositions, reaction mixtures and kits. Also described herein are methods for fluorinating organic compounds using a palladium complex, e.g., a palladium complex described herein. In certain embodiments, the process comprises mixing an organic compound comprising one or more boron, organostannane or silane substituents, a palladium(II) complex, and a fluorinating agent to provide an organic compound wherein a boron, organostannane or silane substituent is replaced with a fluorine substituent. In certain embodiments, the above process is a multi-step process comprising:

(i) mixing an organic compound comprising one or more boron, organostannane or silane substituents and a palladium(II) complex; and

(ii) adding a fluorinating agent (e.g., an electrophilic fluorination reagent) to provide a fluorinated organic compound, whereby the boron, organostannane or silane substituent is replaced with a fluorine substituent.

In certain embodiments, the fluorinating agent is added to the reaction mixture of step (i). In certain embodiments, the reaction mixture of step (i) is added to the fluorinating agent or a solution thereof.

In certain embodiments, the boron, organostannane or silane substituent is replaced with the fluorine substituent regiospecifically (i.e., providing only one product from the reaction process).

In certain embodiments, the boron, organostannane or silane substituent is replaced with the fluorine substituent stereoselectively (i.e., providing a major stereoisomer product from the reaction process).

In certain embodiments, the process of step (i) further comprises providing an intermediate of the palladium(II) complex and the organic compound (“an intermediate palladium(II) complex”). In certain embodiments, the process of step (i) further comprises isolating the intermediate palladium(II) complex.

For example, in certain embodiments, the process comprises the steps of:

(i) mixing an organic compound comprising a boron, organostannane or silane substituent together with a palladium(II) complex to provide an intermediate palladium(II) complex, wherein the boron, organostannane or silane substituent is replaced with palladium,

(ii) optionally isolating the intermediate palladium(II) complex, and

(iii) mixing the intermediate palladium(II) complex and a fluorinating agent to provide a fluorinated organic compound, whereby the palladium is replaced with a fluorine substituent.

In certain embodiments, the process comprises the steps of:

(i) providing an intermediate palladium(II) complex comprising an organic compound conjugated to Pd via a carbon atom; and

(ii) mixing the intermediate palladium complex and a fluorinating agent to provide a fluorinated organic compound whereby Pd is replaced with a fluorine substituent.

In certain embodiments, the mixing step (iii) comprises adding the fluorinating agent to the intermediate palladium(II) complex. In certain embodiments, the mixing step (iii) comprises adding the intermediate palladium(II) complex to the fluorinating agent.

In certain embodiments, prior to step (i), the process comprises adding a boron, organostannane or silane substituent to an organic compound to provide an organic compound comprising a boron, organostannane or silane substituent.

However, in certain embodiments, the entire process is conducted in one-pot (i.e., two or more reaction steps conducted in one reaction vessel).

In certain embodiments, a high-valent palladium fluoride intermediate is produced during the course of the reaction. The high-valent palladium fluoride species is produced upon treatment of the Pd (II) complex with a fluorinating agent. In certain embodiments, the high-valent palladium fluoride intermediate is observable. In certain embodiments, the high-valent palladium fluoride intermediate is isolatable. Formation of the high-valent palladium fluoride intermediate is followed by reductive elimination to form a carbon-fluoride bond. In certain embodiments, the reaction may not proceed through a high-valent palladium fluoride intermediate.

### (i) Palladium(II) Complex

As generally described herein, the fluorination process utilizes a palladium(II) complex (i.e., the palladium has a valency of +2). The palladium(II) complexes described herein are considered to be part of the invention.

In certain embodiments, a stoichiometric amount of the palladium(II) complex is used.

In certain embodiments, the palladium(II) complex comprises a bidentate ligand. In certain embodiments, the palladium(II) complex comprises a tridentate ligand.

In certain embodiments, the palladium(II) complex is crystalline. Alternatively, in certain embodiments, the palladium(II) complex is amorphous.

In certain embodiments, the palladium(II) complex is not a salt. Alternatively, in certain embodiments, the palladium(II) complex is a salt. For example, in certain embodiments, the palladium(II) complex is a salt of tetrafluoroborate (BF₄⁻), tetraphenylborate (BPh₄⁻), hexafluorophosphate (PF₆⁻), tetrakis[3,5-bis(trifluoromethyl)phenyl]borate ([BArF₄]⁻), tetrakis(pentafluorophenyl)borate (B(C₆F₅)₄⁻), antimohexafluoride (SbF₆⁻), or trifluoromethansulfonate (triflate, CF₃SO₃⁻). In certain embodiments, the palladium(II) complex is a salt of tetrafluoroborate (BF₄⁻).

In certain embodiments, the palladium(II) complex is a palladium(II)dimer complex.

In certain embodiments, the palladium(II) complex is generated in situ from a complex in the 0 oxidation state (i.e., a “palladium(0) complex”) and one or more ligands.

Exemplary ligands include, but are not limited to, halogens (e.g., iodide, bromide, chloride, fluoride), solvents (e.g., hydroxide, water, ammonia, acetonitrile, dimethylsulfoxide, dimethylformamide, dimethylacetamide), sulfide, cyanide, carbon monoxide, thiocyanate, isothiocyanate, nitrate, nitrite, azide, oxalate, olefins (e.g., dibenzylidineacetone (dba)), optionally substituted pyridines (py) (e.g., 2,2′,5′,2-terpyridine (terpy), bipyridine (bipy) and other pyridine ligands as described herein), optionally substituted aryl (e.g., phenyl (Ph), phenanthroline (phen), biphenyl), phosphines (e.g., triphenylphosphine (PPh₃), 1,2-bis(diphenylphosphino)ethane (dppe), tricyclohexylphosphine (PCy₃), tri(o-tolyl)phosphine (P(o-tol)₃), tris(2-diphenylphosphineethyl)amine (np3)), amino ligands (e.g., ethylenediamine (en), diethylenetriamine (dien), tris(2-aminoethyl)amine (tren), triethylenetetramine (trien), ethylenediaminetetraacetate (EDTA)), acyloxy ligands (e.g., acetylaceonate (acac), O-acetate (—OAc)), and alkyloxy ligands (e.g., —OMe, OiPr, OtBu).

As one of ordinary skill in the art would understand, the ligands are chosen to satisfy the valency of palladium. Thus, in certain embodiments, the ligands are chosen to satisfy the valency of a palladium complex as +2.

Exemplary palladium(II) complexes include, but are not limited to, palladium(II) bromide, palladium(II) chloride, palladium(II) iodide, palladium(II) fluoride, palladium(II)acetate, palladium(II) acetylacetonate, palladium(II) oxide, palladium(II) cyanide, palladium(II) sulfide, palladium(II) sulfate, palladium(II) 2,4-pentanedionate, allyl palladium(II) chloride dimer, bis(acetonitrile)dichloropalladium(II), trans-bis(benzonitrile)dichloropalladium(II), and trichloro-bis(triphenylphosphine)palladium(II).

Exemplary palladium(0) complexes include, but are not limited to, Pd₂ dba₃, Pd₂ dba₃-CHCl₃, and tetrakis(triphenylphosphine)palladium(0).

Other exemplary ligands are provided as groups R^{L1} and R^{L2}, described below and herein. Furthermore, other exemplary bidentate and tridentate palladium(II) complexes are provided in the following formulae, described below and herein.

For example, in certain embodiments, the palladium(II) complex comprises a bidentate or tridentate ligand to provide a complex of the formula (I):

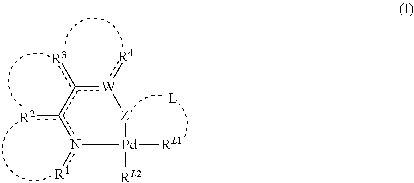

wherein:

Pd represents palladium of valency of +2;

R^{L1} and R^{L2} are, independently, optionally substituted aliphatic, optionally substituted heteroaliphatic, optionally substituted aryl, optionally substituted heteroaryl, halogen, —OR^{a}, —SR^{b}, —N(R^{c})₂, —N(R^{c})₃, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group [symbol] C(R^{c1}), wherein R^{c1} is an optionally substituted aliphatic, optionally substituted R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

wherein each instance of R^{x} is, independently, hydrogen, an optionally substituted aliphatic, optionally substituted alkoxy, optionally substituted heteroaliphatic, optionally substituted aryloxy, optionally substituted heteroaryloxy, optionally substituted aryl, or optionally substituted heteroaryl group;

when W is —C— or —C(R^{d})— then:
(i) Z is a bond, —O—, —S—, —C(R^{d})₂—, —C(R^{d})═C(R^{d})—, —C(R^{d})═N—, or —N(R^{e})—;

or
(ii) Z is —N— joined via a linker group -L- to the group R^{L1} to form a 5- to 7-membered palladacycle, wherein -L- is selected from absent, —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring;

or
(iii) Z is —N—S(O)₂—R^{e3} and the linker group -L- is absent;

or

when W is —N— or —N(R^{e})—, then Z is a bond, —C(R^{d})₂—, —C(R^{d})═C(R^{d})—, or —C(R^{d})═N—;

or

when W is —SO₂— or ═N—, then R₄ is absent;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

R² and R³ are optionally joined to form an optionally substituted 5- to 7-membered heteroaryl, aryl, heterocyclic or carbocyclic ring;

R³ and R⁴ are optionally joined to form an optionally substituted 5- to 7-membered heteroaryl, aryl, heterocyclic or carbocyclic ring,

wherein the each of curved dotted lines [symbol] independently represents optional joining of an optionally substituted 5- to 7-membered ring, and

wherein [symbol] represents a single or double bond.

In certain embodiments, R¹ and R² are joined to form an optionally substituted 5- to 6-membered heteroaryl, aryl, heterocyclic or carbocyclic ring. In certain embodiments, R¹ and R² are joined to form an optionally substituted 5-membered heteroaryl, aryl, heterocyclic or carbocyclic ring. In certain embodiments, R¹ and R² are joined to form an optionally substituted 6-membered heteroaryl, aryl, heterocyclic or carbocyclic ring.

In certain embodiments, R² and R³ are joined to form an optionally substituted 5- to 6-membered heteroaryl, aryl, heterocyclic or carbocyclic ring. In certain embodiments, R² and R³ are joined to form an optionally substituted 5-membered heteroaryl, aryl, heterocyclic or carbocyclic ring. In certain embodiments, R² and R³ are joined to form an optionally substituted 6-membered heteroaryl, aryl, heterocyclic or carbocyclic ring.

In certain embodiments, R³ and R⁴ are joined to form an optionally substituted 5- to 6-membered heteroaryl, aryl, heterocyclic or carbocyclic ring. In certain embodiments, R³ and R⁴ are joined to form an optionally substituted 5-membered heteroaryl, aryl, heterocyclic or carbocyclic ring. In certain embodiments, R³ and R⁴ are joined to form an optionally substituted 6-membered heteroaryl, aryl, heterocyclic or carbocyclic ring.

Any of the optionally substituted 5- to 6-membered heteroaryl, aryl, heterocyclic or carbocyclic rings formed by joining R¹ and R², R² and R³ and/or R³ and R⁴ can be, for example, an optionally substituted 5- to 6-membered heteroaryl, an optionally substituted 6-membered aryl, an optionally substituted 5- to 6-membered heterocyclic or an optionally substituted 5- to 6-membered carbocyclic ring.

Exemplary 5-membered heteroaryl rings include, but are not limited to, optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isoxazolyl, optionally substituted oxadiaziolyl or optionally substituted oxadiaziolyl ring.

Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

Exemplary 5-membered heterocyclic rings include, but are not limited to, optionally substituted pyrrolidinyl, optionally substituted tetrahydrofuranyl, optionally substituted tetrahydrothiophenyl, and optionally substituted 1,3 dithiolanyl.

Exemplary 6-membered heterocyclic rings include, but are not limited to, optionally substituted piperidinyl, optionally substituted piperazinyl, optionally substituted morpholinyl, optionally substituted tetrahydropyranyl and optionally substituted dioxanyl.

Exemplary 5-membered carbocyclic rings include, but are not limited to, optionally substituted cyclopentyl and optionally substituted cyclopentenyl.

Exemplary 6-membered carbocyclic rings include, but are not limited to, optionally substituted cyclohexyl and optionally substituted cyclohexenyl.

In certain embodiments, R² and R³ are not joined together to form a cyclic structure.

In certain embodiments, R³ and R⁴ are not joined together to form a cyclic structure.

In certain embodiments, both R¹ and R² and R² and R³ are joined to form rings, but R³ and R⁴ are not joined together to form a cyclic structure.

In certain embodiments, both R¹ and R² and R³ and R⁴ are joined to form rings, but R² and R³ are not joined together to form a cyclic structure.

In certain embodiments, both R² and R³ and R³ and R⁴ are joined to form rings, but R¹ and R² are not joined together to form a cyclic structure.

Palladium(II) Complexes with Bidentate Ligand

In certain embodiments, Z is not joined via a linker group -L- to the group R^{L1} to form a 5- to 7-membered palladacycle.

For example, in certain embodiments, the palladium(II) complex comprises a bidentate ligand. In certain embodiments, the palladium(II) complex is of the formula (I-a):

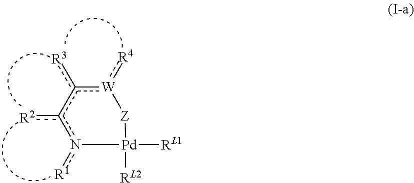

wherein Pd, [symbol] , [symbol] , W, R^{L1}, R^{L2}, Z, R¹, R², R³ and R⁴ are as defined above and herein.

In certain embodiments, R¹ and R² are joined to form an optionally substituted 6-membered pyridinyl ring to provide a palladium(II) complex of the formula (I-b):

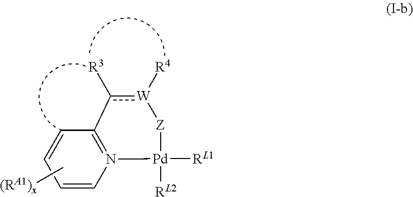

wherein

Pd, [symbol] , [symbol] , W, R^{L1}, R^{L2}, Z, R³, and R⁴ are as defined above and herein;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, —SR^{A1b}, —N(R^{A1c})₂, —C(═O)R^{A1d}, —C(═O)OR^{Ala}, —C(═O)N(R^{A1c})₂, —C(═NR^{A1c})R^{A1d}, —C(═NR^{A1c})OR^{A1a}, —C(═NR^{A1c})N(R^{A1c})₂, —S(O)₂R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group; and

x is an integer between 0-4, inclusive.

In certain embodiments, each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}. In certain embodiments, each instance of R^{A1} is, independently, hydrogen, halogen, optionally substituted C₁₋₆ alkyl, —NO₂, —CF₃, or —OR^{A1a}. In certain embodiments, each instance of R^{A1} is, independently, hydrogen, —CH₃, -tBu, —CN, —NO₂, —CF₃, or —OCH₃. In certain embodiments, each instance of R^{A1} is hydrogen.

In certain embodiments, R³ and R⁴ are joined to form an optionally substituted aryl ring to provide a palladium(II) complex of the formula (I-c):

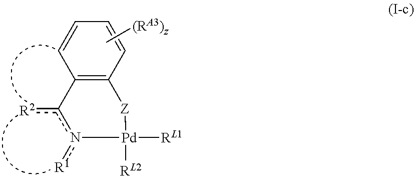

wherein

Pd, [symbol] , [symbol] , R¹, R², R^{L1}, R^{L2} and Z are as defined above and herein;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —C(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3c} is, independently, hydrogen, an optionally substituted aliphatic, optionally substituted heteroaliphatic, optionally substituted aryl, optionally substituted heteroaryl or a suitable amino protecting group, or two R^{A3c} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group; and

z is an integer between 0-3, inclusive.

In certain embodiments, each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}. In certain embodiments, each instance of R^{A3} is, independently, hydrogen, halogen, optionally substituted C₁₋₆ alkyl, —NO₂, —CF₃, or —OR^{A3a}. In certain embodiments, each instance of R^{A3} is, independently, hydrogen, —CH₃, -tBu, —CN, —NO₂, —CF₃, or —OCH₃. In certain embodiments, each instance of R^{A3} is hydrogen.

In certain embodiments, R¹ and R² are joined to form an optionally substituted 6-membered pyridinyl ring and R³ and R⁴ are joined to form an optionally substituted aryl ring to provide a palladium(II) complex of the formula (I-d):

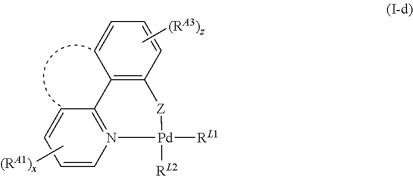

wherein Pd, [symbol] , [symbol] , R^{A1}, R^{A3}, R^{L1}, R^{L2}, x, z, and Z are as defined above and herein.

In certain embodiments, R¹ and R² are joined to form an optionally substituted 6-membered pyridinyl ring and R² and R³ are joined to form an optionally substituted 6-membered aryl ring, to provide a palladium(II) catalyst of the formula (I-e):

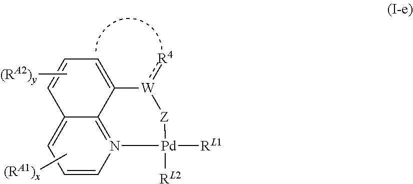

wherein

Pd, [symbol] , [symbol] , W, R^{A1}, R^{L1}, R^{L2}, R⁴, x, and Z are as defined above and herein;

each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}, —SR^{A2b}, —N(R^{A2c})₂, —C(═O)R^{A2d}, —C(═O)OR^{A2a} C(═O)N(R^{A2c})₂, —C(═NR^{A2c})R^{A2d}, —C(═NR^{A2c})OR^{A2a}, —C(═NR^{A2c})N(R^{A2c})₂, —S(O)₂R^{A2d}, —S(O)R^{A2d}, or two R^{A2} groups adjacent to each other are joined to form a 5- to 6-membered aryl, heteroaryl, heterocyclic or carbocyclic ring, wherein R^{A2a} is hydrogen, an optionally substituted aliphatic, optionally substituted heteroaliphatic, optionally substituted aryl, optionally substituted heteroaryl or a suitable hydroxyl protecting group; wherein R^{A2b} is hydrogen, an optionally substituted aliphatic, optionally substituted heteroaliphatic, optionally substituted aryl, optionally substituted heteroaryl or a suitable thiol protecting group; wherein each R^{A2c} is, independently, hydrogen, an optionally substituted aliphatic, optionally substituted heteroaliphatic, optionally substituted aryl, optionally substituted heteroaryl or a suitable amino protecting group, or two R^{A2c} groups are joined together to form a heterocyclic or heteroaryl group; and wherein each R^{A2d} is, independently, an optionally substituted aliphatic, optionally substituted heteroaliphatic, optionally substituted aryl, or an optionally substituted heteroaryl group; and

y is an integer between 0-2, inclusive.

In certain embodiments, each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}. In certain embodiments, each instance of R^{A2} is, independently, hydrogen, halogen, optionally substituted C₁₋₆ alkyl, —NO₂, —CF₃, or —OR^{A2a}. In certain embodiments, each instance of R^{A2} is, independently, hydrogen, —CH₃, -tBu, —CN, —NO₂, —CF₃, or —OCH₃. In certain embodiments, each instance of R^{A2} is hydrogen.

In certain embodiments, R² and R³ are joined to form an optionally substituted 6-membered aryl ring to provide a palladium(II) catalyst of the formula (I-f):

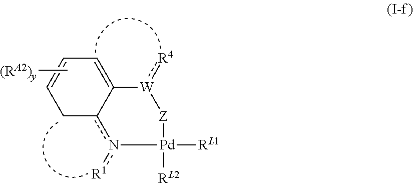

wherein Pd, [symbol] , [symbol] , W, R^{A2}, R¹, R⁴, R^{L1}, R^{L2}, y and Z are as defined above and herein.

In certain embodiments, R¹ and R² are joined to form an optionally substituted pyridinyl ring, R² and R³ are joined to form an optionally substituted 6-membered aryl ring and R³ and R⁴ are joined to form an optionally substituted 6-membered aryl ring to form the bidentate palladium(II) complex of the formula (I-g):

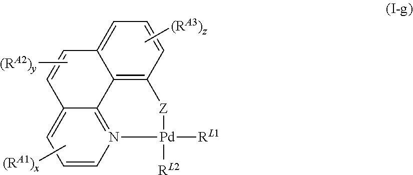

wherein Pd, R^{L1}, R^{L2}, Z, R^{A1}, R^{A2}, R^{A3}, x, y and z are as defined above and herein.

In certain embodiments, wherein R² and R³ are not joined to form an optionally substituted 5- to 6-membered ring, the palladium(II) complex is of the formula (I-h):

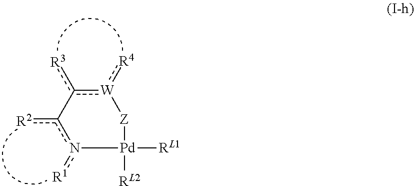

wherein Pd, [symbol] , [symbol] , W, Z, R¹, R², R³, R⁴, R^{L1} and R^{L2} are as defined above and herein; and

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring; and

R³ and R⁴ are optionally joined to form an optionally substituted 5- to 7-membered heteroaryl, aryl, heterocyclic or carbocyclic ring.

In certain embodiments, wherein R² and R³ are not joined to form a cyclic structure, the palladium(II) complex is of the formula (I-i):

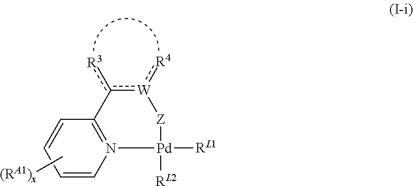

wherein Pd, [symbol] , [symbol] , W, R³, R⁴, R^{L1}, R^{L2}, R^{A1} and x are as defined above and herein.

In certain embodiments, wherein R² and R³ are not joined to form a cyclic structure, the palladium(II) complex is of the formula (I-j):

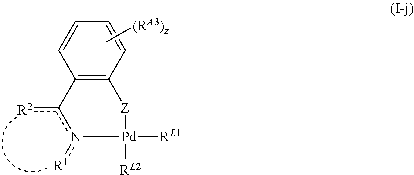

wherein Pd, [symbol] , [symbol] , R¹, R², R^{L1}, R^{L2}, R^{A3}, Z, and z are as defined above and herein.

In certain embodiments, wherein R² and R³ are not joined to form a cyclic structure, the palladium(II) complex is of the formula (I-k):

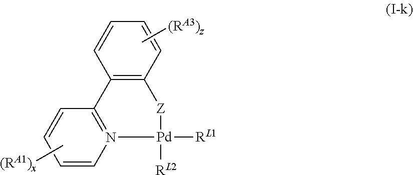

wherein Pd, R^{L1}, R^{L2}, R^{A1}, R^{A3}, Z, z and x are as defined above and herein.

In certain embodiments, in any of the above formulae Z is a bond. In other embodiments, Z is

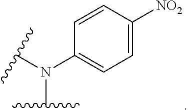 [SMILES: *N(*)c1ccc([N+](=O)[O-])cc1]

In other embodiments, Z is

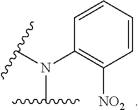 [SMILES: *N(*)c1ccccc1[N+](=O)[O-]]

In certain embodiments, wherein R² and R³ are not joined to form a cyclic structure and Z is a bond, the palladium(II) complex is of the formula (I-l):

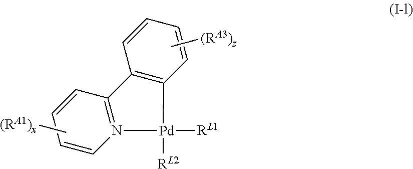

wherein R^{L1}, R^{L2}, R^{A1}, R^{A3}, z and x are as defined above and herein.

In certain embodiments, the palladium(II) complex is of the formula (I-k):

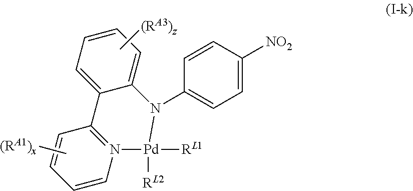

wherein R^{L1}, R^{L2}, R^{A1}, R^{A3}, z, and x are as defined above and herein.

In certain embodiments, the palladium(II) complex is of the formula (I-l′):

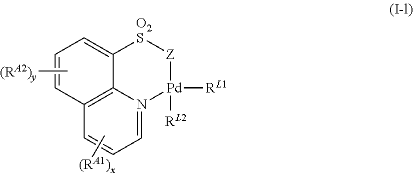

wherein Pd, R^{L1}, R^{L2}, R^{A1}, R^{A2}, x, y, and Z are as defined above and herein.

In certain embodiments, the palladium(II) complex is of the formula (I-m′):

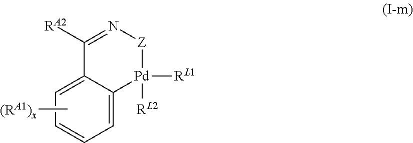

wherein Pd, R^{L1}, R^{L2}, R^{A1}, R^{A2}, x, and Z are as defined above and herein.

In certain embodiments, the palladium(II) complex is of the formula (I-n′):

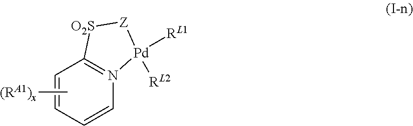

wherein Pd, R^{L1}, R^{L2}, R^{A1}, x, and Z are as defined above and herein.
Palladium(II) Complexes with Tridentate Ligand

In certain embodiments, Z is joined via a linker group -L- to the group R^{L1} to form a 5- to 7-membered palladacycle.

In certain embodiments, the palladium(II) catalyst comprises a tridentate ligand. In certain embodiments, the palladium(II) catalyst of the formula (I-a′):

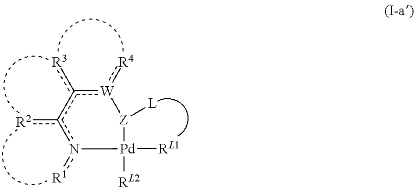

wherein

Pd, [symbol] , [symbol] , W, R^{L1}, R^{L2}, R¹, R², R³, and R⁴ are as defined above and herein;

Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted heterocyclic or heteroaryl ring; and

the curved solid line [symbol] represents joining of the 5- to 7-membered palladacycle.

In certain embodiments, R¹ and R² are joined to form an optionally substituted 6-membered pyridinyl ring to provide a palladium(II) complex of the formula (I-b′):

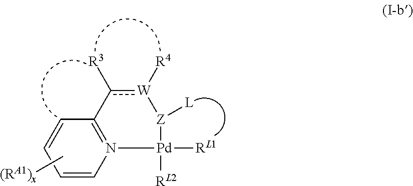

wherein

Pd, [symbol] , [symbol] , [symbol] , W, L, R^{L1}, R^{L2}, Z, R³ and R⁴ are as defined above and herein;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, —SR^{A1b}, —N(R^{A1c})₂, —C(═O)R^{A1d}, —C(═O)OR^{Ala}, —C(═O)N(R^{A1c})₂, —C(═NR^{A1c})R^{A1d}, —C(═NR^{A1c})OR^{A1a}, —C(═NR^{A1c})N(R^{A1c})₂, —S(O) 2R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group; and

x is an integer between 0-4, inclusive.

In certain embodiments, each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}. In certain embodiments, each instance of R^{A1} is, independently, hydrogen, halogen, optionally substituted C₁₋₆ alkyl, —NO₂, —CF₃, or —OR^{A1a}. In certain embodiments, each instance of R^{A1} is, independently, hydrogen, —CH₃, -tBu, —CN, —NO₂, —CF₃, or —OCH₃. In certain embodiments, each instance of R^{A1} is hydrogen.

In certain embodiments, R³ and R⁴ are joined to form an optionally substituted aryl ring to provide a palladium(II) complex of the formula (I-c′):

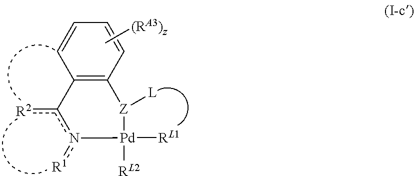

wherein

Pd, [symbol] , [symbol] , [symbol] , L, R¹, R², R^{L1}, R^{L2}, z, and Z are as defined above and herein;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —C(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3c} is, independently, hydrogen, an optionally substituted aliphatic, optionally substituted heteroaliphatic, optionally substituted aryl, optionally substituted heteroaryl or a suitable amino protecting group, or two R^{A3c} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group; and

z is an integer between 0-3, inclusive.

In certain embodiments, each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}. In certain embodiments, each instance of R^{A3} is, independently, hydrogen, halogen, optionally substituted C₁₋₆ alkyl, —NO₂, —CF₃, or —OR^{A3a}. In certain embodiments, each instance of R^{A3} is, independently, hydrogen, —CH₃, -tBu, —CN, —NO₂, —CF₃, or —OCH₃. In certain embodiments, each instance of R^{A3} is hydrogen.

In certain embodiments, R¹ and R² are joined to form an optionally substituted 6-membered pyridinyl ring and R³ and R⁴ are joined to form an optionally substituted aryl ring to provide a palladium(II) complex of the formula (I-d′):

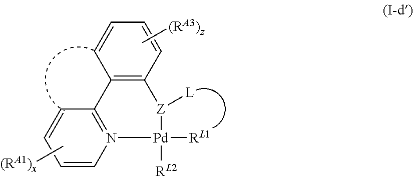

wherein Pd, [symbol] , [symbol] , [symbol] , L, R^{A1}, R^{A3}, R^{L1}, R^{L2}, x, z, and Z are as defined above and herein.

In certain embodiments, R¹ and R² are joined to form an optionally substituted 6-membered pyridinyl ring and R² and R³ are joined to form an optionally substituted 6-membered aryl ring, to provide a palladium(II) catalyst of the formula (I-e′):

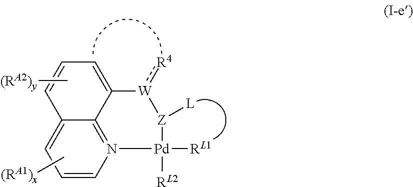

wherein Pd, [symbol] , [symbol] , [symbol] , L, W, R^{A1}, R^{L1}, R^{L2}, R⁴, x and Z are as defined above and herein;

each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}, —SR^{A2b}, —N(R^{A2c})₂, —C(═O)R^{A2d}, —C(═O)OR^{A2a}, —C(═O)N(R^{A2c})₂, —C(═NR^{A2c})R^{A2d}, —C(═NR^{A2c})OR^{A2a}, —C(═NR^{A2c})N(R^{A2c})₂, —S(O)₂R^{A2d}, —S(O)R^{A2d}, or two R^{A2} groups adjacent to each other are joined to form a 5- to 6-membered aryl, heteroaryl, heterocyclic or carbocyclic ring, wherein R^{A2a} is hydrogen, an optionally substituted aliphatic, optionally substituted heteroaliphatic, optionally substituted aryl, optionally substituted heteroaryl or a suitable hydroxyl protecting group; wherein R^{A2b} is hydrogen, an optionally substituted aliphatic, optionally substituted heteroaliphatic, optionally substituted aryl, optionally substituted heteroaryl or a suitable thiol protecting group; wherein each R^{A2c} is, independently, hydrogen, an optionally substituted aliphatic, optionally substituted heteroaliphatic, optionally substituted aryl, optionally substituted heteroaryl or a suitable amino protecting group, or two R^{A2c} groups are joined together to form a heterocyclic or heteroaryl group; and wherein each R^{A2d} is, independently, an optionally substituted aliphatic, optionally substituted heteroaliphatic, optionally substituted aryl, or an optionally substituted heteroaryl group; and

y is an integer between 0-2, inclusive.

In certain embodiments, each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}. In certain embodiments, each instance of R^{A2} is, independently, hydrogen, halogen, optionally substituted C₁₋₆alkyl, —NO₂, —CF₃, or —OR^{A2a}. In certain embodiments, each instance of R^{A2} is, independently, hydrogen, —CH₃, -tBu, —CN, —NO₂, —CF₃, or —OCH₃. In certain embodiments, each instance of R^{A2} is hydrogen.

In certain embodiments, R² and R³ are joined to form an optionally substituted 6-membered aryl ring to provide a palladium(II) catalyst of the formula (I-f′):

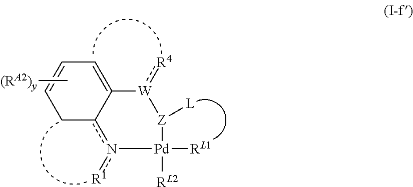

wherein Pd, [symbol] , [symbol] , [symbol] , L, W, R^{A2}, R¹, R⁴, R^{L1}, R^{L2}, y and Z are as defined above and herein.

In certain embodiments, R¹ and R² are joined to form an optionally substituted pyridinyl ring, R² and R³ are joined to form an optionally substituted 6-membered aryl ring and R³ and R⁴ are joined to form an optionally substituted 6-membered aryl ring to form the palladium(II) complex of the formula (I-g′):

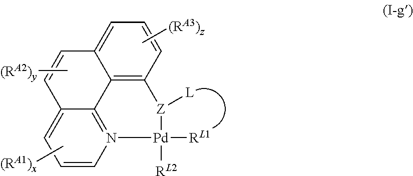

wherein [symbol] , L, R^{L1}, R^{L2}, Z, R^{A1}, R^{A2}, R^{A3}, x, y and z are as defined above and herein.

In certain embodiments, wherein R² and R³ are not joined to form an optionally substituted 5- to 6-membered ring, the palladium(II) complex is of the formula (I-h′):

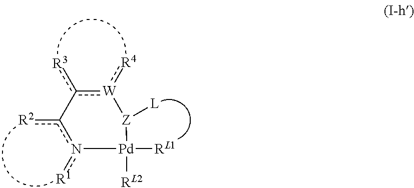

wherein Pd , [symbol] , [symbol] , [symbol] , L, W, Z, R¹, R², R³, R⁴, R^{L1} and R^{L2} are as defined above and herein; and

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

and

R³ and R⁴ are optionally joined to form an optionally substituted 5- to 7-membered heteroaryl, aryl, heterocyclic or carbocyclic ring.

In certain embodiments, wherein R² and R³ are not joined to form a cyclic structure, the palladium(II) complex is of the formula (I-i′):

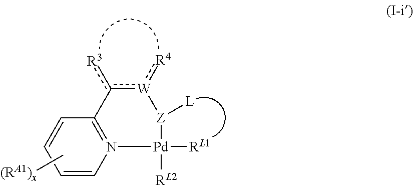

wherein Pd, [symbol] , [symbol] , [symbol] , L, W, R³, R⁴, R^{L1}, R^{L2}, R^{A1} and x are as defined above and herein.

In certain embodiments, wherein R² and R³ are not joined to form a cyclic structure, the palladium(II) complex is of the formula (I-j′):

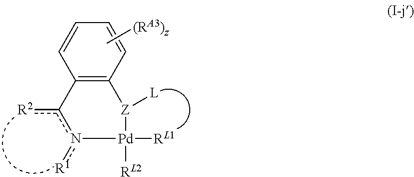

wherein Pd, [symbol] , [symbol] , [symbol] , L, R¹, R², R^{L1}, R^{L2}, R^{A3} and z are as defined above and herein.

In certain embodiments, wherein R² and R³ are not joined to form a cyclic structure, the palladium(II) complex is of the formula (I-k′):

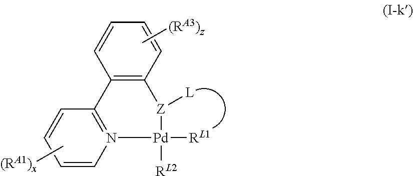

wherein Pd, [symbol] , L, R^{L1}, R^{L2}, R^{A1}, R^{A3}, Z, z and x are as defined above and herein.

### Groups R^{L1} and R^{L2}

As defined generally herein, R^{L1} and R^{L2} are, independently, halogen, optionally substituted aliphatic, optionally substituted heteroaliphatic, optionally substituted aryl, optionally substituted heteroaryl, —OR^{a}, —SR^{b}, —N(R^{c})₃, —N(R^{c})₂, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, heteroaliphatic, aryl, heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, heteroaliphatic, aryl, heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted 5- to 6-membered heterocyclic or heteroaryl ring or the group [symbol] C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring; and

wherein each instance of R^{x} is, independently, hydrogen, an optionally substituted aliphatic, optionally substituted alkoxy, optionally substituted heteroaliphatic, optionally substituted aryloxy, optionally substituted heteroaryloxy, optionally substituted aryl, or optionally substituted heteroaryl group.

In certain embodiments, at least one of R^{L1} and R^{L2} is selected from halogen, —OR^{a}, —SR^{b}, —N(R^{c})₃, —N(R^{c})₂, or —P(R^{x})₃. In certain embodiments, both R^{L1} and R^{L2} are, independently, selected from halogen, —OR^{a}, —SR^{b}, —N(R^{c})₃, —N(R^{c})₂, or —P(R^{x})₃.

In certain embodiments, R^{L1} is halogen, —OR^{a}, —SR^{b}, or —N(R^{c})₂ and R^{L2} is —N(R^{c})₂. In certain embodiments, R^{L1} is halogen, —OR^{a} or —N(R^{c})₂, and R^{L2} is —N(R^{c})₂. In certain embodiments, R^{L1} is halogen or —OR^{a}, and R^{L2} is —N(R^{c})₂. In certain embodiments, R^{L1} is and R^{L2} is —N(R^{c})₂. In certain embodiments, R^{L1} is halogen and R^{L2} is —N(R^{c})₂. In certain embodiments, R^{L1} is —OR^{a} and R^{L2} is —N(R^{c})₂. In certain embodiments, both R^{L1} and R^{L2} are independently-N(R)₂.

In certain embodiments, R^{L1} is halogen. In certain embodiments, R^{L1} is —Cl. In certain embodiments, R^{L1} is —Br. In certain embodiments, R^{L1} is —I. In certain embodiments, R^{L1} is —F.

In certain embodiments, R^{L1} is —OR^{a}.

In certain embodiments, R^{L1} is —OC(═O)R^{a1} wherein R^{a1} is an optionally substituted aliphatic, optionally substituted heteroaliphatic, optionally substituted aryl or optionally substituted heteroaryl group. In certain embodiments, R^{L1} is —OC(═O)R^{a1} wherein R^{a1} is an optionally substituted aliphatic group. In certain embodiments, R^{L1} is —OC(═O)R^{a1} wherein R^{a1} is an optionally substituted C₁₋₆ alkyl group. In certain embodiments, R^{L1} is —OC(═O)R^{a1} wherein R^{a1} is an optionally substituted C₁₋₄ alkyl group. In certain embodiments, R^{L1} is —OC(═O)R^{a1} wherein R^{a1} is an optionally substituted C₁₋₂alkyl group. In certain embodiments, R^{L1} is —OC(═O)CH₃.

In certain embodiments, R^{L1} is —P(R^{X})₃.

In certain embodiments, R^{L2} is —N(R^{c})₂.

In certain embodiments, R^{L2} is —N(R^{c})₂ wherein two R^{c} groups are joined to form the group [symbol] C(R^{c1}), wherein R^{c1} is an optionally substituted aliphatic, optionally substituted heteroaliphatic, optionally substituted aryl or optionally substituted heteroaryl group. In certain embodiments, R^{L2} is —N(R^{c})₂ wherein two R^{c} groups are joined to form the group [symbol] C(R^{c1}), wherein R^{c1} is an optionally substituted aliphatic group. In certain embodiments, R^{L2} is —N(R^{c})₂ wherein two R^{c} groups are joined to form the group [symbol] C(R^{c1}) wherein R^{c1} is an optionally substituted C₁₋₆ alkyl group. In certain embodiments, R^{L2} is —N(R^{c})₂ wherein two R^{c} groups are joined to form the group [symbol] C(CH₃) or [symbol] C(CH₂Ph).

In certain embodiments, R^{L2} is —N(R^{c})₂ wherein two R^{c} groups are joined to form an optionally substituted heterocyclic or heteroaryl ring.

In certain embodiments, R^{L2} is —N(R^{c})₂ wherein two R^{c} groups are joined to form an optionally substituted 5- to 6-membered heterocyclic or heteroaryl ring.

In certain embodiments, R^{L2} is —N(R^{c})₂ wherein two R^{c} groups are joined to form an optionally substituted 5-membered heterocyclic ring. Exemplary 5-membered heterocyclic rings include, but are not limited to, an optionally substituted pyrrolidinyl ring.

In certain embodiments, R^{L2} is —N(R^{c})₂ wherein two R^{c} groups are joined to form an optionally substituted 5-membered heteroaryl ring. Exemplary 5-membered heteroaryl rings include, but are not limited to, an optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isoxazolyl, optionally substituted oxadiaziolyl or optionally substituted oxadiaziolyl ring.

In certain embodiments, R^{L2} is —N(R^{c})₂ wherein two R^{c} groups are joined to form an optionally substituted 6-membered heterocyclic ring. Exemplary 6-membered heterocyclic rings include, but are not limited to, optionally substituted piperidinyl, optionally substituted piperazinyl or optionally substituted morpholinyl ring.

In certain embodiments, R^{L2} is —N(R^{c})₂ wherein two R^{c} groups are joined to form an optionally substituted 6-membered heteroaryl ring. Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

In certain embodiments, R^{L2} is an optionally substituted pyridinyl ring.

In certain embodiments, R^{L1} is —N(R^{c})₂.

In certain embodiments, R^{L1} is —N(R^{c})₂ wherein two R^{c} groups are joined to form the group [symbol] C(R^{c1}), wherein R^{c1} is an optionally substituted aliphatic, optionally substituted heteroaliphatic, optionally substituted aryl or optionally substituted heteroaryl group. In certain embodiments, R^{L1} is —N(R^{c})₂ wherein two R^{c} groups are joined to form the group [symbol] C(R^{c1}), wherein R^{c1} is an optionally substituted aliphatic group. In certain embodiments, R^{L1} is —N(R^{c})₂ wherein two R^{c} groups are joined to form the group [symbol] C(R^{c1}), wherein R^{c1} is an optionally substituted C₁₋₆ alkyl group. In certain embodiments, R^{L1} is —N(R^{c})₂ wherein two R^{c} groups are joined to form the group [symbol] C(CH₃) or [symbol] C(CH₂Ph).

In certain embodiments, R^{L1} is —N(R^{c})₂ wherein two R^{c} groups are joined to form an optionally substituted 5- to 6-membered heterocyclic or heteroaryl ring.

In certain embodiments, R^{L1} is —N(R^{c})₂ wherein two R^{c} groups are joined to form an optionally substituted 5-membered heterocyclic ring. Exemplary 5-membered heterocyclic rings are provided above and herein.

In certain embodiments, R^{L1} is —N(R^{c})₂ wherein two R^{c} groups are joined to form an optionally substituted 5-membered heteroaryl ring. Exemplary 5-membered heteroaryl rings are provided above and herein.

In certain embodiments, R^{L1} is —N(R^{c})₂ wherein two R^{c} groups are joined to form an optionally substituted 6-membered heterocyclic ring. Exemplary 6-membered heterocyclic rings are provided above and herein.

In certain embodiments, R^{L1} is —N(R^{c})₂ wherein two R^{c} groups are joined to form an optionally substituted 6-membered heteroaryl ring. Exemplary 6-membered heteroaryl rings are provided above and herein.

In certain embodiments, R^{L1} is an optionally substituted pyridinyl ring.

Optionally substituted pyridinyl rings include, but are not limited to, rings of the formula:

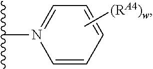

wherein each instance of R^{A4} is, independently, hydrogen, halogen, optionally substituted aliphatic, optionally substituted heteroaliphatic, optionally substituted aryl, optionally substituted heteroaryl, —CN, —NO₂, —NC, —OR^{A4a}, —SR^{A4b}, —N(R^{A4c})₂, —C(═O)R^{A4d}, —C(═O)OR^{A4a}, —C(═O)N(R^{A4c})₂, —C(═NR^{A4c})R^{A4d}, —C(═NR^{A4c})OR^{A4a}, —C(═NR^{A4c})N(R^{A4c})₂, —S(O)₂R^{A4d}, —S(O)R^{A4d}, or two R^{A4} groups adjacent to each other are joined to form a 5- to 6-membered aryl, heteroaryl, heterocyclic or carbocyclic ring, wherein R^{A4a} is hydrogen, an optionally substituted aliphatic, optionally substituted heteroaliphatic, optionally substituted aryl, optionally substituted heteroaryl or a suitable hydroxyl protecting group; wherein R^{A4b} is hydrogen, an optionally substituted aliphatic, optionally substituted heteroaliphatic, optionally substituted aryl, optionally substituted heteroaryl or a suitable thiol protecting group; wherein each R^{A4c} is, independently, hydrogen, an optionally substituted aliphatic, optionally substituted heteroaliphatic, optionally substituted aryl, optionally substituted heteroaryl or a suitable amino protecting group, or two R^{A4c} groups are joined together to form a heterocyclic or heteroaryl group; and wherein each R^{A4d} is, independently, an optionally substituted aliphatic, optionally substituted heteroaliphatic, optionally substituted aryl, or an optionally substituted heteroaryl group, and

w is an integer between 0 to 5, inclusive.

In certain embodiments, the optionally substituted pyridinyl ring is of the formulae:

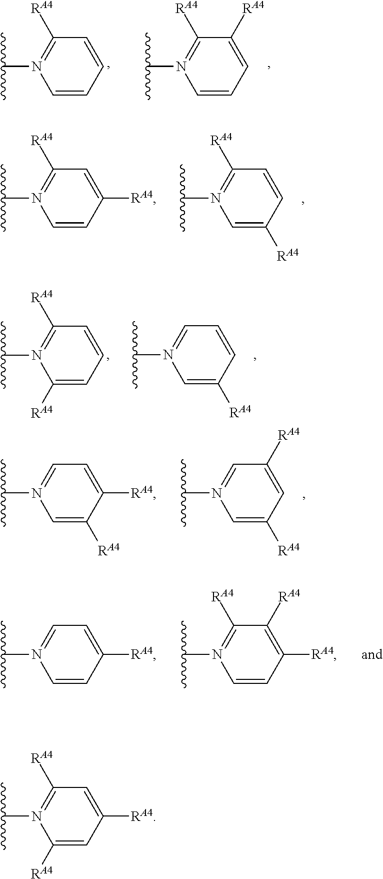

In certain embodiments, the optionally substituted pyridinyl ring is:

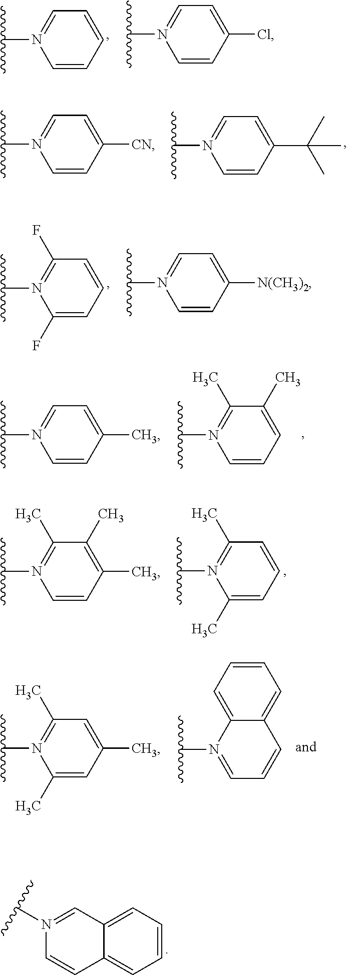

In certain embodiments, R^{L2} is —P(R^{X})₃. In certain embodiments, R^{X} is optionally substituted aliphatic. In certain embodiments, R^{X} is optionally substituted aryl. In certain embodiments, R^{X} is optionally substituted alkoxy. In certain embodiments, R^{X} is optionally substituted aryloxy. In certain embodiments, R^{L2} is —P(Me)₃. In certain embodiments, R^{L2} is —P(Et)₃. In certain embodiments, R^{L2} is —P(tert-Bu)₃. In certain embodiments, R^{L2} is —P(Cy)₃. In certain embodiments, R^{L2} is —P(Ph)₃. In certain embodiments, R^{L2} is —PMe(Ph)₂. In certain embodiments, R^{L2} is —PF₃. In certain embodiments, R^{L2} is —P(OMe)₃. In certain embodiments, R^{L2} is —P(OEt)₃. In certain embodiments, R^{L2} is —P(OPh)₃.

### Z, L, and R^{L1}

As generally defined herein, in certain embodiments, Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, —OR^{a} group or an —N(R^{c})₂ group wherein two R^{c} groups are joined to form an optionally substituted membered heterocyclic or heteroaryl ring.

In certain embodiments, R^{L1} is —N(R^{c})₂ optionally joined to Z via a linker group -L- to form a 5- to 7-membered palladacycle, wherein two R^{c} groups are joined to form an optionally substituted membered heterocyclic or heteroaryl ring.

In certain embodiments, two R^{c} groups are joined to form an optionally substituted 5-membered heterocyclic ring. Exemplary 5-membered heterocyclic rings include, but are not limited to, an optionally substituted pyrrolidinyl ring.

In certain embodiments, two R^{c} groups are joined to form an optionally substituted 5-membered heteroaryl ring. Exemplary 5-membered heteroaryl rings include, but are not limited to, an optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isoxazolyl, optionally substituted oxadiaziolyl or optionally substituted oxadiaziolyl ring.

In certain embodiments, two R^{c} groups are joined to form an optionally substituted 6-membered heterocyclic ring. Exemplary 6-membered heterocyclic rings include, but are not limited to, optionally substituted piperidinyl, optionally substituted piperazinyl or optionally substituted morpholinyl ring.

In certain embodiments, two R^{c} groups are joined to form an optionally substituted 6-membered heteroaryl ring. Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

In certain embodiments, two R^{c} groups are joined to form an optionally substituted bicyclic heteroaryl ring. Exemplary bicyclic heteroaryl rings include, but are not limited to, optionally substituted quinolinyl and optionally substituted isoquinolinyl.

In certain embodiments, two R^{c} groups are joined to form an optionally substituted pyridinyl ring. In certain embodiments, two R^{c} groups are joined to form an optionally substituted quinolinyl ring.

For example, in certain embodiments, wherein two R^{c} groups are joined to form an optionally substituted pyridinyl ring, the group provided by Z, L and R^{L1} is of the formulae:

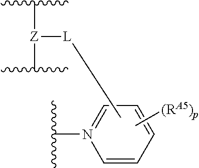

wherein:

Z is —N—;

L is -L- is selected from —C(═O)—, —C(═O)O—, —C(═O)N(R^{e3})—, —C(═NR^{e3})—,

—C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is an integer between 0 to 5, inclusive.

In certain embodiments, wherein two R^{c} groups are joined to form an optionally substituted quinolinyl ring, the group provided by Z, L and R^{L1} is of the formulae:

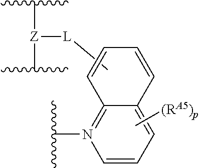

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is an integer between 0 to 5, inclusive.

In certain embodiments, -L- is —C(═O)—.

In certain embodiments, -L- is —C(═O)O—.

In certain embodiments, -L- is —C(═O)N(R^{e3})—.

In certain embodiments, -L- is —C(═NR^{e3})—.

In certain embodiments, -L- is —C(═NR^{e3})O—.

In certain embodiments, -L- is —C(═NR^{e3})N(R^{e3})—.

In certain embodiments, -L- is —S(O)₂—.

In certain embodiments, -L- is —S(O)—.

In certain embodiments, the group provided by Z, L and R^{L1} is of the formulae:

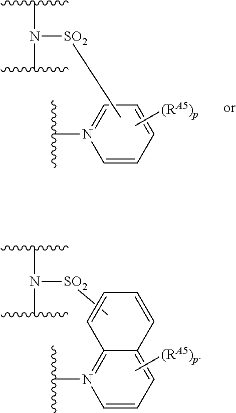

In certain embodiments, the group provided by Z, L and R^{L1} is of the formulae:

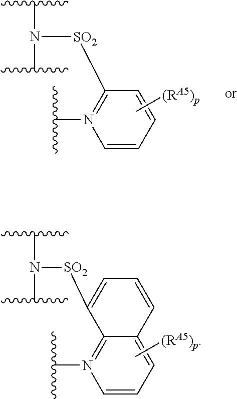

In certain embodiments, the group provided by Z, L and R^{L1} is:

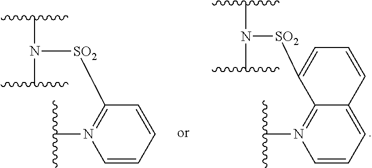

### Group Z

In certain embodiments, Z is not linked to the ligand R^{L1} as in the case of a palladium(II) complex with a bidentate ligand. As defined generally herein, in certain embodiments, Z is a bond, —O—, —S—, —C(R^{d})₂—, —C(R^{d})═C(R^{d})—, —C(R^{d})═N—, or —N(R^{e})—;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, or a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted membered heterocyclic or heteroaryl ring.

In certain embodiments, Z is a bond.

In certain embodiments, Z is —C(R^{d})₂—. In certain embodiments, Z is —CH₂—.

In certain embodiments, Z is —C(R^{d})═C(R^{d})—. In certain embodiments, Z is —CH═CH—.

In certain embodiments, Z is —C(R^{d})═N—. In certain embodiments, Z is —CH═N—

In certain embodiments, Z is —O—.

In certain embodiments, Z is —S—.

In certain embodiments, Z is —NR^{e}—.

In certain embodiments, wherein Z is —NR^{e}—, the R^{e} group is of the formula —S(O)₂R^{e1}, wherein R^{e1} is an optionally substituted aliphatic, optionally substituted heteroaliphatic, optionally substituted aryl or optionally substituted heteroaryl group. In certain embodiments, the R^{e} group is of the formula —S(O)₂R^{e1}, wherein R^{e1} is an optionally substituted aryl or optionally substituted heteroaryl group. In certain embodiments, the R^{e} group is of the formula —S(O)₂R^{e1}, wherein R^{e1} is an optionally substituted heteroaryl group. In certain embodiments, the R^{e} group is of the formula —S(O)₂R^{e1}, wherein R^{e1} is an optionally substituted aryl group.

Exemplary —S(O)₂R^{e1} groups include, but are not limited to:

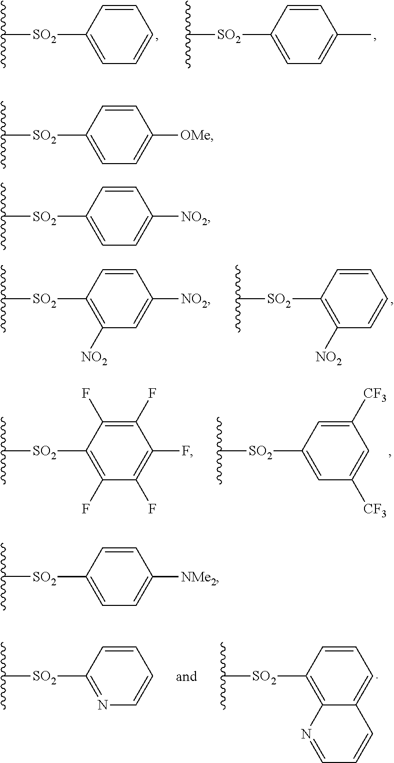

In certain embodiments, Z is of the formula:

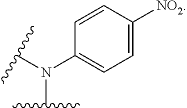 [SMILES: *N(*)c1ccc([N+](=O)[O-])cc1]

In certain embodiments, Z is of the formula:

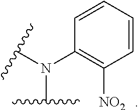 [SMILES: *N(*)c1ccccc1[N+](=O)[O-]]

In certain embodiments, Z is of the formula:

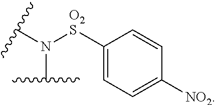 [SMILES: *N(*)S(=O)(=O)c1ccc([N+](=O)[O-])cc1]

In certain embodiments, Z is of the formula:

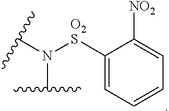 [SMILES: *N(*)S(=O)(=O)c1ccccc1[N+](=O)[O-]]

Exemplary Palladium(II) complexes

In certain embodiments, the palladium(II) complex is selected from any of the following complexes:

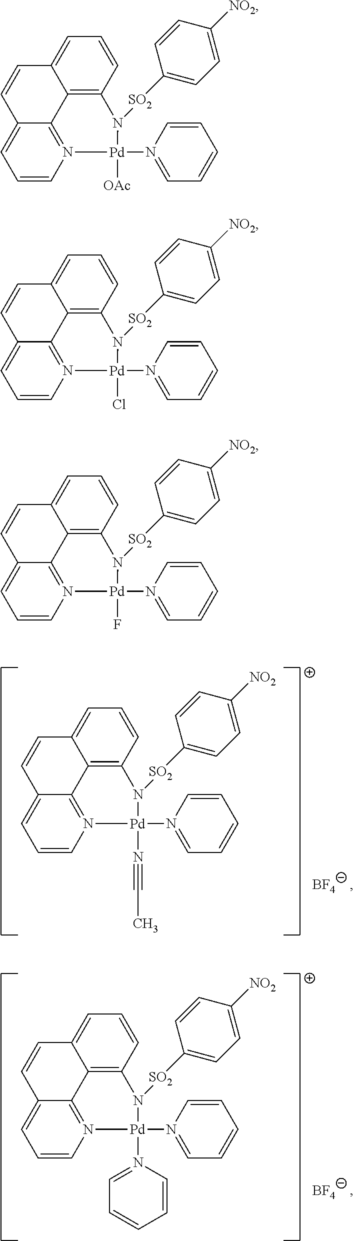

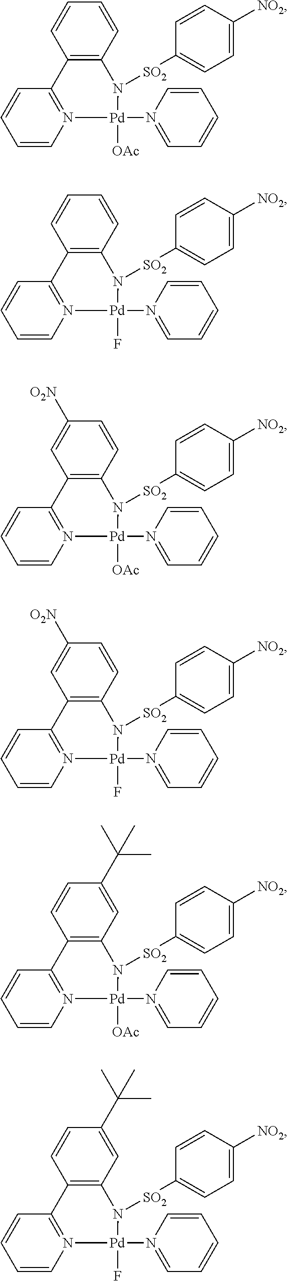

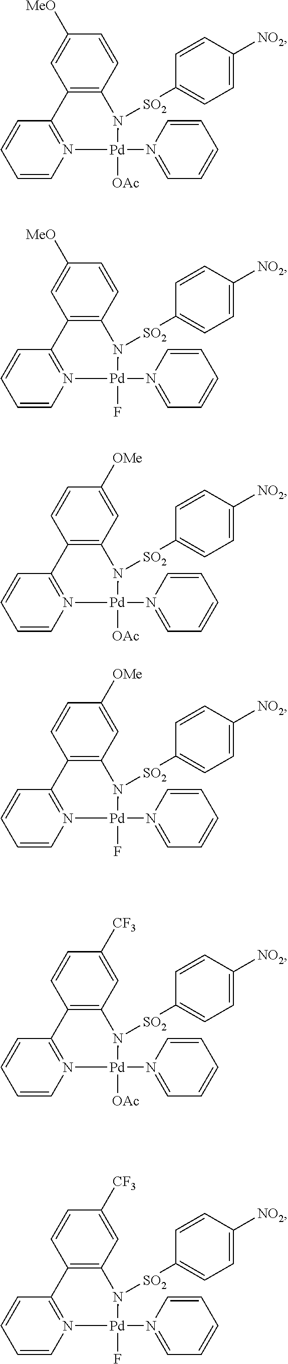

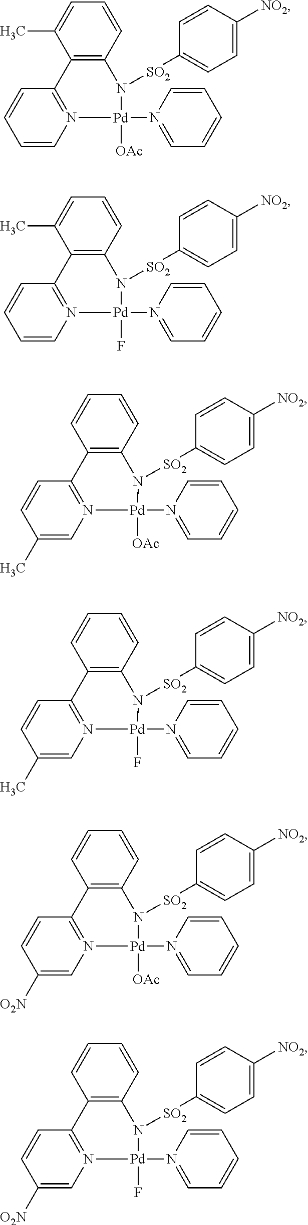

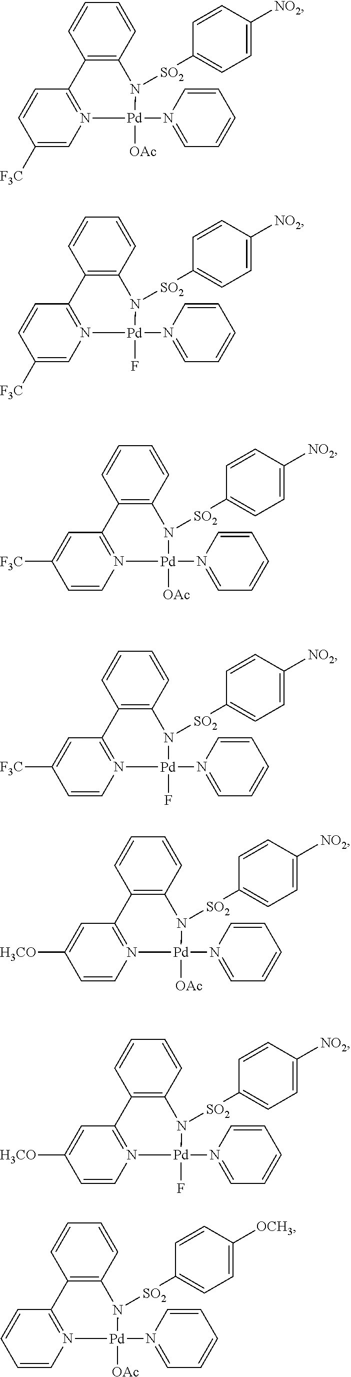

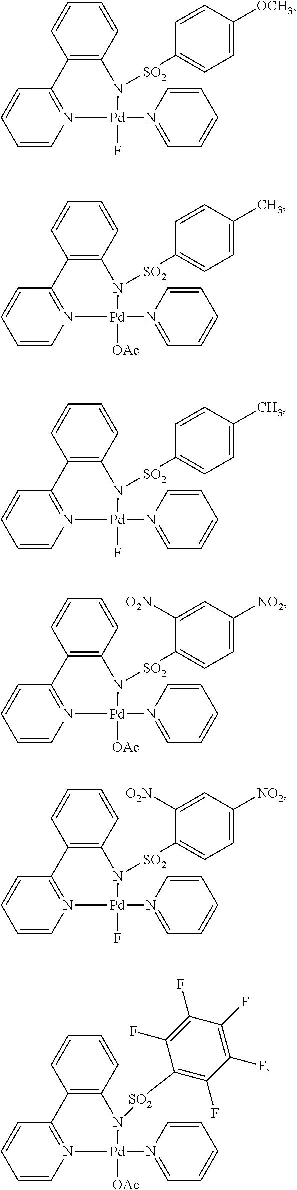

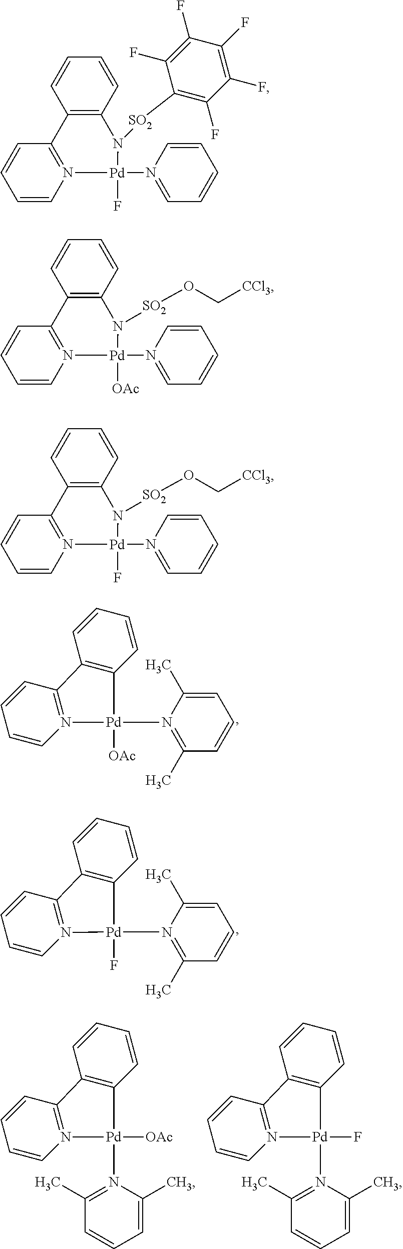

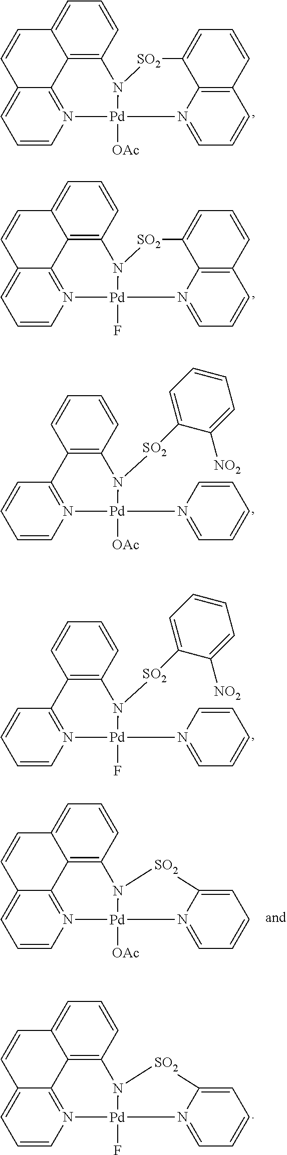

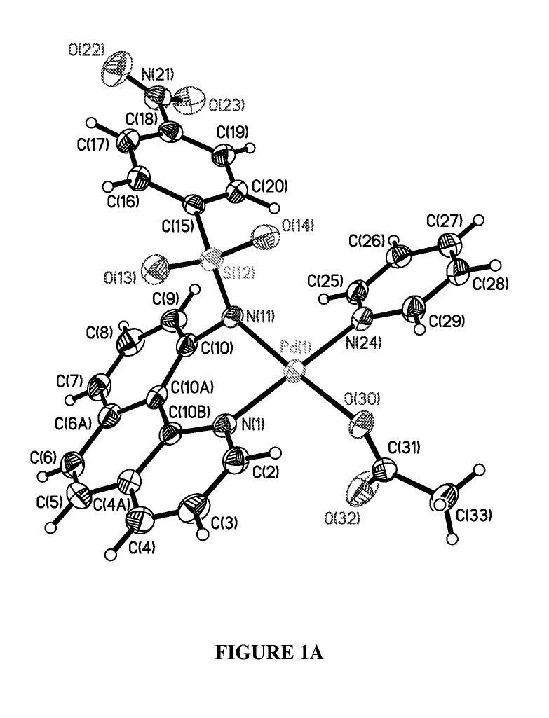

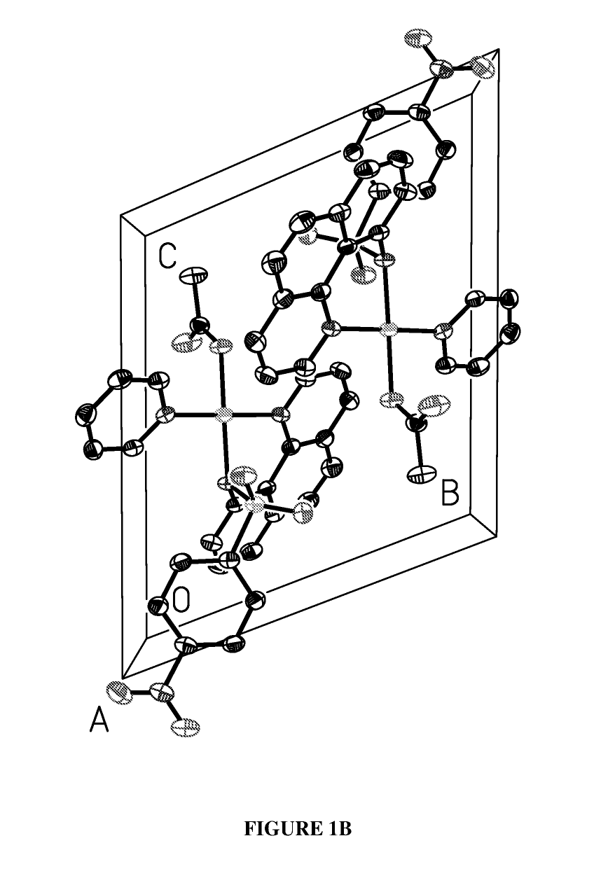

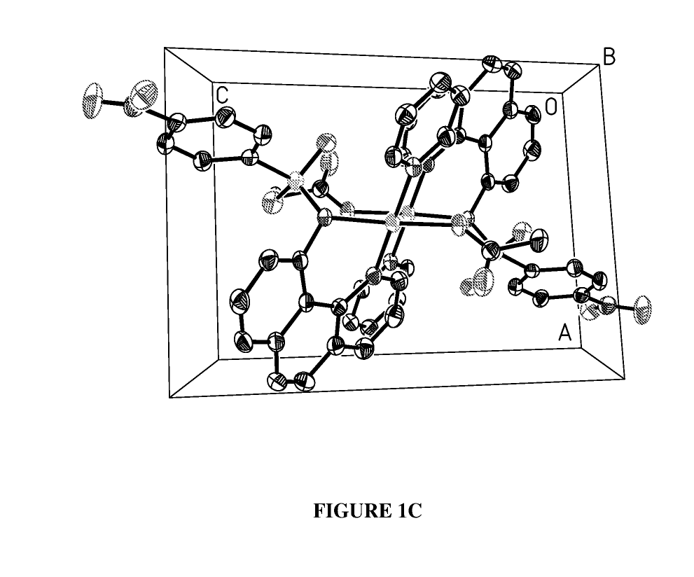

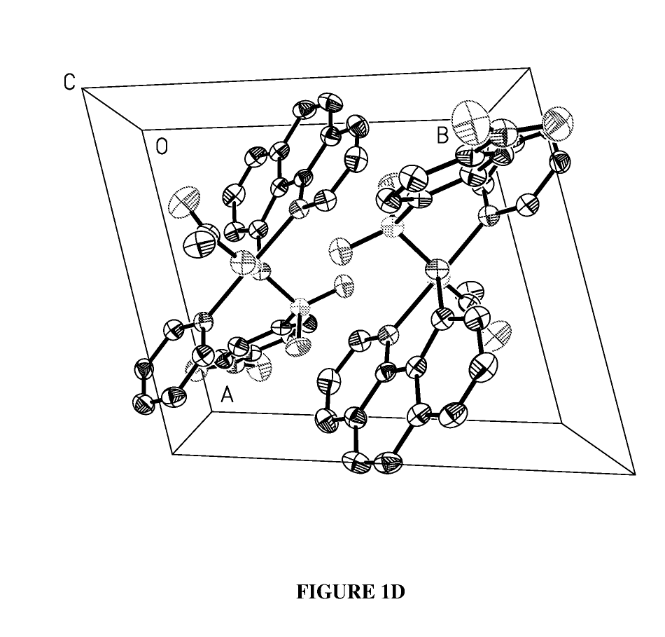

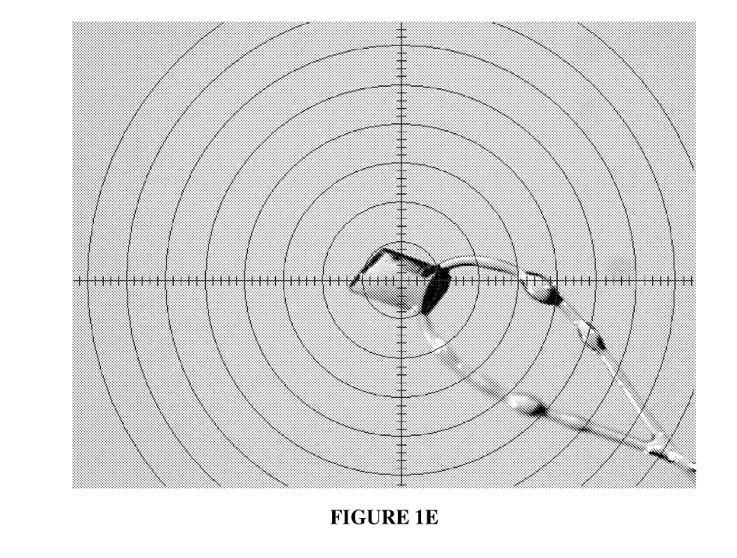

In certain embodiments, the palladium(II) complex is (i.e., the crystalline complex 1 depicted in FIG. 1A):

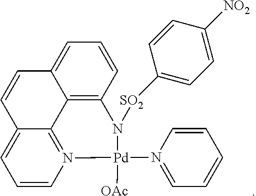

In certain embodiments, the palladium(II) complex is of the formula:

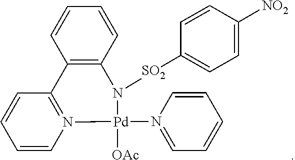

In certain embodiments, the palladium(II) complex is of the formula:

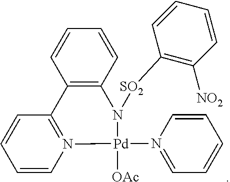

In certain embodiments, the palladium(II) complex is of the formula:

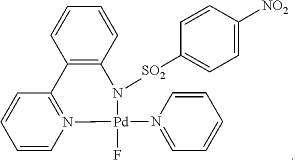

### (ii) Fluorinating Agent

As generally described herein, the process utilizes a fluorinating agent. In certain emboidments, the fluorinating agent is an electrophilic fluorinating agent. In certain embodiments, the fluorinating agent is commercially available. In certain embodiments, the electrophilic fluorinating agent is also an inorganic fluorinating agent. Exemplary electrophilic fluorinating agents include, but are not limited to, N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, an N-fluoroarylsulfonimide (e.g., N-fluorobenzenesulfonimide), N-chloromethyl-N′-fluorotriethylenediammonium bis(tetrafluoroborate) (SELECTFLUOR®), N-chloromethyl-N′-fluorotriethylenediammonium bis(hexafluorophosphate), N-chloromethyl-N′-fluorotriethylenediammonium bis(triflate), and XeF₂. In certain embodiments, the fluorinating agent is SELECTFLUOR®. In certain embodiments, the fluorinating agent is N-fluoropyridinium triflate. In certain embodiments, the fluorinating agent is N-fluoro-2,4,6-trimethylpyridinium triflate. In certain embodiments, the fluorinating agent is N-fluoro-2,4,6-trimethylpyridinium tetrafluoroborate. In certain embodiments, the fluorinating agent is N-fluorobenzenesulfonimide. In certain embodiments, the fluorinating agent is xenon difluoride.

The fluorinating agent may be enriched with a particular isoptope of fluorine. In certain embodiments, the fluorinating agent is labeled with ¹⁹F (i.e., transfers an ¹⁹F fluorine substituent to the organic compound). In certain embodiments, reaction of the ¹⁹F fluorinating agent in the process provides a fluorinated ¹⁹F-labeled organic compound.

In certain embodiments, the fluorinating agent is labeled with ¹⁸F (i.e., transfers an ¹⁸F fluorine substituent to the organic compound). In certain embodiments, reaction of the ¹⁸F fluorinating agent in the process provides a fluorinated ¹⁸F-labeled organic compound.

However, in certain embodiments, the fluorinating agent is labeled with a mixture of ¹⁸F and ¹⁹F. In certain embodiments, reaction of the mixture of ¹⁹F and ¹⁸F fluorinating agent in the process provides a mixture of fluorinated ¹⁹F-labeled organic compound and fluorinated ¹⁸F-labeled organic compound.

Any of the above fluorinated agents may be labeled as ¹⁹F or ¹⁸F.

For example, in certain embodiments, the fluorinating agent is ¹⁹F-labeled N-fluoro-N′-(chloromethyl)triethylenediamine bis(tetrafluoroborate) (SELECTFLUOR®) or ¹⁹F-labeled XeF₂. In certain embodiments, the fluorinating agent is ¹⁹F-labeled N-fluoro-N′-(chloromethyl)triethylenediamine bis(tetrafluoroborate) (SELECTFLUOR®). In certain embodiments, the fluorinating agent is ¹⁹F-labeled XeF₂.

In certain embodiments, the fluorinating agent is ¹⁸F-labeled N-fluoro-N′-(chloromethyl)triethylenediamine bis(tetrafluoroborate) (SELECTFLUOR®) or ¹⁸F-labeled XeF₂. In certain embodiments, the fluorinating agent is ¹⁸F-labeled N-fluoro-N′-(chloromethyl)triethylenediamine bis(tetrafluoroborate) (SELECTFLUOR®). In certain embodiments, the fluorinating agent is ¹⁸F-labeled XeF₂.

(iii) Boron Substituent

As generally described herein, in some embodiments the process involves fluorination of an organic compound comprising one or more boron substituents.

In certain embodiments, the organic compound comprises one boron substituent. In certain embodiments, the organic compound comprises two boron substituents.

For example, in certain embodiments, a boron substituent is a group of the formula:

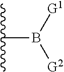

wherein G¹ and G² are, independently, —OH, —OR^{G}, or —R^{G}, each R^{G} is, independently, optionally substituted aliphatic, optionally substituted heteroaliphatic, optionally substituted aryl, or optionally substituted heteroaryl, or G¹ and G² are joined to form a 5- to 8-membered ring having at least one O atom directly attached to B, wherein the ring is comprised of carbon atoms and optionally one or more additional heteroatoms independently selected from the group consisting of N, S, and O.

As used herein, a boron substituent is intended to encompass free boronic acid substituents (i.e., wherein G¹ and G² are both —OH) and oligomeric anhydrides thereof (including, but not limited to, dimers, trimers, and tetramers, and mixtures thereof), boronic ester substituents (i.e., wherein G¹ is —OH or —OR^{G} and G² is —OR^{G}), borinic acid substituents (i.e., wherein G¹ is —OH and G² is —R^{G}), and borinic ester substituents (i.e., wherein G¹ is —OR^{G} and G² is —R^{G}).

In certain embodiments, G¹ and G² are, independently, —OH, —OR^{G}, or —R^{G}.

In certain embodiments, G¹ is —OH and G² is —OR^{G}.

In certain embodiments, G¹ is —OR^{G} and G² is —OR^{G}.

In certain embodiments, G¹ is —OH and G² is —R^{G}.

In certain embodiments, G¹ is —OR^{G} and G² is —R^{G}.

In certain embodiments, G¹ and G² are both —OH.

In certain embodiments, G¹ and G² are, independently, —OR^{G}.

In certain embodiments, G¹ and G² are, independently, —R^{G}.

In certain embodiments, G¹ and G² are joined to form a 5- to 8-membered ring.

In certain embodiments, G¹ and G² are joined to form a 5-membered ring. Exemplary 5-membered rings include, but are not limited to:

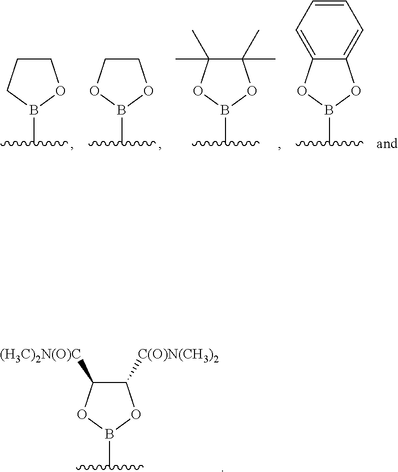

In certain embodiments, G¹ and G² are joined to form a 6-membered ring. Exemplary 6-membered rings include, but are not limited to:

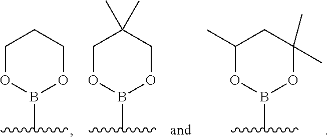

In certain embodiments, G¹ and G² are joined to form an 8-membered ring. Exemplary 8-membered rings include, but are not limited to:

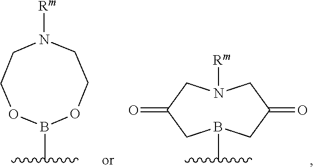

wherein R^{m} is hydrogen, a suitable amino protecting group, or an optionally substituted aliphatic, optionally substituted heteroaliphatic, optionally substituted aryl, or optionally substituted heteroaryl group.

Furthermore, as used herein, a boron substituent is also intended to encompass a trihydroxyboronate substituent.

For example, in certain embodiments, a boron substituent is a group of the formula:

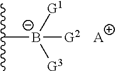

wherein G¹, G² and G³ are, independently, —OH, —OR, or —R, wherein each R is, independently, optionally substituted aliphatic, optionally substituted heteroaliphatic, optionally substituted aryl, or optionally substituted heteroaryl, and wherein A^{⊕} is a metal cation or ammonium.

Exemplary metal cations include lithium, sodium, potassium, magnesium, and calcium cations. In certain embodiments, the metal cation is a potassium cation.

Furthermore, as used herein, a boron substituent is also intended to encompass a trifluoroborate substituent.

For example, in certain embodiments, a boron substituent is a group of the formula:

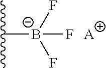

wherein A^{⊕} is a metal cation or ammonium.

Exemplary metal cations include lithium, sodium, potassium, magnesium, and calcium cations. In certain embodiments, the metal cation is a potassium cation.

### (iv) Organostannane Substituent

As generally described herein, in some embodiments the process involves fluorination of an organic compound comprising one or more organostannane substituents.

In certain embodiments, the organic compound comprises one organostannane substituent. In certain embodiments, the organic compound comprises two organostannane substituents.

In certain embodiments, the organostannane may be a trialkylstannane, e.g., trimethylstannane or tributylstannane.

### (v) Silane Substituent

As generally described herein, in some embodiments the process involves fluorination of an organic compound comprising one or more silane substituents.

In certain embodiments, the organic compound comprises one silane substituent. In certain embodiments, the organic compound comprises two silane substituents.

In certain embodiments, the silane has the formula —Si(OG⁴)₃, wherein G⁴ is an alkyl group, e.g., methyl or ethyl.

### (vi) Organic Compound

As generally described herein, the process utilizes an organic compound comprising one or more boron, organostannane or silane substituents, and provides, upon reaction with a fluorinating agent, a fluorinated organic compound wherein the boron, organostannane or silane substituent is replaced with a fluorine substituent.

An organic compound includes, but is not limited to, small organic molecules and/or large organic molecules. A small organic molecule include any molecule having a molecular weight of less than 1000 g/mol, of less than 900 g/mol, of less than 800 g/mol, of less than 700 g/mol, of less than 600 g/mol, of less than 500 g/mol, of less than 400 g/mol, of less than 300 g/mol, of less than 200 g/mol or of less than 100 g/mol. A large organic molecule include any molecule of between 1000 g/mol to 5000 g/mol, of between 1000 g/mol to 4000 g/mol, of between 1000 g/mol to 3000 g/mol, of between 1000 g/mol to 2000 g/mol, or of between 1000 g/mol to 1500 g/mol. Organic compounds include, but are not limited to, aryl compounds, heteroaryl compounds, carbocyclic compounds, heterocyclic compounds, aliphatic compounds, heteroaliphatic compounds, as well as hormones, polymers, peptides, polypeptides, proteins, glycopeptides, and the like.

In certain embodiments, an organic compound is an optionally substituted aliphatic, optionally substituted heteroaliphatic, optionally substituted aryl, or optionally substituted heteroaryl compound.

In certain embodiments, an organic compound is a polymer.

In certain embodiments, an organic compound is a peptide, polypeptide or protein, e.g., an antibody or antigen.

In certain embodiments, an organic compound is biologically active.

For example, in certain embodiments, the organic compound is an agrochemical. In certain embodiments, the organic compound is an insecticide or a pheromone of insect origin.

In certain embodiments, the organic compound is pharmaceutical agent.

For example, in certain embodiments, the organic compound is an anti-emetic, anti-coagulant, anti-platelet, anti-arrhythmic, anti-hypertensive, anti-anginal, a lipid-modifying drug, sex hormone, anti-diabetic, antibiotic, anti-viral, anti-fungal, anti-cancer, immunostimulant, immunosuppressant, anti-inflammatory, anti-rheumatic, anesthetic, analgesic, anticonvulsant, hypnotic, anxiolytic, anti-psychotic, barbituate, antidepressant, sedative, anti-obesity, antihistamine, anti-epileptic, anti-manic, opioid, anti-Parkinson, anti-Alzheimers, anti-dementia, an anti-substance dependence drug, cannabinoid, 5HT-3 antagonist, monoamine oxidase inhibitor (MAOI), selective serotonin reuptake inhibitor (SSRI) or stimulant.

In certain embodiments, an organic compound is any pharmaceutical agent approved by the United States Food and Drug Administration FDA for administration to a human (see, for example, http://www.accessdata.fda.gov/scripts/cder/drugsatfda/).

In certain embodiments, the pharmaceutical agent is an antibiotic. In certain embodiments, the pharmaceutical agent is a lipid modifying drug. In certain embodiments, the pharmaceutical agent is a CNS drug (i.e., drug acting on the Central Nervous System). CNS drugs include, but are not limited to, hypnotics, anxiolytics, anti-psychotics, barbituates, antidepressants, anti-obesity, antihistamines, anti-epileptics, anti-manics, opioids, analgesics, anti-Parkinson, anti-Alzheimers, anti-dementia, anti-substance dependance drugs, cannabinoids, 5HT-3 antagonists, monoamine oxidase inhibitors (MAOIs), selective serotonin reuptake inhibitors (SSRIs) and stimulants. Exemplary antibiotics, lipid modifying drugs and CNS drugs are provided below in Table 1.

TABLE 1

| TYPE | CLASS | DRUG NAME |
|---|---|---|
| Antibiotic | Beta-lactam | AMOXICILLIN |
| Antibiotic | aminoglycoside | AMIKACIN |
| Antibiotic | Beta-lactam | AMPICILLIN |
| Antibiotic | Beta-lactam | AZTREONAM |
| Antibiotic | Carboxypenicillin | CARBENICILLIN |
| Antibiotic | 2^{nd} generation cephalosporin | CEFACLOR |
| Antibiotic | cephalosporin | CEFAMANDOLE |
| Antibiotic | cephalosporin | CEFAZOLIN |
| Antibiotic | cephalosporin | CEFEPIME |
| Antibiotic | 3^{rd} generation cephalosporin | CEFIXIME |
| Antibiotic | cephamycin | CEFMETAZOLE |
| Antibiotic | 2^{nd} generation cephalosporin | CEFONICID |
| Antibiotic | 3^{rd} generation cephalosporin | CEFOPERAZONE |
| Antibiotic | 3^{rd} generation cephalosporin | CEFOTAXIME |
| Antibiotic | 2^{nd} generation cephalosporin | CEFOXITIN |
| Antibiotic | 3^{rd} generation cephalosporin | CEFTAZIDIME |
| Antibiotic | cephalosporin | CEFTIZOXIME |
| Antibiotic | 3^{rd} generation cephalosporin | CEFTRIAXONE |
| Antibiotic | 2^{nd} generation cephalosporin | CEFUROXIME |
| Antibiotic | cephalosporin | CEPHALOTHIN |
| Antibiotic | fluoroquinolone | CIPROFLOXACIN |
| Antibiotic | lincosamide | CLINDAMYCIN |
| Antibiotic | cephalosporin | CEFOTETAN |
| Antibiotic | macrolide | ERYTHROMYCIN |
| Antibiotic | aminoglycoside | GENTAMICIN |
| Antibiotic | Beta-lactam | IMIPENEM |
| Antibiotic | aminoglycoside | KANAMYCIN |
| Antibiotic | Beta-lactam | MEROPENEM |
| Antibiotic | beta-lactam | METHICILLIN |
| Antibiotic | nitroimidazole | METRONIDAZOLE |
| Antibiotic | beta-lactam | NAFCILLIN |
| Antibiotic | quinolone | NALIDIXIC ACID |
| Antibiotic | aminoglycoside | NETILMICIN |
| Antibiotic | | NITROFURANTOIN |
| Antibiotic | fluoroquinolone | NORFLOXACIN |
| Antibiotic | fluoroquinolone | OFLOXACIN |
| Antibiotic | beta-lactam | OXACILLIN |
| Antibiotic | beta-lactam | PIPERACILLIN |
| Antibiotic | rifamycin | RIFAMPIN |
| Antibiotic | sulfa drug | SULFISOXAZOLE |
| Antibiotic | glycopeptide | TRIMETHOPRIM |
| Antibiotic | glycopeptide | TEICOPLANIN |
| Antibiotic | carboxypenicillin | TICARCILLIN |
| Antibiotic | glycopeptide | TEICOPLANIN |
| Antibiotic | tetracyclines | TETRACYCLINE |
| Antibiotic | carboxypenicillin | TICARCILLIN |
| Antibiotic | aminoglycoside | TOBRAMYCIN |
| Antibiotic | glycopeptide | VANCOMYCIN |
| Lipid modifying | Statins | ATORVASTATIN (LIPITOR) |
| Lipid modifying | Statins | CERIVASTATIN |
| | | (LIPOBAY) |
| Lipid modifying | Statins | FLUVASTATIN |
| | | (LESCOL) |
| Lipid modifying | Statins | LOVASTATIN (STATOSAN) |
| Lipid modifying | Statins | PITAVASTATIN |
| Lipid modifying | Statins | PRAVASTATIN (PRAVACHOL) |
| Lipid modifying | Statins | ROSUVASTATIN (CRESTOR) |
| Lipid modifying | Statins | SIMVASTATIN (ZOCOR) |
| Lipid modifying | Fibrates | CLOFIBRATE |
| Lipid modifying | Fibrates | GEMFIBROZIL (LOPID) |
| Lipid modifying | Fibrates | FENOFIBRATE (TRICORE) |
| Lipid modifying | Fibrates | SIMFIBRATE |
| Lipid modifying | Fibrates | RONIFIBRATE |
| Lipid modifying | Fibrates | CIPROFIBRATE |
| Lipid modifying | Fibrates | CLOFIBRIDE |
| Lipid modifying | Bile acid sequestrants | COLESTYRAMINE (QUESTRAN) |
| Lipid modifying | Bile acid sequestrants | COLESTIPOL |
| Lipid modifying | Bile acid sequestrants | COLEXTRAN |
| Lipid modifying | Bile acid sequestrants | COLESEVELAM |
| Lipid modifying | Niacin | NIACIN |
| Lipid modifying | Niacin derivative | NICOFURANOSE |
| Lipid modifying | Other | DEXTROTHYROXINE |
| Lipid modifying | Other | PROBUCOL |
| Lipid modifying | Other | TIADENOL |
| Lipid modifying | Other | BENFLUOREX |
| Lipid modifying | Other | MEGLUTOL |
| Lipid modifying | Other | MAGNESIUM PYRIDOXAL 5-PHOSPHATE |
| | | GLUTAMATE |
| Lipid modifying | Other | EZETIMIBE (ZETIA) |
| CNS | Hypnotics | NITRAZEPAM |
| CNS | Hypnotics | NITRAZEPAM |
| CNS | Hypnotics | FLUNITRAZEPAM |
| CNS | Hypnotics | FLURAZEPAM |
| CNS | Hypnotics | LOPRAZOLAM |
| CNS | Hypnotics | TEMAZEPAM |
| CNS | Hypnotics | ZALEPLON |
| CNS | Hypnotics | ZOLPIDEM TARTRATE |
| CNS | Hypnotics | ZOPICLONE |
| CNS | Hypnotics | CHLORAL HYDRATE |
| CNS | Hypnotics | CLOMETHIAZOLE |
| CNS | Hypnotics | PROMETHAZINE HYDROCHLORIDE |
| CNS | Anxiolytics | DIAZEPAM |
| CNS | Anxiolytics | ALPRAZOLAM |
| CNS | Anxiolytics | CHLORDIAZEPOXIDE |
| CNS | Anxiolytics | CLORAZEPATE DIPOTASSIUM |
| CNS | Anxiolytics | LORAZEPAM |
| CNS | Anxiolytics | OXAZEPAM |
| CNS | Anxiolytics | BUSPIRONE HYDROCHLORIDE |
| CNS | Anxiolytics | MEPROBAMATE |
| CNS | Anxiolytics | BETA-BLOCKERS |
| CNS | Barbiturates | BARBITURATES |
| CNS | Barbiturates | BARBITURATES |
| CNS | Barbiturates | BARBITURATES |
| CNS | Antipsychotic drugs | BENPERIDOL |
| CNS | Antipsychotic drugs | CHLORPROMAZINE HYDROCHLORIDE |
| CNS | Antipsychotic drugs | FLUPENTIXOL |
| CNS | Antipsychotic drugs | FLUPHENAZINE HYDROCHLORIDE |
| CNS | Antipsychotic drugs | FLUPHENAZINE HYDROCHLORIDE |
| CNS | Antipsychotic drugs | HALOPERIDOL |
| CNS | Antipsychotic drugs | LEVOMEPROMAZINE/METHOTRIMEPRAZINE |
| CNS | Antipsychotic drugs | LOXAPINE |
| CNS | Antipsychotic drugs | OXYPERTINE |
| CNS | Antipsychotic drugs | PERICYAZINE |
| CNS | Antipsychotic drugs | PERPHENAZINE |
| CNS | Antipsychotic drugs | PIMOZIDE |
| CNS | Antipsychotic drugs | PROCHLORPERAZINE |
| CNS | Antipsychotic drugs | PROMAZINE HYDROCHLORIDE |
| CNS | Antipsychotic drugs | SULPIRIDE |
| CNS | Antipsychotic drugs | THIORIDAZINE |
| CNS | Antipsychotic drugs | TRIFLUOPERAZINE |
| CNS | Antipsychotic drugs | ZUCLOPENTHIXOL ACETATE |
| CNS | Antipsychotic drugs | ZUCLOPENTHIXOL DIHYDROCHLORIDE |
| CNS | Atypical antipsychotics | AMISULPRIDE |
| CNS | Atypical antipsychotics | CLOZAPINE |
| CNS | Atypical antipsychotics | OLANZAPINE |
| CNS | Atypical antipsychotics | QUETIAPINE |
| CNS | Atypical antipsychotics | RISPERIDONE |
| CNS | Atypical antipsychotics | ZOTEPINE |
| CNS | Antipsychotic | FLUPENTIXOL DECANOATE |
| CNS | Antipsychotic | HALOPERIDOL DECANOATE |
| CNS | Antipsychotic | PIPOTIAZINE PALMITATE |
| CNS | Antipsychotic | ZUCLOPENTHIXOL DECANOATE |
| CNS | Antipsychotic | ZUCLOPENTHIXOL DECANOATE |
| CNS | Antipsychotic | ZYPREXA |
| CNS | Antimanic drugs | BENZODIAZEPINES |
| CNS | Antimanic drugs | ANTIPSYCHOTIC DRUGS |
| CNS | Antimanic drugs | CARBAMAZEPINE |
| CNS | Antimanic drugs | VALPROIC ACID |
| CNS | Tricyclic antidepressant drugs | AMITRIPTYLINE HYDROCHLORIDE |
| CNS | Tricyclic antidepressant drugs | AMOXAPINE |
| CNS | Tricyclic antidepressant drugs | CLOMIPRAMINE HYDROCHLORIDE |
| CNS | Tricyclic antidepressant drugs | DOSULEPIN HYDROCHLORIDE/DOTHIEPIN |
| | | HYDROCHLORIDE |
| CNS | Tricyclic antidepressant drugs | DOXEPIN |
| CNS | Tricyclic antidepressant drugs | IMIPRAMINE HYDROCHLORIDE |
| CNS | Tricyclic antidepressant drugs | IMIPRAMINE HYDROCHLORIDE |
| CNS | Tricyclic antidepressant drugs | LOFEPRAMINE |
| CNS | Tricyclic antidepressant drugs | NORTRIPTYLINE |
| CNS | Tricyclic antidepressant drugs | TRIMIPRAMINE |
| CNS | Related antidepressant | MAPROTILINE HYDROCHLORIDE |
| CNS | Related antidepressant | MIANSERIN HYDROCHLORIDE |
| CNS | Related antidepressant | TRAZODONE HYDROCHLORIDE |
| CNS | Related antidepressant | TRAZODONE HYDROCHLORIDE |
| CNS | Antidepressant | ESCITALOPRAM OXALATE (LEXPRO) |
| CNS | Monoamine-oxidase inhibitors | PHENELZINE |
| | (MAOIs) | |
| CNS | Monoamine-oxidase inhibitors | ISOCARBOXAZID |
| | (MAOIs) | |
| CNS | Monoamine-oxidase inhibitors | TRANYLCYPROMINE |
| | (MAOIs) | |
| CNS | Reversible MAOIs | MOCLOBEMIDE |
| CNS | Selective serotonin re-uptake | CITALOPRAM |
| | inhibitors | |
| CNS | Selective serotonin re-uptake | FLUOXETINE |
| | inhibitors | |
| CNS | Selective serotonin re-uptake | FLUVOXAMINE MALEATE |
| | inhibitors | |
| CNS | Selective serotonin re-uptake | PAROXETINE (Paxil) |
| | inhibitors | |
| CNS | Selective serotonin re-uptake | SERTRALINE |
| | inhibitors | |
| CNS | Other antidepressant drugs | FLUPENTIXOL |
| CNS | Other antidepressant drugs | MIRTAZAPINE |
| CNS | Other antidepressant drugs | NEFAZODONE HYDROCHLORIDE |
| CNS | Other antidepressant drugs | REBOXETINE |
| CNS | Other antidepressant drugs | TRYPTOPHAN (L-Tryptophan) |
| CNS | Other antidepressant drugs | VENLAFAXINE |
| CNS | Central nervous system | DEXAMFETAMINE SULPHATE |
| | stimulants | |
| CNS | Central nervous system | METHYLPHENIDATE HYDROCHLORIDE |
| | stimulants | |
| CNS | Central nervous system | METHYLPHENIDATE HYDROCHLORIDE |
| | stimulants | |
| CNS | Central nervous system | MODAFINIL |
| | stimulants | |
| CNS | Anti-obesity drugs acting on the | ORLISTAT |
| | gastro-intestinal tract | |
| CNS | Anti-obesity drugs (Centrally | SIBUTRAMINE HYDROCHLORIDE |
| | acting appetite suppressants) | |
| CNS | Antihistamines | CINNARIZINE |
| CNS | Antihistamines | CYCLIZINE |
| CNS | Antihistamines | MECLOZINE HYDROCHLORIDE |
| CNS | Antihistamines | PROMETHAZINE HYDROCHLORIDE |
| CNS | Antihistamines | PROMETHAZINE TEOCLATE |
| CNS | Phenothiazines and related drugs | CHLORPROMAZINE HYDROCHLORIDE |
| CNS | Phenothiazines and related drugs | PERPHENAZINE |
| CNS | Phenothiazines and related drugs | PROCHLORPERAZINE |
| CNS | Phenothiazines and related drugs | TRIFLUOPERAZINE |
| CNS | Domperidone and | DOMPERIDONE |
| | metoclopramide | |
| CNS | Domperidone and | METOCLOPRAMIDE HYDROCHLORIDE |
| | metoclopramide | |
| CNS | Domperidone and | METOCLOPRAMIDE HYDROCHLORIDE |
| | metoclopramide | |
| CNS | 5HT3 antagonists | GRANISETRON |
| CNS | 5HT3 antagonists | ONDANSETRON |
| CNS | 5HT3 antagonists | TROPISETRON |
| CNS | Cannabinoid | NABILONE |
| CNS | Non-opioid analgesics | ASPIRIN (Acetylsalicylic Acid) |
| CNS | Non-opioid analgesics | PARACETAMOL (Acetaminophen) |
| CNS | Opioid analgesics | MORPHINE |
| CNS | Opioid analgesics | BUPRENORPHINE |
| CNS | Opioid analgesics | CODEINE PHOSPHATE |
| CNS | Opioid analgesics | DEXTROMORAMIDE |
| CNS | Opioid analgesics | DEXTROPROPOXYPHENE HYDROCHLORIDE |
| CNS | Opioid analgesics | DIAMORPHINE HYDROCHLORIDE (Heroin |
| | | Hydrochloride) |
| CNS | Opioid analgesics | DIHYDROCODEINE TARTRATE |
| CNS | Opioid analgesics | DIPIPANONE HYDROCHLORIDE |
| CNS | Opioid analgesics | FENTANYL |
| CNS | Opioid analgesics | HYDROMORPHONE HYDROCHLORIDE |
| CNS | Opioid analgesics | MEPTAZINOL |
| CNS | Opioid analgesics | METHADONE HYDROCHLORIDE |
| CNS | Opioid analgesics | NALBUPHINE HYDROCHLORIDE |
| CNS | Opioid analgesics | OXYCODONE HYDROCHLORIDE |
| CNS | Opioid analgesics | PENTAZOCINE |
| CNS | Opioid analgesics | PETHIDINE HYDROCHLORIDE |
| CNS | Opioid analgesics | PETHIDINE HYDROCHLORIDE |
| CNS | Opioid analgesics | PHENAZOCINE HYDROBROMIDE |
| CNS | Opioid analgesics | TRAMADOL HYDROCHLORIDE |
| CNS | Neuropathic pain | DEXTROPROPOXYPHENE |
| CNS | Neuropathic pain | METHADONE |
| CNS | Neuropathic pain | OXYCODONE |
| CNS | Neuropathic pain | AMITRIPTYLINE |
| CNS | Neuropathic pain | NORTRIPTYLINE |
| CNS | Neuropathic pain | GABAPENTIN |
| CNS | Neuropathic pain | SODIUM VALPROATE |
| CNS | Neuropathic pain | PHENYTOIN |
| CNS | Neuropathic pain | KETAMINE |
| CNS | Neuropathic pain (Trigeminal | CARBAMAZEPINE |
| | neuralgia) | |
| CNS | Neuropathic pain (Trigeminal | OXCARBAZEPINE |
| | neuralgia) | |
| CNS | Neuropathic pain (Trigeminal | GABAPENTIN |
| | neuralgia) | |
| CNS | Neuropathic pain (Trigeminal | LAMOTRIGINE |
| | neuralgia) | |
| CNS | Neuropathic pain (Trigeminal | FOSPHENYTOIN SODIUM |
| | neuralgia) | |
| CNS | Neuropathic pain (Postherpetic | AMITRIPTYLINE |
| | neuralgia) | |
| CNS | Neuropathic pain (Postherpetic | GABAPENTIN |
| | neuralgia) | |
| CNS | Analgesics | MEPROBAMATE |
| CNS | Analgesics | PARACETAMOL |
| CNS | Analgesics | METHIONINE (CO-METHIAMOL) |
| CNS | Analgesics | DIHYDROCODEINE TARTRATE |
| CNS | Analgesics | IBUPROFEN |
| CNS | Analgesics | FLURBIPROFEN |
| CNS | Analgesics | DICLOFENAC POTASSIUM |
| CNS | Analgesics | NAPROXEN |
| CNS | Analgesics | TOLFENAMIC ACID |
| CNS | 5HT1 agonists | ALMOTRIPTAN |
| CNS | 5HT1 agonists | NARATRIPTAN |
| CNS | 5HT1 agonists | RIZATRIPTAN |
| CNS | 5HT1 agonists | SUMATRIPTAN |
| CNS | 5HT1 agonists | ZOLMITRIPTAN |
| CNS | Ergot alkaloids | ERGOTAMINE TARTRATE |
| CNS | Ergot alkaloids | ERGOTAMINE TARTRATE |
| CNS | Other drugs | ISOMETHEPTENE MUCATE |
| CNS | Other drugs | Pizotifen |
| CNS | Other drugs | PIZOTIFEN |
| CNS | Other drugs | CLONIDINE HYDROCHLORIDE |
| CNS | Other drugs | METHYSERGIDE |
| CNS | Antiepileptics (control of | CARBAMAZEPINE |
| | Epilepsy) | |
| CNS | Antiepileptics (control of | CARBAMAZEPINE |
| | Epilepsy) | |
| CNS | Antiepileptics (control of | OXCARBAZEPINE |
| | Epilepsy) | |
| CNS | Antiepileptics (control of | ETHOSUXIMIDE |
| | Epilepsy) | |
| CNS | Antiepileptics (control of | ETHOSUXIMIDE |
| | Epilepsy) | |
| CNS | Antiepileptics (control of | GABAPENTIN |
| | Epilepsy) | |
| CNS | Antiepileptics (control of | LAMOTRIGINE |
| | Epilepsy) | |
| CNS | Antiepileptics (control of | LEVETIRACETAM |
| | Epilepsy) | |
| CNS | Antiepileptics (control of | PHENOBARBITAL (Phenobarbitone) |
| | Epilepsy) | |
| CNS | Antiepileptics (control of | PRIMIDONE |
| | Epilepsy) | |
| CNS | Antiepileptics (control of | PHENYTOIN |
| | Epilepsy) | |
| CNS | Antiepileptics (control of | TIAGABINE |
| | Epilepsy) | |
| CNS | Antiepileptics (control of | TOPIRAMATE |
| | Epilepsy) | |
| CNS | Antiepileptics (control of | SODIUM VALPROATE |
| | Epilepsy) | |
| CNS | Antiepileptics (control of | SODIUM VALPROATE |
| | Epilepsy) | |
| CNS | Antiepileptics (control of | VALPROIC ACID |
| | Epilepsy) | |
| CNS | Antiepileptics (control of | VIGABATRIN |
| | Epilepsy) | |
| CNS | Antiepileptics (control of | CLOBAZAM |
| | Epilepsy) | |
| CNS | Antiepileptics (control of | CLONAZEPAM |
| | Epilepsy) | |
| CNS | Antiepileptics (control of | ACETAZOLAMIDE |
| | Epilepsy) | |
| CNS | Antiepileptics (control of | PIRACETAM |
| | Epilepsy) | |
| CNS | Antiepileptics (control of Status | DIAZEPAM |
| | Epilepticus) | |
| CNS | Antiepileptics (control of Status | CLONAZEPAM |
| | Epilepticus) | |
| CNS | Antiepileptics (control of Status | FOSPHENYTOIN SODIUM |
| | Epilepticus) | |
| CNS | Antiepileptics (control of Status | LORAZEPAM |
| | Epilepticus) | |
| CNS | Antiepileptics (control of Status | PARALDEHYDE |
| | Epilepticus) | |
| CNS | Antiepileptics (control of Status | PHENYTOIN |
| | Epilepticus) | |
| CNS | Antiepileptics (control of Status | PHENYTOIN |
| | Epilepticus) | |
| CNS | Dopaminergic drugs used in | LEVODOPA |
| | parkinsonism | |
| CNS | Dopaminergic drugs used in | CO-BENELDOPA |
| | parkinsonism | |
| CNS | Dopaminergic drugs used in | CO-CARELDOPA |
| | parkinsonism | |
| CNS | Dopaminergic drugs used in | AMANTADINE HYDROCHLORIDE |
| | parkinsonism | |
| CNS | Dopaminergic drugs used in | BROMOCRIPTINE |
| | parkinsonism | |
| CNS | Dopaminergic drugs used in | BROMOCRIPTINE |
| | parkinsonism | |
| CNS | Dopaminergic drugs used in | CABERGOLINE |
| | parkinsonism | |
| CNS | Dopaminergic drugs used in | ENTACAPONE |
| | parkinsonism | |
| CNS | Dopaminergic drugs used in | LISURIDE MALEATE (Lysuride Maleate) |
| | parkinsonism | |
| CNS | Dopaminergic drugs used in | PERGOLIDE |
| | parkinsonism | |
| CNS | Dopaminergic drugs used in | PRAMIPEXOLE |
| | parkinsonism | |
| CNS | Dopaminergic drugs used in | ROPINIROLE |
| | parkinsonism | |
| CNS | Dopaminergic drugs used in | SELEGILINE HYDROCHLORIDE |
| | parkinsonism | |
| CNS | Antimuscarinic drugs used in | BENZATROPINE MESILATE |
| | parkinsonism | |
| CNS | Antimuscarinic drugs used in | BIPERIDEN HYDROCHLORIDE |
| | parkinsonism | |
| CNS | Antimuscarinic drugs used in | ORPHENADRINE HYDROCHLORIDE |
| | parkinsonism | |
| CNS | Antimuscarinic drugs used in | ORPHENADRINE HYDROCHLORIDE |
| | parkinsonism | |
| CNS | Antimuscarinic drugs used in | PROCYCLIDINE HYDROCHLORIDE |
| | parkinsonism | |
| CNS | Antimuscarinic drugs used in | TRIHEXYPHENIDYL |
| | parkinsonism | HYDROCHLORIDE/BENZHEXOL |
| | | HYDROCHLORIDE |
| CNS | Drugs used in essential tremor, | HALOPERIDOL |
| | chorea, tics, and related disorders | |
| CNS | Drugs used in essential tremor, | PIRACETAM |
| | chorea, tics, and related disorders | |
| CNS | Drugs used in essential tremor, | RILUZOLE |
| | chorea, tics, and related disorders | |
| CNS | Drugs used in essential tremor, | TETRABENAZINE |
| | chorea, tics, and related disorders | |
| CNS | Alcohol dependence | ACAMPROSATE CALCIUM |
| CNS | Alcohol dependence | DISULFIRAM |
| CNS | Cigarette smoking | BUPROPION |
| CNS | Cigarette smoking | NICOTINE |
| CNS | Opioid dependence | BUPRENORPHINE |
| CNS | Opioid dependence | LOFEXIDINE HYDROCHLORIDE |
| CNS | Opioid dependence | METHADONE HYDROCHLORIDE |
| CNS | Opioid dependence | NALTREXONE HYDROCHLORIDE |
| CNS | Drugs for dementia | DONEPEZIL HYDROCHLORIDE |
| CNS | Drugs for dementia | GALANTAMINE |
| CNS | Drugs for dementia | RIVASTIGMINE |

In certain embodiments, the organic compound, after fluorination, is biologically active. In certain embodiments, the organic compound, prior to fluorinated, is also biologically active.

In certain embodiments, the process provides after fluorination of the organic compound a known biologically active fluorinated compound, such as a fluorinated agrochemical or fluorinated pharmaceutical agent.

For example, in certain embodiments, the process provides after fluorination of the organic compound the known fluorinated pharmaceutical agent LIPITOR:

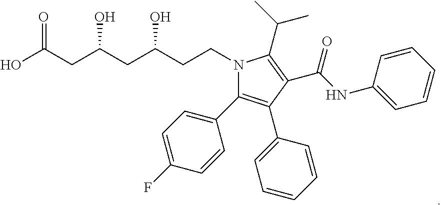 [SMILES: CC(C)c1c(C(=O)Nc2ccccc2)c(-c2ccccc2)c(-c2ccc(F)cc2)n1CC[C@@H](O)C[C@@H](O)CC(=O)O]

In certain embodiments, the process provides after fluorination of the organic compound the known fluorinated pharmaceutical agent PAXIL:

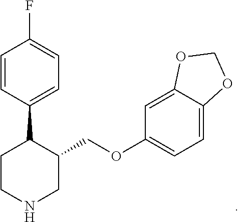

In certain embodiments, the process provides after fluorination of the organic compound the known fluorinated pharmaceutical agent LEXAPRO:

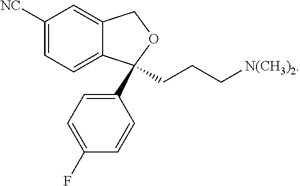 [SMILES: CN(C)CCC[C@@]1(c2ccc(F)cc2)OCc2cc(C#N)ccc21]

However, in certain embodiments, the process provides after fluorination of the organic compound a new biologically active fluorinated compound, such as a fluorinated derivative of a known agrochemical or pharmaceutical agent. In this context, a “fluorinated derivative of a known compound” is a known compound which is labeled with fluorine (i.e., one or more substituents of a known compound are replaced with fluorine).

For example, in certain embodiments, the process provides after fluorination of the organic compound a fluorinated derivative of the pharmaceutical agent vancomycin:

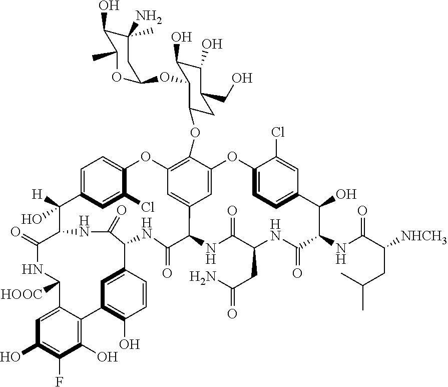

In certain embodiments, the process provides after fluorination of the organic compound a fluorinated derivative of the pharmaceutical agent MORPHINE:

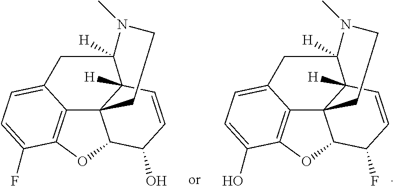 [SMILES: CN1CC[C@]23c4c5ccc(F)c4O[C@H]2[C@@H](O)C=C[C@H]3[C@H]1C5 ; "or" ; CN1CC[C@]23c4c5ccc(O)c4O[C@H]2[C@@H](F)C=C[C@H]3[C@H]1C5 ; "."]

In certain embodiments, the process provides after fluorination of the organic compound a fluorinated derivative of the pharmaceutical agent ZYPREXA:

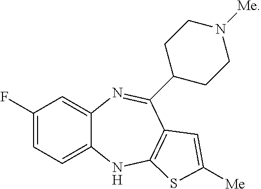 [SMILES: Cc1cc2c(s1)Nc1ccc(F)cc1N=C2C1CCN(C)CC1]

### (v) Intermediate Palladium(II) Complex

An intermediate palladium complex may be formed during the process. The intermediate complex comprises the palladium(II) complex and the organic compound to be fluorinated. The intermediate forms by addition of the organic compound comprising one or more boron, organostannane or silane substituents to the palladium complex, wherein one boron, organostannane or silane is exchanged with palladium. The intermediate is typically formed by transmetallation of the acetato form of the palladium complex since it has been found to proceed quickly and in high yield. Other forms such as the chloro form or other halogen forms may be used as well.

Thus, in certain embodiments, the process of step (i) further comprises providing an intermediate of the palladium(II) complex and the organic compound (“an intermediate palladium complex”). In certain embodiments, the process of step (i) further comprises isolating the intermediate palladium(II) complex.

As used herein, an intermediate palladium(II) complex is any palladium(II) complex, as described herein, with the proviso that at least one ligand R^{L1} or R^{L2} is an organic compound, as described herein, coordinated to the palladium by a carbon atom.

In certain embodiments, the intermediate palladium complex is any palladium complex of the above formulae, with the proviso that R^{L2} is an organic compound coordinated to the palladium by a carbon atom, and R^{L1} is selected from halogen, —OR^{a}, —SR^{b}, or —N(R^{c})₂. In certain embodiments, R^{L2} is an organic compound coordinated to the palladium by a carbon atom, and R^{L1} is a neutral ligand.

For example, in certain embodiments, the intermediate palladium complex is of the formula (II):

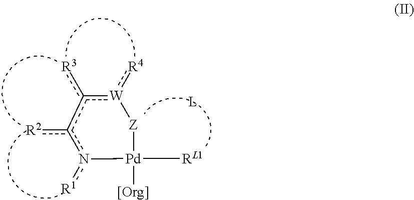

wherein Pd, [symbol] , [symbol] , L, W, R^{L1}, R^{L2}, Z, R¹, R², R³ and R⁴ are as defined above and herein; and

[Org] is an organic compound, as described herein, coordinated to Pd by a carbon atom.

In certain embodiments, the intermediate palladium complex is of the formula (II-a):

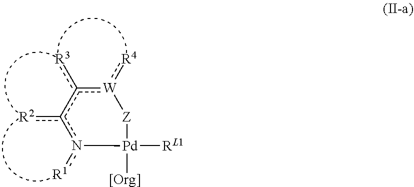

wherein Pd, [symbol] , [symbol] , W, R^{L1}, R^{L2}, Z, R¹, R², R³ and R⁴ are as defined above and herein.

In certain embodiments, the intermediate palladium complex is of the formula (II-b):

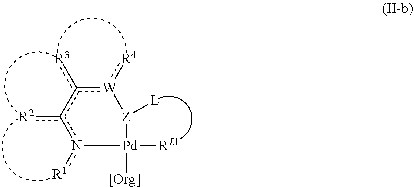

wherein Pd, [symbol] , [symbol] , [symbol] , L, W, R^{L1}, R^{L2}, Z, R¹, R², R³ and R⁴ are as defined above and herein.

As depicted above, the intermediate palladium(II) complex is a palladium(II) complex, as described herein, wherein the ligand R^{L2} is replaced with the group [Org]. Any of the palladium(II) complexes, as provided herein, can be so modified to provide an intermediate palladium(II) complex.

In certain embodiments, [Org] is an optionally substituted aliphatic, optionally substituted heteroaliphatic, optionally substituted aryl or optionally substituted heteroaryl compound coordinated to Pd by a carbon atom.

In certain embodiments, [Org] is an optionally substituted aliphatic, compound coordinated to Pd by a carbon atom.

In certain embodiments, [Org] is an optionally substituted heteroaliphatic compound coordinated to Pd by a carbon atom.

In certain embodiments, [Org] is an optionally substituted heteroaryl compound coordinated to Pd by a carbon atom.

In certain embodiments, [Org] is an optionally substituted aryl compound coordinated to Pd by a carbon atom.

For example, in certain embodiments, the intermediate palladium(II) complex is of the formula (II-c):

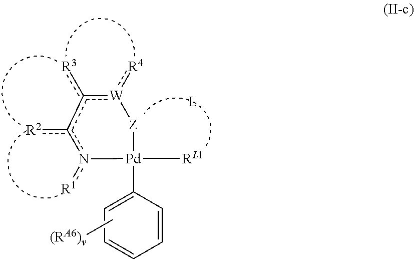

wherein [symbol] , [symbol] , Pd, W, L, R^{L1}, Z, R¹, R², R³ and R⁴ are as defined above and herein;

each instance of R^{A6} is, independently, hydrogen, halogen, optionally substituted aliphatic, optionally substituted heteroaliphatic, optionally substituted aryl, optionally substituted heteroaryl, —CN, —NO₂, —NC, —OR^{A6a}, —SR^{A6b}, —N(R^{A6c})₂, —C(═O)R^{A6d}, —C(═O)OR^{A6a}, —C(═O)N(R^{A6c})₂, —C(═NR^{A6c})R^{A6d}, —C(═NR^{A6c})OR^{A6a}, —C(═NR^{A6c})N(R^{A6c})₂, —S(O)₂R^{A6d}, —S(O)R^{A6d}, or two R^{A6} groups adjacent to each other are joined to form an optionally substituted aryl, optionally substituted heteroaryl, optionally substituted heterocyclic or optionally substituted carbocyclic ring; wherein R^{A6a} is hydrogen, an optionally substituted aliphatic, optionally substituted heteroaliphatic, optionally substituted aryl, optionally substituted heteroaryl or a suitable hydroxyl protecting group; wherein R^{A6b} is hydrogen, an optionally substituted aliphatic, optionally substituted heteroaliphatic, optionally substituted aryl, optionally substituted heteroaryl or a suitable thiol protecting group; wherein each R^{A6c} is, independently, hydrogen, an optionally substituted aliphatic, optionally substituted heteroaliphatic, optionally substituted aryl, optionally substituted heteroaryl or a suitable amino protecting group, or two R^{A6c} groups are joined together to form a heterocyclic or heteroaryl group; and wherein each R^{A6d} is, independently, an optionally substituted aliphatic, optionally substituted heteroaliphatic, optionally substituted aryl, or an optionally substituted heteroaryl group; and

v is an integer between 0-5, inclusive.

In certain embodiments, the intermediate complex is of the formula (II-d):

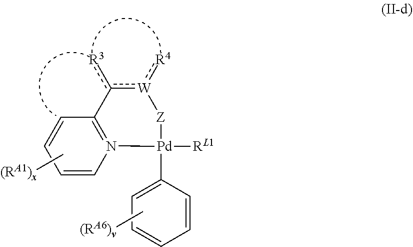

wherein [symbol] , [symbol] , Pd, W, L, R^{L1}, Z, R³, R⁴ R^{A1}, R^{A6}, x and v are as defined above and herein.

In certain embodiments, the intermediate complex is of the formula (II-e):

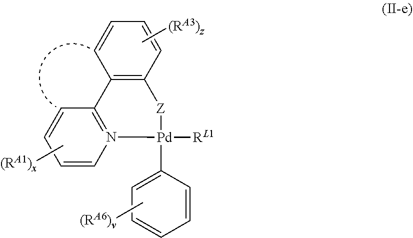

wherein R^{L1}, Z, R³, R⁴ R^{A1}, R^{A3}, R^{A6}, z, x, and v are as defined above and herein.

In certain embodiments, the intermediate complex is of the formula (II-f):

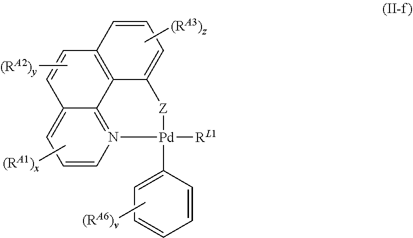

wherein R^{L1}, Z, R³, R⁴ R^{A1}, R^{A2}, R^{A3}, R^{A6}, y, z, x and v are as defined above and herein.

In certain embodiments, the intermediate complex is of the formula (II-g):

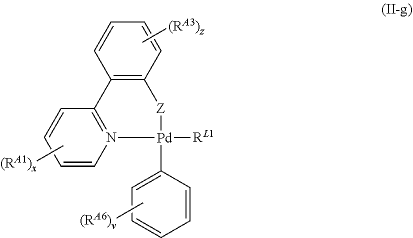

wherein R^{L1}, Z, R³, R⁴ R^{A1}, R^{A3}, R^{A6}, z, x and v are as defined above and herein.

In certain embodiments, R^{A5} is, independently, hydrogen, -Me, —CF₃, -Et, -iPr, -tBu, -Ph, —CHO, —C(═O)OH, —C(═O)OCH₃, —C(O)NH₂, —C(O)NHCH₃, —C(O)N(CH₃)₂, —OH, —OCH₃, —OCF₃, —CH₂OH, —Br, —Cl, —I, —F, or two R^{A5} groups are joined to form a 5-membered heteroaryl ring.

In certain embodiments, v is 0 to 2. In certain embodiments, v is 0. In certain embodiments, v is 1. In certain embodiments, v is 2.

In certain embodiments, the intermediate complex is selected from any of the following complexes:

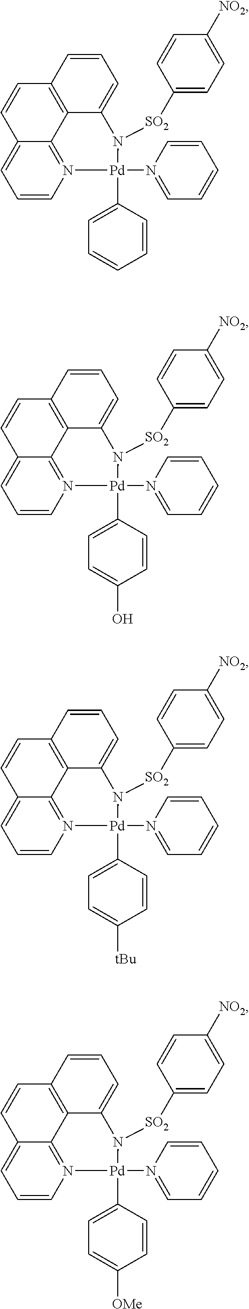

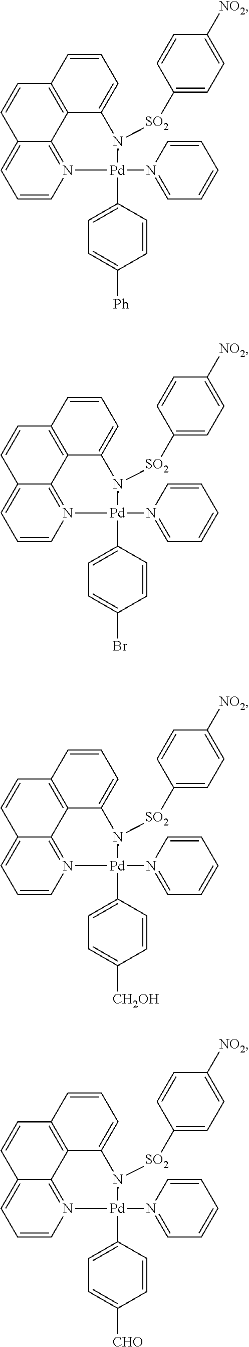

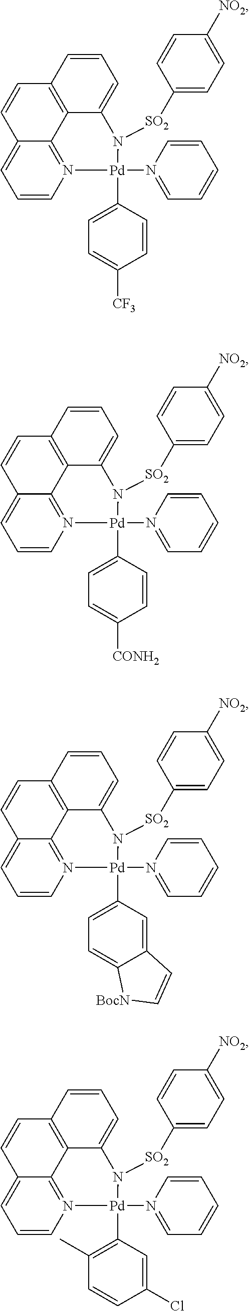

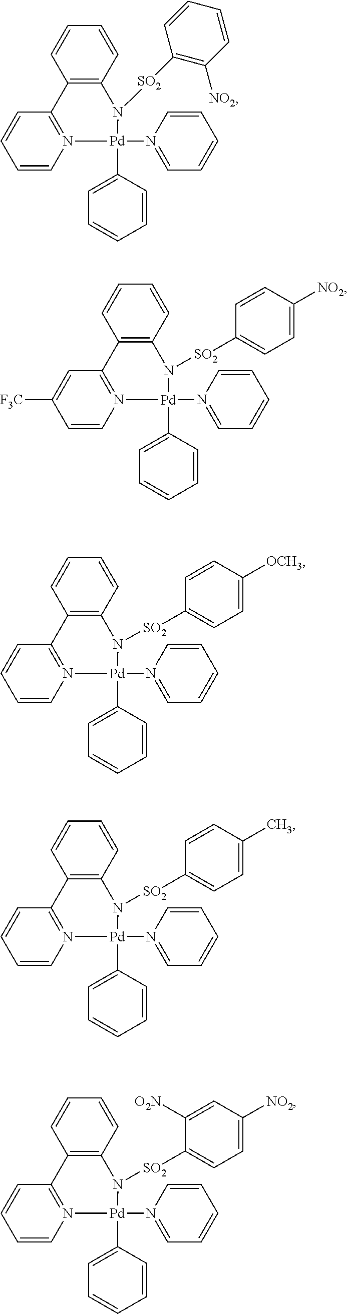

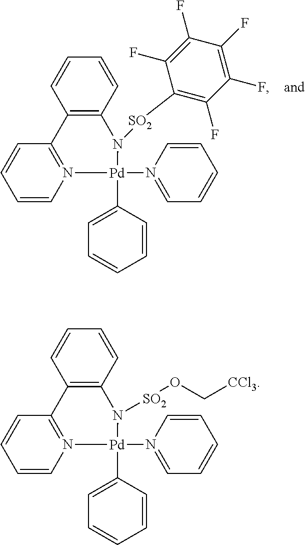

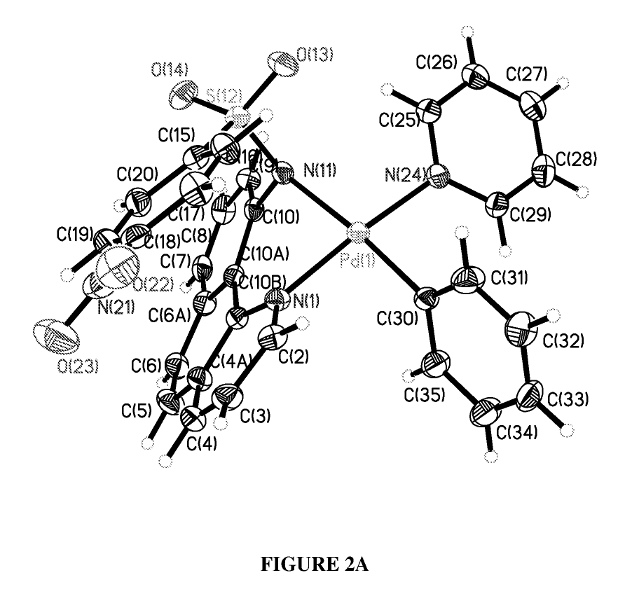

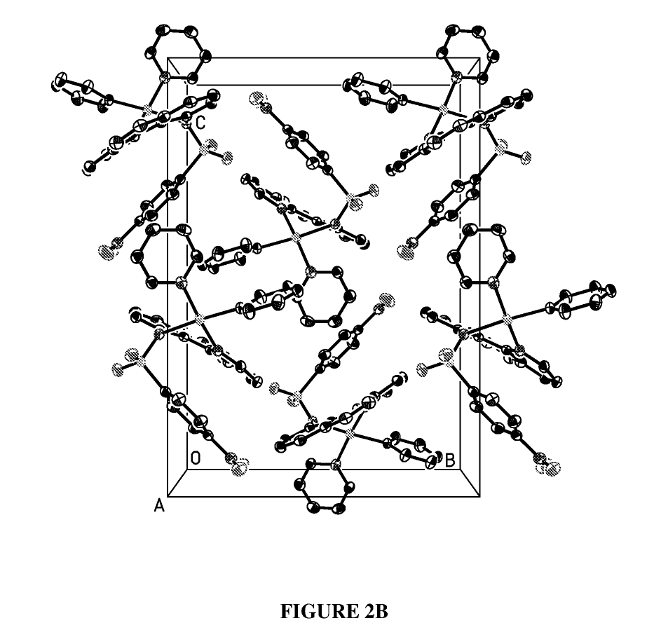

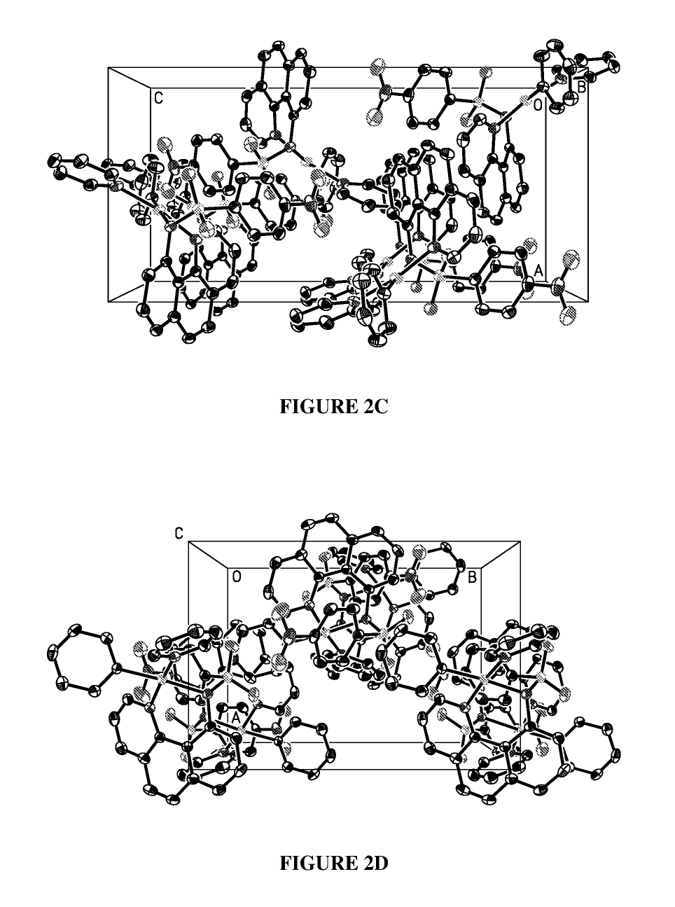

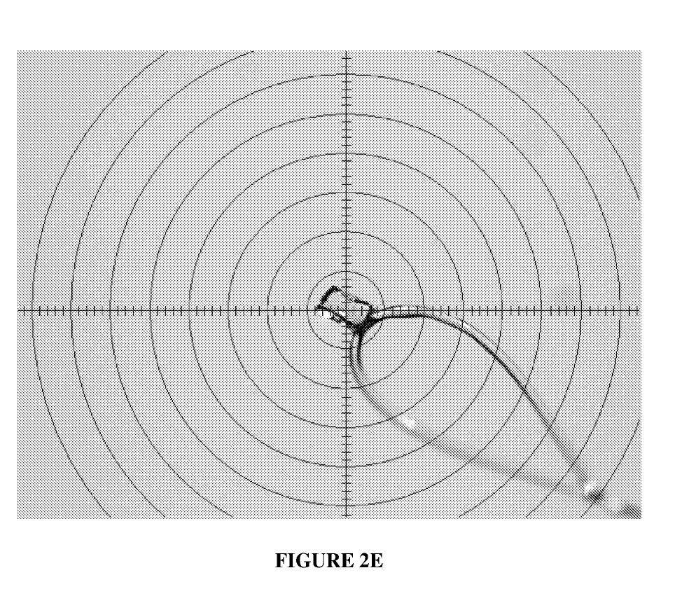

In certain embodiments, the intermediate palladium(II) complex is (i.e., the crystalline complex 4a depicted in FIG. 2A):

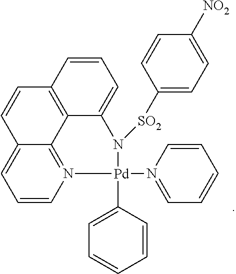

In certain embodiments, the [Org] is biologically active compound that, upon fluorination, provides a known pharmaceutical agent or fluorinated derivative thereof.

For example, in certain embodiments, when the pharmaceutical agent is LIPITOR, [Org] is the group coordinated to Pd as provided below:

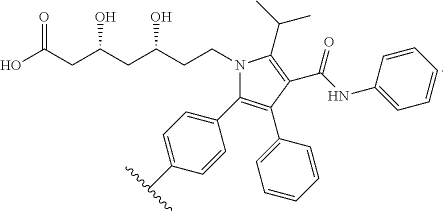 [SMILES: *c1ccc(-c2c(-c3ccccc3)c(C(=O)Nc3ccccc3)c(C(C)C)n2CC[C@@H](O)C[C@@H](O)CC(=O)O)cc1]

In certain embodiments, when the pharmaceutical agent is PAXIL, [Org] is the group coordinated to Pd as provided below:

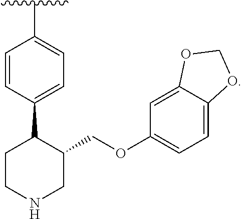

In certain embodiments, the pharmaceutical agent is LEXAPRO, [Org] is the group coordinated to Pd as provided below:

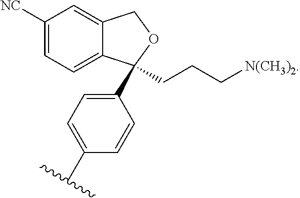 [SMILES: *c1ccc([C@]2(CCCN(C)C)OCc3cc(C#N)ccc32)cc1]

In certain embodiments, when the pharmaceutical agent is a fluorinated derivative of VANCOMYCIN, [Org] is the group coordinated to Pd as provided below:

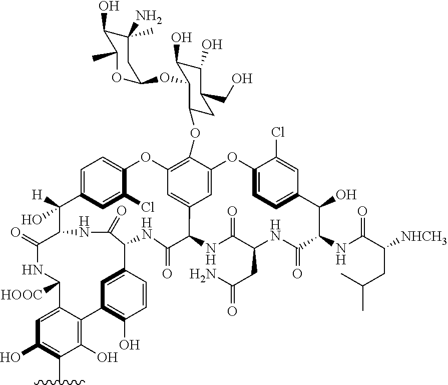

In certain embodiments, when the pharmaceutical agent is a fluorinated derivative of MOZYPREXAPHINE, [Org] is the group coordinated to Pd as provided below:

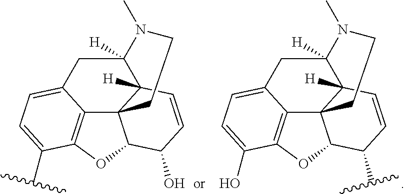

In certain embodiments, when the pharmaceutical agent is a fluorinated derivative of ZYPREXA, [Org] is the group coordinated to Pd as provided below:

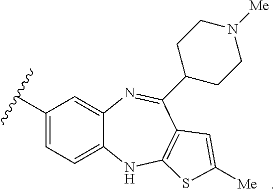 [SMILES: *c1ccc2c(c1)N=C(C1CCN(C)CC1)c1cc(C)sc1N2]

### (vi) Intermediate Palladium(IV) Complex

Without wishing to be bound by any particular theory, an intermediate palladium(IV) complex may be formed during the process upon treatment of the palladium(II) complex with a fluorinating agent. The intermediate complex comprises the palladium(IV) with the organic compound to be fluorinated, a bidentate ligand, and at least one fluoride. The other coordination site may be occupied with a ligand such as a halogen or a solvent molecule. The intermediate is formed by the addition of a fluorinating agent to the palladium(II) complex with the organic compound to be fluorinated, as described above.

Thus, in certain embodiments, the process of step (ii) further comprises providing a palladium(IV) fluoride complex with the organic compound to be fluorinated. In certain embodiments, the process of step (ii) further comprises isolating the intermediate palladium(IV) fluoride complex. In certain embodiments, the intermediate palladium(IV) fluoride complex is not isolatable.

In certain embodiments, the palladium(IV) fluoride complex is of the formula:

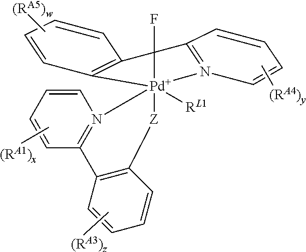

wherein

R^{L1} is optionally substituted aliphatic, optionally substituted heteroaliphatic, optionally substituted aryl, optionally substituted heteroaryl, halogen, a solvent molecule, —OR^{a}, —SR^{b}, —N(R^{c})₂, or —P(R^{x})₃;

each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group ≡C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

each instance of R^{x} is, independently, hydrogen, an optionally substituted aliphatic, optionally substituted heteroaliphatic, optionally substituted aryl, or optionally substituted heteroaryl group;

w is an integer between 0 and 4, inclusive;

x is an integer between 0 and 4, inclusive;

y is an integer between 0 and 4, inclusive;

z is an integer between 0 and 4, inclusive;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, —SR^{A1b}, —N(R^{A1c})₂, —C(═O)R^{A1d}, —C(═O)OR^{Ala}, —C(═O)N(R^{A1c})₂, —C(═NR^{A1c})R^{A1d}, —C(═NR^{A1c})OR^{A1a}, —C(═NR^{A1c})N(R^{A1c})₂, —S(O)₂R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —C(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3} is, independently, hydrogen, an optionally substituted aliphatic, optionally substituted heteroaliphatic, optionally substituted aryl, optionally substituted heteroaryl or a suitable amino protecting group, or two R^{A3c} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group;

each instance of R^{A4} is, independently, hydrogen, halogen, optionally substituted aliphatic, optionally substituted heteroaliphatic, optionally substituted aryl, optionally substituted heteroaryl, —CN, —NO₂, —NC, —OR^{A4a}, —SR^{A4b}, —N(R^{A4c})₂, —C(═O)R^{A4d}, —C(═O)OR^{A4a}, —C(═O)N(R^{A4c})₂, —C(═NR^{A4c})R^{A4d}, —C(═NR^{A4c})OR^{A4a}, —C(═NR^{A4c})N(R^{A4c})₂, —S(O)₂R^{A4d}, —S(O)R^{A4d}, or two R^{A4} groups adjacent to each other are joined to form a 5- to 6-membered aryl, heteroaryl, heterocyclic or carbocyclic ring, wherein R^{A4a} is hydrogen, an optionally substituted aliphatic, optionally substituted heteroaliphatic, optionally substituted aryl, optionally substituted heteroaryl or a suitable hydroxyl protecting group; wherein R^{A4b} is hydrogen, an optionally substituted aliphatic, optionally substituted heteroaliphatic, optionally substituted aryl, optionally substituted heteroaryl or a suitable thiol protecting group; wherein each R^{A4c} is, independently, hydrogen, an optionally substituted aliphatic, optionally substituted heteroaliphatic, optionally substituted aryl, optionally substituted heteroaryl or a suitable amino protecting group, or two R^{A4c} groups are joined together to form a heterocyclic or heteroaryl group; and wherein each R^{A4d} is, independently, an optionally substituted aliphatic, optionally substituted heteroaliphatic, optionally substituted aryl, or an optionally substituted heteroaryl group;

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, or an R^{A5} group and an R^{A4} group are joined to form a 5- to 6-membered aryl, heteroaryl, heterocylic, or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group; and a suitable counteranion.

In certain embodiments, R^{L1} is halogen. In certain embodiments, R^{L1} is fluorine. In certain embodiments, R^{L1} is solvent. In certain embodiments, R^{L1} is CH₃CN. In certain embodiments, R^{L1} is —N(R^{c})₂.

In certain embodiments, Z is not linked to the ligand R^{L1} as in the case of a palladium(II) complex with a bidentate ligand. As defined generally above, in certain embodiments, Z is a bond, —O—, —S—, —C(R^{d})₂—, —C(R^{d})═C(R^{d})—, —C(R^{d})═N—, or —N(R^{e})—;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, or a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted membered heterocyclic or heteroaryl ring.

In certain embodiments, Z is a bond.

In certain embodiments, Z is —C(R^{d})₂—. In certain embodiments, Z is —CH₂—.

In certain embodiments, Z is —C(R^{d})═C(R^{d})—. In certain embodiments, Z is —CH═CH—.

In certain embodiments, Z is —C(R^{d})═N—. In certain embodiments, Z is —CH═N—

In certain embodiments, Z is —O—.

In certain embodiments, Z is —S—.

In certain embodiments, Z is —NR^{e}—.

In certain embodiments, wherein Z is —NR^{e}—, the R^{e} group is of the formula —S(O)₂R^{e1}, wherein R^{e1} is an optionally substituted aliphatic, optionally substituted heteroaliphatic, optionally substituted aryl, or optionally substituted heteroaryl group. In certain embodiments, the R^{e} group is of the formula —S(O)₂R^{e1}, wherein R^{e1} is an optionally substituted aryl or optionally substituted heteroaryl group. In certain embodiments, the R^{e} group is of the formula —S(O)₂R^{e1}, wherein R^{e1} is an optionally substituted heteroaryl group. In certain embodiments, the R^{e} group is of the formula —S(O)₂R^{e1}, wherein R^{e1} is an optionally substituted aryl group.

Exemplary —S(O)₂R^{e1} groups include, but are not limited to:

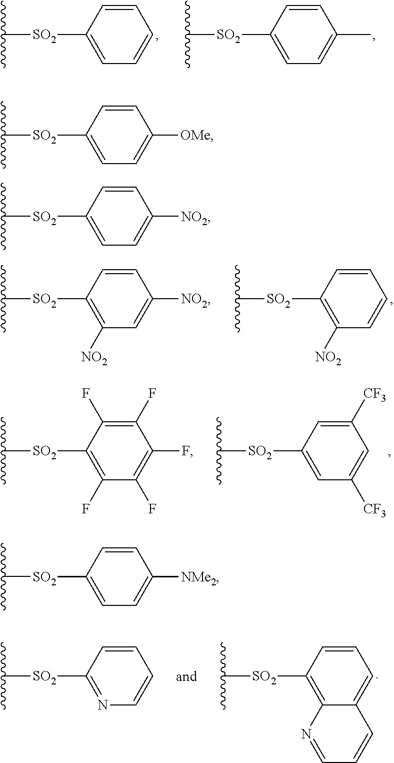

In certain embodiments, Z is of the formula:

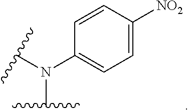 [SMILES: *N(*)c1ccc([N+](=O)[O-])cc1]

In certain embodiments, Z is of the formula:

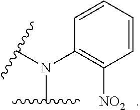 [SMILES: *N(*)c1ccccc1[N+](=O)[O-]]

In certain embodiments, Z is of the formula:

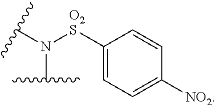 [SMILES: *N(*)S(=O)(=O)c1ccc([N+](=O)[O-])cc1]

In certain embodiments, Z is of the formula:

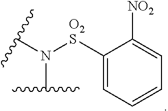 [SMILES: *N(*)S(=O)(=O)c1ccccc1[N+](=O)[O-]]

In certain embodiments, w is 0. In certain embodiments, w is 1. In certain embodiments, w is 2. In certain embodiments, w is 3. In certain embodiments, w is 4.

In certain embodiments, x is 0. In certain embodiments, x is 1. In certain embodiments, x is 2. In certain embodiments, x is 3. In certain embodiments, x is 4.

In certain embodiments, y is 0. In certain embodiments, y is 1. In certain embodiments, y is 2. In certain embodiments, y is 3. In certain embodiments, y is 4.

In certain embodiments, z is 0. In certain embodiments, z is 1. In certain embodiments, z is 2. In certain embodiments, z is 3. In certain embodiments, z is 4.

The counteranion may be any suitable anion. In certain embodiments, the counteranion has a charge of −1. In certain embodiments, the counteranion has a charge of −2. In certain embodiments, the counteranion has a charge of −3. The counteranion may be an organic or inorganic anion. In certain embodiments, the counteranion is an inorganic anion such as phosphate, borate, chloride, bromide, iodide, etc. In other embodiments, the counteranion is an organic anion such as a carboxylic acid, sulfonate, phosphonate, boronate, etc. In certain embodiments, the counteranion is triflate. In certain embodiments, the counteranion is tosylate. In certain embodiments, the counteranion is mesylate. In certain embodiments, the counteranion is hexafluorophosphate. In certain embodiments, the counteranion is tetraphenylborate. In certain embodiments, the counteranion is tetrafluoroborate. In certain embodiments, the counteranion is hexafluoroantimonate. In certain embodiments, the counteranion is [B[3,5-(CF₃)₂C₆H₃]₄]⁻, commonly abbreviated as [BArF₄]⁻.

(vii) Exemplary Reaction Conditions

Described herein are compositions comprising a palladium complex described herein, including a reaction mixture, e.g., a reaction mixture that is present during a method or process described herein. As defined generally herein, in certain embodiments, the process comprises (i) mixing an organic compound comprising one or more boron, organostannane or silane substituents and a palladium(II) complex (i.e., the transmetallation step), and further (ii) mixing a fluorinating agent (i.e., the fluorination step), to provide a fluorinated organic compound wherein the boron, organostannane or silane substituent is replaced with a fluorine substituent.

In certain embodiments, the palladium complex is bound to a solid support.

In certain embodiments, the step (i) further comprises a base. In certain embodiments, the base is an inorganic base. Exemplary inorganic bases include, but are not limited to, K₂CO₃, Na₂CO₃, Ca₂CO₃, NaHCO₃, NaOH, KOH, and LiOH. In certain embodiments, the inorganic base is K₂CO₃.

In certain embodiments, the step (i) further comprises a solvent. In certain embodiments, step (ii) further comprises a solvent.

In certain embodiments, the solvent is an organic solvent. In certain embodiments, the solvent is an aprotic solvent. Exemplary organic solvents include, but are not limited to, benzene, toluene, xylenes, methanol, ethanol, isopropanol, acetonitrile, acetone, ethyl acetate, ethyl ether, dichloromethane and chloroform, or a mixture thereof. In certain embodiments, the solvent is acetone. In certain embodiments, the solvent is acetonitrile. In certain embodiments, the solvent is a mixture of acetone and acetonitrile.

In certain embodiments, step (i) further comprises a solvent selected from methanol and benzene, or a mixture thereof. In certain embodiments, step (i) further comprises a solvent selected from a 1:1 mixture of methanol and benzene.

In certain embodiments, step (ii) further comprises a solvent selected from acetonitrile and acetone, or a mixture thereof. In certain embodiments, step (ii) further comprises a solvent selected from acetonitrile. In certain embodiments, step (ii) further comprises a solvent selected from acetone.

In certain embodiments, step (i) further comprises heating. Alternatively, in certain embodiments, step (i) further comprises cooling.

In certain embodiments, step (i) is not heated or cooled. In certain embodiments, step (i) is performed at room temperature (i.e., 23° C.).

In certain embodiments, step (ii) further comprises heating. In certain embodiments, step (ii) is heated between the temperatures of about 23° C. to about 80° C., of about 30° C. to about 70° C., of about 35° C. to about 60° C., of about 40° C. to about 55° C., of about 45° C. to about 50° C. In certain embodiments, step (ii) is heated to about 50° C.

Alternatively, in certain embodiments, step (ii) further comprises cooling.

In certain embodiments, step (ii) is not heated or cooled. In certain embodiments, step (ii) is performed at room temperature (i.e., 23° C.).

In certain embodiments, the reaction time of step (ii) is less than 20 minutes, less than 15 minutes, less than 10 minutes, less than 5 minutes, or less than 1 minute.

### Applications

The present invention provides a process for fluorination of organic compounds, and, as such, has many useful applications. In certain embodiments, the fluorination reaction is regiospecific.

Introduction of fluorine into a certain position of bioactive compound such as a pharmaceutical agent and an agricultural chemical may remarkably reduce the toxicity of the compound. This is due to the mimic and blocking effect characterized by fluorine. Many compounds, such as 5-fluorouracil, have been reported as successful examples.

Attempts to efficiently synthesize fluorine-containing compounds are performed in many fields. Methods to introduce fluorine into a certain position through the use of fluorinating agents or the use of fluorine-containing building blocks have been reported (see, for example, Liu et al., J. Am. Chem. Soc. (1981) 103:7195; Lovey et al., J. Med. Chem. (1982) 25:71; and Kikuchi et al., Yuki Gosei Kagaku Kyokaishi (1997) 55:88).

Organofluorine compounds are emerging as chemical specialties of significant and increasing commercial interest. A major driver has been the development of fluorine-containing bio-active molecules for use as medicinal and plant-protection agents. Other new applications involving organofluorine chemistry are in the synthesis of liquid crystals, surface active agents, specialty coatings, reactive dyes, and even olefin polymerization catalysts.

¹⁹F-fluorinated organic compounds may be useful for magnetic resonance imaging (MRI) technology. MRI is a is primarily a medical imaging technique most commonly used in radiology to visualize the structure and function of the body. It provides detailed images of the body in any plane. MRI contrast agents are a group of contrast media used to improve the visibility of internal body structures in MRI. Contrast agents alter the relaxation times of tissues and body cavities where they are present, which depending on the image weighting can give a higher or lower signal. Fluorine-containing contrast agents may be especially useful due to the lack of fluorine chemistry in the human body. This could, for example provide a detailed view of acidic regions, such as those containing cancer cells. ¹⁹F-labeled MRI contrast agents may add chemical sensitivity to MRI and could be used to track disease progression without the need to take tissue or fluid samples.

¹⁹F-fluorinated organic compounds may also be useful as probes for nuclear magnetic resonance (NMR) spectroscopy. Fluorine has many advantages as a probe for NMR spectroscopy of biopolymers. ¹⁹F has a spin of one-half, and its high gyromagnetic ratio contributes to its high sensitivity (approximately 83% of the sensitivity of ¹H). It also facilitates long-range distance measurements through dipolar-dipolar coupling. Moreover, the near-nonexistence of fluorine atoms in biological systems enables ¹⁹F NMR studies without background signal interference. Furthermore, the chemical shift of ¹⁹F has been shown to be very sensitive to its environment.

¹⁸F-fluorinated organic compounds are particularly useful for positron-emission tomography (PET) imaging technology. PET is a noninvasive imaging technology that is currently used in the clinic to image cancers and neurological disorders at an early stage of illness. PET tracers are molecules which incorporate a PET-active nucleus and can therefore be visualized by their positron emission in the body. The fluorine isotope ¹⁸F is the most common nucleus for PET imaging because of its superior properties to other nuclei.

A commonly used PET tracer is 2-deoxy-2-fluoroglucose (FDG), which behaves like glucose in the body and is transported to sites of high metabolism such as cancer cells. FDG is not itself metabolized and therefore accumulates in cancer tissues, which in turn can be visualized. The non-invasive nature and the high sensitivity render PET a powerful method for early cancer identification using FDG.

The ¹⁸F radioisotope has a half-life of 109 minutes. The short half-life dictates restrictions on chemical synthesis of PET tracers, because introduction of the fluorine atom has to take place at a very late stage of the synthesis to avoid the unproductive decay of ¹⁸F before it is injected into the body. Fluoride ion is the most common reagent to introduce ¹⁸F but the specific chemical properties of the fluoride ion currently limit the available pool of PET tracers. Due to the narrow functional group compatibility of the strongly basic fluoride ion, only a limited set of chemical reactions can be employed for fluorination, and hence the synthesis of PET tracers is limited to fairly simple molecules such as FDG. The field of PET imaging would benefit from the availability of a new method that is capable of introducing radiolabeled fluoride into structurally more complex organic molecules. An easy access to drug-based PET tracers would simplify determining the fate of such drugs in the body and thereby help to identify and understand their mode of action, bioavailability and time-dependent biodistribution.

### Methods of Treatment

A fluorinated compound described herein, such as a fluorinated pharmaceutical agent, can be administered to cells in culture, e.g. in vitro or ex vivo, or to a subject, e.g., in vivo, to treat, prevent, and/or diagnose a variety of disorders, including those described herein below. In some embodiments, the fluorinated compound is made by a method described herein.

As used herein, the term “treat” or “treatment” is defined as the application or administration of a compound, alone or in combination with, a second compound to a subject, e.g., a patient, or application or administration of the compound to an isolated tissue or cell, e.g., cell line, from a subject, e.g., a patient, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, an amount of a compound effective to prevent a disorder, or a “a prophylactically effective amount” of the compound refers to an amount effective, upon single- or multiple-dose administration to the subject, in preventing or delaying the occurrence of the onset or recurrence of a disorder or a symptom of the disorder.

As used herein, the term “subject” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

Described herein are compounds and compositions useful in the treatment of a disorder. In general, the compounds described herein are fluorinated derivatives of a pharmaceutical agent (e.g., a fluorinated estrone). Also envisioned herein are other compounds, wherein one or more fluorine moieties have been added to the pharmaceutical agent, e.g., replacing a hydrogen or functional group such as an —OH with a fluorine.

### Compositions and Routes of Administration

The compositions delineated herein include the fluorinated compounds delineated herein, such as fluorinated pharmaceutical agents, as well as additional therapeutic agents if present, in amounts effective for achieving a modulation of disease or disease symptoms, including those described herein. In some embodiments, the fluorinated compound is made by a method described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### Kits

A compound described herein (e.g., a palladium complex described herein, an organic compound comprising a boron, organostannane or silane substituent, a fluorinating agent, or a fluorinated compound, such as a fluorinated pharmaceutical agent) may be provided in a kit. The kit includes (a) a compound used in a method described herein, and, optionally (b) informational material. The informational material can be descriptive, instructional, marketing or other material that relates to the methods described herein and/or the use of the compounds for the methods described herein. In some embodiments, the palladium complex is bound to a solid support.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound described herein in a suitable manner to perform the methods described herein, e.g., in a suitable dose, dosage form, or mode of administration (e.g., a dose, dosage form, or mode of administration described herein). In another embodiment, the informational material can include instructions to administer a compound described herein to a suitable subject, e.g., a human, e.g., a human having or at risk for a disorder described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound described herein, the composition of the kit can include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

In some embodiments, the components of the kit are stored under inert conditions

(e.g., under Nitrogen or another inert gas such as Argon). In some embodiments, the components of the kit are stored under anhydrous conditions (e.g., with a desiccant). In some embodiments, the components are stored in a light blocking container such as an amber vial.

A compound described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

### EXAMPLES

The invention now being generally described, it will be more readily understood by reference to the following examples, which are included merely for purposes of illustration of certain aspects and embodiments of the present invention, and are not intended to limit the invention.

### Example 1

### Fluorination of Arylboronic Acids Via Palladium Complexes

The present invention is based, in part, on the discovery of a mild, regiospecific, and functional-group-tolerant fluorination reaction of arylboronic acids. The strategy is illustrated in Scheme 1 and comprises the synthesis of new palladium complexes that subsequently react with the electrophilic fluorination reagent SELECTFLUOR® to afford fluoroarenes.

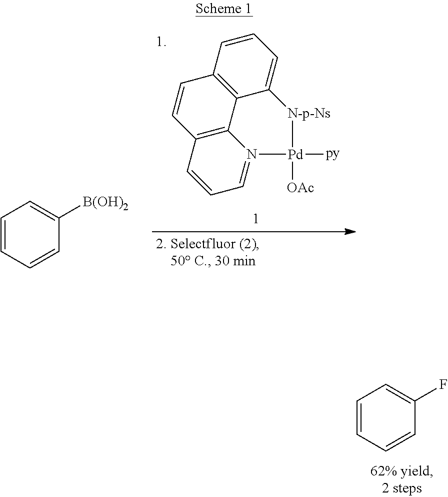

Arylboronic acids were selected as aryl starting materials, because they are readily available, tolerant toward many functional groups, and competent nucleophiles for transmetallation to late transition-metals. Nitrogenous ligands can provide a suitable platform to stabilize palladium(II) without being susceptible to oxidation.

The synthesis of the new palladium acetate complex 1 commenced with sulfamide insertion of the benzoquinoline-derived palladacycle 3 followed by chloride-acetate exchange (Scheme 2 and FIG. 1A). The palladium acetate complex 1 crystallized in a standard square planar geometry with the acetyl ligand residing trans to the κ¹-sulfamidate ligand. Transmetallation from 12 different arylboronic acids in a basic methanol/benzene solution afforded the palladium aryl complexes 4a-m analytically pure as moisture and air stable yellow solids following purification by column chromatography on silica gel in 65-91% yield on a 400 mg scale. The phenylpalladium sulfamidate complex 4a (Ar═Ph) crystallized analogously to 1 in a square planar geometry with the aryl group trans to the κ¹-sulfamidate ligand (FIG. 2A). Methanol was found to be an important cosolvent to obtain complete transmetallation from boron to palladium. During this investigation it was also observed that the use of the palladium acetate complex 1 was superior compared to the corresponding chloride complex in terms of reaction rate and yield of product for transmetallation.

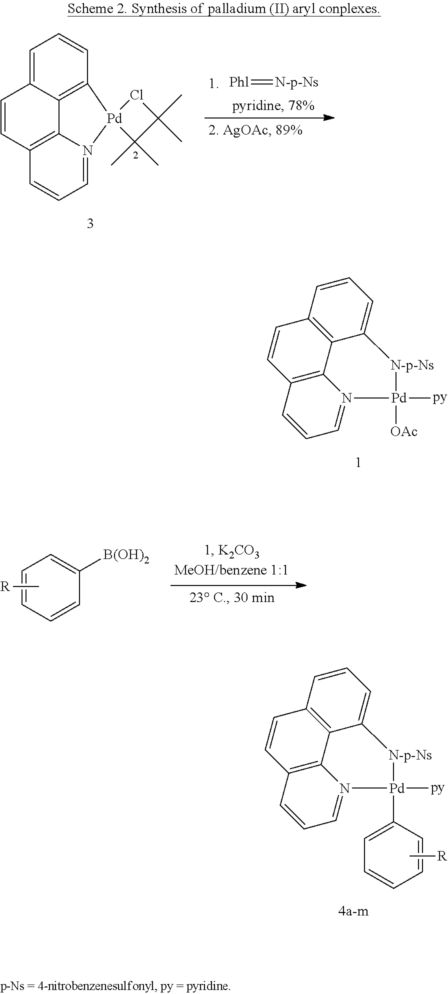

TABLE 2

| R | Yield | # |
|---|---|---|
| H | 76% | (4a) |
| 4-^{t}Bu | 85% | (4b) |
| 4-Ph | 91% | (4c) |
| 4-CH₂OH | 80% | (4d) |
| 4-CHO | 71% | (4e) |
| 4-C:(O)NH₂ | 73% | (4f), |
| 4-OH | 70% | (4g) |
| 4-OMe | 70% | (4h) |
| 4-Br | 65% | (4i), |
| 5-Cl-2-Me | 90% | (4k) |
| 4-CF₃ | 88% | (4l) |
| N-Boc-indole-5-yl | 76% | (4m) |
| 2-OMe | 92% | (4n) |

With the arylpalladium(II) complexes in hand, the electrophilic reagent SELECTFLUOR® (2) was determined to be the most suitable fluorination source to obtain the arylfluorides 5a-m in stoichiometric reactions from 4a-m regiospecifically in 31-82% isolated yield (Table 3). The scope of this fluorination reaction includes a variety of functional-group-containing arenes, most notably arenes with protic functionality (5d, 5g) that is not compatible with nucleophilic aromatic substitution reactions due to the high basicity of the fluoride ion in anhydrous solvents. Additionally, electron-rich arenes (5b, 5g, 5h), which cannot be synthesized through nucleophilic displacement, are accessible. Electrophilic aromatic fluorination has been reported using conventional fluorination regimes, such as the use of elemental fluorine, but the regioselectivity in these cases is typically poor. The fluorination reaction presented herein affords electron-rich arylfluorides regiospecifically. The scope was further extended to electron-poor (5e, 5l) and heteroarenes (5m) and tolerates ortho substitution (5k). The reaction proceeds in 30 minutes under mild conditions (acetonitrile, 50° C.). Acetonitrile can be substituted for acetone as reaction solvent and the yields remain similar. No special care was taken to exclude moisture or air during manipulation; the reactions can be performed in open containers and the yields of the isolated products remained the same. The optimal temperature for the fluorination reaction was determined to be 50° C.; the reactions proceed at 23° C., but inferior yields of product were obtained.

TABLE 3
Electrophilic fluorination of arylpalladium complexes.

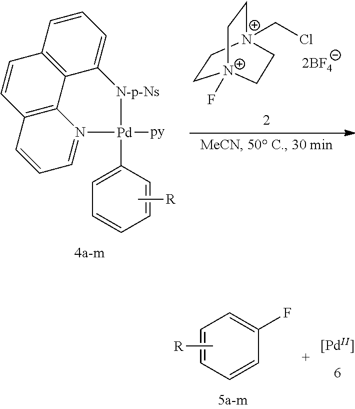

| 4 | product | Yield |
|---|---|---|
| 4a | 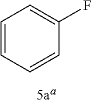 | 81% |
| 4b | 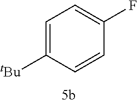 [SMILES: CC(C)(C)c1ccc(F)cc1] | 79% |
| 4c | 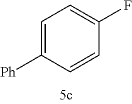 [SMILES: Fc1ccc(-c2ccccc2)cc1] | 72% |
| 4d | 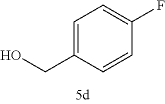 | 75% |
| 4e | 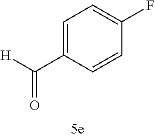 | 61% |
| 4f | 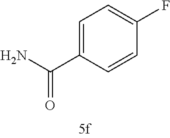 | 75% |
| 4g | 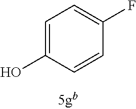 | 31% |
| 4h | 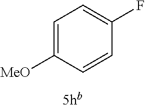 | 50% |
| 4i | 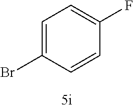 | 57% |
| 4k | 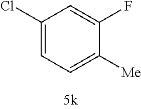 | 82% |
| 4l | 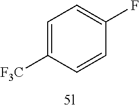 [SMILES: Fc1ccc(C(F)(F)F)cc1] | 62% |
| 4m | 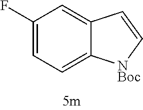 [SMILES: CC(C)(C)OC(=O)n1ccc2cc(F)ccc21] | 51% |
| 4n | 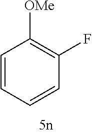 | 29% |

^{a}Yield for this entry determined by ¹⁹F NMR analysis using internal standard.
^{b}Acetone used as solvent.

To determine the fate of the palladium after fluorination byproduct 6 in the reaction mixture (Scheme 3) was studied. We independently synthesized 6 by treatment of palladium chloride 7 with silver tetrafluoroborate in acetonitrile. Subsequent reaction of 6 with one equivalent of pyridine afforded the stable palladium tetrafluoroborate salt 8 that we could isolate and characterize. Addition of pyridine to the reaction displayed in Scheme 3 also afforded 8, which suggests that the benzoquinolinesulfamide ligand remains associated with palladium throughout the reaction.

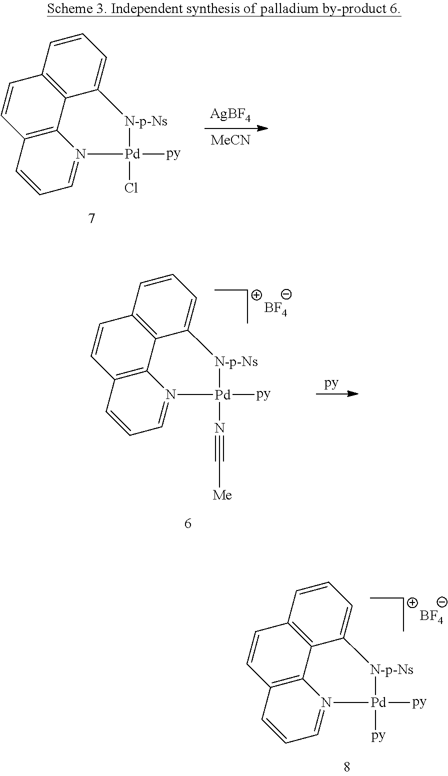

In conclusion we report a two-step synthesis of fluoroarenes from boronic acids via novel arylpalladium complexes. The functional group tolerance, broad substrate scope, and regiospecificity of the fluorination reaction presented herein are superior to those of other fluorination regimes reported.

### Materials and Methods

All reactions were carried out under an ambient atmosphere. Except as indicated otherwise, reactions were magnetically stirred and monitored by thin layer chromatography (TLC) using EMD TLC plates pre-coated with 250 μm thickness silica gel 60 F254 plates and visualized by fluorescence quenching under UV light. In addition, TLC plates were stained using ceric ammonium molybdate or potassium permanganate stain. Flash chromatography was performed on Dynamic Adsorbents Silica Gel 40-63 m particle size using a forced flow of eluant at 0.3-0.5 bar pressure (Still, W. C.; Kahn, M.; Mitra, A. J. Org. Chem. 1978, 43, 2925). Concentration under reduced pressure was performed by rotary evaporation at 25-30° C. at appropriate pressure. Purified compounds were further dried under high vacuum (0.01-0.05 Torr). Yields refer to purified and spectroscopically pure compounds. Melting points were measured on a Buchi 510 apparatus. All melting points were measured in open capillaries and were uncorrected. NMR spectra were recorded on a Varian Unity/Inova 500 spectrometer operating at 500 MHz and 125 MHz for ¹H and ¹³C acquisitions respectively, or on a Varian Mercury 400 spectrometer operating at 375 MHz for ¹⁹F acquisition. Chemical shifts are reported in ppm with a solvent resonance as an internal standard. Data are reported as follows: s=singlet, br=broad, d=doublet, t=triplet, q=quartet, m=multiplet; coupling constants in Hz. High-resolution mass spectra were obtained at the Harvard University Mass Spectrometry Facilities.

### Synthesis of [{(4-Nitrophenyl)sulfonyl}imino]phenyliodinane

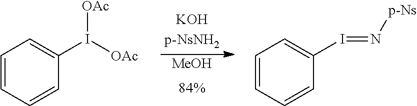

To 4-nitrobenzenesulfonyl amide (5.00 g, 24.8 mmol, 1.00 equiv) in methanol (100 mL) at 23° C. is added potassium hydroxide (3.48 g, 62.0 mmol, 2.50 equiv). The reaction mixture is stirred at 23° C. for 10 min and cooled to 0° C. To the reaction mixture at 0° C. is added iodobenzene diacetate (7.98 g, 24.8 mmol, 1.00 equiv). The reaction mixture is stirred at 0° C. for 10 min and further stirred at 23° C. for 2.0 h. The reaction mixture is poured into cold water (700 mL) and kept at 0° C. for 4 h. The suspension is filtered and washed with water (2×200 mL) and methanol (2×200 mL) to afford 8.39 g of the title compound as a white solid (84% yield). NMR Spectroscopy: ¹H NMR (500 MHz, DMSO-d-6 23° C., δ): 8.02 (d, J=9.0 Hz, 2H), 7.73 (d, J=9.0 Hz, 2H), 7.71 (d, J=6.5 Hz, 2H), 7.41 (t, J=7.0 Hz, 1H), 7.26 (dd, J=8.0 Hz, J=7.5 Hz, 2H). ¹³C NMR (125 MHz, DMSO-d-6, 23° C., δ): 151.7, 148.6, 134.4, 131.4, 130.9, 128.2, 124.3, 117.9.

### Synthesis of Chloro Palladium Complex 7

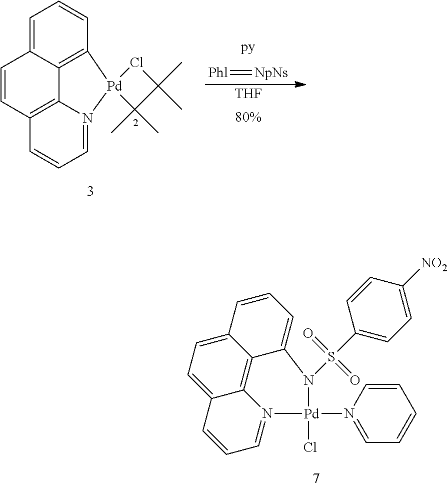

To chloropalladium dimer 3 (1.60 g, 5.00 mmol, 1.00 equiv) in THF (75.0 mL) at 23° C. is added pyridine (3.20 mL, 40.0 mmol, 8.00 equiv) and PhI═N-p-Ns (3.00 g, 7.50 mmol, 1.50 equiv). The reaction mixture is stirred at 23° C. for 17 h. The reaction mixture is filtered and washed with Et₂O (2×10 mL) to afford 2.40 g of the title compound as a light brown solid (78% yield). NMR Spectroscopy: ¹H NMR (500 MHz, CDCl₃, 23° C., δ): 9.20 (dd, J=4.5 Hz, 1.0 Hz, 1H), 8.97 (d, J=4.5 Hz, 2H), 8.07 (dd, J=6.5 Hz, 1.0 Hz, 1H), 7.92-7.82 (m, 5H), 7.53-7.45 (m, 5H), 7.39 (dd, J=6.5 Hz, 4.5 Hz, 1H), 7.32 (d, J=6.0 Hz, 2H). ¹³C NMR (125 MHz, CDCl₃, 23° C., δ): 154.1, 152.5, 148.3, 147.3, 141.6, 138.9, 137.8 (two peaks overlapping), 136.1, 130.7, 130.1, 128.3, 127.1, 126.9, 126.8, 126.2, 125.3, 124.5, 122.5, 122.3 (see Dick, A. R.; Remy, M. S.; Kampf, J. W.; Sanford, M. S. Organometallics 2007, 26, 1365-1370).

### Synthesis of Acetato Palladium Complex 1

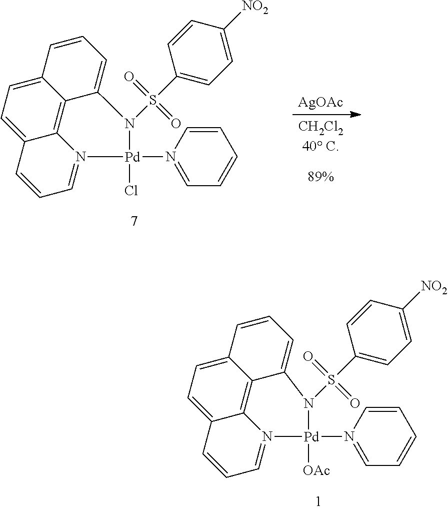

To chloro palladium complex 7 (2.22 g, 3.70 mmol, 1.00 equiv) in CH₂Cl₂ (74.0 mL) at 23° C. is added AgOAc (3.09 g, 18.5 mmol, 5.00 equiv). The suspension is stirred at 40° C. for 2.0 h. After cooling to 23° C., the suspension is filtered through a pad of celite. The filtrate is concentrated in vacuo and the residue is triturated with Et₂O (50 mL). The solids are filtered off and washed with Et₂O (2×50 mL) to afford 2.04 g of the title compound as an orange yellow solid (89% yield). Melting Point: 211° C. (decomp.). NMR Spectroscopy: ¹H NMR (500 MHz, CDCl₃, 23° C., δ): 8.93 (d, J=4.5 Hz, 2H), 8.71 (dd, J=4.5 Hz, 1.5 Hz, 1H), 8.06 (d, J=6.5 Hz, 1H), 7.90-7.76 (m, 5H), 7.52 (d, J=7.0 Hz, 2H) 7.48-7.41 (m, 5H), 7.34 (dd, J=6.5 Hz, 4.5 Hz, 1H), 1.79 (s, 1H). ¹³C NMR (125 MHz, CDCl₃, 23° C., δ): 177.8, 152.0, 151.4, 148.4, 147.9, 141.8, 139.0, 138.8, 138.1, 136.2, 130.8, 130.5, 129.1, 127.5, 127.0, 126.8, 126.3, 125.3, 124.5, 122.6, 122.2, 24.0.

### Synthesis of Aryl Palladium Complex 4a

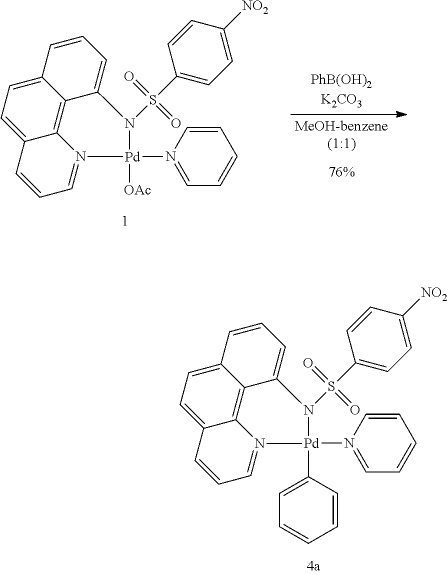

To acetato palladium complex 1 (400 mg, 0.642 mmol, 1.00 equiv) in MeOH (12.8 mL) and benzene (12.8 mL) at 23° C. is added phenylboronic acid (86.0 mg, 0.706 mmol, 1.10 equiv) and K₂CO₃ (133 mg, 0.963 mmol, 1.50 equiv). The reaction mixture is stirred at 23° C. for 2.5 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 1:1 (v/v) to afford 314 mg of the title compound as a pale yellow solid (76% yield). R_{f}=0.23 (hexane/EtOAc 1:1 (v/v)). Melting Point: 205° C. (decomp.). NMR Spectroscopy: ¹H NMR (500 MHz, CDCl₃, 23° C., δ): 9.00 (d, J=6.5 Hz, 2H), 8.27 (dd, J=5.5 Hz, 1.5 Hz, 1H), 7.93 (dd, J=8.0 Hz, 1.5 Hz, 1H), 7.79-7.69 (m, 5H), 7.48 (d, J=9.0 Hz, 2H), 7.38 (d, J=9.0 Hz, 2H), 7.35-7.28 (m, 4H), 7.03 (dd, J=8.0 Hz, 6.5 Hz, 1H), 6.84-6.76 (m, 4H). ¹³C NMR (125 MHz, CDCl₃, 23° C., δ): 155.3, 153.9, 153.3, 149.4, 147.8, 144.6, 144.3, 138.0 (two peaks overlapping), 136.5, 134.8, 130.5, 130.2, 128.5, 127.6, 127.2, 127.0, 126.8, 125.2, 124.7, 124.4, 123.8, 122.4, 121.5.

### Synthesis of Aryl Palladium Complex 4b

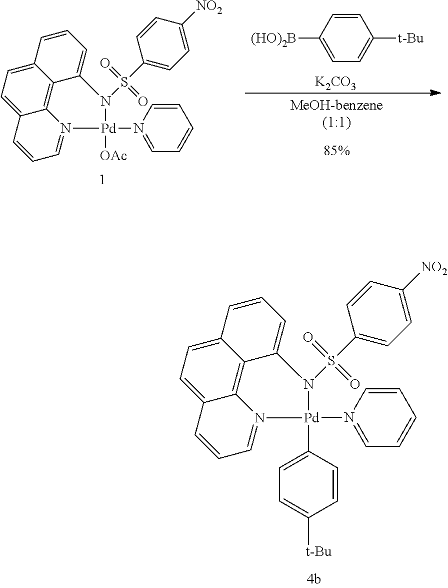

To acetato palladium complex 1 (400 mg, 0.642 mmol, 1.00 equiv) in MeOH (12.8 mL) and benzene (12.8 mL) at 23° C. is added 4-tert-butylphenylboronic acid (126 mg, 0.706 mmol, 1.10 equiv) and K₂CO₃ (133 mg, 0.963 mmol, 1.50 equiv). The reaction mixture is stirred at 23° C. for 13 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 3:2 (v/v) to afford 381 mg of the title compound as a yellow solid (85% yield). R_{f}=0.49 (hexane/EtOAc 1:1 (v/v)). Melting Point: 171° C. (decomp.). NMR Spectroscopy: ¹H NMR (500 MHz, CDCl₃, 23° C., δ): 9.00 (d, J=5.0 Hz, 2H), 8.27 (dd, J=5.5 Hz 1.5 Hz, 1H), 7.92 (dd, J=8.0 Hz, 1.5 Hz, 1H), 7.80-7.70 (m, 5H), 7.48 (d, J=9.0 Hz, 2H), 7.38 (d, J=8.5 Hz, 1H), 7.36-7.30 (m, 4H), 7.03 (dd, J=8.0 Hz, 5.0 Hz, 1H), 6.81 (d, J=9.0 Hz, 2H), 6.70 (d, J=8.5 Hz, 2H), 1.19 (s, 9H). ¹³C NMR (125 MHz, CDCl₃, 23° C., δ): 154.0, 153.4, 150.5, 149.5, 147.8, 146.4, 144.6, 142.3, 137.9 (two peaks overlapping), 136.4, 134.0, 130.4, 130.1, 128.5, 127.4, 126.9, 126.8, 125.1, 124.6, 124.4, 124.2, 122.4, 121.4, 34.1, 31.7. Mass Spectrometry: HRMS-FIA (m/z): Calcd for [C₃₄H₃₀N₄O₄PdS+H], 697.1095. Found, 697.1082.

### Synthesis of Aryl Palladium Complex 4c

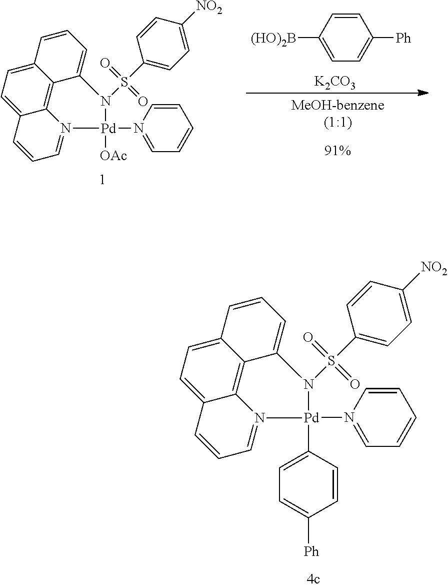

To acetato palladium complex 1 (400 mg, 0.642 mmol, 1.00 equiv) in MeOH (12.8 mL) and benzene (12.8 mL) at 23° C. is added 4-biphenyl boronic acid (140 mg, 0.706 mmol, 1.10 equiv) and K₂CO₃ (133 mg, 0.963 mmol, 1.50 equiv). The reaction mixture is stirred at 23° C. for 11 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 1:1 (v/v) to afford 418 mg of the title compound as a yellow solid (91% yield). R_{f}=0.79 (hexane/EtOAc 3:7 (v/v)). Melting Point: 180° C. (decomp.). NMR Spectroscopy: ¹H NMR (500 MHz, CDCl₃, 23° C., δ): 9.04 (d, J=6.5 Hz, 2H), 8.32 (dd, J=5.0 Hz, 2.0 Hz, 1H), 7.95 (dd, J=8.0 Hz, 1.5 Hz, 1H), 7.81-7.71 (m, 5H), 7.50-7.45 (m, 4H), 7.40 (d, J=9.0 Hz, 1H), 7.38-7.29 (m, 6H), 7.24 (t, J=7.5 Hz, 1H), 7.09-7.05 (m, 3H), 6.88 (d, J=8.0 Hz, 2H). ¹³C NMR (125 MHz, CDCl₃, 23° C., δ): 154.6, 154.1, 153.4, 149.3, 147.8, 144.6, 142.2, 141.4, 138.1, 138.0, 136.5, 135.1, 130.5, 130.2, 128.9, 128.6, 127.6, 127.1, 127.0-126.7 (five peaks overlapping), 125.6, 125.2, 124.7, 124.4, 122.4, 121.6. Mass Spectrometry: HRMS-FIA (m/z): Calcd for [C₃₆H₂₆N₄O₄PdS+H], 717.0782. Found, 717.0786.

### Synthesis of Aryl Palladium Complex 4d

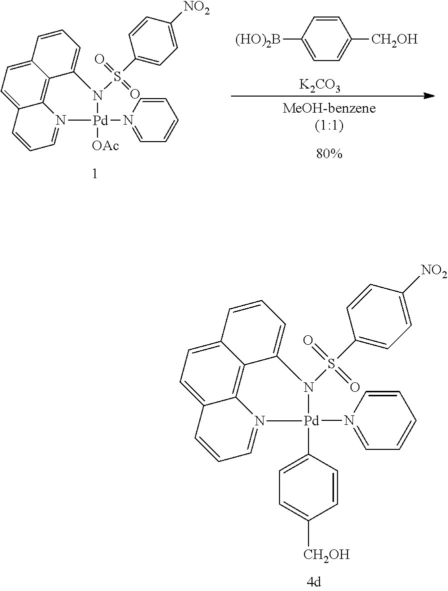

To acetato palladium complex 1 (400 mg, 0.642 mmol, 1.00 equiv) in MeOH (12.8 mL) and benzene (12.8 mL) at 23° C. is added 4-(hydroxymethyl)phenylboronic acid (133 mg, 0.706 mmol, 1.10 equiv) and K₂CO₃ (133 mg, 0.963 mmol, 1.50 equiv). The reaction mixture is stirred at 23° C. for 11 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 1:4 (v/v) to afford 344 mg of the title compound as a yellow solid (80% yield). R_{f}=0.37 (hexane/EtOAc 3:7 (v/v)). Melting Point: 158° C. (decomp.). NMR Spectroscopy: ¹H NMR (500 MHz, CDCl₃, 23° C., δ): 8.99 (d, J=6.5 Hz, 2H), 8.25 (dd, J=5.5 Hz, 1.5 Hz, 1H), 7.94 (dd, J=8.5 Hz, 2.0 Hz, 1H), 7.80-7.69 (m, 5H), 7.47 (d, J=9.0 Hz, 2H), 7.39 (d, J=9.0 Hz, 1H), 7.36-7.27 (m, 4H), 7.04 (dd, J=8.5 Hz, 6.5 Hz, 1H), 6.81 (m, 4H), 4.50 (d, J=4.0 Hz, 2H), 1.49 (t, J=4.0 Hz, 1H). ¹³C NMR (125 MHz, CDCl₃, 23° C., δ): 154.6, 153.9, 153.3, 149.3, 147.8, 144.5, 142.2, 138.0 (two peaks overlapping), 136.5, 136.2, 134.8, 130.5, 130.2, 128.5, 127.5, 126.9, 126.8, 126.2, 125.2, 124.7, 124.4, 121.5, 122.4, 65.5. Mass Spectrometry: HRMS-FIA (m/z): Calcd for [C₃₁H₂₄N₄O₅PdS+H], 671.0575. Found, 617.0598.

### Synthesis of Aryl Palladium Complex 4e

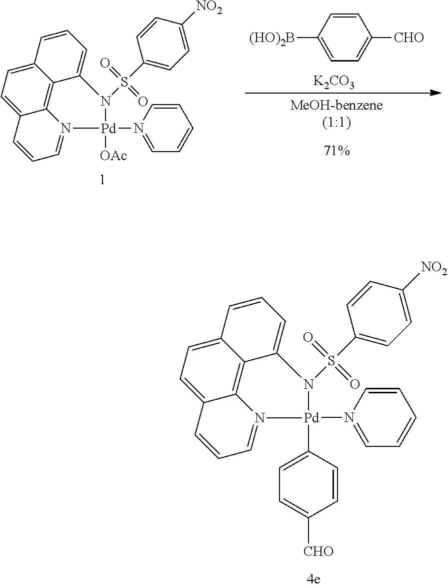

To acetato palladium complex 1 (400 mg, 0.642 mmol, 1.00 equiv) in MeOH (12.8 mL) and benzene (12.8 mL) at 23° C. is added 4-formylphenylboronic acid (133 mg, 0.706 mmol, 1.10 equiv) and K₂CO₃ (133 mg, 0.963 mmol, 1.50 equiv). The reaction mixture is stirred at 23° C. for 18 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 1:1 (v/v) to afford 304 mg of the title compound as a yellow solid (71% yield). R_{f}=0.40 (hexane/EtOAc 3:7 (v/v)). Melting Point: 166° C. (decomp.). NMR Spectroscopy: ¹H NMR (500 MHz, CDCl₃, 23° C., δ): 9.77 (s, 1H), 8.97 (d, J=6.0 Hz, 2H), 8.17 (dd, J=6.5 Hz, 1.5 Hz, 1H), 7.98 (dd, J=7.5 Hz, 1.5 Hz, 1H), 7.84-7.79 (m, 2H), 7.76-7.71 (m, 3H), 7.48 (d, J=8.0 Hz, 2H), 7.44-7.36 (m, 3H), 7.31-7.25 (m, 4H), 7.12 (d, J=7.5 Hz, 2H), 7.07 (dd, J=8.0 Hz, 5.5 Hz, 1H). ¹³C NMR (125 MHz, CDCl₃, 23° C., δ): 192.9, 169.1, 153.7, 153.2, 149.0, 147.9, 144.4, 141.9, 138.4, 138.3, 136.5, 135.5, 133.2, 130.7, 130.4, 128.5, 127.7, 127.6, 126.9, 126.8, 125.4, 124.8, 124.4, 122.4, 121.7. Mass Spectrometry: HRMS-FIA (m/z): Calcd for [C₃₁H₂₂N₄O₅PdS+H], 669.0419.0138. Found, 669.0426.

### Synthesis of Aryl Palladium Complex 4f

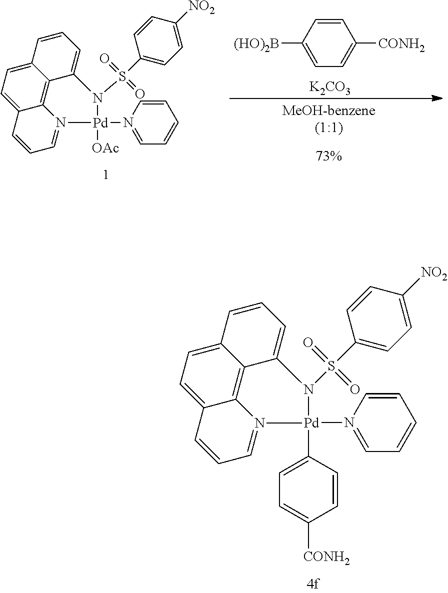

To acetato palladium complex 1 (400 mg, 0.642 mmol, 1.00 equiv) in MeOH (12.8 mL) and benzene (12.8 mL) at 23° C. is added 4-aminocarbonylphenylboronic acid (116 mg, 0.706 mmol, 1.10 equiv) and K₂CO₃ (133 mg, 0.963 mmol, 1.50 equiv). The reaction mixture is stirred at 23° C. for 11 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with EtOAc to afford 319 mg of the title compound as a yellow solid (73% yield). R_{f}=0.21 (EtOAc). Melting Point: 175° C. (decomp.). NMR Spectroscopy: ¹H NMR (500 MHz, CDCl₃, 23° C., δ): 8.97 (d, J=5.5 Hz, 2H), 8.19 (dd, J=6.5 Hz, 1.5 Hz, 1H), 7.97 (dd, J=7.5 Hz, 1.5 Hz, 1H), 7.83-7.70 (m, 5H), 7.47 (d, J=7.0 Hz, 2H), 7.43-7.30 (m, 3H), 7.28 (dd, J=9.0 Hz, 1.5 Hz, 2H), 7.23 (d, J=8.5 Hz, 2H), 7.06 (dd, J=8.5 Hz, 5.5 Hz, 1H), 6.89 (d, J=7.5 Hz, 2H), 5.88 (br, 1H), 5.40 (br, 1H). ¹³C NMR (125 MHz, CDCl₃, 23° C., δ): 163.3, 153.8, 153.3, 149.0, 144.4, 143.1, 142.0, 138.3, 138.2, 136.5, 135.1, 130.6, 130.3, 129.0, 128.5, 127.6, 126.9, 126.8, 126.0, 125.5, 125.4, 124.8, 124.4, 122.4, 121.6. Mass Spectrometry: HRMS-FIA (m/z): Calcd for [C₃₁H₂₃N₅O₅PdS+H], 684.0528. Found, 684.0537.

### Synthesis of Aryl Palladium Complex 4g

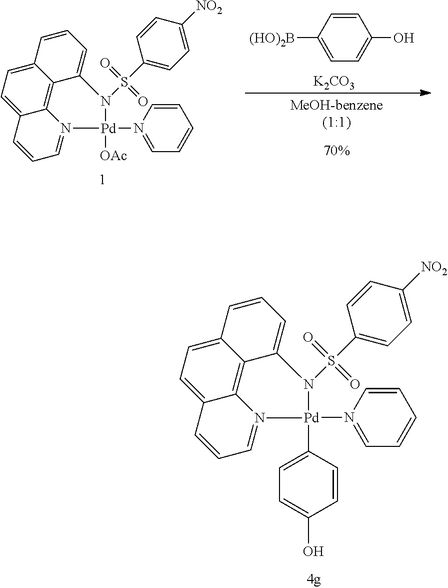

To acetato palladium complex 1 (400 mg, 0.642 mmol, 1.00 equiv) in MeOH (12.8 mL) and benzene (12.8 mL) at 23° C. is added 4-hydroxyphenylboronic acid (97 mg, 0.706 mmol, 1.10 equiv) and K₂CO₃ (133 mg, 0.963 mmol, 1.50 equiv). The reaction mixture is stirred at 23° C. for 15 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 2:3 (v/v) to afford 295 mg of the title compound as a yellow solid (70% yield). R_{f}=0.17 (hexane/EtOAc 1:1 (v/v)). Melting Point: 174° C. (decomp.). NMR Spectroscopy: ¹H NMR (500 MHz, CDCl₃, 23° C., δ): 8.99 (d, J=6.5 Hz, 2H), 8.27 (dd, J=5.0 Hz, 1.5 Hz, 1H), 7.94 (dd, J=7.5 Hz, 1.5 Hz, 1H), 7.79-7.68 (m, 5H), 7.47 (d, J=9.0 Hz, 2H), 7.40-7.27 (m, 5H), 7.04 (dd, J=7.5 Hz, 5.5 Hz, 1H), 6.60 (d, J=8.0 Hz, 2H), 6.38 (d, J=8.0 Hz, 2H), 4.40 (s, 1H). ¹³C NMR (125 MHz, CDCl₃, 23° C., δ): 154.1, 153.4, 152.7, 149.2, 147.8, 147.4, 144.6, 143.4, 142.2, 137.9, 136.4, 134.8, 130.5, 130.1, 128.5, 127.5, 127.0, 126.8, 125.1, 124.7, 124.3, 122.4, 121.4, 114.5. Mass Spectrometry: HRMS-FIA (m/z): Calcd for [C₃₀H₂₂N₄O₅PdS+H], 657.0419. Found, 657.0433.

### Synthesis of Aryl palladium complex 4h

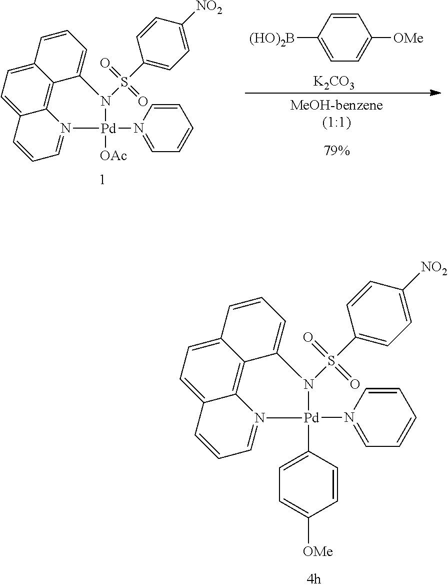

To acetato palladium complex 1 (400 mg, 0.642 mmol, 1.00 equiv) in MeOH (12.8 mL) and benzene (12.8 mL) at 23° C. is added 4-methoxyphenylboronic acid (107 mg, 0.706 mmol, 1.10 equiv) and K₂CO₃ (133 mg, 0.963 mmol, 1.50 equiv). The reaction mixture is stirred at 23° C. for 3.0 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 1:1 (v/v) to afford 340 mg of the title compound as a yellow solid (79% yield). R_{f}=0.29 (hexane/EtOAc 1:1 (v/v)). Melting Point: 154° C. (decomp.). NMR Spectroscopy: ¹H NMR (500 MHz, CDCl₃, 23° C., δ): 8.99 (d, J=5.5 Hz, 2H), 8.27 (d, J=5.5 Hz, 1H), 7.94 (dd, J=8.0 Hz, 1.5 Hz, 1H), 7.80-7.68 (m, 5H), 7.47 (d, J=6.0 Hz, 2H), 7.38 (d, J=8.5 Hz, 1H), 7.35-7.28 (m, 4H), 7.04 (dd, J=8.0 Hz, 5.5 Hz, 1H), 6.64 (d, J=8.0 Hz, 2H), 6.44 (d, J=8.0 Hz, 2H), 3.65 (s, 3H). ¹³C NMR (125 MHz, CDCl₃, 23° C., δ): 156.9, 154.1, 153.5, 149.3, 147.8, 144.6, 143.5, 142.3, 137.9 (two peaks overlapping), 136.5, 134.7, 130.5, 130.1, 128.6, 127.5, 127.0, 126.8, 125.1, 124.7, 124.3, 122.4, 121.5, 113.1, 55.1. Mass Spectrometry: HRMS-FIA (m/z): Calcd for [C₃₁H₂₄N₄O₅PdS+H], 671.0575. Found, 671.0598.

### Synthesis of Aryl Palladium Complex 41

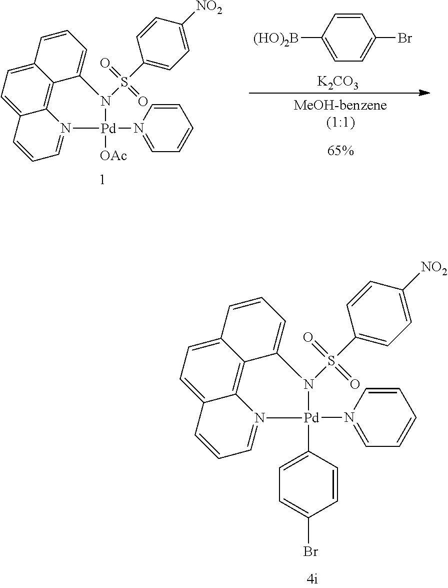

To acetato palladium complex 1 (400 mg, 0.642 mmol, 1.00 equiv) in MeOH (12.8 mL) and benzene (12.8 mL) at 23° C. is added 4-bromophenylboronic acid (142 mg, 0.706 mmol, 1.10 equiv) and K₂CO₃ (133 mg, 0.963 mmol, 1.50 equiv). The reaction mixture is stirred at 23° C. for 3.5 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 3:2 (v/v) to afford 300 mg of the title compound as a yellow solid (65% yield). R_{f}=0.79 (hexane/EtOAc 1:1 (v/v)). Melting Point: 201° C. (decomp.). NMR Spectroscopy: ¹H NMR (500 MHz, CDCl₃, 23° C., δ): 8.96 (d, J=5.0 Hz, 2H), 8.22 (d, J=5.0 Hz, 1H), 7.96 (d, J=8.0 Hz, 1H), 7.82-7.68 (m, 5H), 7.47 (d, J=9.0 Hz, 2H) 7.42-7.26 (m, 5H), 7.09 (dd, J=7.5 Hz, 5.0 Hz, 1H), 6.92 (d, J=8.0 Hz, 2H), 6.70 (d, J=8.0 Hz, 2H). ¹³C NMR (125 MHz, CDCl₃, 23° C., δ): 154.0, 153.5, 153.3, 149.1, 147.9, 142.0, 138.2, 138.1, 136.5, 136.3, 130.6, 130.3, 129.9, 128.5, 127.6, 126.9, 126.8, 125.3, 124.8, 124.4, 122.8, 122.4, 121.7, 118.3. Mass Spectrometry: HRMS-FIA (m/z): Calcd for [C₃₀H₂₁BrN₄O₄PdS+H], 718.9575. Found, 718.9578.

### Synthesis of Aryl palladium complex 4k

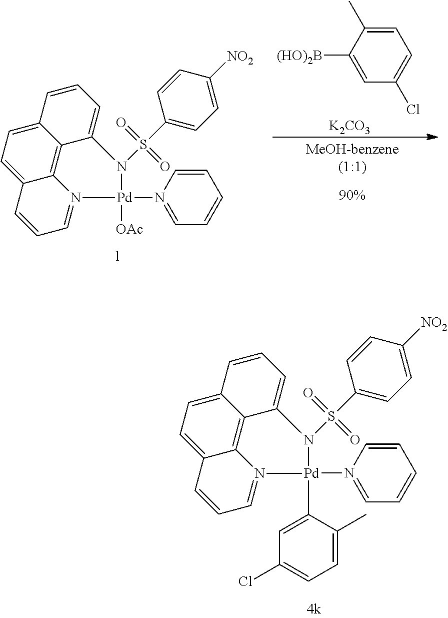

To acetato palladium complex 1 (400 mg, 0.642 mmol, 1.00 equiv) in MeOH (12.8 mL) and benzene (12.8 mL) at 23° C. is added 5-chloro-2-methylphenylboronic acid (120 mg, 0.706 mmol, 1.10 equiv) and K₂CO₃ (133 mg, 0.963 mmol, 1.50 equiv). The reaction mixture is stirred at 23° C. for 10 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 3:2 (v/v) to afford 398 mg of the title compound as a yellow solid (90% yield, 1:1.3 atropisomeric mixture). R_{f}=0.37 (hexane/EtOAc 1:1 (v/v)). Melting Point: 178° C. (decomp.). NMR Spectroscopy: ¹H NMR (500 MHz, CDCl₃, 23° C., δ): 8.98 (d, J=5.5 Hz), 8.91 (d, J=5.5 Hz), 8.28 (d, J=5.0 Hz), 7.96-7.90 (m), 7.81-7.66 (m), 7.55-7.46 (m), 7.40-7.28 (m), 7.08-6.98 (m), 6.81 (d, J=8.0 Hz), 6.74 (dd, J=8.0 Hz, 2.0 Hz), 6.62 (d, J=2.0 Hz), 6.44 (d, J=8.0 Hz), 2.99 (s), 1.69 (s). ¹³C NMR (125 MHz, CDCl₃, 23° C., δ): 159.6, 159.1, 153.6, 153.4, 152.9, 152.8, 149.4, 147.9, 144.7, 144.6, 142.0, 141.8, 140.1, 139.1, 138.2, 138.1, 138.0, 136.5, 133.4, 132.8, 130.7, 130.6, 130.4, 130.3, 130.2, 129.9, 129.2, 129.0, 128.5, 128.4, 127.8, 127.3, 127.0, 126.8, 126.7, 125.4, 125.2, 125.0, 124.8, 124.5, 124.3, 123.9, 123.8, 122.5, 122.4, 121.6, 24.5, 24.2. Mass Spectrometry: HRMS-FIA (m/z): Calcd for [C₃₁H₂₃ClN₄O₄PdS+H], 689.0236. Found, 689.0251.

### Synthesis of Aryl palladium complex 4l

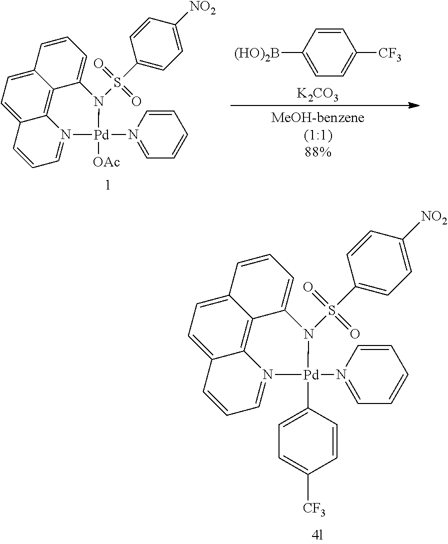

To acetato palladium complex 1 (400 mg, 0.642 mmol, 1.00 equiv) in MeOH (12.8 mL) and benzene (12.8 mL) at 23° C. is added 4-(trifluoromethyl)phenylboronic acid (134 mg, 0.706 mmol, 1.10 equiv) and K₂CO₃ (133 mg, 0.963 mmol, 1.50 equiv). The reaction mixture is stirred at 23° C. for 10 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 3:2 (v/v) to afford 400 mg of the title compound as a yellow solid (88% yield). R_{f}=0.43 (hexane/EtOAc 1:1 (v/v)). Melting Point: 171° C. (decomp.). NMR Spectroscopy: ¹H NMR (500 MHz, CDCl₃, 23° C., δ): 8.97 (d, J=5.5 Hz, 2H), 8.18 (dd, J=4.5 Hz, 1.5 Hz, 1H), 7.97 (dd, J=7.5 Hz, 1.5 Hz, 1H), 7.82-7.70 (m, 5H), 7.48 (d, J=7.0 Hz, 2H), 7.42-7.26 (m, 5H), 7.09 (dd, J=8.0 Hz, 5.0 Hz, 1H), 7.02 (d, J=8.0 Hz, 2H), 6.99 (d, J=8.0 Hz, 2H). ¹³C NMR (125 MHz, CDCl₃, 23° C., δ): 161.3, 153.9, 153.3, 149.0, 147.9, 144.4, 141.9, 138.3, 138.2, 136.5, 135.0, 130.6, 129.5 (q, J=238 Hz), 127.6, 126.9, 126.8, 126.2 (q, J=23 Hz), 125.4, 124.8, 124.4, 123.9, 123.2, 122.4, 121.7. ¹⁹F NMR (375 MHz, CDCl₃, 23° C., δ): −62.5. Mass Spectrometry: HRMS-FIA (m/z): Calcd for [C₃₁H₂₁F3N₄O₄PdS+H], 709.0343. Found, 709.0321

### Synthesis of Aryl Palladium Complex 4m

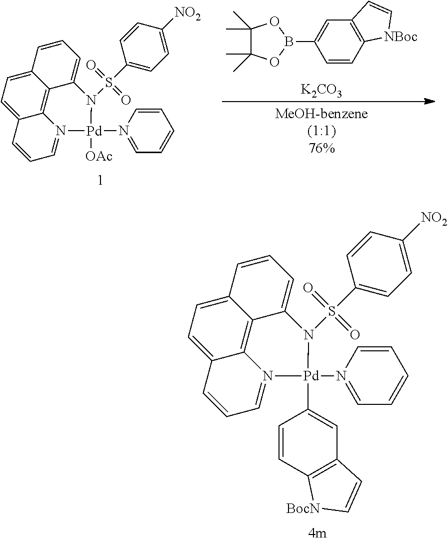

To acetato palladium complex 1 (400 mg, 0.642 mmol, 1.00 equiv) in MeOH (12.8 mL) and benzene (12.8 mL) at 23° C. is added 1-Boc-indole-5-boronic acid pinacol ester (242 mg, 0.706 mmol, 1.10 equiv) and K₂CO₃ (133 mg, 0.963 mmol, 1.50 equiv). The reaction mixture is stirred at 23° C. for 6.0 h. After filtered through a pad of celite, the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexane/EtOAc 1:1 (v/v) to afford 380 mg of the title compound as a yellow solid (76% yield). R_{f}=0.26 (hexane/EtOAc 3:7 (v/v)). Melting Point: 175° C. (decomp.). NMR Spectroscopy: ¹H NMR (500 MHz, CDCl₃, 23° C., δ): 9.01 (d, J=5.0 Hz, 2H), 8.28 (dd, J=5.0 Hz, 1.5 Hz, 1H), 7.91 (dd, J=8.5 Hz, 1.5 Hz, 1H), 7.80-7.70 (m, 5H), 7.61 (br, 1H) 7.47 (d, J=9.0 Hz, 2H), 7.38 (d, J=9.0 Hz, 2H), 7.33-7.28 (m, 4H), 7.00-6.95 (m, 2H), 6.81 (d, J=8.0 Hz, 1H), 6.25 (d, J=2.0 Hz, 1H), 1.60 (s, 9H). ¹³C NMR (125 MHz, CDCl₃, 23° C., δ): 153.9, 153.4, 150.1, 149.3, 147.8 (two peaks overlapping), 144.6, 142.3, 137.9, 136.5, 130.5, 130.1 (two peaks overlapping), 128.6, 127.5, 127.0, 126.8, 126.0, 125.1, 125.0, 124.7, 124.6, 124.4, 122.4, 121.5, 119.9, 113.8, 106.8, 83.4, 28.4. Mass Spectrometry: HRMS-FIA (m/z): Calcd for [C₃₇H₃₁N₅O₆PdS+Na], 802.0922. Found, 802.0895

### Synthesis of Fluorobenzene 5a

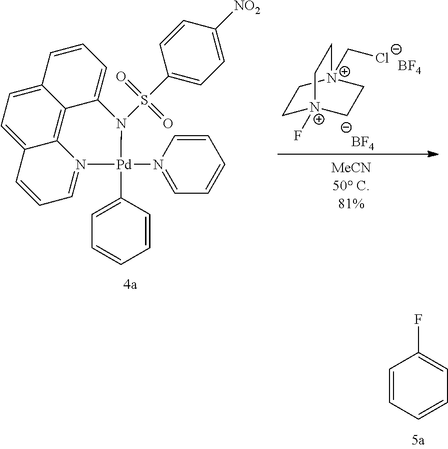

To 1-chloromethyl-4-fluoro-1,4-diazoniabicyclo [2.2.2]octane bis(tetrafluoroborate) (4.3 mg, 0.012 mmol, 1.2 equiv) in Acetonitrile-d-3 (0.3 mL) at 50° C. is added aryl palladium complex 4a (6.4 mg, 0.010 mmol, 1.0 equiv) portionwise over 10 min. The reaction mixture is stirred at 50° C. for 20 min. The reaction mixture is cooled to 23° C., and the yield is determined by comparing integration of the ¹⁹F NMR (375 MHz, acetonitrile-d-3, 23° C.) resonance of fluorobenzene (−115.3 ppm) and that of 3-nitrofluorobenzene (−112.0 ppm, 2.00 μL, 0.0188 mmol). (81% yield). The ¹⁹F NMR chemical shift of the product corresponds to that of authentic sample purchase from Aldrich.

### Synthesis of 1-tert-Butyl-4-fluorobenzene 5b

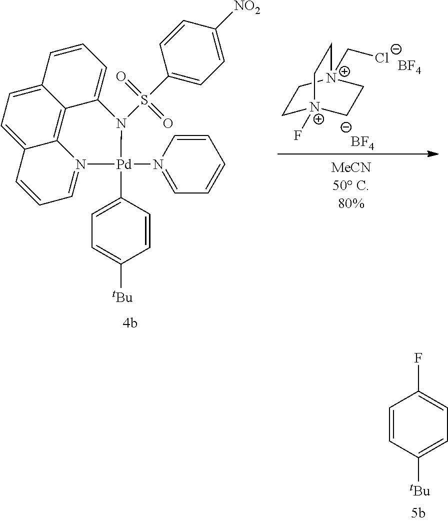

To 1-chloromethyl-4-fluoro-1,4-diazoniabicyclo [2.2.2]octane bis(tetrafluoroborate) (4.3 mg, 0.012 mmol, 1.2 equiv) in acetonitrile-d-3 (0.3 mL) at 50° C. is added aryl palladium complex 4b (7.0 mg, 0.010 mmol, 1.0 equiv) portionwise over 10 min. The reaction mixture is stirred at 50° C. for 20 min. The reaction mixture is cooled to 23° C., and the yield is determined by comparing integration of the ¹⁹F NMR (375 MHz, acetonitrile-d-3, 23° C.) resonance of 1-tert-butyl-4-fluorobenzene (−120.7 ppm) and that of 3-nitrofluorobenzene (−112.0 ppm, 2.00 μL, 0.0188 mmol). (79% yield). The ¹⁹F NMR chemical shift of the product corresponds to that of reported data (Laali et al., J. Organic Chem. (2007) 72:6758-6762).

### Synthesis of 4-Fluorobiphenyl 5c

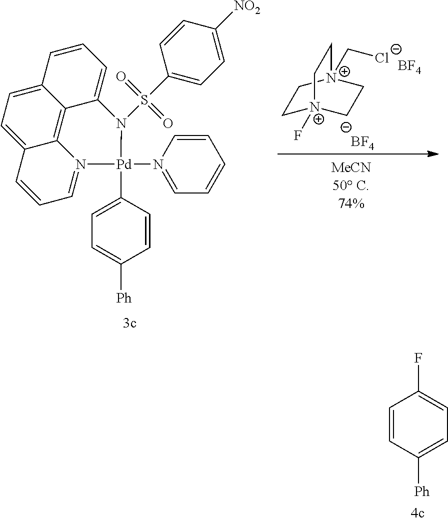

To 1-Chloromethyl-4-fluoro-1,4-diazoniabicyclo [2.2.2]octane bis(tetrafluoroborate) (85.0 mg, 0.240 mmol, 1.20 equiv) in acetonitrile (6.0 mL) at 50° C. is added aryl palladium complex 4c (143 mg, 0.200 mmol, 1.00 equiv) portionwise over 10 min. The reaction mixture is stirred at 50° C. for 20 min. After cooled to 23° C., to the reaction mixture is added pyridine (8.1 μL, 0.10 mmol, 1.0 equiv), and filtered through a pad of celite. The filtrate is concentrated in vacuo and the residue is purified by chromatography on silica gel eluting with hexane/EtOAc 99:1 (v/v) to afford 24.8 mg of the title compound as a white solid (72% yield). R_{f}=0.60 (hexane/EtOAc 19:1 (v/v)). NMR Spectroscopy: ¹H NMR (500 MHz, CDCl₃, 23° C., δ): 7.60-7.54 (m, 4H), 7.47 (dd, J=7.5 Hz, 7.0 Hz, 2H), 7.36 (t, J=7.5 Hz, 1H), 7.14 (dd, J=8.0 Hz, 7.5 Hz, 2H). ¹³C NMR (125 MHz, CDCl₃, 23° C., δ): 162.7 (d, J=244 Hz), 140.5, 137.6, 129.0, 128.9 (d, J=8.5 Hz), 127.5, 127.3, 115.8 (d, J=21 Hz). ¹⁹F NMR (375 MHz, CDCl₃, 23° C., δ): −116.2. These spectroscopic data correspond to those of authentic sample purchase from Alfa Aesar.

### Synthesis of 4-Fluorobiphenyl 5d

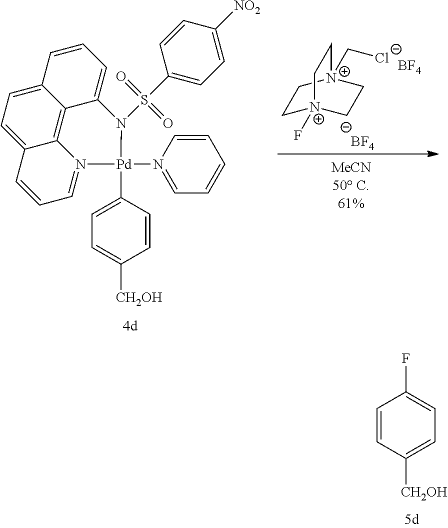

To 1-Chloromethyl-4-fluoro-1,4-diazoniabicyclo [2.2.2]octane bis(tetrafluoroborate) (42.5 mg, 0.120 mmol, 1.20 equiv) in acetonitrile (3.0 mL) at 50° C. is added aryl palladium complex 4d (67.1 mg, 0.100 mmol, 1.00 equiv) portionwise over 10 min. The reaction mixture is stirred at 50° C. for 20 min. After cooled to 23° C., to the reaction mixture is added pyridine (8.1 μL, 0.10 mmol, 1.0 equiv). After concentrated in vacuo, the residue is purified by preparative TLC eluting with pentane/Et₂O 7:3 (v/v) to afford 8.8 mg of the title compound as colorless oil (70% yield). R_{f}=0.61 (hexane/EtOAc 7:3 (v/v)). NMR Spectroscopy: ¹H NMR (500 MHz, CDCl₃, 23° C., δ): 7.29-7.25 (m, 2H), 7.05-7.00 (dd, J=8.0 Hz, 7.5 Hz, 2H), 4.55 (s, 2H), 3.10 (br, 1H). ¹³C NMR (125 MHz, CDCl₃, 23° C., δ): 162.5 (d, J=243 Hz), 136.8, 129.0 (d, J=8.3 Hz), 115.6 (d, J=21 Hz), 64.5. ¹⁹F NMR (375 MHz, CDCl₃, 23° C., δ): −115.4. These spectroscopic data correspond to those of authentic sample purchase from Alfa Aesar.

### Synthesis of 4-Fluorobenzaldehyde 5e

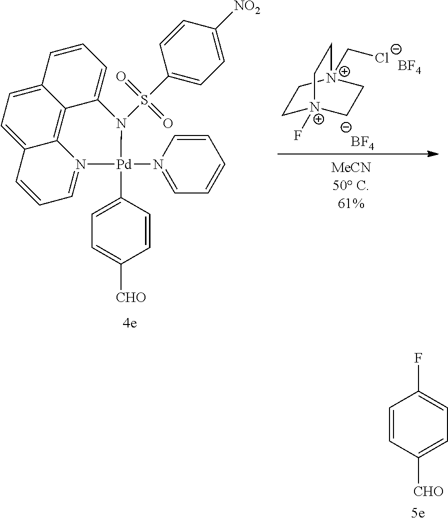

To 1-Chloromethyl-4-fluoro-1,4-diazoniabicyclo [2.2.2]octane bis(tetrafluoroborate) (42.5 mg, 0.120 mmol, 1.20 equiv) in acetonitrile (3.0 mL) at 50° C. is added aryl palladium complex 4e (66.9 mg, 0.100 mmol, 1.00 equiv) portionwise over 10 min. The reaction mixture is stirred at 50° C. for 20 min. After cooled to 23° C., to the reaction mixture is added pyridine (8.1 μL, 0.10 mmol, 1.0 equiv). After concentrated in vacuo, the residue is purified by preparative TLC eluting with pentane/Et₂O 7:3 (v/v) to afford 8.8 mg of the title compound as colorless oil (61% yield). R_{f}=0.77 (hexane/EtOAc 7:3 (v/v)). NMR Spectroscopy: ¹H NMR (500 MHz, CDCl₃ 23° C., δ): 9.95 (s, 1H), 7.92-7.88 (m, 2H), 7.22-7.18 (dd, J=8.0 Hz, 7.5 Hz, 2H). ¹³C NMR (125 MHz, CDCl₃, 23° C., δ): 190.7, 166.7 (d, J=255 Hz), 133.2, 132.5 (d, J=9.9 Hz), 116.6 (d, J=22 Hz). ¹⁹F NMR (375 MHz, CDCl₃, 23° C., δ): −102.9. These spectroscopic data correspond to those of authentic sample purchase from Aldrich.

### Synthesis of 4-Fluorobenzmide 5f

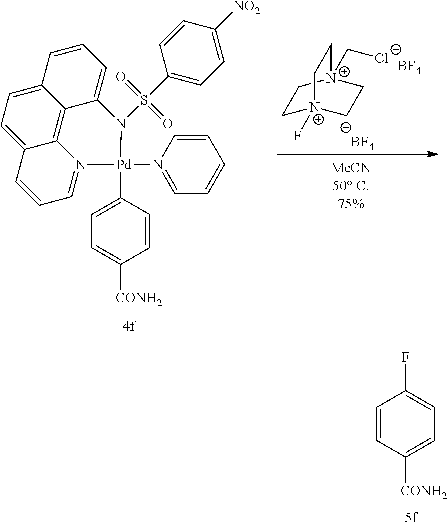

To 1-Chloromethyl-4-fluoro-1,4-diazoniabicyclo [2.2.2]octane bis(tetrafluoroborate) (42.5 mg, 0.120 mmol, 1.20 equiv) in acetonitrile (3.0 mL) at 50° C. is added aryl palladium complex 4f (68.4 mg, 0.100 mmol, 1.00 equiv) portionwise over 10 min. The reaction mixture is stirred at 50° C. for 20 min. After cooled to 23° C., to the reaction mixture is added pyridine (8.1 μL, 0.10 mmol, 1.0 equiv). After concentrated in vacuo, the residue is purified by preparative TLC eluting with EtOAc to afford 10.3 mg of the title compound as colorless oil (74% yield). R=0.40 (EtOAc). NMR Spectroscopy: ¹H NMR (500 MHz, DMSO-d-6, 23° C., δ): 8.02 (br, 1H), 7.95 (dd, J=9.0 Hz, 6.0 Hz, 2H), 7.42 (br, 1H), 7.26 (dd, J=7.5 Hz, 7.0 Hz, 2H). ¹³C NMR (125 MHz, DMSO-d-6, 23° C., δ): 167.6, 164.6 (d, J=247 Hz), 131.4, 130.8 (d, J=14 Hz), 115.8 (d, J=21 Hz). ¹⁹F NMR (375 MHz, DMSO-d-6, 23° C., δ): −110.0. These spectroscopic data correspond to those of authentic sample purchase from Alfa Aesar.

### Synthesis of 4-Fluorophenol 5g

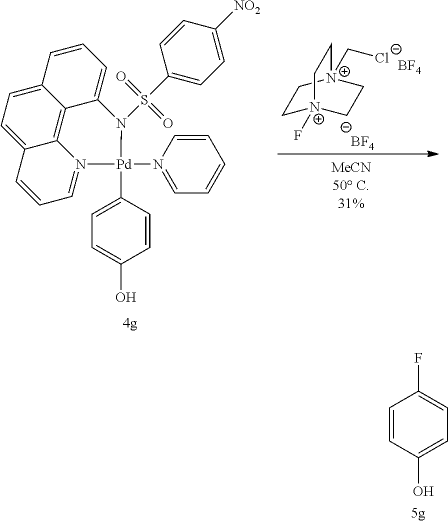

To 1-Chloromethyl-4-fluoro-1,4-diazoniabicyclo [2.2.2]octane bis(tetrafluoroborate) (85.0 mg, 0.240 mmol, 1.20 equiv) in acetonitrile (6.0 mL) at 50° C. is added aryl palladium complex 4g (131 mg, 0.200 mmol, 1.00 equiv) portionwise over 10 min. The reaction mixture is stirred at 50° C. for 20 min. After cooled to 23° C., to the reaction mixture is added pyridine (16 μL, 0.20 mmol, 1.0 equiv). After concentrated in vacuo, the residue is purified by preparative TLC eluting with Hexane/EtOAc 7:3 (v/v) to afford 6.9 mg of the title compound as a white solid (31% yield). R_{f}=0.58 (hexane/EtOAc 7:3 (v/v)). NMR Spectroscopy: ¹H NMR (500 MHz, CDCl₃, 23° C., δ): 6.95-6.95 (dd, J=8.0 Hz, 7.5 Hz, 2H), 6.80-6.76 (m, 2H), 5.41 (s, 1H). ¹³C NMR (125 MHz, CDCl₃, 23° C., δ): 157.6 (d, J=237 Hz), 151.5, 116.5 (d, J=8.0 Hz), 116.3 (d, J=21 Hz). ¹⁹F NMR (375 MHz, CDCl₃, 23° C., δ): −124.3. These spectroscopic data correspond to those of authentic sample purchase from Aldrich.

### Synthesis of 4-Fluoroanisole 5h

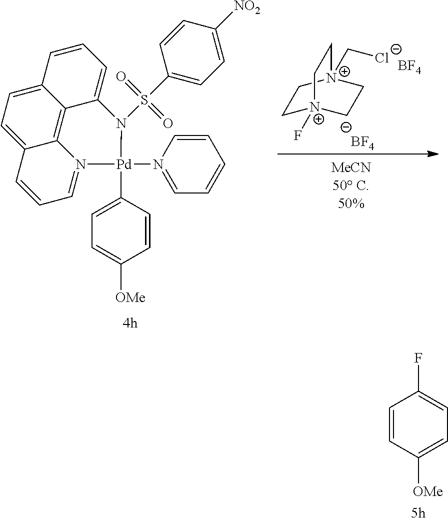

To 1-Chloromethyl-4-fluoro-1,4-diazoniabicyclo [2.2.2]octane bis(tetrafluoroborate) (85.0 mg, 0.240 mmol, 1.20 equiv) in acetonitrile (6.0 mL) at 50° C. is added aryl palladium complex 4h (134 mg, 0.200 mmol, 1.00 equiv) portionwise over 10 min. The reaction mixture is stirred at 50° C. for 20 min. After cooled to 23° C., to the reaction mixture is added pyridine (16 μL, 0.20 mmol, 1.0 equiv). After concentrated in vacuo, the residue is purified by preparative TLC eluting with pentane/Et₂O 9:1 (v/v) to afford 11.6 mg of the title compound as colorless oil (46% yield). R_{f}=0.55 (hexane/EtOAc 9:1 (v/v)). NMR Spectroscopy: ¹H NMR (500 MHz, CDCl₃, 23° C., δ): 7.01-6.95 (m, 2H), 6.87-6.81 (m, 2H), 3.79 (s, 3H). ¹³C NMR (125 MHz, CDCl₃, 23° C., δ): 157.4 (d, J=247 Hz), 155.9, 116.0 (d, J=23 Hz), 115.0 (d, J=7.7 Hz), 56.0. ¹⁹F NMR (375 MHz, CDCl₃, 23° C., δ): −124.8. These spectroscopic data correspond to those of authentic sample purchase from Alfa Aesar.

### Synthesis of 1-Bromo-4-fluorobenzene 5i

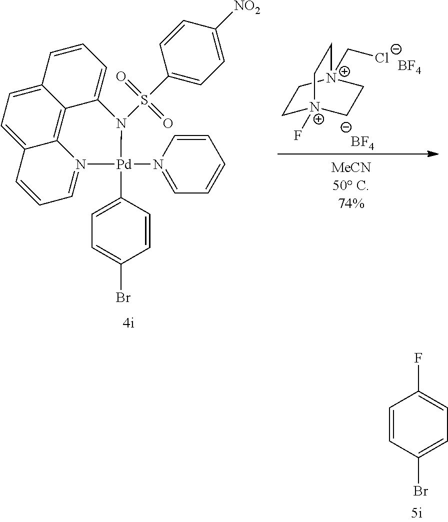

To 1-Chloromethyl-4-fluoro-1,4-diazoniabicyclo [2.2.2]octane bis(tetrafluoroborate) (42.5 mg, 0.120 mmol, 1.20 equiv) in acetonitrile (3.0 mL) at 50° C. is added aryl palladium complex 4i (72.0 mg, 0.100 mmol, 1.00 equiv) portionwise over 10 min. The reaction mixture is stirred at 50° C. for 20 min. After cooled to 23° C., to the reaction mixture is added pyridine (8.1 μL, 0.10 mmol, 1.0 equiv). After concentrated in vacuo, the residue is purified by preparative TLC eluting with pentane/Et₂O 19:1 (v/v) to afford 12.8 mg of the title compound as colorless oil (73% yield). R_{f}=0.70 (hexane/EtOAc 19:1 (v/v)). NMR Spectroscopy: ¹H NMR (500 MHz, CDCl₃, 23° C., δ): 7.47-7.42 (m, 2H), 6.98-6.92 (m, 2H). ¹³C NMR (125 MHz, CDCl₃, 23° C., δ): 162.1 (d, J=245 Hz), 133.2, (d, J=8.5 Hz), 117.5 (d, J=23 Hz), 116.8. ¹⁹F NMR (375 MHz, CDCl₃, 23° C., δ): −115.7. These spectroscopic data correspond to those of authentic sample purchase from Alfa Aesar.

### Synthesis of 4-Chloro-2-fluorotoluene 5k

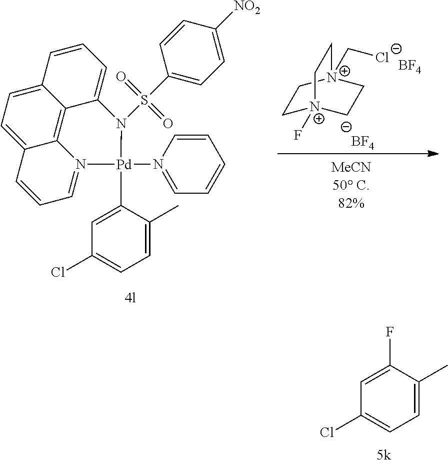

To 1-Chloromethyl-4-fluoro-1,4-diazoniabicyclo [2.2.2]octane bis(tetrafluoroborate) (42.5 mg, 0.120 mmol, 1.20 equiv) in acetonitrile (3.0 mL) at 50° C. is added aryl palladium complex 4k (68.9 mg, 0.100 mmol, 1.00 equiv) portionwise over 10 min. The reaction mixture is stirred at 50° C. for 20 min. After cooled to 23° C., to the reaction mixture is added pyridine (8.1 μL, 0.10 mmol, 1.0 equiv). After concentrated in vacuo, the residue is purified by preparative TLC eluting with pentane/Et₂O 9:1 (v/v) to afford 11.9 mg of the title compound as colorless oil (82% yield). R_{f}=0.72 (hexane/EtOAc 9:1 (v/v)). NMR Spectroscopy: ¹H NMR (500 MHz, CDCl₃, 23° C., δ): 7.13-7.08 (dd, J=7.5 Hz, 7.0 Hz, 2H), 7.05-7.01 (m, 2H). ¹³C NMR (125 MHz, CDCl₃, 23° C., δ): 161.3 (d, J=246 Hz), 132.3, 132.2 (d, J=5.9 Hz), 124.3, 123.6 (d, J=17 Hz), 116.0 (d, J=26 Hz), 14.4. ¹⁹F NMR (375 MHz, CDCl₃, 23° C., δ): −115.1. These spectroscopic data correspond to those of authentic sample purchase from Alfa Aesar.

### Synthesis of 4-Fluorobenzotrifluoride 5l

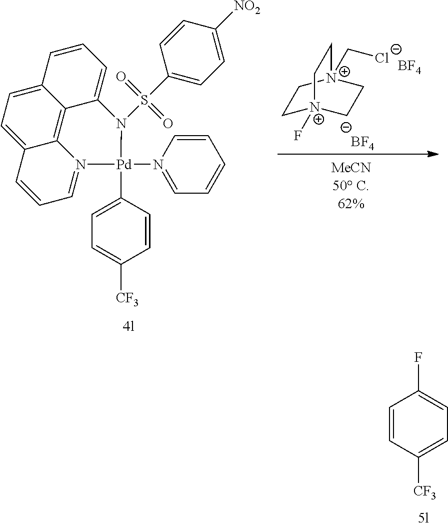

To 1-chloromethyl-4-fluoro-1,4-diazoniabicyclo [2.2.2]octane bis(tetrafluoroborate) (4.3 mg, 0.012 mmol, 1.2 equiv) in acetonitrile-d-3 (0.3 mL) at 50° C. is added aryl palladium complex 41 (6.4 mg, 0.010 mmol, 1.0 equiv) portionwise over 10 min. The reaction mixture is stirred at 50° C. for 20 min. The reaction mixture is cooled to 23° C., and the yield is determined by comparing integration of the ¹⁹F NMR (375 MHz, acetonitrile-d-3, 23° C.) resonance of 4-fluorobenzotrifluoride (−109.4 ppm) and that of 3-nitrofluorobenzene (−112.0 ppm, 2.00 μL, 0.0188 mmol). (54% yield). The ¹⁹F NMR chemical shift of the product corresponds to that of authentic sample purchase from Alfa Aesar.

### Synthesis of 4-Fluorobenzaldehyde 5m

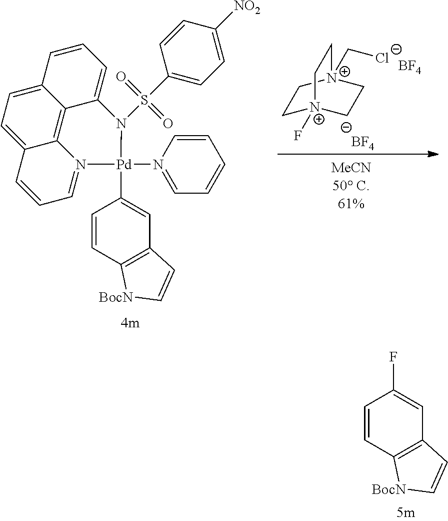

To 1-Chloromethyl-4-fluoro-1,4-diazoniabicyclo [2.2.2]octane bis(tetrafluoroborate) (42.5 mg, 0.120 mmol, 1.20 equiv) in acetonitrile (3.0 mL) at 50° C. is added aryl palladium complex 4m (78.0 mg, 0.100 mmol, 1.00 equiv) portionwise over 10 min. The reaction mixture is stirred at 50° C. for 20 min. After cooled to 23° C., to the reaction mixture is added pyridine (8.1 μL, 0.10 mmol, 1.0 equiv). After concentrated in vacuo, the residue is purified by preparative TLC eluting with hexane/EtOAc 7:3 (v/v) to afford 14.1 mg of the title compound as colorless oil (60% yield). R_{f}=0.75 (hexane/EtOAc 7:3 (v/v)). NMR Spectroscopy: ¹H NMR (500 MHz, CDCl₃, 23° C., δ): 8.08 (br, 1H), 7.62 (d, J=4.0 Hz, 1H), 7.20 (dd, J=6.5 Hz, J=2.0 Hz, 1H), 7.03 (ddd, J=7.0 Hz, 6.5 Hz, 2.0 Hz, 1H), 6.52 (d, J=4.0 Hz, 1H), 1.68 (s, 9H). ¹³C NMR (125 MHz, CDCl₃, 23° C., δ): 159.5 (d, J=238 Hz), 149.7, 131.8, 131.6 (d, J=10 Hz), 127.7, 116.3 (d, J=9.1 Hz), 112.2 (d, J=24 Hz), 107.2, 106.5 (d, J=24 Hz), 84.1, 28.4. ¹⁹F NMR (375 MHz, CDCl₃, 23° C., δ): −121.7. These spectroscopic data correspond to those of authentic sample independently synthesized from 5-fluoroinodole and Boc₂O.

### Synthesis of Bispyridine palladium tetrafluoroborate salt 8

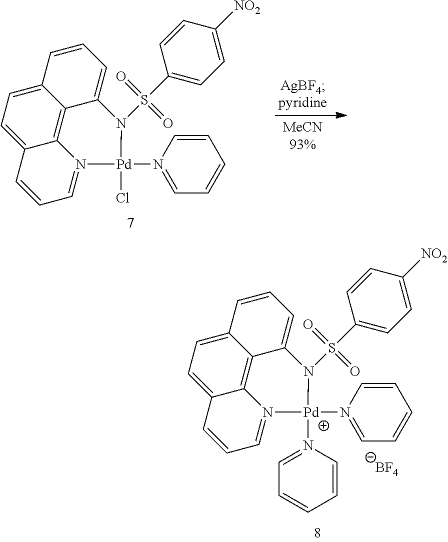

To chloro palladium complex 7 (59.9 mg, 0.100 mmol, 1.00 equiv) in acetonitrile (1.0 mL) at 23° C. is added AgBF₄ (38.8 mg, 0.200 mmol, 2.00 equiv). The suspension is stirred at 23° C. for 1.0 hour and to the suspension is added pyridine (8.1 μL, 0.10 mmol, 1.0 equiv). The suspension is filtered through a pad of celite and the filtrate is concentrated in vacuo to afford 67.9 mg of the title compound as an orange oil (67.9 mg, 93% yield). NMR Spectroscopy: ¹H NMR (500 MHz, acetone-d-6, 23° C., δ): 9.29 (d, J=5.5 Hz, 2H), 8.99 (d, J=5.5 Hz, 2H), 8.51 (dd, J=5.5 Hz, 1.5 Hz, 1H), 8.44 (dd, J=7.5 Hz, 1.0 Hz, 1H), 8.15-8.08 (m, 3H), 8.01 (dd, J=8.0 Hz, 7.5 Hz, 1H), 7.89 (t, J=7.5 Hz, 1H), 7.80-7.70 (m, 4H), 7.66 (d, J=9.0 Hz, 2H), 7.59-7.52 (m, 4H), 7.48 (dd, J=8.0 Hz, 5.5 Hz, 1H). ¹³C NMR (125 MHz, CDCl₃, 23° C., δ): 152.6, 152.4, 152.3, 152.2, 152.9, 152.8, 148.7, 147.2, 141.4, 140.8, 140.7, 140.6, 140.5, 140.3, 140.2, 137.7, 136.5, 130.8, 130.6, 130.3, 129.2, 128.8, 127.9, 127.8, 127.4, 127.2, 126.9, 126.8, 126.7, 126.5, 125.2, 124.9, 123.9, 123.8, 123.1, 122.9, 118.4. Note: The complicated ¹³C NMR spectrum is probably due to ¹³C—¹⁹F couplings. ¹⁹F NMR (375 MHz, acetone-d-6, 23° C., δ): −151.5. Mass Spectrometry: HRMS-FIA (m/z): Calcd for [C₃₁H₂₄N₄O₅PdS—C₅H₅N+C₂H₃N], 604.0265. Found, 604.0228.

### Example 2

### Influence of Substituents on the Sulfonyl Moiety

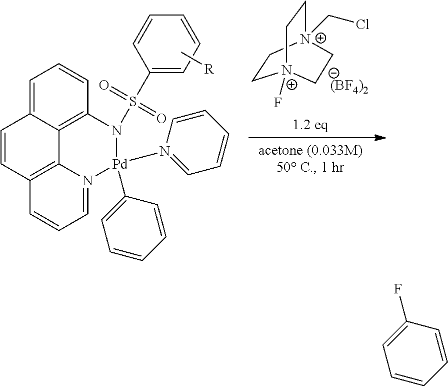

TABLE 4

| R | yield |
|---|---|
| 4-Me | 39% |
| 4-OMe | 20% |
| 4-NO₂ | 57% |
| 2-NO₂ | 57% |
| 3,5-(CF₃) | 55% |

Three additional nitrene-inserted complexes have been synthesized which have 3,5-bis(CF₃)phenyl, pentafluorophenyl, or 2,4-diNO₂ phenyl sulfonyl group on the amide moiety respectively. However, none of them gave significant increase in the fluorination yield.

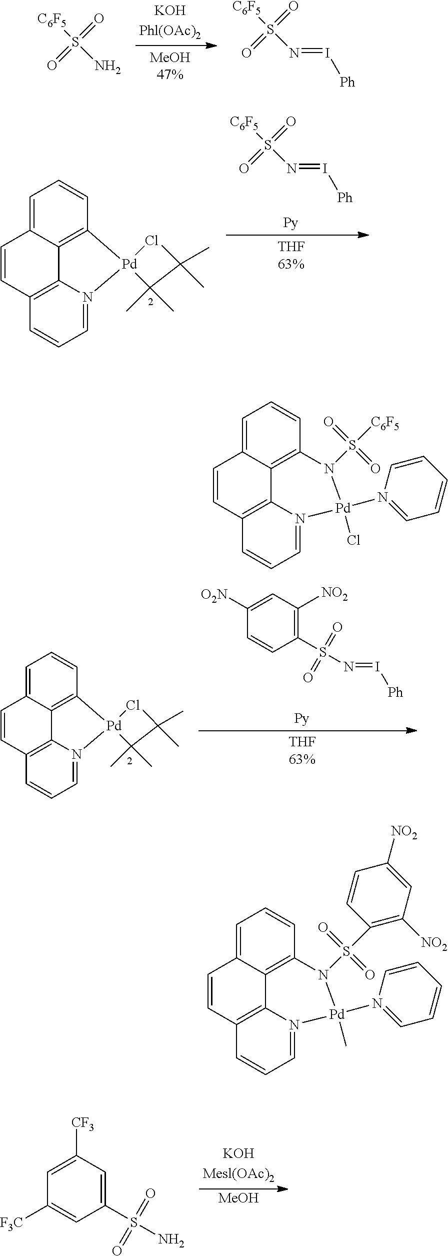

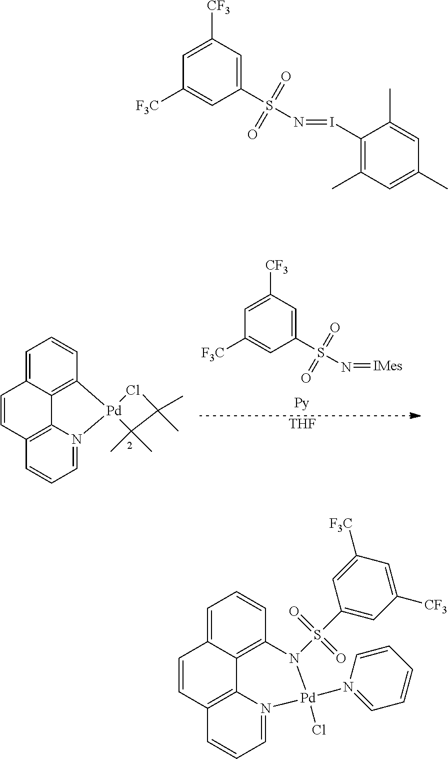

### Example 3

### Influence of Substituents on the Pyridinyl Moiety

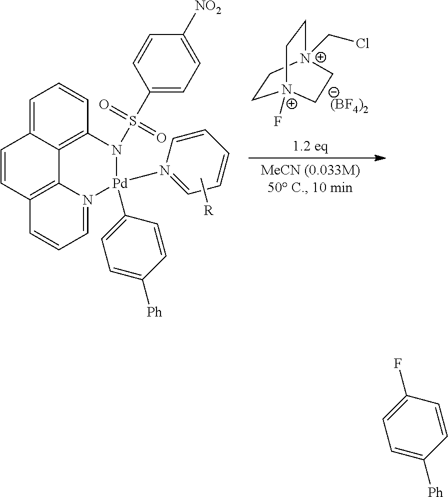

TABLE 5

| R | Yield |
|---|---|
| H | 63% |
| 4-Cl | 33% |
| 4-CN | 27% |
| 4-tBu | 52% |
| 4-NMe₂ | 4% |

### Example 4

### Influence of Substituents on the Organic Compound on Fluorination

Pd-complexes were prepared where the phenylpyridine moiety bears an electron-withdrawing Trifluoromethyl-Nitro-group and carried out fluorination reactions with these complexes.

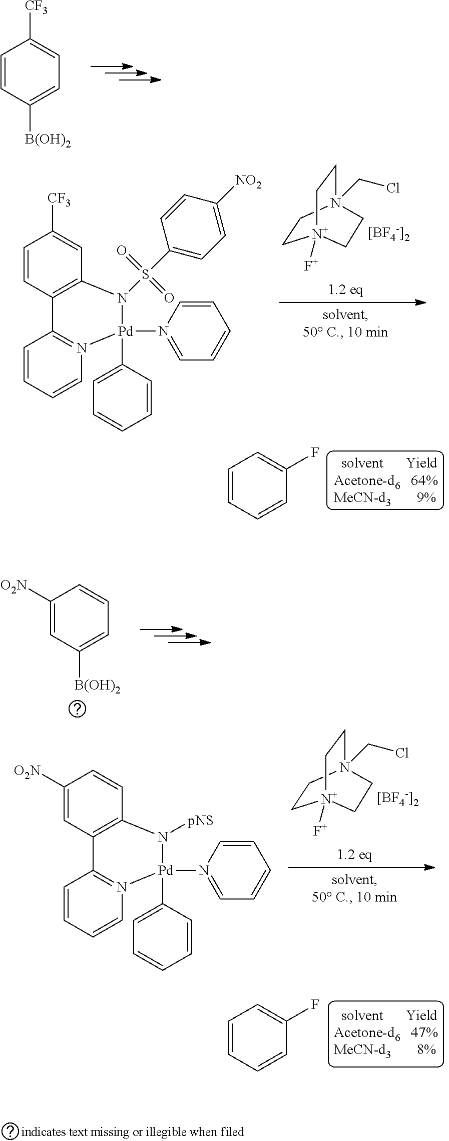

Other analogous complexes with an electron-donating tert-butyl group have also been synthesized.

### Example 5

### Solvent/Oxidant Screen in Fluorination Reactions

After fluorination had been carried out, all volatiles from the sample were removed on high-vac and the Pd-residue was analyzed by NMR.

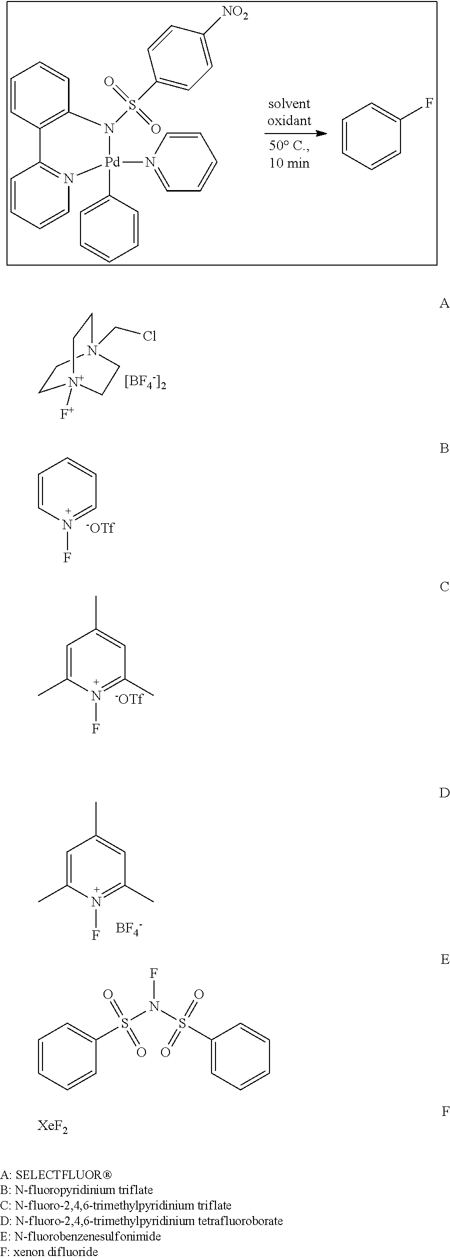

TABLE 6

| | Entry | Solvent | Oxidant | Yield [%] |
|---|---|---|---|---|
| Solvent | 1 | Acetone-d₆ | A | 82 |
| | 2 | Acetonitrile-d₃ | A | 44 |
| | 3 | Chloroform-d₁ | A | 0 |
| | 4 | DMF-d₇ | A | 0 |
| | 5 | Benzene-d₆ | A | 0 |
| | 6 | CD₂Cl₂ | A | 0 |
| | 7 | Methanol-d₄ | A | 0 |
| Oxidant | 8 | Acetone-d₆ | A | 82 |
| | 9 | Acetone-d₆ | B | 76 |
| | 10 | Acetone-d₆ | C | 17 |
| | 11 | Acetone-d₆ | D | 22 |
| | 12 | Acetone-d₆ | E | 30 |
| | 13 | Acetone-d₆ | | 11 |
| | 14 | Acetonitrile-d₃ | F | 20 |

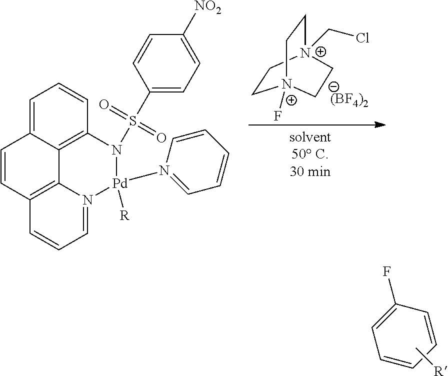

TABLE 7

| product | yield | solvent |
|---|---|---|
| 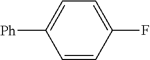 [SMILES: Fc1ccc(-c2ccccc2)cc1] | 76% | MeCN |
| 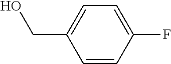 | 78% | MeCN |
| 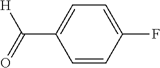 | 65% | MeCN |
| 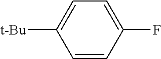 [SMILES: CC(C)(C)c1ccc(F)cc1] | 82% | MeCN |
| 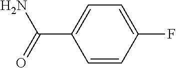 | 78% | MeCN |
| 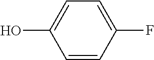 | 31% | acetone |
| 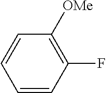 | 29% | acetone |
| 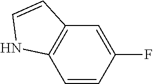 | 0% | acetone |
| 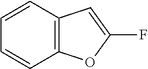 | 8% | MeCN |

### Example 6

### Mechanistic Studies

In order to get any useful information about palladium-mediated C—F bond formation process, isolation of Pd(IV)-F complex was attempted. Hoping to get crystalline compound, the dimethyl(naphthalenylmethyl)amine palladium complex was synthesized with tetrapyrazoylborate.

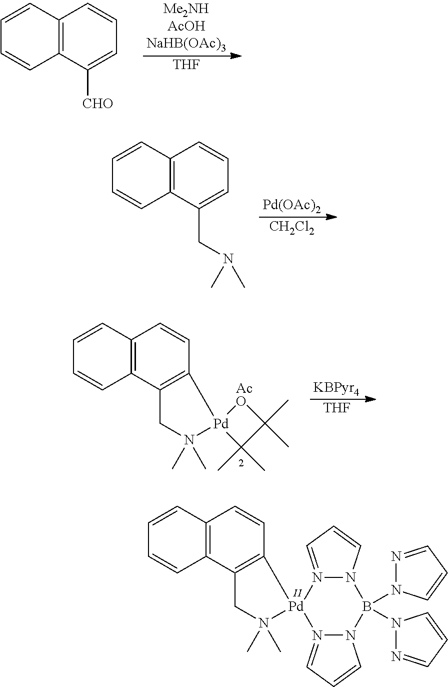

Upon treatment with N-fluoropyridinium triflate, formation of Pd(IV)-F complex was confirmed by ¹H and ¹⁹F NMR.

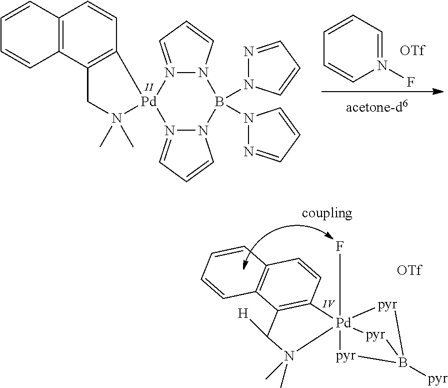

Several N-fluoropyridinium salts have been synthesized with different counter anions. At the time of filing of the present application, only 14 crystal structures of organopalladium fluorine complexes have been reported on the cambridge crystal structure database and all of them are Pd(II) complexes. The complex shown below is the first organopalladium(IV) fluorine complex ever isolated and characterized by x-ray crystarography. As we expected, the bond length of this complex is much shorter than that of Pd(II) complexes.

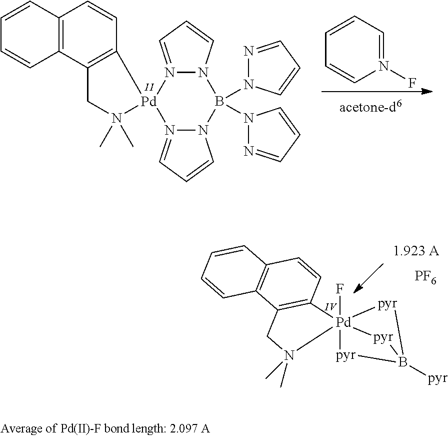

### Example 7

### Fluorination with N-fluorobenzenesulfonimide or XeF₂

### Synthesis of fluorobiphenyl

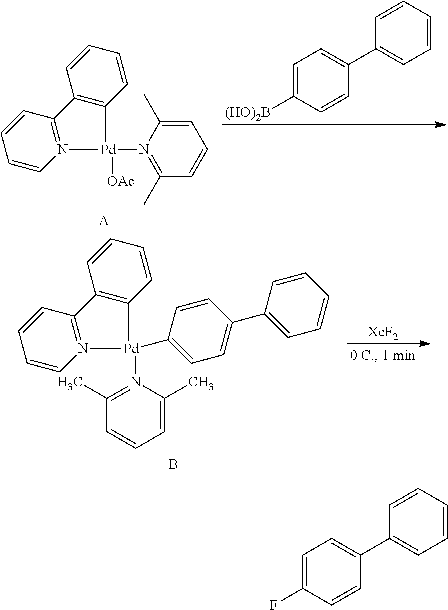

Under an inert atmosphere of N₂, to a stirred solution of Pd complex (B) (5.2 mg, 0.01 mmol, 1.00 equiv) in CH₂Cl₂ (00.1 mL) at room temperature was added XeF₂ (1.7 mg, 0.01 mmol, 1.00 equiv) in one portion. After stirring for one minute, the solution was concentrated and products were isolated by preparative TLC (50% yield).

Proposed Synthesis of ¹⁸F labeled fluorobiphenyl from ¹⁸F labeled XeF₂.

The above method can be modified by using ¹⁸F labeled XeF₂. 18F labeled XeF₂ can be prepared by any of the methods described in Constaminou et al., J. Am. Chem. Soc. (2001) 123:1780-1781 and Vasdev et al., J. Am. Chem. Soc. (2002) 124:12863-12868, incorporated herein by reference.

Proposed Synthesis of ¹⁸F labeled fluorobiphenyl from ¹⁸F labeled N-fluorobenzenesulfonamide.

The above method can be modified by using ¹⁸F labeled N-fluorobenzenesulfonamide instead of ¹⁸F labeled XeF₂. ¹⁸F labeled N-fluorobenzenesulfonamide can be prepared by the method of Teare et al., Chem. Comm. (2007) 2330-2332, incorporated herein by reference.

### Proposed Synthesis of LIPITOR and ¹⁸F-labeled LIPITOR

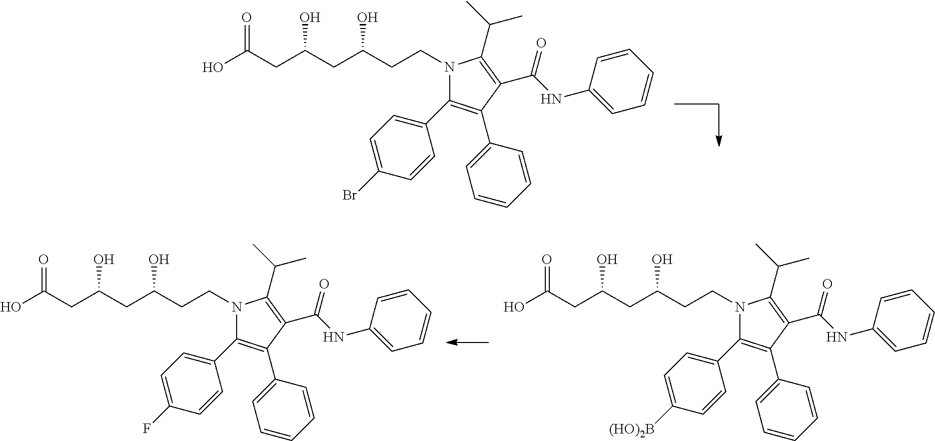 [SMILES: CC(C)c1c(C(=O)Nc2ccccc2)c(-c2ccccc2)c(-c2ccc(Br)cc2)n1CC[C@@H](O)C[C@@H](O)CC(=O)O ; CC(C)c1c(C(=O)Nc2ccccc2)c(-c2ccccc2)c(-c2ccc(F)cc2)n1CC[C@@H](O)C[C@@H](O)CC(=O)O ; CC(C)c1c(C(=O)Nc2ccccc2)c(-c2ccccc2)c(-c2ccc(B(O)O)cc2)n1CC[C@@H](O)C[C@@H](O)CC(=O)O]

LIPITOR can be prepared by borylating the starting material aryl bromide or chloride (see Billingsley et al., Angew. Chem. Int. Ed. (2007) 46:5359-5363, Ishiyama et al., JACS (2002) 124:390-391; Murphy et al., Organic Letters (2007) 9:757-760, for exemplary borylations of arenes, aryl bromides and aryl chlorides, each incorporated herein by reference). The boronic acid compound is then treated using any of the above disclosed methods to provide LIPITOR or ¹⁸F-labeled LIPITOR.

### Example 8

### Crystal Structure of (Acetato){benzo[h]quinolin-10-yl(4-nitrophenylsulfonyl)amide}(pyridine) palladium(II) (complex 1)

The compound was crystallized from a dichloromethane/diethyl ether solution as pale yellow plates. A crystal 0.025 mm×0.150 mm×0.175 mm in size was selected, mounted on a nylon loop with Paratone-N oil, and transferred to a Bruker SMART APEX II diffractometer equipped with an Oxford Cryosystems 700 Series Cryostream Cooler and Mo Kα radiation (λ=0.71073 Å). A total of 1601 frames were collected at 193 (2) K to θₘₐₓ=27.50° with an oscillation range of 0.5°/frame, and an exposure time of 10 s/frame using the APEX2 suite of software. (Bruker AXS, 2006a) Unit cell refinement on all observed reflections, and data reduction with corrections for Lp and decay were performed using SAINT. (Bruker AXS, 2006b) Scaling and a numerical absorption correction were done using SADABS. (Bruker AXS, 2004) The minimum and maximum transmission factors were 0.8562 and 0.9775, respectively. A total of 17370 reflections were collected, 5486 were unique (Rᵢₙₜ=0.0586), and 4388 had I>2σ(I). The lack of systematic absences was consistent with the compound having crystallized in the triclinic space group P1 or P 1̅. The centrosymmetric space group P 1̅ (No. 2) was selected. The observed mean |E²−1| value was 0.831 (versus the expectation values of 0.968 and 0.736 for centric and noncentric data, respectively).

The structure was solved by direct methods and refined by full-matrix least-squares on F² using SHELXTL. (Bruker AXS, 2001) The asymmetric unit was found to contain a single molecule of (Acetato){benzo[h]quinolin-10-yl(4-nitrophenylsulfonyl)amide}(pyridine)- palladium(II). All of the nonhydrogen atoms were refined with anisotropic displacement coefficients. The hydrogen atoms were assigned isotropic displacement coefficients U(H)=1.2U(C) or 1.5 U(C_{methyl}), and their coordinates were allowed to ride on their respective carbons. The refinement converged to R(F)=0.0376, wR(F²)=0.0859, and S=1.030 for 4388 reflections with I>2σ(I), and R(F)=0.0518, wR(F²)=0.0935, and S=1.030 for 5486 unique reflections and 344 parameter. The maximum |Δ/σ| in the final cycle of least-squares was 0.001, and the residual peaks on the final difference-Fourier map ranged from −0.543 to 0.525 eÅ⁻³. Scattering factors were taken from the International Tables for Crystallography, Volume C. (Maslen et al., 1992, and Creagh & McAuley, 1992). R(F)=R1=Σ∥Fₒ|−|F_{c}∥/Σ|Fₒ|, wR(F²)=wR2=[Σw(Fₒ²−F_{c}²)²/τw(Fₒ²)²]^{1/2}, and S=Goodness-of-fit on F²=[Σw(Fₒ²−F_{c}²)²/(n−p)]^{1/2}, where n is the number of reflections and p is the number of parameters refined.

### REFERENCES

Bruker AXS (2001). SHELXTL v6.12. Bruker Analytical X-ray Systems Inc., Madison, Wis., USA; Bruker AXS (2004). SADABS. Bruker Analytical X-ray Systems Inc., Madison, Wis., USA; Bruker AXS (2006a). APEX2 v2.1-0. Bruker Analytical X-ray Systems Inc., Madison, Wis., USA; Bruker AXS (2006b). SAINT V7.34A. Bruker Analytical X-ray Systems Inc., Madison, Wis., USA; Creagh, D. C. & McAuley, W. J. (1992). International Tables for Crystallography: Mathematical, Physical and Chemical Tables, Vol C, edited by A. J. C. Wilson, pp. 206-222; Dordrecht, The Netherlands: Kluwer; Maslen, E. N., Fox, A. G. & O'Keefe, M. A. (1992). International Tables for Crystallography: Mathematical, Physical and Chemical Tables, Vol C, edited by A. J. C. Wilson, pp. 476-516. Dordrecht, The Netherlands: Kluwer.

TABLE 8
Crystal data and structure refinement for complex 1.

| Identification code | tr019 = | |
|---|---|---|
| | [Pd(C₅H₅N)(C₂H₃O₂)(C₁₉H₁₂N₃O₄S)] | |
| Formula | C26 H20 N4 O6 Pd S | |
| Formula weight | 622.92 | |
| Temperature | 193(2) K | |
| Wavelength | 0.71073 Å | |
| Crystal system | Triclinic | |
| Space group | P [symbol] (No. 2) | |
| Unit cell dimensions | a = 9.1803(2) Å | α = 67.735(1)° |
| | b = 11.3199(2) Å | β = 87.215(1)° |
| | c = 12.8456(2) Å | γ = 75.798(1)° |

| | |
|---|---|
| Volume | 1196.16(4) Å³ |
| Z | 2 |
| Density (calculated) | 1.730 Mg/m³ |
| Absorption coefficient | 0.916 mm⁻¹ |
| F(000) | 628 |
| Crystal size | 0.175 × 0.150 × 0.025 mm³ |
| Theta range for data collection | 1.72 to 27.50° |
| Index ranges | −11 <= h <= 11, −14 <= k <= |
| | 14, −16 <= l <= 16 |
| Reflections collected | 17370 |
| Independent reflections | 5486 [R(int) = 0.0586] |
| Completeness to theta = 27.50° | 100.0% |
| Absorption correction | Numerical |
| Max. and min. transmission | 0.9775 and 0.8562 |
| Refinement method | Full-matrix least-squares on F² |
| Data/restraints/parameters | 5486/0/344 |
| Goodness-of-fit on F² | 1.030 |
| Final R indices [I > 2sigma(I)] | R1 = 0.0376, wR2 = 0.0859 |
| R indices (all data) | R1 = 0.0518, wR2 = 0.0935 |
| Largest diff. peak and hole | 0.525 and −0.543 e.Å⁻³ |

[symbol]

indicates data missing or illegible when filed

TABLE 9
Atomic coordinates (×10⁴) and equivalent isotropic displacement
parameters (Å² × 10³) for complex 1. U(eq) is defined as
one third of the trace of the orthogonalized Uᵢⱼ tensor.

| | x | y | z | U(eq) |
|---|---|---|---|---|
| Pd(1) | 4840(1) | 2637(1) | 4798(1) | 26(1) |
| N(1) | 3102(3) | 4272(2) | 4259(2) | 26(1) |
| C(2) | 2890(4) | 5012(3) | 4873(3) | 32(1) |
| C(3) | 1661(4) | 6063(3) | 4707(3) | 39(1) |
| C(4) | 580(4) | 6343(3) | 3902(3) | 38(1) |
| C(4A) | 775(4) | 5593(3) | 3228(3) | 32(1) |
| C(5) | −380(4) | 5824(4) | 2426(3) | 41(1) |
| C(6) | −261(4) | 5076(4) | 1826(3) | 39(1) |
| C(6A) | 1080(4) | 4075(3) | 1900(3) | 31(1) |
| C(7) | 1180(4) | 3344(4) | 1223(3) | 37(1) |
| C(8) | 2461(4) | 2415(4) | 1242(3) | 40(1) |
| C(9) | 3712(4) | 2224(3) | 1921(3) | 34(1) |
| C(10) | 3666(4) | 2950(3) | 2584(3) | 28(1) |
| C(10A) | 2312(4) | 3847(3) | 2642(2) | 26(1) |
| C(10B) | 2095(3) | 4574(3) | 3386(3) | 27(1) |
| N(11) | 5028(3) | 2751(3) | 3197(2) | 26(1) |
| S(12) | 6202(1) | 3573(1) | 2506(1) | 27(1) |
| O(13) | 5514(3) | 4932(2) | 1859(2) | 38(1) |
| O(14) | 7439(3) | 3279(2) | 3280(2) | 37(1) |
| C(15) | 6910(3) | 2852(3) | 1516(3) | 27(1) |
| C(16) | 6808(4) | 3595(3) | 376(3) | 32(1) |
| C(17) | 7311(4) | 2987(3) | −372(3) | 35(1) |
| C(18) | 7891(4) | 1658(3) | 42(3) | 32(1) |
| C(19) | 8031(4) | 878(3) | 1168(3) | 34(1) |
| C(20) | 7551(4) | 1496(3) | 1922(3) | 33(1) |
| N(21) | 8339(3) | 988(3) | −762(3) | 39(1) |
| O(22) | 8495(4) | 1662(3) | −1729(2) | 63(1) |
| O(23) | 8554(3) | −199(3) | −403(2) | 50(1) |
| N(24) | 6597(3) | 1019(3) | 5347(2) | 28(1) |
| C(25) | 6873(4) | 95(3) | 4898(3) | 30(1) |
| C(26) | 8107(4) | −960(3) | 5225(3) | 33(1) |
| C(27) | 9115(4) | −1073(3) | 6046(3) | 38(1) |
| C(28) | 8817(4) | −127(3) | 6519(3) | 37(1) |
| C(29) | 7574(4) | 883(3) | 6161(3) | 32(1) |
| O(30) | 4770(3) | 2551(2) | 6401(2) | 35(1) |
| C(31) | 3952(4) | 1895(3) | 7113(3) | 32(1) |
| O(32) | 2993(3) | 1461(3) | 6865(2) | 59(1) |
| C(33) | 4271(4) | 1725(4) | 8314(3) | 47(1) |

TABLE 10
Bond lengths [Å] and angles [°] for complex 1.

| | | | |
|---|---|---|---|
| Pd(1)—N(11) | 2.013(2) | C(7)—C(8) | 1.366(5) |
| Pd(1)—O(30) | 2.023(2) | C(7)—H(7) | 0.9500 |
| Pd(1)—N(1) | 2.033(3) | C(8)—C(9) | 1.400(5) |
| Pd(1)—N(24) | 2.034(3) | C(8)—H(8) | 0.9500 |
| N(1)—C(2) | 1.330(4) | C(9)—C(10) | 1.383(4) |
| N(1)—C(10B) | 1.372(4) | C(9)—H(9) | 0.9500 |
| C(2)—C(3) | 1.381(5) | C(10)—C(10A) | 1.417(4) |
| C(2)—H(2) | 0.9500 | C(10)—N(11) | 1.434(4) |
| C(3)—C(4) | 1.361(5) | C(10A)—C(10B) | 1.460(4) |
| C(3)—H(3) | 0.9500 | N(11)—S(12) | 1.609(3) |
| C(4)—C(4A) | 1.403(5) | S(12)—O(14) | 1.436(2) |
| C(4)—H(4) | 0.9500 | S(12)—O(13) | 1.436(2) |
| C(4A)—C(10B) | 1.414(4) | S(12)—C(15) | 1.775(3) |
| C(4A)—C(5) | 1.422(5) | C(15)—C(16) | 1.380(4) |
| C(5)—C(6) | 1.328(5) | C(15)—C(20) | 1.398(4) |
| C(5)—H(5) | 0.9500 | C(16)—C(17) | 1.383(4) |
| C(6)—C(6A) | 1.433(5) | C(16)—H(16) | 0.9500 |
| C(6)—H(6) | 0.9500 | C(17)—C(18) | 1.363(5) |
| C(6A)—C(7) | 1.396(5) | C(17)—H(17) | 0.9500 |
| C(6A)—C(10A) | 1.424(4) | C(18)—C(19) | 1.372(5) |
| C(18)—N(21) | 1.486(4) | C(4)—C(3)—H(3) | 120.5 |
| C(19)—C(20) | 1.393(5) | C(2)—C(3)—H(3) | 120.5 |
| C(19)—H(19) | 0.9500 | C(3)—C(4)—C(4A) | 119.2(3) |
| C(20)—H(20) | 0.9500 | C(3)—C(4)—H(4) | 120.4 |
| N(21)—O(22) | 1.210(4) | C(4A)—C(4)—H(4) | 120.4 |
| N(21)—O(23) | 1.210(4) | C(4)—C(4A)—C(10B) | 119.6(3) |
| N(24)—C(25) | 1.343(4) | C(4)—C(4A)—C(5) | 119.9(3) |
| N(24)—C(29) | 1.351(4) | C(10B)—C(4A)—C(5) | 120.4(3) |
| C(25)—C(26) | 1.374(4) | C(6)—C(5)—C(4A) | 121.0(3) |
| C(25)—H(25) | 0.9500 | C(6)—C(5)—H(5) | 119.5 |
| C(26)—C(27) | 1.386(5) | C(4A)—C(5)—H(5) | 119.5 |
| C(26)—H(26) | 0.9500 | C(5)—C(6)—C(6A) | 121.3(3) |
| C(27)—C(28) | 1.387(5) | C(5)—C(6)—H(6) | 119.3 |
| C(27)—H(27) | 0.9500 | C(6A)—C(6)—H(6) | 119.3 |
| C(28)—C(29) | 1.353(5) | C(7)—C(6A)—C(10A) | 120.3(3) |
| C(28)—H(28) | 0.9500 | C(7)—C(6A)—C(6) | 119.5(3) |
| C(29)—H(29) | 0.9500 | C(10A)—C(6A)—C(6) | 120.2(3) |
| O(30)—C(31) | 1.282(4) | C(8)—C(7)—C(6A) | 121.0(3) |
| C(31)—O(32) | 1.215(4) | C(8)—C(7)—H(7) | 119.5 |
| C(31)—C(33) | 1.515(5) | C(6A)—C(7)—H(7) | 119.5 |
| C(33)—H(33A) | 0.9800 | C(7)—C(8)—C(9) | 119.5(3) |
| C(33)—H(33B) | 0.9800 | C(7)—C(8)—H(8) | 120.2 |
| C(33)—H(33C) | 0.9800 | C(9)—C(8)—H(8) | 120.2 |
| N(11)—Pd(1)—O(30) | 176.58(10) | C(10)—C(9)—C(8) | 121.1(3) |
| N(11)—Pd(1)—N(1) | 88.69(10) | C(10)—C(9)—H(9) | 119.5 |
| O(30)—Pd(1)—N(1) | 92.58(10) | C(8)—C(9)—H(9) | 119.5 |
| N(11)—Pd(1)—N(24) | 91.77(10) | C(9)—C(10)—C(10A) | 120.2(3) |
| O(30)—Pd(1)—N(24) | 86.91(10) | C(9)—C(10)—N(11) | 117.3(3) |
| N(1)—Pd(1)—N(24) | 179.06(11) | C(10A)—C(10)—N(11) | 122.5(3) |
| C(2)—N(1)—C(10B) | 119.6(3) | C(10)—C(10A)—C(6A) | 117.5(3) |
| C(2)—N(1)—Pd(1) | 115.8(2) | C(10)—C(10A)—C(10B) | 125.0(3) |
| C(10B)—N(1)—Pd(1) | 124.3(2) | C(6A)—C(10A)—C(10B) | 117.5(3) |
| N(1)—C(2)—C(3) | 123.3(3) | N(1)—C(10B)—C(4A) | 118.9(3) |
| N(1)—C(2)—H(2) | 118.4 | N(1)—C(10B)—C(10A) | 122.2(3) |
| C(3)—C(2)—H(2) | 118.4 | C(4A)—C(10B)—C(10A) | 118.9(3) |
| C(4)—C(3)—C(2) | 119.1(3) | C(10)—N(11)—S(12) | 115.9(2) |
| C(10)—N(11)—Pd(1) | 116.91(19) | C(25)—N(24)—C(29) | 117.9(3) |
| S(12)—N(11)—Pd(1) | 114.09(14) | C(25)—N(24)—Pd(1) | 122.7(2) |
| O(14)—S(12)—O(13) | 117.99(15) | C(29)—N(24)—Pd(1) | 119.4(2) |
| O(14)—S(12)—N(11) | 106.82(14) | N(24)—C(25)—C(26) | 122.7(3) |
| O(13)—S(12)—N(11) | 113.57(14) | N(24)—C(25)—H(25) | 118.7 |
| O(14)—S(12)—C(15) | 106.43(14) | C(26)—C(25)—H(25) | 118.7 |
| O(13)—S(12)—C(15) | 106.12(15) | C(25)—C(26)—C(27) | 118.8(3) |
| N(11)—S(12)—C(15) | 104.92(14) | C(25)—C(26)—H(26) | 120.6 |
| C(16)—C(15)—C(20) | 120.5(3) | C(27)—C(26)—H(26) | 120.6 |
| C(16)—C(15)—S(12) | 121.4(3) | C(26)—C(27)—C(28) | 118.4(3) |
| C(20)—C(15)—S(12) | 118.1(2) | C(26)—C(27)—H(27) | 120.8 |
| C(15)—C(16)—C(17) | 119.7(3) | C(28)—C(27)—H(27) | 120.8 |
| C(15)—C(16)—H(16) | 120.2 | C(29)—C(28)—C(27) | 119.7(3) |
| C(17)—C(16)—H(16) | 120.2 | C(29)—C(28)—H(28) | 120.1 |
| C(18)—C(17)—C(16) | 118.8(3) | C(27)—C(28)—H(28) | 120.1 |
| C(18)—C(17)—H(17) | 120.6 | N(24)—C(29)—C(28) | 122.5(3) |
| C(16)—C(17)—H(17) | 120.6 | N(24)—C(29)—H(29) | 118.7 |
| C(17)—C(18)—C(19) | 123.8(3) | C(28)—C(29)—H(29) | 118.7 |
| C(17)—C(18)—N(21) | 118.9(3) | C(31)—O(30)—Pd(1) | 121.2(2) |
| C(19)—C(18)—N(21) | 117.2(3) | O(32)—C(31)—O(30) | 124.3(3) |
| C(18)—C(19)—C(20) | 117.4(3) | O(32)—C(31)—C(33) | 122.2(3) |
| C(18)—C(19)—H(19) | 121.3 | O(30)—C(31)—C(33) | 113.4(3) |
| C(20)—C(19)—H(19) | 121.3 | C(31)—C(33)—H(33A) | 109.5 |
| C(19)—C(20)—C(15) | 119.9(3) | C(31)—C(33)—H(33B) | 109.5 |
| C(19)—C(20)—H(20) | 120.1 | H(33A)—C(33)—H(33B) | 109.5 |
| C(15)—C(20)—H(20) | 120.1 | C(31)—C(33)—H(33C) | 109.5 |
| O(22)—N(21)—O(23) | 124.2(3) | H(33A)—C(33)—H(33C) | 109.5 |
| O(22)—N(21)—C(18) | 117.8(3) | H(33B)—C(33)—H(33C) | 109.5 |
| O(23)—N(21)—C(18) | 118.0(3) | | |

TABLE 11
Anisotropic displacement parameters (Å² × 10³) for complex 1.
The anisotropic displacement factor exponent takes the form:
−2π²[h²a*²U₁₁ + . . . + 2 h k a* b* U₁₂]

| | U₁₁ | U₂₂ | U₃₃ | U₂₃ | U₁₃ | U₁₂ |
|---|---|---|---|---|---|---|
| Pd(1) | 28(1) | 31(1) | 19(1) | −11(1) | 0(1) | −5(1) |
| N(1) | 29(1) | 31(1) | 22(1) | −13(1) | 2(1) | −9(1) |
| C(2) | 34(2) | 37(2) | 27(2) | −15(2) | 0(1) | −10(2) |
| C(3) | 51(2) | 41(2) | 34(2) | −23(2) | 8(2) | −13(2) |
| C(4) | 42(2) | 35(2) | 36(2) | −16(2) | 5(2) | −3(2) |
| C(4A) | 32(2) | 33(2) | 27(2) | −8(1) | 2(1) | −5(1) |
| C(5) | 30(2) | 45(2) | 38(2) | −12(2) | −3(2) | 3(2) |
| C(6) | 29(2) | 49(2) | 31(2) | −8(2) | −3(2) | −10(2) |
| C(6A) | 33(2) | 38(2) | 23(2) | −9(1) | 1(1) | −15(2) |
| C(7) | 38(2) | 50(2) | 26(2) | −13(2) | −5(2) | −17(2) |
| C(8) | 51(2) | 51(2) | 32(2) | −25(2) | 3(2) | −21(2) |
| C(9) | 35(2) | 42(2) | 31(2) | −21(2) | 6(2) | −11(2) |
| C(10) | 31(2) | 34(2) | 19(2) | −9(1) | 0(1) | −10(1) |
| C(10A) | 32(2) | 30(2) | 18(2) | −8(1) | 2(1) | −13(1) |
| C(10B) | 26(2) | 31(2) | 23(2) | −8(1) | 4(1) | −11(1) |
| N(11) | 26(1) | 35(1) | 18(1) | −13(1) | 1(1) | −7(1) |
| S(12) | 28(1) | 33(1) | 23(1) | −13(1) | 2(1) | −7(1) |
| O(13) | 45(2) | 32(1) | 37(1) | −13(1) | 4(1) | −8(1) |
| O(14) | 30(1) | 56(2) | 33(1) | −21(1) | 3(1) | −16(1) |
| C(15) | 21(2) | 37(2) | 24(2) | −13(1) | 1(1) | −5(1) |
| C(16) | 34(2) | 33(2) | 27(2) | −10(1) | 1(1) | −4(1) |
| C(17) | 37(2) | 41(2) | 23(2) | −12(2) | 4(1) | −5(2) |
| C(18) | 27(2) | 43(2) | 29(2) | −20(2) | 3(1) | −6(2) |
| C(19) | 34(2) | 34(2) | 30(2) | −11(2) | 6(2) | −3(2) |
| C(20) | 33(2) | 38(2) | 24(2) | −9(1) | 3(1) | −4(2) |
| N(21) | 38(2) | 49(2) | 37(2) | −26(2) | 6(1) | −9(2) |
| O(22) | 94(2) | 65(2) | 33(2) | −26(2) | 13(2) | −14(2) |
| O(23) | 57(2) | 53(2) | 54(2) | −34(1) | 12(1) | −17(1) |
| N(24) | 31(1) | 31(1) | 21(1) | −8(1) | 1(1) | −8(1) |
| C(25) | 34(2) | 35(2) | 21(2) | −9(1) | 2(1) | −10(2) |
| C(26) | 35(2) | 29(2) | 34(2) | −11(2) | 8(2) | −9(1) |
| C(27) | 33(2) | 36(2) | 34(2) | −6(2) | 3(2) | −2(2) |
| C(28) | 31(2) | 48(2) | 28(2) | −11(2) | −2(2) | −10(2) |
| C(29) | 35(2) | 37(2) | 25(2) | −12(1) | −1(1) | −9(2) |
| O(30) | 40(1) | 45(1) | 21(1) | −15(1) | 1(1) | −10(1) |
| C(31) | 32(2) | 35(2) | 28(2) | −14(2) | 2(1) | −1(2) |
| O(32) | 70(2) | 83(2) | 34(2) | −22(2) | 8(1) | −41(2) |
| C(33) | 47(2) | 70(3) | 27(2) | −20(2) | 5(2) | −18(2) |

TABLE 12
Hydrogen coordinates (×10⁴) and isotropic displacement
parameters (Å² × 10³) for complex 1.

| | x | y | z | U(eq) |
|---|---|---|---|---|
| H(2) | 3620 | 4807 | 5455 | 38 |
| H(3) | 1571 | 6584 | 5149 | 47 |
| H(4) | −295 | 7037 | 3798 | 46 |
| H(5) | −1251 | 6524 | 2317 | 49 |
| H(6) | −1082 | 5206 | 1339 | 46 |
| H(7) | 343 | 3495 | 742 | 44 |
| H(8) | 2503 | 1903 | 797 | 48 |
| H(9) | 4606 | 1585 | 1927 | 40 |
| H(16) | 6394 | 4519 | 105 | 39 |
| H(17) | 7254 | 3486 | −1160 | 42 |
| H(19) | 8440 | −46 | 1424 | 41 |
| H(20) | 7659 | 997 | 2710 | 40 |
| H(25) | 6188 | 174 | 4330 | 36 |
| H(26) | 8266 | −1600 | 4894 | 40 |
| H(27) | 9990 | −1781 | 6280 | 45 |
| H(28) | 9481 | −188 | 7091 | 44 |
| H(29) | 7382 | 1520 | 6496 | 39 |
| H(33A) | 3442 | 1452 | 8777 | 70 |
| H(33B) | 4367 | 2562 | 8328 | 70 |
| H(33C) | 5210 | 1051 | 8615 | 70 |

TABLE 13
Torsion angles [°] for complex 1.

| | | | |
|---|---|---|---|
| N(11)—Pd(1)—N(1)—C(2) | −152.3(2) | C(4)—C(4A)—C(10B)—C(10A) | −177.1(3) |
| O(30)—Pd(1)—N(1)—C(2) | 24.5(2) | C(5)—C(4A)—C(10B)—C(10A) | 5.7(5) |
| N(11)—Pd(1)—N(1)—C(10B) | 33.5(2) | C(10)—C(10A)—C(10B)—N(1) | −12.6(5) |
| O(30)—Pd(1)—N(1)—C(10B) | −149.6(2) | C(6A)—C(10A)—C(10B)—N(1) | 168.7(3) |
| C(10B)—N(1)—C(2)—C(3) | 2.4(5) | C(10)—C(10A)—C(10B)—C(4A) | 169.7(3) |
| Pd(1)—N(1)—C(2)—C(3) | −172.0(3) | C(6A)—C(10A)—C(10B)—C(4A) | −8.9(4) |
| N(1)—C(2)—C(3)—C(4) | 2.1(5) | C(9)—C(10)—N(11)—S(12) | 83.4(3) |
| C(2)—C(3)—C(4)—C(4A) | −2.8(5) | C(10A)—C(10)—N(11)—S(12) | −96.7(3) |
| C(3)—C(4)—C(4A)—C(10B) | −0.8(5) | C(9)—C(10)—N(11)—Pd(1) | −137.6(3) |
| C(3)—C(4)—C(4A)—C(5) | 176.5(4) | C(10A)—C(10)—N(11)—Pd(1) | 42.3(4) |
| C(4)—C(4A)—C(5)—C(6) | −175.9(4) | N(1)—Pd(1)—N(11)—C(10) | −47.3(2) |
| C(10B)—C(4A)—C(5)—C(6) | 1.4(5) | N(24)—Pd(1)—N(11)—C(10) | 133.5(2) |
| C(4A)—C(5)—C(6)—C(6A) | −4.9(6) | N(1)—Pd(1)—N(11)—S(12) | 92.38(15) |
| C(5)—C(6)—C(6A)—C(7) | −177.6(3) | N(24)—Pd(1)—N(11)—S(12) | −86.80(15) |
| C(5)—C(6)—C(6A)—C(10A) | 1.2(5) | C(10)—N(11)—S(12)—O(14) | 177.5(2) |
| C(10A)—C(6A)—C(7)—C(8) | −1.2(5) | Pd(1)—N(11)—S(12)—O(14) | 37.32(18) |
| C(6)—C(6A)—C(7)—C(8) | 177.7(3) | C(10)—N(11)—S(12)—O(13) | 45.6(3) |
| C(6A)—C(7)—C(8)—C(9) | −2.2(5) | Pd(1)—N(11)—S(12)—O(13) | −94.50(17) |
| C(7)—C(8)—C(9)—C(10) | 0.7(5) | C(10)—N(11)—S(12)—C(15) | −69.8(2) |
| C(8)—C(9)—C(10)—C(10A) | 4.0(5) | Pd(1)—N(11)—S(12)—C(15) | 150.05(14) |
| C(8)—C(9)—C(10)—N(11) | −176.0(3) | O(14)—S(12)—C(15)—C(16) | −123.4(3) |
| C(9)—C(10)—C(10A)—C(6A) | −7.1(4) | O(13)—S(12)—C(15)—C(16) | 3.1(3) |
| N(11)—C(10)—C(10A)—C(6A) | 172.9(3) | N(11)—S(12)—C(15)—C(16) | 123.6(3) |
| C(9)—C(10)—C(10A)—C(10B) | 174.2(3) | O(14)—S(12)—C(15)—C(20) | 58.0(3) |
| N(11)—C(10)—C(10A)—C(10B) | −5.7(5) | O(13)—S(12)—C(15)—C(20) | −175.6(3) |
| C(7)—C(6A)—C(10A)—C(10) | 5.7(5) | N(11)—S(12)—C(15)—C(20) | −55.0(3) |
| C(6)—C(6A)—C(10A)—C(10) | −173.1(3) | C(20)—C(15)—C(16)—C(17) | 1.5(5) |
| C(7)—C(6A)—C(10A)—C(10B) | −175.5(3) | S(12)—C(15)—C(16)—C(17) | −177.1(3) |
| C(6)—C(6A)—C(10A)—C(10B) | 5.7(4) | C(15)—C(16)—C(17)—C(18) | 0.4(5) |
| C(2)—N(1)—C(10B)—C(4A) | −6.0(4) | C(16)—C(17)—C(18)—C(19) | −1.1(6) |
| Pd(1)—N(1)—C(10B)—C(4A) | 168.0(2) | C(16)—C(17)—C(18)—N(21) | 176.0(3) |
| C(2)—N(1)—C(10B)—C(10A) | 176.4(3) | C(17)—C(18)—C(19)—C(20) | −0.1(6) |
| Pd(1)—N(1)—C(10B)—C(10A) | −9.7(4) | N(21)—C(18)—C(19)—C(20) | −177.3(3) |
| C(4)—C(4A)—C(10B)—N(1) | 5.2(5) | C(18)—C(19)—C(20)—C(15) | 2.1(5) |
| C(5)—C(4A)—C(10B)—N(1) | −172.1(3) | C(16)—C(15)—C(20)—C(19) | −2.8(5) |
| S(12)—C(15)—C(20)—C(19) | 175.8(3) | N(11)—Pd(1)—N(24)—C(25) | −35.1(3) |
| C(17)—C(18)—N(21)—O(22) | 16.5(5) | O(30)—Pd(1)—N(24)—C(25) | 148.1(3) |
| C(19)—C(18)—N(21)—O(22) | −166.3(3) | N(11)—Pd(1)—N(24)—C(29) | 141.9(2) |
| C(17)—C(18)—N(21)—O(23) | −165.1(3) | O(30)—Pd(1)—N(24)—C(29) | −35.0(2) |
| C(19)—C(18)—N(21)—O(23) | 12.2(5) | C(29)—N(24)—C(25)—C(26) | −0.8(5) |
| | | Pd(1)—N(24)—C(25)—C(26) | 176.3(2) |
| | | N(24)—C(25)—C(26)—C(27) | −0.5(5) |
| | | C(25)—C(26)—C(27)—C(28) | 1.3(5) |
| | | C(26)—C(27)—C(28)—C(29) | −0.9(5) |
| | | C(25)—N(24)—C(29)—C(28) | 1.2(5) |
| | | Pd(1)—N(24)—C(29)—C(28) | −176.0(3) |
| | | C(27)—C(28)—C(29)—N(24) | −0.3(5) |
| | | N(1)—Pd(1)—O(30)—C(31) | 90.2(2) |
| | | N(24)—Pd(1)—O(30)—C(31) | −90.6(2) |
| | | Pd(1)—O(30)—C(31)—O(32) | −12.2(5) |
| | | Pd(1)—O(30)—C(31)—C(33) | 168.8(2) |

### Example 9

### Crystal Structure of (Phenyl) {benzo[h]quinolin-10-yl(4-nitrophenylsulfonyl)amide}(pyridine) palladium(II) (complex 4a)

The compound was crystallized from a dichloromethane/pentane solution as pale yellow prisms. A crystal 0.050 mm×0.075 mm×0.125 mm in size was selected, mounted on a nylon loop with Paratone-N oil, and transferred to a Bruker SMART APEX II diffractometer equipped with an Oxford Cryosystems 700 Series Cryostream Cooler and Mo Kα radiation (λ=0.71073 Å). A total of 3201 frames were collected at 193 (2) K to θₘₐₓ=27.500 with an oscillation range of 0.5°/frame, and an exposure time of 10 s/frame using the APEX2 suite of software. (Bruker AXS, 2006a) Unit cell refinement on all observed reflections, and data reduction with corrections for Lp and decay were performed using SAINT. (Bruker AXS, 2006b) Scaling and a multi-scan absorption correction were done using SADABS. (Bruker AXS, 2004) The minimum and maximum transmission factors were 0.9016 and 0.9589, respectively. A total of 67549 reflections were collected, 5932 were unique (Rᵢₙₜ=0.0494), and 5158 had I>2σ(I). Systematic absences were consistent with the compound having crystallized in the orthorhombic space group P2₁2₁2₁. The chiral space group P2₁2₁2₁ (No. 19) was selected based on an observed mean |E²−1| value of 0.758 (versus the expectation values of 0.968 and 0.736 for centric and noncentric data, respectively).

The structure was solved by direct methods and refined by full-matrix least-squares on F² using SHELXTL. (Bruker AXS, 2001) The asymmetric unit was found to contain a single molecule of (Phenyl)-{benzo[h]quinolin-10-yl(4-nitrophenylsulfonyl)amide}(pyridine)- palladium(II). All of the nonhydrogen atoms were refined with anisotropic displacement coefficients. The hydrogen atoms were assigned isotropic displacement coefficients U(H)=1.2U(C), and their coordinates were allowed to ride on their respective carbons. The refinement converged to R(F)=0.0329, wR(F²)=0.0657, and S=1.050 for 5158 reflections with I>2σ(I), and R(F)=0.0427, wR(F²)=0.0698, and S=1.050 for 5932 unique reflections and 361 parameter. The maximum |Δ/σ| in the final cycle of least-squares was 0.001, and the residual peaks on the final difference-Fourier map ranged from −0.576 to 0.488 eÅ⁻³. Scattering factors were taken from the International Tables for Crystallography, Volume C. (Maslen et al., 1992, and Creagh & McAuley, 1992).

The Flack absolute structure parameter refined to x=−0.03 (2) [versus the expectation values of 0 (within 3 esd's) for correct and +1 for inverted absolute structure] indicating that the coordinates provided below are for the correct hand of the molecule. (Flack, 1983).

R(F)=R1=Σ∥Fₒ|−|F_{c}∥/Σ⊕Fₒ|, wR(F²)=wR2=[Σw(Fₒ²−F_{c}²)₂/Σw(Fₒ²)₂]^{1/2}, and S=Goodness-of-fit on F²=[Σw(Fₒ²−F_{c}²)²/(n−p)]^{1/2}, where n is the number of reflections and p is the number of parameters refined.

### REFERENCES

Bruker AXS (2001). SHELXTL v6.12. Bruker Analytical X-ray Systems Inc., Madison, Wis., USA; Bruker AXS (2004). SADABS. Bruker Analytical X-ray Systems Inc., Madison, Wis., USA; Bruker AXS (2006a). APEX2 v2.1-0. Bruker Analytical X-ray Systems Inc., Madison, Wis., USA; Bruker AXS (2006b). SAINT V7.34A. Bruker Analytical X-ray Systems Inc., Madison, Wis., USA; Creagh, D. C. & McAuley, W. J. (1992). International Tables for Crystallography: Mathematical, Physical and Chemical Tables, Vol C, edited by A. J. C. Wilson, pp. 206-222; Dordrecht, The Netherlands: Kluwer; Maslen, E. N., Fox, A. G. & O'Keefe, M. A. (1992). International Tables for Crystallography: Mathematical, Physical and Chemical Tables, Vol C, edited by A. J. C. Wilson, pp. 476-516. Dordrecht, The Netherlands: Kluwer.

TABLE 14
Crystal data and structure refinement for complex 4a.

| Identification code | tr020 = | |
|---|---|---|
| | [Pd(C₅H₅N)(C₆H₅)(C₁₉H₁₂N₃O₄S)] | |
| Empirical formula | C30 H22 N4 O4 Pd S | |
| Formula weight | 640.98 | |
| Temperature | 193(2) K | |
| Wavelength | 0.71073 Å | |
| Crystal system | Orthorhombic | |
| Space group | P2₁2₁2₁ (No. 19) | |
| Unit cell dimensions | a = 9.5439(2) Å | α = 90° |
| | b = 13.8697(2) Å | β = 90° |
| | c = 19.5047(3) Å | γ = 90° |

| | |
|---|---|
| Volume | 2581.86(8) Å³ |
| Z | 4 |
| Density (calculated) | 1.649 Mg/m³ |
| Absorption coefficient | 0.846 mm⁻¹ |
| F(000) | 1296 |
| Crystal size | 0.125 × 0.075 × 0.050 mm³ |
| Theta range for data collection | 1.80 to 27.50° |
| Index ranges | −12 <= h <= 12, −18 <= k <= |
| | 18, −25 <= l <= 25 |
| Reflections collected | 67549 |
| Independent reflections | 5932 [R(int) = 0.1052] |
| Completeness to theta = 27.50° | 100.0% |
| Absorption correction | Semi-empirical from equivalents |
| Max. and min. transmission | 0.9589 and 0.9016 |
| Refinement method | Full-matrix least-squares on F² |
| Data/restraints/parameters | 5932/0/361 |
| Goodness-of-fit on F² | 1.050 |
| Final R indices [I > 2sigma(I)] | R1 = 0.0329, wR2 = 0.0657 |
| R indices (all data) | R1 = 0.0427, wR2 = 0.0698 |
| Absolute structure parameter | −0.03(2) |
| Largest diff. peak and hole | 0.488 and −0.576 e.Å⁻³ |

TABLE 15
Atomic coordinates (×10⁴) and equivalent isotropic displacement
parameters (Å² × 10³) for complex 4a. U(eq) is defined as
one third of the trace of the orthogonalized Uᵢⱼ tensor.

| | x | y | z | U(eq) |
|---|---|---|---|---|
| Pd(1) | 3903(1) | 4037(1) | 5980(1) | 21(1) |
| N(1) | 2588(3) | 3510(2) | 6728(2) | 22(1) |
| C(2) | 2947(4) | 2736(2) | 7096(2) | 26(1) |
| C(3) | 2005(4) | 2215(3) | 7492(2) | 31(1) |
| C(4) | 623(4) | 2450(3) | 7456(2) | 29(1) |
| C(4A) | 197(4) | 3258(3) | 7070(2) | 26(1) |
| C(5) | −1264(4) | 3483(3) | 6991(2) | 33(1) |
| C(6) | −1650(4) | 4263(3) | 6630(2) | 29(1) |
| C(6A) | −641(4) | 4937(3) | 6373(2) | 26(1) |
| C(7) | −1098(4) | 5796(2) | 6072(2) | 27(1) |
| C(8) | −174(4) | 6485(3) | 5863(2) | 30(1) |
| C(9) | 1256(4) | 6334(2) | 5961(2) | 26(1) |
| C(10) | 1760(4) | 5495(2) | 6248(2) | 20(1) |
| C(10A) | 819(3) | 4746(2) | 6442(2) | 21(1) |
| C(10B) | 1218(4) | 3829(2) | 6748(2) | 21(1) |
| N(11) | 3238(3) | 5400(2) | 6333(1) | 21(1) |
| S(12) | 3974(1) | 5953(1) | 6941(1) | 23(1) |
| O(13) | 5418(3) | 6114(2) | 6757(1) | 31(1) |
| O(14) | 3162(3) | 6770(2) | 7169(1) | 34(1) |
| C(15) | 4042(4) | 5134(2) | 7648(2) | 25(1) |
| C(16) | 5271(4) | 4623(3) | 7772(2) | 30(1) |
| C(17) | 5305(4) | 3918(3) | 8270(2) | 32(1) |
| C(18) | 4117(4) | 3750(2) | 8644(2) | 28(1) |
| C(19) | 2889(4) | 4255(3) | 8540(2) | 31(1) |
| C(20) | 2856(4) | 4963(3) | 8037(2) | 29(1) |
| N(21) | 4161(4) | 3002(2) | 9176(2) | 36(1) |
| O(22) | 5307(4) | 2691(2) | 9349(2) | 46(1) |
| O(23) | 3048(4) | 2732(2) | 9418(2) | 53(1) |
| N(24) | 4937(3) | 4560(2) | 5126(2) | 25(1) |
| C(25) | 5429(4) | 5474(3) | 5118(2) | 28(1) |
| C(26) | 5983(4) | 5888(3) | 4538(2) | 34(1) |
| C(27) | 6023(4) | 5385(3) | 3935(2) | 37(1) |
| C(28) | 5517(4) | 4448(3) | 3932(2) | 36(1) |
| C(29) | 4992(3) | 4060(3) | 4525(2) | 28(1) |
| C(30) | 4412(4) | 2693(2) | 5709(2) | 22(1) |
| C(31) | 5765(4) | 2365(3) | 5768(2) | 35(1) |
| C(32) | 6117(5) | 1430(3) | 5564(2) | 37(1) |
| C(33) | 5121(4) | 827(3) | 5307(2) | 35(1) |
| C(34) | 3767(5) | 1136(3) | 5257(2) | 37(1) |
| C(35) | 3411(4) | 2068(3) | 5459(2) | 32(1) |

TABLE 16
Bond lengths [Å] and angles [°] for complex 4a.

| | | | |
|---|---|---|---|
| Pd(1)—C(30) | 1.997(3) | C(16)—C(17) | 1.378(5) |
| Pd(1)—N(1) | 2.059(3) | C(16)—H(16) | 0.9500 |
| Pd(1)—N(24) | 2.067(3) | C(17)—C(18) | 1.368(5) |
| Pd(1)—N(11) | 2.109(3) | C(17)—H(17) | 0.9500 |
| N(1)—C(2) | 1.336(4) | C(18)—C(19) | 1.380(5) |
| N(1)—C(10B) | 1.381(4) | C(18)—N(21) | 1.468(4) |
| C(2)—C(3) | 1.388(5) | C(19)—C(20) | 1.388(5) |
| C(2)—H(2) | 0.9500 | C(19)—H(19) | 0.9500 |
| C(3)—C(4) | 1.361(5) | C(20)—H(20) | 0.9500 |
| C(3)—H(3) | 0.9500 | N(21)—O(23) | 1.222(4) |
| C(4)—C(4A) | 1.410(5) | N(21)—O(22) | 1.222(4) |
| C(4)—H(4) | 0.9500 | N(24)—C(25) | 1.351(4) |
| C(4A)—C(10B) | 1.404(5) | N(24)—C(29) | 1.363(4) |
| C(4A)—C(5) | 1.437(5) | C(25)—C(26) | 1.374(5) |
| C(5)—C(6) | 1.342(5) | C(25)—H(25) | 0.9500 |
| C(5)—H(5) | 0.9500 | C(26)—C(27) | 1.369(5) |
| C(6)—C(6A) | 1.434(5) | C(26)—H(26) | 0.9500 |
| C(6)—H(6) | 0.9500 | C(27)—C(28) | 1.386(5) |
| C(6A)—C(7) | 1.398(5) | C(27)—H(27) | 0.9500 |
| C(6A)—C(10A) | 1.425(5) | C(28)—C(29) | 1.370(5) |
| C(7)—C(8) | 1.363(5) | C(28)—H(28) | 0.9500 |
| C(7)—H(7) | 0.9500 | C(29)—H(29) | 0.9500 |
| C(8)—C(9) | 1.394(5) | C(30)—C(31) | 1.374(5) |
| C(8)—H(8) | 0.9500 | C(30)—C(35) | 1.379(5) |
| C(9)—C(10) | 1.378(4) | C(31)—C(32) | 1.397(5) |
| C(9)—H(9) | 0.9500 | C(31)—H(31) | 0.9500 |
| C(10)—C(10A) | 1.423(5) | C(32)—C(33) | 1.362(6) |
| C(10)—N(11) | 1.427(5) | C(32)—H(32) | 0.9500 |
| C(10A)—C(10B) | 1.456(4) | C(33)—C(34) | 1.364(6) |
| N(11)—S(12) | 1.578(3) | C(33)—H(33) | 0.9500 |
| S(12)—O(13) | 1.441(3) | C(34)—C(35) | 1.394(5) |
| S(12)—O(14) | 1.443(3) | C(34)—H(34) | 0.9500 |
| S(12)—C(15) | 1.787(3) | C(35)—H(35) | 0.9500 |
| C(15)—C(20) | 1.384(5) | C(30)—Pd(1)—N(1) | 90.27(13) |
| C(15)—C(16) | 1.392(5) | C(30)—Pd(1)—N(24) | 89.90(12) |
| N(1)—Pd(1)—N(24) | 170.63(12) | C(8)—C(9)—H(9) | 119.2 |
| C(30)—Pd(1)—N(11) | 174.73(13) | C(9)—C(10)—C(10A) | 120.2(3) |
| N(1)—Pd(1)—N(11) | 84.46(11) | C(9)—C(10)—N(11) | 118.0(3) |
| N(24)—Pd(1)—N(11) | 95.26(11) | C(10A)—C(10)—N(11) | 121.7(3) |
| C(2)—N(1)—C(10B) | 119.0(3) | C(10)—C(10A)—C(6A) | 117.1(3) |
| C(2)—N(1)—Pd(1) | 120.7(2) | C(10)—C(10A)—C(10B) | 125.5(3) |
| C(10B)—N(1)—Pd(1) | 118.9(2) | C(6A)—C(10A)—C(10B) | 117.2(3) |
| N(1)—C(2)—C(3) | 123.5(3) | N(1)—C(10B)—C(4A) | 119.3(3) |
| N(1)—C(2)—H(2) | 118.2 | N(1)—C(10B)—C(10A) | 121.0(3) |
| C(3)—C(2)—H(2) | 118.2 | C(4A)—C(10B)—C(10A) | 119.7(3) |
| C(4)—C(3)—C(2) | 118.3(3) | C(10)—N(11)—S(12) | 118.8(2) |
| C(4)—C(3)—H(3) | 120.9 | C(10)—N(11)—Pd(1) | 110.0(2) |
| C(2)—C(3)—H(3) | 120.9 | S(12)—N(11)—Pd(1) | 123.21(16) |
| C(3)—C(4)—C(4A) | 119.9(3) | O(13)—S(12)—O(14) | 117.96(16) |
| C(3)—C(4)—H(4) | 120.1 | O(13)—S(12)—N(11) | 108.27(15) |
| C(4A)—C(4)—H(4) | 120.1 | O(14)—S(12)—N(11) | 112.04(16) |
| C(10B)—C(4A)—C(4) | 119.1(3) | O(13)—S(12)—C(15) | 104.79(16) |
| C(10B)—C(4A)—C(5) | 120.3(3) | O(14)—S(12)—C(15) | 106.30(16) |
| C(4)—C(4A)—C(5) | 120.6(3) | N(11)—S(12)—C(15) | 106.67(15) |
| C(6)—C(5)—C(4A) | 119.8(3) | C(20)—C(15)—C(16) | 120.4(3) |
| C(6)—C(5)—H(5) | 120.1 | C(20)—C(15)—S(12) | 120.2(3) |
| C(4A)—C(5)—H(5) | 120.1 | C(16)—C(15)—S(12) | 119.3(3) |
| C(5)—C(6)—C(6A) | 121.7(3) | C(17)—C(16)—C(15) | 120.3(4) |
| C(5)—C(6)—H(6) | 119.1 | C(17)—C(16)—H(16) | 119.9 |
| C(6A)—C(6)—H(6) | 119.1 | C(15)—C(16)—H(16) | 119.9 |
| C(7)—C(6A)—C(10A) | 120.3(3) | C(18)—C(17)—C(16) | 118.5(4) |
| C(7)—C(6A)—C(6) | 119.5(3) | C(18)—C(17)—H(17) | 120.7 |
| C(10A)—C(6A)—C(6) | 120.2(3) | C(16)—C(17)—H(17) | 120.7 |
| C(8)—C(7)—C(6A) | 121.4(3) | C(17)—C(18)—C(19) | 122.6(3) |
| C(8)—C(7)—H(7) | 119.3 | C(17)—C(18)—N(21) | 118.3(4) |
| C(6A)—C(7)—H(7) | 119.3 | C(19)—C(18)—N(21) | 119.2(3) |
| C(7)—C(8)—C(9) | 119.2(3) | C(18)—C(19)—C(20) | 118.9(3) |
| C(7)—C(8)—H(8) | 120.4 | C(18)—C(19)—H(19) | 120.6 |
| C(9)—C(8)—H(8) | 120.4 | C(20)—C(19)—H(19) | 120.6 |
| C(10)—C(9)—C(8) | 121.6(3) | C(15)—C(20)—C(19) | 119.4(4) |
| C(10)—C(9)—H(9) | 119.2 | C(15)—C(20)—H(20) | 120.3 |
| C(19)—C(20)—H(20) | 120.3 | N(24)—C(29)—H(29) | 118.7 |
| O(23)—N(21)—O(22) | 124.3(3) | C(28)—C(29)—H(29) | 118.7 |
| O(23)—N(21)—C(18) | 117.7(3) | C(31)—C(30)—C(35) | 118.2(3) |
| O(22)—N(21)—C(18) | 118.0(3) | C(31)—C(30)—Pd(1) | 121.0(3) |
| C(25)—N(24)—C(29) | 116.9(3) | C(35)—C(30)—Pd(1) | 120.8(3) |
| C(25)—N(24)—Pd(1) | 120.4(2) | C(30)—C(31)—C(32) | 120.6(4) |
| C(29)—N(24)—Pd(1) | 122.2(2) | C(30)—C(31)—H(31) | 119.7 |
| N(24)—C(25)—C(26) | 122.4(3) | C(32)—C(31)—H(31) | 119.7 |
| N(24)—C(25)—H(25) | 118.8 | C(33)—C(32)—C(31) | 120.5(4) |
| C(26)—C(25)—H(25) | 118.8 | C(33)—C(32)—H(32) | 119.8 |
| C(27)—C(26)—C(25) | 120.3(4) | C(31)—C(32)—H(32) | 119.8 |
| C(27)—C(26)—H(26) | 119.8 | C(32)—C(33)—C(34) | 119.7(4) |
| C(25)—C(26)—H(26) | 119.8 | C(32)—C(33)—H(33) | 120.2 |
| C(26)—C(27)—C(28) | 118.1(4) | C(34)—C(33)—H(33) | 120.2 |
| C(26)—C(27)—H(27) | 120.9 | C(33)—C(34)—C(35) | 120.1(4) |
| C(28)—C(27)—H(27) | 120.9 | C(33)—C(34)—H(34) | 119.9 |
| C(29)—C(28)—C(27) | 119.5(4) | C(35)—C(34)—H(34) | 119.9 |
| C(29)—C(28)—H(28) | 120.3 | C(30)—C(35)—C(34) | 120.9(4) |
| C(27)—C(28)—H(28) | 120.3 | C(30)—C(35)—H(35) | 119.5 |
| N(24)—C(29)—C(28) | 122.7(4) | C(34)—C(35)—H(35) | 119.5 |

TABLE 17
Anisotropic displacement parameters (Å² × 10³) for complex 4a.
The anisotropic displacement factor exponent takes the form:
−2π²[h²a*²U₁₁ + . . . + 2 h k a* b* U₁₂]

| | U₁₁ | U₂₂ | U₃₃ | U₂₃ | U₁₃ | U₁₂ |
|---|---|---|---|---|---|---|
| Pd(1) | 18(1) | 18(1) | 26(1) | −1(1) | 3(1) | 0(1) |
| N(1) | 20(2) | 21(2) | 26(2) | −1(1) | −1(1) | −1(1) |
| C(2) | 26(2) | 22(2) | 30(2) | 0(2) | −3(2) | −1(2) |
| C(3) | 44(3) | 21(2) | 28(2) | 5(2) | −1(2) | −2(2) |
| C(4) | 34(2) | 28(2) | 24(2) | 1(2) | 6(2) | −6(2) |
| C(4A) | 24(2) | 29(2) | 26(2) | −2(2) | 2(2) | −6(2) |
| C(5) | 23(2) | 41(2) | 33(2) | 1(2) | 3(2) | −9(2) |
| C(6) | 19(2) | 39(2) | 29(2) | −5(2) | 1(2) | −2(2) |
| C(6A) | 22(2) | 30(2) | 26(2) | −4(2) | −1(1) | 1(1) |
| C(7) | 22(2) | 35(2) | 24(2) | −5(1) | 1(2) | 5(2) |
| C(8) | 33(2) | 29(2) | 28(2) | 3(2) | −2(2) | 12(2) |
| C(9) | 27(2) | 24(1) | 27(2) | 1(2) | 1(2) | −2(1) |
| C(10) | 18(2) | 23(2) | 20(2) | −3(1) | 2(1) | 3(1) |
| C(10A) | 20(2) | 23(2) | 19(2) | −4(1) | 0(1) | 1(1) |
| C(10B) | 21(2) | 22(2) | 20(2) | −2(1) | 0(2) | −2(1) |
| N(11) | 20(2) | 20(1) | 24(2) | 0(1) | 4(1) | 1(1) |
| S(12) | 24(1) | 20(1) | 26(1) | −1(1) | 1(1) | −3(1) |
| O(13) | 26(1) | 37(2) | 31(1) | 5(1) | 1(1) | −10(1) |
| O(14) | 43(2) | 22(1) | 36(2) | −5(1) | −1(1) | 5(1) |
| C(15) | 25(2) | 24(2) | 24(2) | −4(1) | −2(2) | −3(2) |
| C(16) | 22(2) | 36(2) | 33(2) | 2(2) | 3(2) | 2(2) |
| C(17) | 27(2) | 36(2) | 33(2) | 4(2) | −3(2) | 4(2) |
| C(18) | 39(2) | 22(2) | 22(2) | −2(1) | −5(2) | −9(2) |
| C(19) | 31(2) | 36(2) | 25(2) | O(2) | 3(2) | −8(2) |
| C(20) | 25(2) | 35(2) | 27(2) | −3(2) | 2(2) | −1(2) |
| N(21) | 51(2) | 28(2) | 28(2) | −1(1) | −8(2) | −10(2) |
| O(22) | 55(2) | 42(2) | 39(2) | 5(1) | −10(2) | 3(2) |
| O(23) | 57(2) | 57(2) | 46(2) | 18(2) | −3(2) | −24(2) |
| N(24) | 19(2) | 26(2) | 32(2) | −3(1) | 4(1) | 1(1) |
| C(25) | 26(2) | 26(2) | 31(2) | −2(2) | 3(2) | −2(2) |
| C(26) | 34(2) | 31(2) | 37(2) | 2(2) | 7(2) | −5(2) |
| C(27) | 35(2) | 43(2) | 32(2) | 5(2) | 9(2) | −2(2) |
| C(28) | 35(2) | 45(2) | 27(2) | −6(2) | 4(2) | 6(2) |
| C(29) | 25(2) | 28(2) | 32(2) | −7(2) | 4(2) | 0(2) |
| C(30) | 22(2) | 18(2) | 27(2) | 0(1) | 1(1) | 1(1) |
| C(31) | 26(2) | 28(2) | 50(3) | −5(2) | −4(2) | −1(2) |
| C(32) | 29(2) | 32(2) | 49(2) | 1(2) | 1(2) | 11(2) |
| C(33) | 50(3) | 22(2) | 33(2) | −5(2) | 9(2) | 5(2) |
| C(34) | 37(2) | 29(2) | 45(2) | −10(2) | −2(2) | −1(2) |
| C(35) | 25(2) | 29(2) | 43(2) | −8(2) | 0(2) | 0(2) |

TABLE 18
Hydrogen coordinates (×10⁴) and isotropic displacement
parameters (Å² × 10³) for complex 4a.

| | x | y | z | U(eq) |
|---|---|---|---|---|
| H(2) | 3897 | 2532 | 7087 | 32 |
| H(3) | 2318 | 1706 | 7780 | 37 |
| H(4) | −53 | 2071 | 7690 | 35 |
| H(5) | −1955 | 3081 | 7194 | 39 |
| H(6) | −2616 | 4371 | 6543 | 35 |
| H(7) | −2074 | 5902 | 6012 | 32 |
| H(8) | −500 | 7061 | 5653 | 36 |
| H(9) | 1899 | 6821 | 5826 | 31 |
| H(16) | 6089 | 4759 | 7512 | 36 |
| H(17) | 6135 | 3558 | 8352 | 38 |
| H(19) | 2081 | 4120 | 8807 | 37 |
| H(20) | 2027 | 5327 | 7962 | 35 |
| H(25) | 5391 | 5843 | 5528 | 33 |
| H(26) | 6340 | 6527 | 4556 | 40 |
| H(27) | 6387 | 5670 | 3529 | 44 |
| H(28) | 5535 | 4077 | 3523 | 43 |
| H(29) | 4651 | 3417 | 4517 | 34 |
| H(31) | 6468 | 2778 | 5948 | 41 |
| H(32) | 7057 | 1213 | 5605 | 44 |
| H(33) | 5367 | 195 | 5164 | 42 |
| H(34) | 3067 | 714 | 5083 | 45 |
| H(35) | 2465 | 2276 | 5424 | 39 |

TABLE 19
Torsion angles [°] for complex 4a.

| | | | |
|---|---|---|---|
| C(30)—Pd(1)—N(1)—C(2) | 38.6(3) | C(4A)—C(5)—C(6)—C(6A) | −6.1(6) |
| N(11)—Pd(1)—N(1)—C(2) | −141.6(3) | C(5)—C(6)—C(6A)—C(7) | −172.3(3) |
| C(30)—Pd(1)—N(1)—C(10B) | −127.6(3) | C(5)—C(6)—C(6A)—C(10A) | 5.7(5) |
| N(11)—Pd(1)—N(1)—C(10B) | 52.2(2) | C(10A)—C(6A)—C(7)—C(8) | −2.6(5) |
| C(10B)—N(1)—C(2)—C(3) | 1.4(5) | C(6)—C(6A)—C(7)—C(8) | 175.5(3) |
| Pd(1)—N(1)—C(2)—C(3) | −164.8(3) | C(6A)—C(7)—C(8)—C(9) | −0.9(5) |
| N(1)—C(2)—C(3)—C(4) | 6.0(6) | C(7)—C(8)—C(9)—C(10) | 1.6(5) |
| C(2)—C(3)—C(4)—C(4A) | −5.4(6) | C(8)—C(9)—C(10)—C(10A) | 1.3(5) |
| C(3)—C(4)—C(4A)—C(10B) | −2.2(5) | C(8)—C(9)—C(10)—N(11) | −179.7(3) |
| C(3)—C(4)—C(4A)—C(5) | 176.1(4) | C(9)—C(10)—C(10A)—C(6A) | −4.6(5) |
| C(10B)—C(4A)—C(5)—C(6) | −2.6(5) | N(11)—C(10)—C(10A)—C(6A) | 176.5(3) |
| C(4)—C(4A)—C(5)—C(6) | 179.1(3) | C(9)—C(10)—C(10A)—C(10B) | 179.9(3) |
| N(11)—C(10)—C(10A)—C(10B) | 0.9(5) | N(11)—S(12)—C(15)—C(16) | 98.1(3) |
| C(7)—C(6A)—C(10A)—C(10) | 5.3(5) | C(20)—C(15)—C(16)—C(17) | 2.1(6) |
| C(6)—C(6A)—C(10A)—C(10) | −172.8(3) | S(12)—C(15)—C(16)—C(17) | −173.5(3) |
| C(7)—C(6A)—C(10A)—C(10B) | −178.8(3) | C(15)—C(16)—C(17)—C(18) | −1.2(5) |
| C(6)—C(6A)—C(10A)—C(10B) | 3.1(5) | C(16)—C(17)—C(18)—C(19) | 0.3(6) |
| C(2)—N(1)—C(10B)—C(4A) | −9.2(5) | C(16)—C(17)—C(18)—N(21) | −179.8(3) |
| Pd(1)—N(1)—C(10B)—C(4A) | 157.2(2) | C(17)—C(18)—C(19)—C(20) | −0.2(5) |
| C(2)—N(1)—C(10B)—C(10A) | 170.6(3) | N(21)—C(18)—C(19)—C(20) | 179.9(3) |
| Pd(1)—N(1)—C(10B)—C(10A) | −22.9(4) | C(16)—C(15)—C(20)—C(19) | −2.0(5) |
| C(4)—C(4A)—C(10B)—N(1) | 9.7(5) | S(12)—C(15)—C(20)—C(19) | 173.6(3) |
| C(5)—C(4A)—C(10B)—N(1) | −168.7(3) | C(18)—C(19)—C(20)—C(15) | 1.0(5) |
| C(4)—C(4A)—C(10B)—C(10A) | −170.2(3) | C(17)—C(18)—N(21)—O(23) | −166.8(3) |
| C(5)—C(4A)—C(10B)—C(10A) | 11.4(5) | C(19)—C(18)—N(21)—O(23) | 13.2(5) |
| C(10)—C(10A)—C(10B)—N(1) | −15.8(5) | C(17)—C(18)—N(21)—O(22) | 13.5(5) |
| C(6A)—C(10A)—C(10B)—N(1) | 168.7(3) | C(19)—C(18)—N(21)—O(22) | −166.6(3) |
| C(10)—C(10A)—C(10B)—C(4A) | 164.1(3) | C(30)—Pd(1)—N(24)—C(25) | −155.9(3) |
| C(6A)—C(10A)—C(10B)—C(4A) | −11.4(4) | N(11)—Pd(1)—N(24)—C(25) | 25.1(3) |
| C(9)—C(10)—N(11)—S(12) | 76.9(3) | C(30)—Pd(1)—N(24)—C(29) | 32.9(3) |
| C(10A)—C(10)—N(11)—S(12) | −104.2(3) | N(11)—Pd(1)—N(24)—C(29) | −146.0(3) |
| C(9)—C(10)—N(11)—Pd(1) | −133.2(3) | C(29)—N(24)—C(25)—C(26) | −1.0(5) |
| C(10A)—C(10)—N(11)—Pd(1) | 45.8(3) | Pd(1)—N(24)—C(25)—C(26) | −172.6(3) |
| N(1)—Pd(1)—N(11)—C(10) | −60.9(2) | N(24)—C(25)—C(26)—C(27) | 1.6(6) |
| N(24)—Pd(1)—N(11)—C(10) | 109.7(2) | C(25)—C(26)—C(27)—C(28) | −1.2(6) |
| N(1)—Pd(1)—N(11)—S(12) | 87.49(19) | C(26)—C(27)—C(28)—C(29) | 0.4(6) |
| N(24)—Pd(1)—N(11)—S(12) | −101.92(19) | C(25)—N(24)—C(29)—C(28) | 0.1(5) |
| C(10)—N(11)—S(12)—O(13) | −154.6(2) | Pd(1)—N(24)—C(29)—C(28) | 171.5(3) |
| Pd(1)—N(11)—S(12)—O(13) | 59.6(2) | C(27)—C(28)—C(29)—N(24) | 0.2(6) |
| C(10)—N(11)—S(12)—O(14) | −22.8(3) | N(1)—Pd(1)—C(30)—C(31) | −117.3(3) |
| Pd(1)—N(11)—S(12)—O(14) | −168.61(16) | N(24)—Pd(1)—C(30)—C(31) | 72.0(3) |
| C(10)—N(11)—S(12)—C(15) | 93.1(3) | N(1)—Pd(1)—C(30)—C(35) | 62.5(3) |
| Pd(1)—N(11)—S(12)—C(15) | −52.7(2) | N(24)—Pd(1)—C(30)—C(35) | −108.2(3) |
| O(13)—S(12)—C(15)—C(20) | 167.8(3) | C(35)—C(30)—C(31)—C(32) | 1.5(6) |
| O(14)—S(12)—C(15)—C(20) | 42.2(3) | Pd(1)—C(30)—C(31)—C(32) | −178.7(3) |
| N(11)—S(12)—C(15)—C(20) | −77.6(3) | C(30)—C(31)—C(32)—C(33) | −0.4(6) |
| O(13)—S(12)—C(15)—C(16) | −16.6(3) | C(31)—C(32)—C(33)—C(34) | −0.8(6) |
| O(14)—S(12)—C(15)—C(16) | −142.2(3) | C(32)—C(33)—C(34)—C(35) | 0.9(6) |
| C(31)—C(30)—C(35)—C(34) | −1.4(6) | | |
| Pd(1)—C(30)—C(35)—C(34) | 178.8(3) | | |
| C(33)—C(34)—C(35)—C(30) | 0.3(6) | | |

### Example 10

### Carbon-Fluorine Reductive Elimination from a High-Valent Palladium Fluoride

To address the unsolved problem of late-stage fluorination of functionalized molecules, we have described herein that aryl boronic acids can be converted into aryl fluorides via reaction of stoichiometric aryl palladium complexes with the electrophilic fluorination reagent SELECTFLUOR® (1) (eq 1) (Singh, R. P.; Shreeve, J. M. Acc. Chem. Res. 2004, 37, 31-44; (b) Nyffeler, P. T.; Duron, S. G.; Burkart, M. D.; Vincent, S. P.; Wong, C. H. Angew. Chem., Int. Ed. 2005, 44, 192-212; each of which is incorporated herein by reference). Two potential mechanisms for carbon-fluorine bond formation are palladium-carbon bond cleavage by the electrophilic fluorination reagent and oxidation of the palladium center to form a discrete high-valent palladium fluoride followed by reductive elimination to form a carbon-fluorine bond. In this Example we present the carbon-fluorine bond formation from two discrete high-valent aryl palladium fluoride complexes. The observation of discrete high-valent palladium fluorides may afford valuable mechanistic insight to better understand carbon-fluorine bond formation mediated by transition metals.

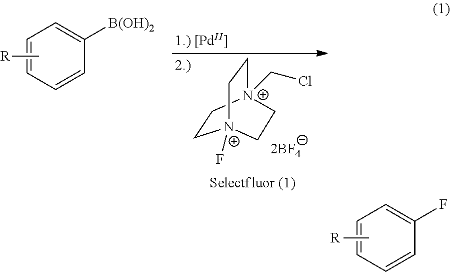

Transition-metal-mediated carbon-fluorine bond formations are rare. Three processes, including our own work, have been reported using palladium complexes and electrophilic fluorination sources. For all three processes, the intermediacy of a high-valent palladium fluoride followed by reductive elimination to form the carbon-fluorine bond and a palladium (II) complex was discussed as a potential reaction pathway. In none of the cases, however, was a high-valent palladium intermediate characterized or observed. In fact, a concerted carbon-fluorine reductive elimination has never been substantiated in the literature from any transition metal (Grushin, V. V. Chem.-Eur. J. 2002, 8, 1006-1014; Yandulov, D. V.; Tran, N. T. J. Am. Chem. Soc. 2007, 129, 1342-1358; Grushin, V. V.; Marshall, W. J. Organometallics 2007, 26, 4997-5002; each of which is incorporated herein by reference).

Scheme 10-1 shows a reaction sequence to regiospecifically convert a boronic acid into the corresponding arylfluoride. We found that pyridine-sulfonamide ligands such as 2 are well suited to support arylpalladium complexes and can afford arylfluorides upon treatment with SELECTFLUOR® in high yield (87% in the presented case). The palladium (II) acetate complex 3 was obtained in 99% yield from pyridine-sulfonamide 2 and palladium (II)acetate. Transmetallation using 4-tert-butylphenylboronic acid (4) afforded the air- and water-stable yellow aryl palladium complex 5 in 80% yield. Fluorination of 5 with SELECTFLUOR® in acetone at 50° C. gave 4-tert-butylfluorobenzene (6) in 87% yield within 30 min.

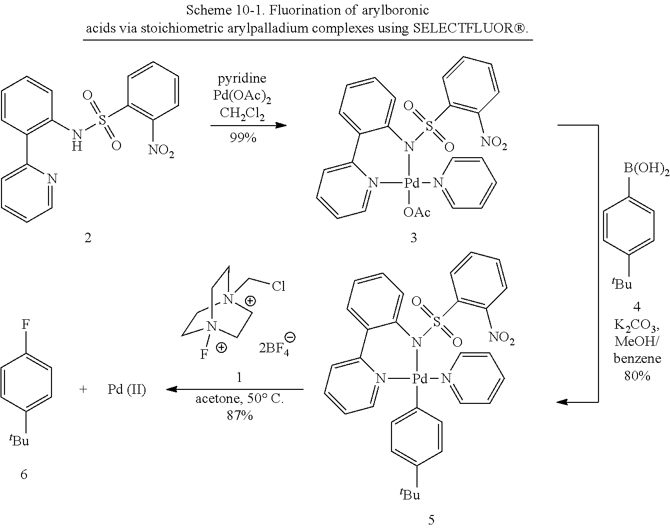

Under the reaction conditions that afforded 87% yield of 6 (acetone, 50° C.), we did not observe a high-valent palladium fluoride intermediate by NMR, but a reversible color change from yellow to orange upon addition of 5 to SELECTFLUOR® suggested the formation of a discrete intermediate. To evaluate the mechanistic hypothesis that pyridine-sulfonamide-stabilized aryl palladium complexes such as 5 can afford carbon-fluorine bond formation via well-defined discrete palladium fluorides, we sought to design an analog of 5 that would afford an observable palladium (IV) fluoride upon oxidation with SELECTFLUOR®. Rigid ligands have been shown to stabilize high-valent metal centers including palladium (IV) (Canty, A. J.; Jin, H.; Roberts, A. S.; Skelton, B. W.; Traill, P. R.; White, A. H. Organometallics 1995, 14, 199-206; Canty, A. J.; Denney, M. C.; van Koten, G.; Skelton, B. W.; White, A. H. Organometallics 2004, 23, 5432-5439; Campora, J.; Palma, P.; del R10, D.; Lopez, J. A.; Alvarez, E.; Connelly, N. G. Organometallics 2005, 24, 3624-3628; Dick, A. R.; Kampf, J. W.; Sanford, M. S. J. Am. Chem. Soc. 2005, 127, 12790-12791; each of which is incorporated herein by reference). We therefore synthesized the palladium (II) derivative 8, in which a rigid, chelating benzoquinolinyl ligand replaces the aryl and pyridyl ligands of 5 (eq 2). Treatment of the benzoquinolinyl palladium acetate dimer 7 (Dick et al., J. Am. Chem. Soc. 2004, 126, 2300-2301; which is incorporated herein by reference) with one equivalent of the pyridine-sulfonamide ligand 2 in methylene chloride at room temperature afforded the aryl palladium complex 8 in 95% yield as an analytically pure yellow solid within 20 min.

Fluorination of 8 in acetonitrile at 50° C. afforded 10-fluorobenzo[h]quinoline (10) in 94% yield (Scheme 10-2). Moreover, we observed a deep purple, well-defined intermediate at 23° C. by ¹H and ¹³C NMR which was stable in acetonitrile solution at 23° C. for 1 hour and did not contain either 8 or 10. The NMR resonances, including an ¹⁹F NMR resonance at −278 ppm, are consistent with the terminal palladium (IV) fluoride structure 9. When the acetonitrile solution of 9 was subsequently heated to 50° C., reductive elimination occurred to form 10. We assigned the cationic octahedral structure 9 to the intermediate that includes an acetonitrile molecule trans to the most trans-influencing ligand (aryl) on the palladium. Additional evidence for the formation of a high-valent palladium fluoride was obtained, when the intermediate 9 was treated with tetramethylammonium fluoride tetrahydrate at room temperature to form the palladium (IV)difluoride 11 that we independently synthesized by oxidation of 8 with XeF₂.

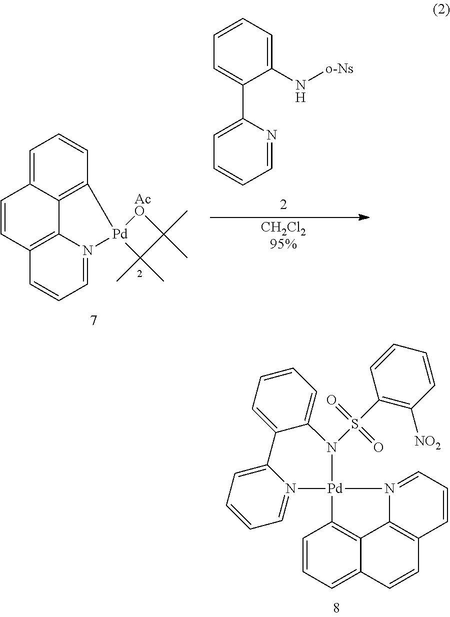

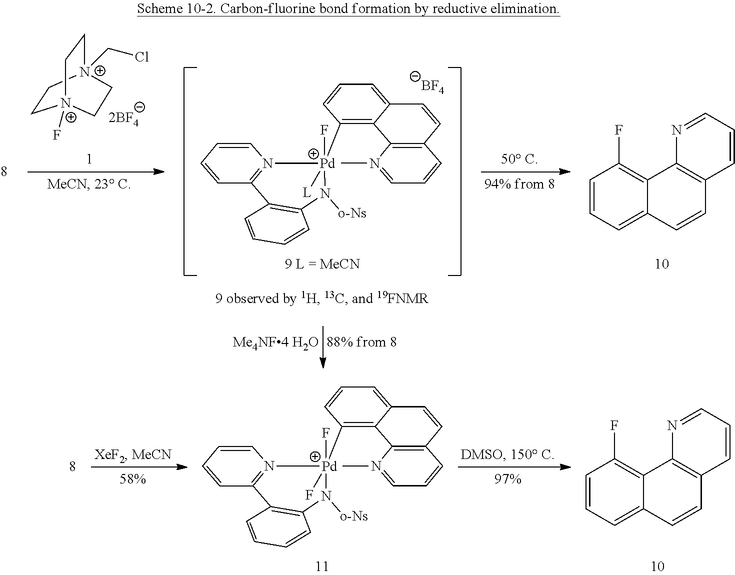

Reductive elimination from 9 afforded a cationic palladium (II)tetrafluoroborate that was trapped with pyridine to afford the cationic palladium bispyridine tetrafluoroborate 12 that we independently synthesized from the palladium acetate 3 in 94% yield (Scheme 10-3). The isolation of 12 with the pyridine-sulfonamide ligand coordinated to palladium is consistent with reductive elimination from 9.

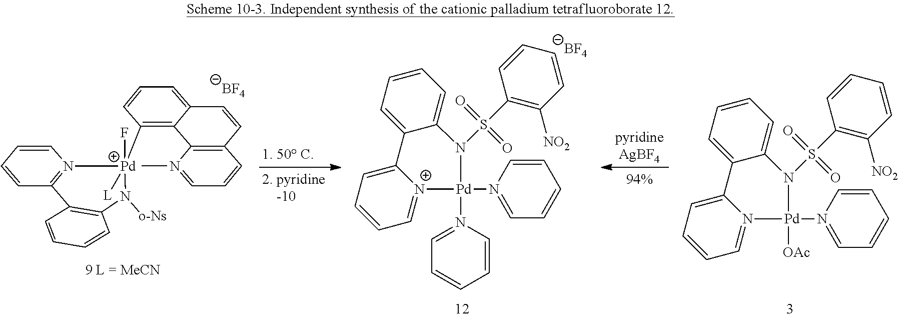

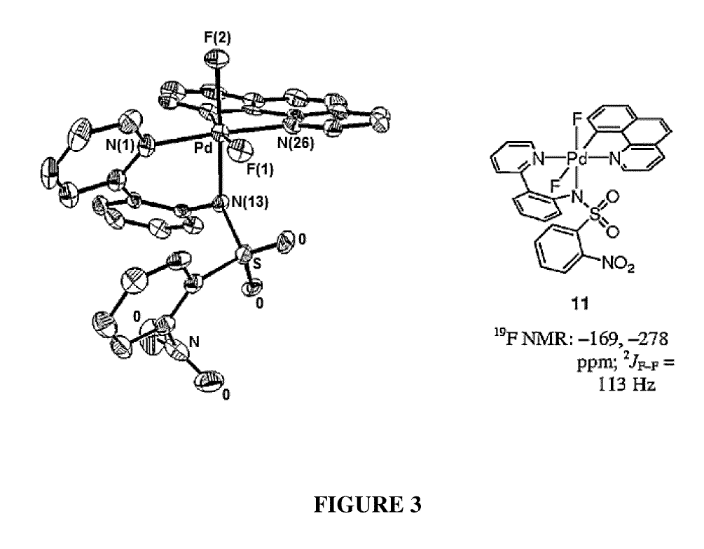

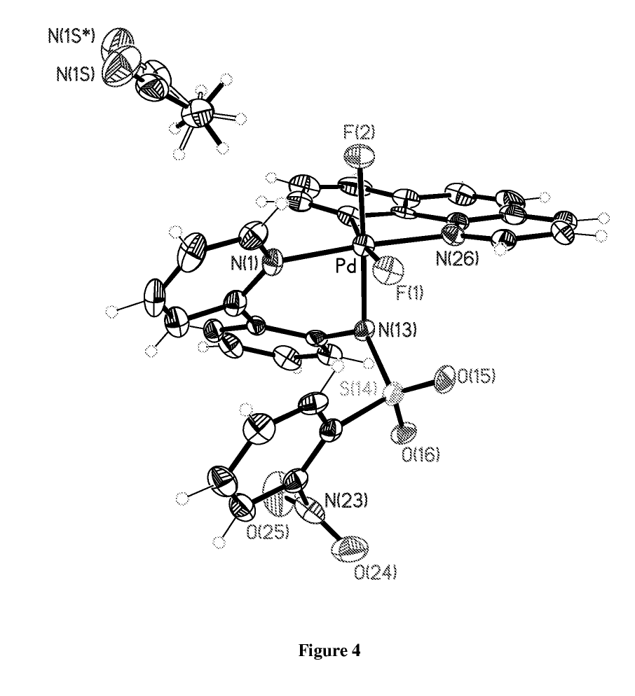

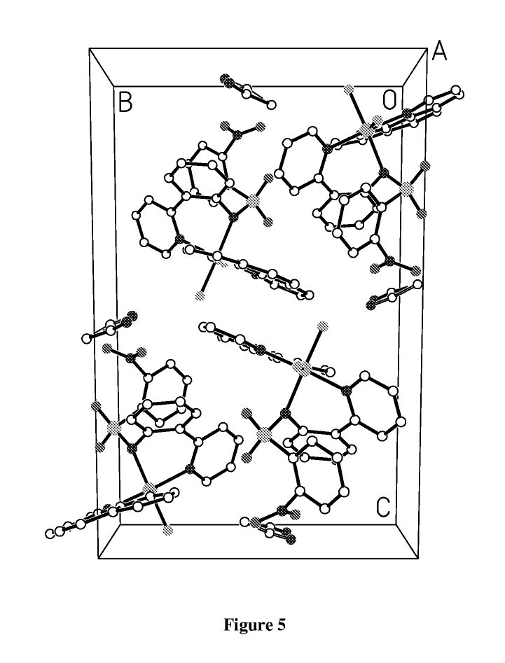

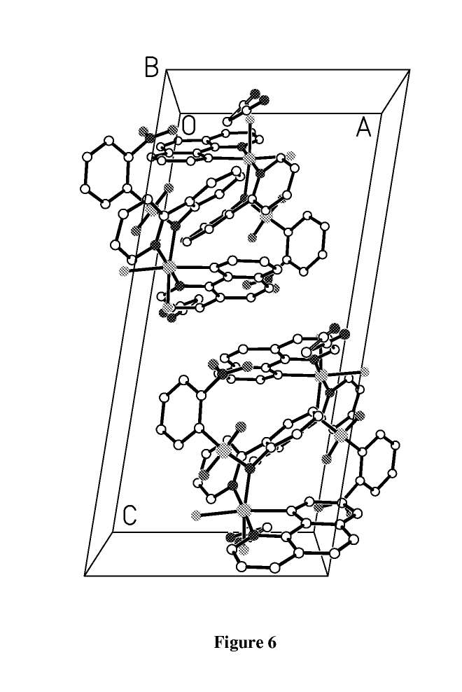

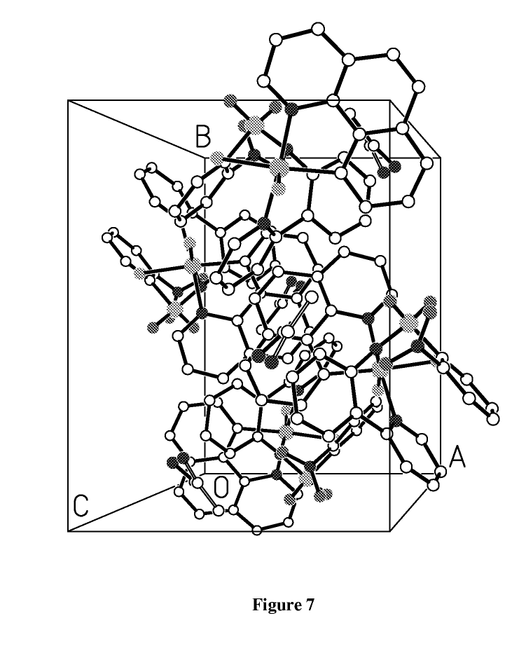

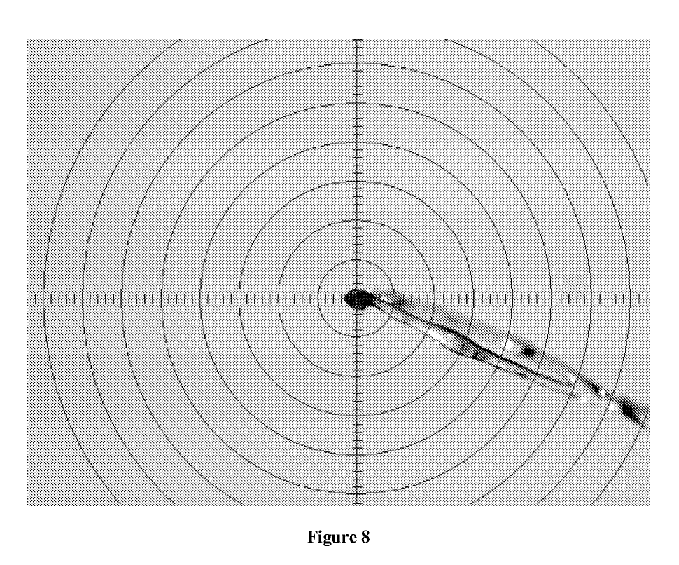

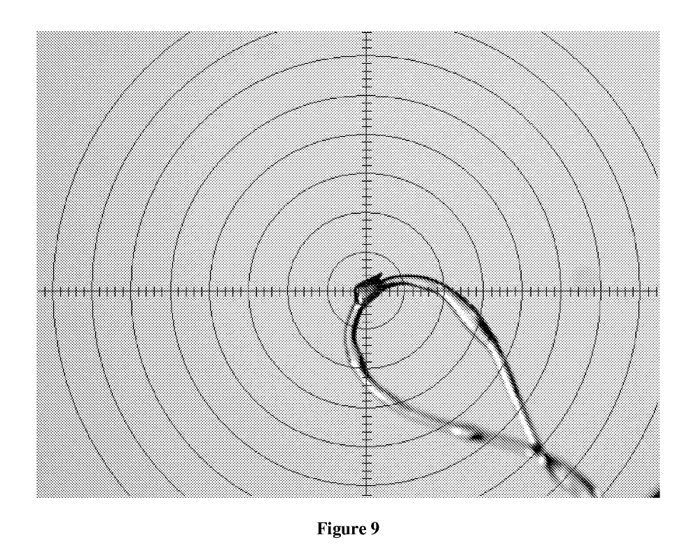

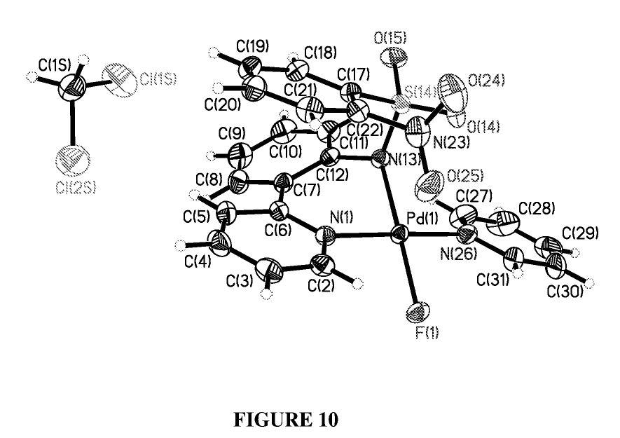

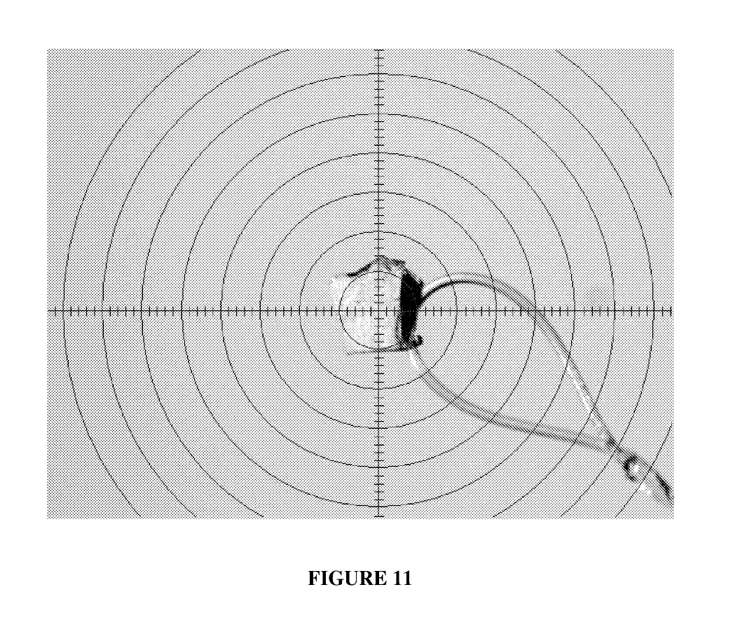

The neutral palladium difluoride 11 was thermally more stable than the monofluoride 9, could be isolated, and afforded 10 in 97% yield when heated in DMSO at 150° C. for 10 minutes (Scheme 10-2). The palladium (IV)difluoride 11 is an air and moisture stable bright orange solid that is stable at 23° C. for at least 1 week and in chloroform solution at 50° C. for at least 2 hours. A ²J_{F-F} coupling constant of 113 Hz indicates that both fluorine atoms are associated with the palladium atom in solution. The palladium (IV)difluoride crystallized from an acetonitrile solution as orange prisms and was analyzed by X-ray crystallography (FIG. 3). The two fluoride substituents are mutually cis, one trans to the aryl ligand, the other trans to the sulfonamide ligand and have bond lengths to palladium of 1.955(3)Å (F2) and 2.040(3)Å (F1), respectively.

In conclusion, we have shown carbon-fluorine bond formation from two discrete palladium (IV) fluoride complexes. Our data is consistent with reductive elimination and provides insight into carbon-fluorine bond formation from arylpalladium complexes.

### Experimentals

### Materials and Methods

All reactions were carried out under an ambient atmosphere unless otherwise indicated. Solvents were dried by passage through alumina (Pangborn, A. B.; Giardello, M. A.; Grubbs, R. H.; Rosen, R. K.; Timmers, F. J. Organometallics 1996, 15:1518-1520; which is incorporated herein by reference). Except as indicated otherwise, reactions were magnetically stirred and monitored by thin layer chromatography (TLC) using EMD TLC plates pre-coated with 250 μm thickness silica gel 60 F254 plates and visualized by fluorescence quenching under UV light. In addition, TLC plates were stained using ceric ammonium molybdate or potassium permanganate stain. Flash chromatography was performed on Dynamic Adsorbents Silica Gel 40-63 μm particle size using a forced flow of eluant at 0.3-0.5 bar pressure (Still, W. C.; Kahn, M.; Mitra, A. J. Org. Chem. 1978, 43, 2925-2927; incorporated herein by reference). Concentration under reduced pressure was performed by rotary evaporation at 25-30° C. at appropriate pressure. Purified compounds were further dried under high vacuum (0.01-0.05 Torr). Melting points were measured on a Buchi 510 apparatus. All melting points were measured in open capillaries and are uncorrected. NMR spectra were recorded on a Varian Unity/Inova 500 spectrometer operating at 500 MHz and 125 MHz for ¹H and ¹³C acquisitions, respectively, or on a Varian Mercury 400 spectrometer operating at 375 MHz for ¹⁹F acquisition. Chemical shifts are reported in ppm with the solvent resonance as the internal standard. Data is reported as follows: s=singlet, br=broad, d=doublet, t=triplet, q=quartet, m=multiplet; coupling constants in Hz; integration. High-resolution mass spectra were obtained on Jeol AX-505 or SX-102 spectrometers at the Harvard University Mass Spectrometry Facilities. Triethylamine was distilled over calcium hydride. Benzo[h]quinoline was purchased from TCI America. 2-Nitrobenzenesulfonyl chloride, 2-bromoaniline, pinacolborane, [1,1′-biphenyl]-2-yldicyclohexylphosphine, barium hydroxide octahydrate, 2-bromopyridine, tetramethylammonium fluoride tetrahydrate, and anhydrous dioxane were purchased from Aldrich. 1-Chloromethyl-4-fluoro-1,4-diazoniabicyclo[2.2.2]octane bis(tetrafluoroborate) was purchased from Alfar Aesar. Palladium acetate and silver tetrafluoroborate were purchased from Strem. Xenon difluoride was purchased from Matrix Scientific. 4-tert-Butylphenylboronic acid was purchased from Frontier Scientific and used as received.

### Experimental Data

### Experimental Procedures and Compound Characterization

### Benzo[h]quinolinyl palladium acetate dimer (7)

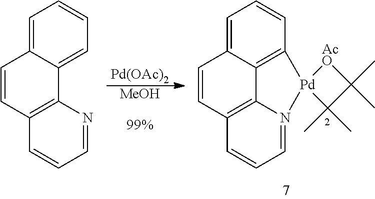

To benzo[h]quinoline (1.79 g, 10.0 mmol, 1.00 equiv) in MeOH (100 mL) at 23° C. is added palladium acetate (2.25 g, 10.0 mmol, 1.00 equiv). After stirring for 17 h, the suspension is filtered off and washed with MeOH (50 mL) and Et₂O (50 mL) to afford 3.19 g of the title compound as a yellow solid (99% yield).

NMR Spectroscopy: ¹H NMR (500 MHz, CDCl₃, 23° C., δ): 7.80 (dd, J=5.5 Hz, 1.5 Hz, 1H), 7.43 (dd, J=8.0 Hz, 1.5 Hz, 1H), 7.24-7.18 (m, 3H), 7.08 (dd, J=7.0 Hz, J=1.5 Hz, 1H), 6.97 (d, J=9.0 Hz, 1H), 6.46 (dd, J=7.5 Hz, 5.0 Hz, 1H), 2.38 (s, 3H). ¹³C NMR (125 MHz, CDCl₃, 23° C., δ): 182.5, 153.2, 148.9, 148.8, 140.0, 135.3, 132.4, 129.0, 127.9, 127.7, 125.0, 122.9, 122.1, 119.8, 25.2. These spectroscopic data correspond to the reported data in Dick, A. R.; Hull , K. L.; Sanford, M. S. J. Am. Chem. Soc. 2004, 126, 2300-2301; incorporated herein by reference.

### 2-(2-Pyridyl)aniline

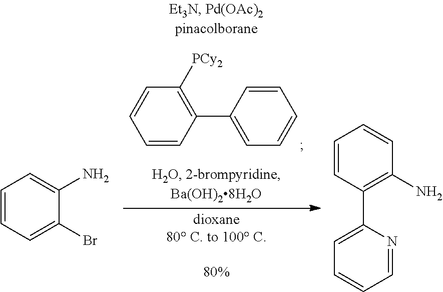

Under nitrogen atmosphere, to 2-bromoaniline (1.50 g, 1.55 mL, 8.72 mmol, 1.00 equiv) in anhydrous dioxane (18 mL) at 23° C. is added Et₃N (4.06 mL, 34.9 mmol, 4.00 equiv), palladium acetate (97.9 mg, 0.440 mmol, 5.00 mol %), [1,1′-biphenyl]-2-yldicyclohexylphosphine (458 mg, 1.31 mmol, 15.0 mol %) and pinacolborane (3.83 mL, 26.2 mmol, 3.00 equiv). The reaction mixture is stirred at 80° C. for 1.0 h before the addition of water (3.80 mL), Ba(OH)₂.8H₂O (8.25 g, 26.2 mmol, 3.00 equiv), and 2-bromopyridine (1.38 g, 0.850 mL, 8.72 mmol, 1.00 equiv). The suspension is heated at 100° C. for 4.0 h. After cooling to 23° C., the reaction mixture is filtered through celite and brine (50 mL) is added to the filtrate. The phases are separated and the aqueous phase is extracted with CH₂Cl₂ (3×50 mL). The combined organic phases are washed with brine (30 mL) and dried (Na₂SO₄). The filtrate is concentrated in vacuo and the residue is purified by chromatography on silica gel eluting with hexanes/EtOAc 3:1 (v/v) to afford 1.18 g of the title compound as red-brown oil (80% yield).

R_{f}=0.38 (hexanes/EtOAc 3:1 (v/v)). NMR Spectroscopy: ¹H NMR (500 MHz, CDCl₃, 23° C., δ): 8.61-8.60 (m, 1H), 7.78-7.75 (m, 1H), 7.65 (d, J=7.9 Hz, 1H), 7.51 (dd, J=7.6 Hz, 1.4 Hz, 1H), 7.19-7.16 (m, 2H), 6.80-6.76 (m, 2H), 5.72 (br s, 2H). ¹³C NMR (125 MHz, CDCl₃, 23° C., δ): 159.2, 147.6, 146.3, 136.6, 129.6, 129.1, 121.9, 120.7, 117.3, 116.9. Mass Spectrometry: HRMS-FIA (m/z): Calcd for [C₁₁H₁₀N₂+H], 171.0917. Found, 171.0923. This spectroscopic data corresponds to the reported data in Rebstock, A. S.; Mongin, F.; Trecourt, F.; Queguiner, G. Org. Biomol. Chem. 2003, 1, 3064-3068; which is incorporated herein by reference.

### 2-(2-Pyridinyl)phenyl-2-nitrobenzenesulfonamide (2)

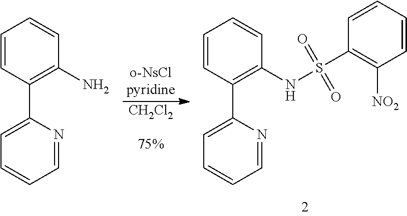

To 2-(2-Pyridyl)aniline (851 mg, 5.00 mmol, 1.00 equiv) in CH₂Cl₂ (10 mL) at 0° C. is added pyridine (1.60 mL, 20.0 mmol, 4.00 equiv) and 2-nitrobenzenesulfonyl chloride (2.20 g, 10.0 mmol, 2.00 equiv). The reaction mixture is warmed to 23° C. and stirred for 2.0 hr before the addition of water (10 mL). The phases are separated and the aqueous layer is extracted with CH₂Cl₂ (3×8 mL). The combined organic phases are washed with brine (30 mL) and dried (Na₂SO₄). The filtrate is concentrated in vacuo and the residue is purified by chromatography on silica gel eluting with hexanes/EtOAc 3:7 (v/v) to afford 1.33 g of the title compound as a pale-yellow solid (75% yield).

R_{f}=0.12 (hexanes/EtOAc 7:3 (v/v)). Melting Point: 91-94° C. NMR Spectroscopy: ¹H NMR (500 MHz, CDCl₃, 23° C., δ): 8.73 (d, J=5.0 Hz, 1H), 7.94 (dd, J=7.5 Hz, 2.0 Hz, 1H), 7.82 (dd, J=8.0 Hz, 1.0 Hz, 1H), 7.74 (ddd, J=7.5 Hz, 7.5 Hz, 2.0 Hz, 1H), 7.63-7.52 (m, 5H), 7.38 (ddd, J=7.5 Hz, 7.5 Hz, 1.5 Hz, 1H), 7.27-7.24 (m, 1H), 7.18 (ddd, J=7.5 Hz, 7.5 Hz, 1.0 Hz, 1H). ¹³C NMR (125 MHz, CDCl₃, 23° C., δ): 156.9, 156.2, 148.0, 137.9, 136.4, 133.6, 132.2, 131.0, 130.0, 129.0, 127.1, 125.0, 124.7, 122.4, 121.9, 121.9, 110.9. Mass Spectrometry: HRMS-FIA (m/z): Calcd for [C₁₇H₁₃N₃O₄S+H], 356.0700. Found, 356.0701.

### Acetato palladium complex 3

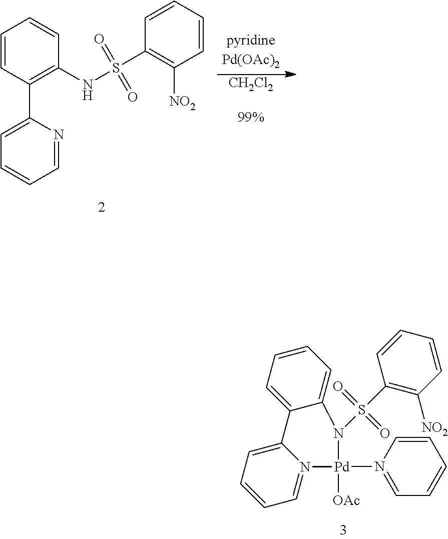

To palladium acetate (448 mg, 2.00 mmol, 1.00 equiv) in CH₂Cl₂ (20 mL) at 23° C. is added pyridine (485 μL, 6.00 mmol, 3.00 equiv) and 2-(2-pyridinyl)phenyl-2-nitrobenzenesulfonamide (2) (711 mg, 2.00 mmol, 1.00 equiv). After stirring for 20 min, the solution is concentrated in vacuo. The resulting residue is triturated with Et₂O (3×1 mL) to afford 1.19 g of the title compound as a pale-yellow solid (99% yield).

Melting Point: 195° C. (decomp.). NMR Spectroscopy: ¹H NMR (500 MHz, CDCl₃, 23° C., δ): 8.79 (d, J=6.5 Hz, 2H), 8.58 (d, J=5.5 Hz, 1H), 7.80 (dd, J=7.5 Hz, 7.5 Hz, 1H), 7.61 (d, J=7.5 Hz, 2H), 7.57-7.52 (m, 2H), 7.48 (d, J=8.0 Hz, 1H), 7.39-7.33 (m, 3H), 7.27 (d, J=8.0 Hz, 1H), 7.21-7.15 (m, 2H), 7.06-7.03 (m, 2H), 1.85 (s, 3H). ¹³C NMR (125 MHz, CDCl₃, 23° C., δ): 177.6, 154.7, 151.8, 151.1, 146.9, 139.9, 138.6, 138.4, 136.3, 134.8, 131.7, 131.1, 130.3, 129.9, 129.6, 125.8, 124.8, 123.3, 122.8, 122.3, 110.7, 23.5. Mass Spectrometry: HRMS-FIA (m/z): Calcd for [C₂₄H₂₀N₄O₆PdS+NH₄], 616.0476. Found, 616.0473.

### Aryl palladium complex 5

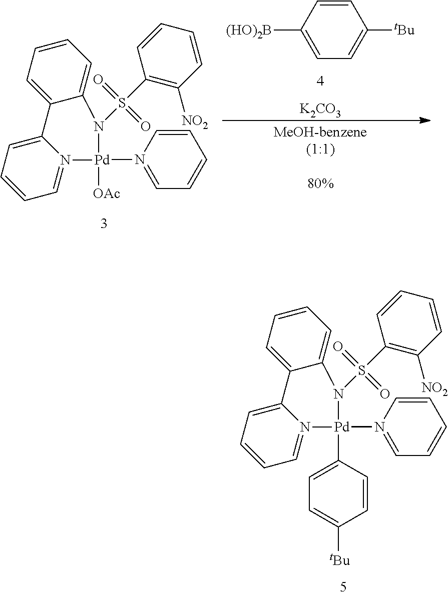

To the acetato palladium complex 3 (300 mg, 0.501 mmol, 1.00 equiv) in MeOH (5.0 mL) and benzene (5.0 mL) at 23° C. is added 4-tert-butylphenylboronic acid (98.0 mg, 0.551 mmol, 1.10 equiv) and K₂CO₃ (138 mg, 1.00 mmol, 2.00 equiv). The reaction mixture is stirred at 23° C. for 3.0 h, and the solvent is removed in vacuo. To the solid residue is added CHCl₃ (5 mL) and water (5 mL). The phases are separated and the aqueous phase is extracted with CHCl₃ (3×5 mL). The combined organic phases are washed with brine (5 mL) and dried (Na₂SO₄). The filtrate is concentrated in vacuo and the residue is purified by chromatography on silica gel eluting with hexanes/EtOAc 2:3 (v/v) to afford 270 mg of the title compound as a colorless solid (80% yield).

R_{f}=0.13 (hexanes/EtOAc 1:1). Melting Point: 145° C. (decomp.). NMR Spectroscopy: ¹H NMR (500 MHz, CDCl₃, 8): 8.85 (dd, J=6.0 Hz, 1.5 Hz, 2H), 8.18 (d, J=5.5 Hz, 1H), 7.66 (dd, J=8.0 Hz, 8.0 Hz, 1H), 7.52 (d, J=8.0 Hz, 1H), 7.48-7.42 (m, 2H), 7.38 (dd, J=7.5 Hz, 1.5 Hz, 1H), 7.29 (d, J=7.0 Hz, 1H), 7.26-7.20 (m, 3H), 7.18-7.12 (m, 3H), 7.10-7.00 (m, 3H), 6.92 (d, J=8.0 Hz, 2H), 6.79 (dd, J=7.5 Hz, 6.0 Hz, 1H), 1.21 (s, 9H). ¹³C NMR (125 MHz, CDCl₃, 8): 157.6, 153.2, 153.1, 149.7, 147.2, 146.2, 143.1, 138.0, 137.7, 136.5, 136.3, 134.1, 131.4, 130.4, 130.2, 129.8, 129.5, 129.4, 124.9, 124.8, 124.2, 124.1, 122.6, 122.3, 34.1, 31.7. Mass Spectrometry: HRMS-FIA (m/z): Calcd for [C₃₂H₃₀N₄O₄PdS+H], 673.1101. Found, 673.1111.

### 1-tert-Butyl-4-fluorobenzene (6)

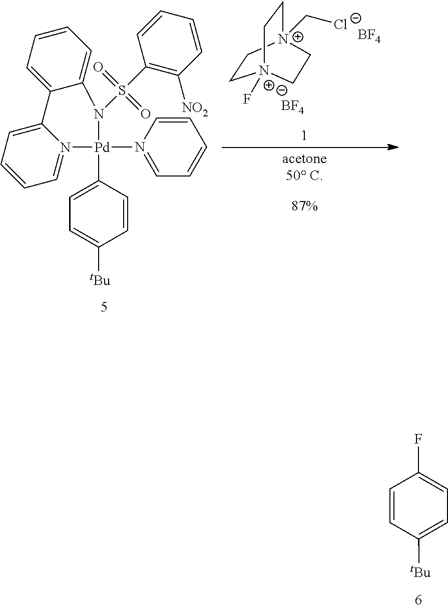

To 1-chloromethyl-4-fluoro-1,4-diazoniabicyclo [2.2.2]octane bis(tetrafluoroborate) (1) (4.3 mg, 0.012 mmol, 1.2 equiv) in acetone-d₆ (0.3 mL) at 50° C. is added aryl palladium complex 5 (6.7 mg, 0.010 mmol, 1.0 equiv) in 10 portions over 10 min. The reaction mixture is stirred at 50° C. for 10 min. The reaction mixture is cooled to 23° C., at which temperature 3-nitrofluorobenzene (2.65 mg, 2.00 μL, 0.0188 mmol) is added. The yield is determined by comparing the integration of the ¹⁹F NMR (375 MHz, acetone-d₆, 23° C.) resonance of 1-tert-butyl-4-fluorobenzene (−120.6 ppm) and that of 3-nitro-fluorobenzene (−111.8 ppm) (87% yield). The ¹⁹F NMR chemical shift of the product corresponds to that of reported data (Laali, K. K.; Okazaki, T.; Bunge, S. D. J. Org. Chem. 2007, 72, 6758-6762; which is incorporated herein by reference).

### Benzo[h]quinolinyl palladium(II) pyrdine-sulfonamido complex 8

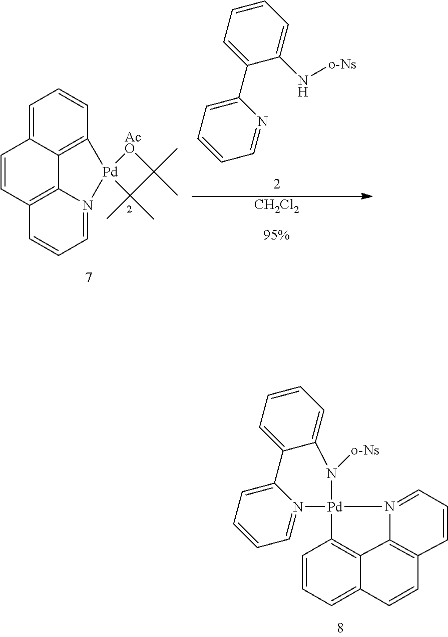

To the benzo[h]quinolinyl palladium acetate dimer (7) (342 mg, 1.00 mmol, 1.00 equiv) in CH₂Cl₂ (100 mL) at 23° C. is added 2-(2-pyridinyl)phenyl-2-nitrobenzenesulfonamide (2) (342 mg, 1.00 mmol, 1.00 equiv). After stirring for 20 min the reaction mixture is concentrated in vacuo. The resulting residue is triturated with Et₂O (3×1 mL) to afford 606 mg of the title compound as a colorless solid (95% yield).

Melting Point: >260° C. (decomp.). NMR Spectroscopy: ¹H NMR (500 MHz, CDCl₃, 23° C., δ): 9.55 (dd, J=5.5 Hz, 1.5 Hz, 1H), 8.99 (dd, J=5.5 Hz, 1.0 Hz, 1H), 8.30 (dd, J=8.5 Hz, 1.5 Hz, 1H), 7.76-7.71 (m, 2H), 7.64-7.54 ^{(m, 5H), 7.49 (dd}d^{, J=9.5 Hz,} 8^{.5 Hz}, 1.5 Hz, ^{1H), 7.41 (d}d^{, J=}7.5 ^{Hz,} 1.5 Hz, ^{1H)}, ^{7.36 (d}d^{, J=}8.0 ^{Hz}, 8.0 Hz, ^{1H)}, 7.26-7.13 ^{(m, 5H),} 7.04 (dd, J=8.0 Hz, 1.5 Hz, 1H), 7.00 (d, J=7.5 Hz, 1H). ¹³C NMR (125 MHz, CDCl₃, 23° C., δ): 176.0, 174.7, 168.4, 162.3, 160.4, 158.3, 155.5, 154.3, 151.3, 144.2, 142.3, 138.4, 137.5, 136.4, 134.9, 132.0, 131.1, 130.4, 130.1, 129.3, 128.9, 128.4, 126.9, 124.8, 124.6, 123.8, 123.4, 123.3, 122.6, 122.0. Mass Spectrometry: HRMS-FIA (m/z): Calcd for [C₃₀H₂₀N₄O₄S+H], 639.0313. Found, 639.0331.

### 10-fluorobenzo[h]quinoline (10)

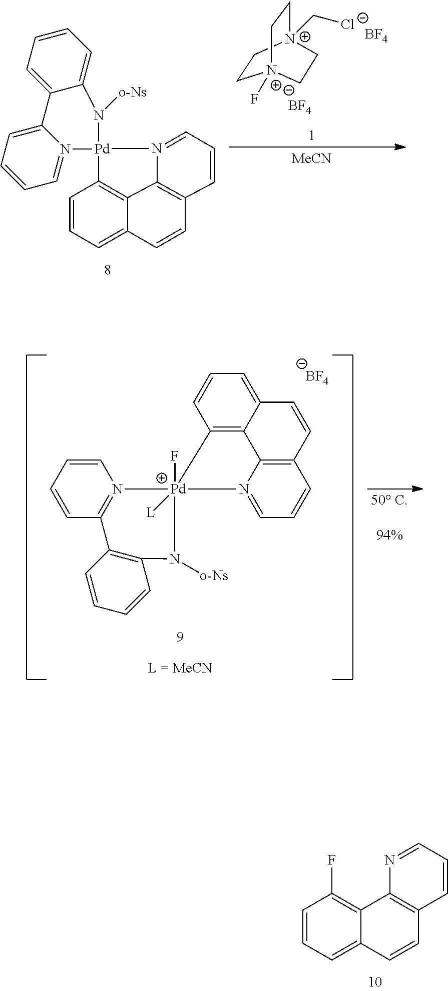

—0.0100 mmol Scale—

To the benzo[h]quinolinyl palladium(II) pyrdine-sulfonamido complex 8 (6.39 mg, 0.0100 mmol, 1.00 equiv) in MeCN (0.5 mL) at 23° C. is added 1-chloromethyl-4-fluoro-1,4-diazoniabicyclo [2.2.2]octane bis(tetrafluoroborate) (1) (3.90 mg, 0.0110 mmol, 1.10 equiv). After stirring for 10 min at 23° C., the reaction mixture has a dark purple color. The reaction mixture is warmed to 50° C. and stirred for 30 min. After cooling to 23° C., the reaction mixture is concentrated in vacuo. The resulting solid is purified by preparative TLC eluting with hexanes/EtOAc 7:3 (v/v) to afford 1.86 mg of the title compound as a colorless solid (94% yield, average of two runs).

—0.200 mmol Scale—

To the benzo[h]quinolinyl palladium(II) pyrdine-sulfonamido complex 8 (128 mg, 0.200 mmol, 1.00 equiv) in MeCN (2.0 mL) at 23° C. is added 1-chloromethyl-4-fluoro-1,4-diazoniabicyclo[2.2.2]octane bis(tetrafluoroborate) (1) (77.9 mg, 0.220 mmol, 1.10 equiv). After stirring for 10 min at 23° C., the reaction mixture has a dark purple color. The reaction mixture is warmed to 50° C. and stirred for 1.5 hr. After cooling to 23° C., the reaction mixture is concentrated in vacuo. The resulting solid is dissolved in CH₂Cl₂ and filtered through a pad of celite. The filtrate is concentrated in vacuo and the residue is purified by chromatography on silica gel eluting with hexanes/EtOAc 9:1 (v/v) to afford 27.4 mg of the title compound as a colorless solid (70% yield). The fluorination yield is temperature-dependent and afforded lower yields at lower temperature. The lower yield on 0.200 mmol scale may be explicable due to slower heating on larger scale.

R_{f}=0.79 (hexanes/EtOAc 7:3 (v/v)). NMR Spectroscopy: ¹H NMR (500 MHz, CDCl₃, 23° C., δ): 9.12 (dd, J=4.0 Hz, 1.0 Hz, 1H), 8.17 (d, J=7.5 Hz, 1H), 7.79 (d, J=9.0 Hz, 1H), 7.71 (d, J=8.0 Hz, 1H), 7.68 (d, J=8.0 Hz, 1H), ^{7.36 (}(d^{d}d^{, J=}8.0 ^{Hz,} 7.5 Hz, 4.5 Hz, ^{1H)}, 7.54 (dd, J=7.0 Hz, 4.5 Hz, 1H), 7.44 (dd, J=13.0 Hz, 8.0 Hz, 1H). ^{13C NMR (125 MHz, CDCl}3^{, 23° C.,} δ^{): 1}6¹.4 (d, J=259 Hz), 149.4, 146.3 (d, J=7.4 Hz), 136.5, 136.0, 128.6 (d, J=9.1 Hz), 127.8, 127.4, 126.9, 124.4, 121.9, 120.5 (d, J=6.4 Hz), 114.8 (d, J=24 Hz). ¹⁹F NMR (375 MHz, CDCl₃, 23° C., δ): −109.4 (d, J=11 Hz). Mass Spectrometry: HRMS-FIA (m/z): Calcd for [C₁₃H₈FN+H], 198.0714. Found, 198.0719.

### Difluoro palladium(IV) complex 11 by XeF₂ Oxidation

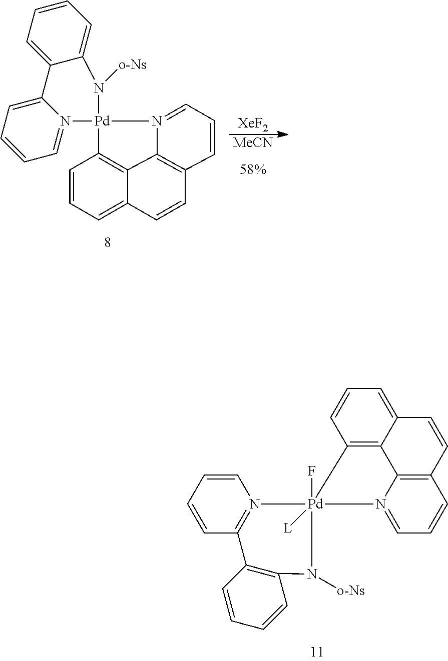

Under nitrogen atmosphere, to the benzo[h]quinolinyl palladium(II) pyrdine-sulfonamido complex 8 (128 mg, 0.200 mmol, 1.00 equiv) in anhydrous MeCN (2.0 mL) at 23° C. is added xenone difluoride (81.1 mg, 0.480 mmol, 2.40 equiv). After stirring for 1.0 hr at 23° C., the precipitate is filtered off and washed with acetone (5×1 mL). The solid is dissolved in CH₂Cl₂ and filtered through a pad of celite. The filtrate is concentrated in vacuo to afford 79.1 mg of the title compound as an orange solid (58% yield).

Melting Point: 143° C. (decomp.). NMR Spectroscopy: ¹H NMR (500 MHz, DMSO-d6, 23° C., δ): 9.72 (d, J=5.0 Hz, 1H), 9.28 (d, J=5.0 Hz, 1H), 9.18 (dd, J=17.5 Hz, 8.0 Hz, 1H), 8.94 (d, J=8.0 Hz, 1H), 8.20 (dd, J=8.0 Hz, 8.0 Hz, 1H), 8.12 (dd, J=8.0 Hz, 5.5 Hz, 1H), 8.07 (d, J=9.0 Hz, 1H), 8.03 (d, J=9.0 Hz, 1H), 7.89 (dd, J=7.0 Hz, 7.0 Hz, 1H), 7.86 (d, J=8.0 Hz, 1H), 7.75 (d, J=7.5 Hz, 1H), 7.73 (d, J=8.0 Hz, 1H), 7.44 (dd, J=7.5 Hz, 7.5 Hz, 1H), 7.35 (d, J=8.0 Hz, 1H), 7.31 (dd, J=7.5 Hz, 7.5 Hz, 1H), 7.14 (dd, J=8.0 Hz, 8.0 Hz, 1H), 7.07 (dd, J=8.0 Hz, 7.5 Hz, 1H), 7.01 (dd, J=7.5 Hz, 7.5 Hz, 1H), 6.36 (d, J=8.0 Hz, 1H), 6.21 (dd, J=7.5 Hz, 5.0 Hz, 1H). ¹³C NMR (125 MHz, DMSO-d₆, 23° C., δ): 161.1, 160.6, 152.8, 152.0, 151.0, 148.3, 142.9, 141.0, 139.6, 136.7, 135.3, 135.0, 133.4, 133.1, 132.3, 132.0, 131.9, 131.7, 131.4, 131.0, 129.2, 128.7, 128.3, 127.7, 127.5, 125.5, 125.4, 125.2, 124.1, 122.6. ¹⁹F NMR (375 MHz, DMSO-d6, 23° C., δ): −169.2 (d, J=113 Hz, 1F), −277.8 (d, J=113 Hz, 1F). The crystal structure is shown in the X-ray Crystallographic Analysis section below.

### Difluoro palladium(IV) complex 11 by SELECTFLUOR® (1) oxidation

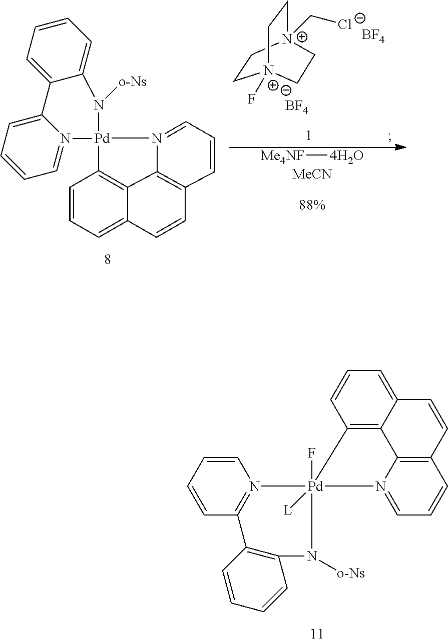

To the benzo[h]quinolinyl palladium(II) pyrdine-sulfonamido complex 8 (128 mg, 0.200 mmol, 1.00 equiv) in MeCN (2.0 mL) at 23° C. is added 1-chloromethyl-4-fluoro-1,4-diazoniabicyclo[2.2.2]octane bis(tetrafluoroborate) (1) (77.9 mg, 0.220 mmol, 1.10 equiv). After stirring for 10 min at 23° C., tetramethylammonium fluoride tetrahydrate (72.6 mg, 0.440 mmol, 2.20 equiv) is added to the reaction mixture. After stirring for 20 min at 23° C., the precipitate is filtered off and washed with acetone (5×2 mL). The solid is dissolved in CH₂Cl₂ and filtered through a pad of celite. The filtrate is concentrated in vacuo to afford 119 mg of the title compound as an orange solid (88% yield).

### Decomposition of palladium(IV)difluoride 11

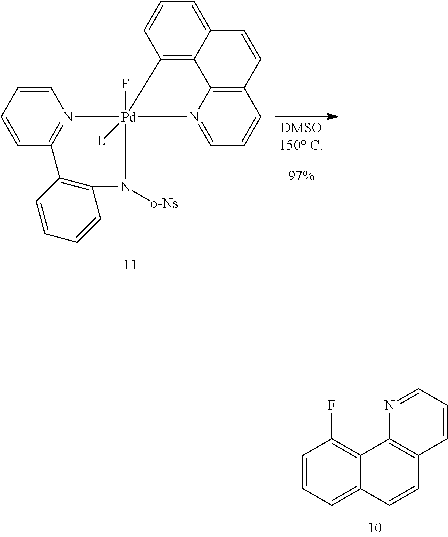

—0.0100 mmol Scale—

To DMSO-d₆ (0.5 mL) at 150° C. is added palladium(IV)difluoride complex 11 (6.76 mg, 0.0100 mmol, 1.00 equiv) in 5 portions over 5 min. After stirring for 10 min at 150° C., the reaction mixture is cooled to 23° C., at which temperature fluorobenzene (2.05 mg, 2.00 μL, 0.0213 mmol) is added. The yield is determined by comparing the integration of the ¹⁹F NMR (375 MHz, DMSO-d₆, 23° C.) resonance of 10-fluorobenzo[h]quinoline (−108.2 ppm) and that of fluorobenzene (−113.4 ppm) (97% yield, average of three runs). The fluorination yield is temperature-dependent and afforded lower yields at lower temperature. The lower yield on 0.100 mmol scale may be explicable due to slower heating on larger scale.

—0.100 mmol Scale—

To DMSO (5.0 mL) at 150° C. is added palladium(IV)difluoride complex 11 (67.6 mg, 0.100 mmol, 1.00 equiv) in 20 portions over 10 min. After stirring for 10 min at 150° C., the reaction mixture is cooled to 23° C. and half of the solvent is removed in vacuo. To the solution is added water (5.0 mL) and the aqueous phase is extracted with Et₂O (7×3 mL). The combined organic phases are washed with brine (3 mL) and dried (Na₂SO₄). The filtrate is concentrated in vacuo and the residue is purified by preparative TLC eluting with hexanes/EtOAc 4:1 (v/v) to afford 14.1 mg of the title compound as a colorless solid. (71% yield).

### Fluoro palladium(IV)tetrafluoroborate complex 9

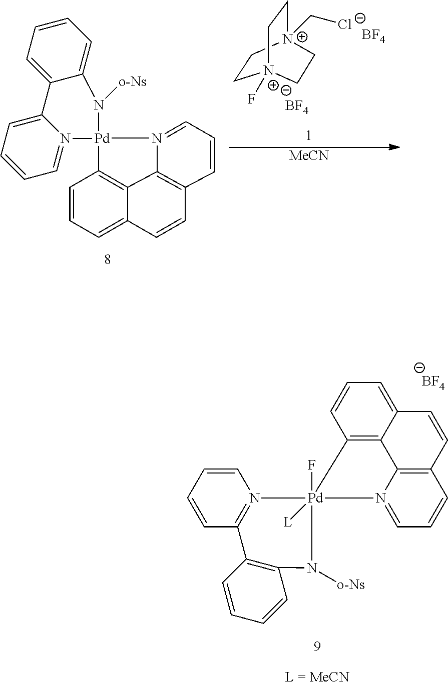

To the benzo[h]quinolinyl palladium(II) pyrdine-sulfonamido complex 8 (6.4 mg, 0.010 mmol, 1.0 equiv) in acetonitrile-d3 (0.5 mL) at 23° C. is added 1-chloromethyl-4-fluoro-1,4-diazoniabicyclo[2.2.2]octane bis(tetrafluoroborate) (1) (3.9 mg, 0.011 mmol, 1.1 equiv). After stirring for 10 min at 23° C., the colorless suspension forms a dark purple solution. Compound 9 was characterized by NMR in acetonitrile solution without purification.

NMR Spectroscopy: ¹H NMR (500 MHz, acetonitrile-d₃, 23° C., δ): 9.60 (d, J=6.0 Hz, 1H), 9.46 (d, J=6.0 Hz, 1H), 8.89 (dd, J=8.0 Hz, 1.0 Hz, 1H), 8.48 (dd, J=7.5 Hz, 7.5 Hz, 1H), 8.40 (d, J=8.0 Hz, 1H), 8.10-8.00 (m, 3H), 7.95 (dd, J=7.0 Hz, 6.5 Hz, 1H), 7.80-7.75 (m, 2H), 7.66-7.56 (m, 2H), 7.47-7.40 (m, 2H), 7.20 (dd, J=7.5 Hz, 7.5 Hz, 1H), 7.06 (dd, J=8.0 Hz, 8.0 Hz, 1H), 6.89 (dd, J=8.0 Hz, 7.5 Hz, 1H), 6.78 (d, J=8.0 Hz, 1H), 6.31 (d, J=9.0 Hz, 1H). ¹³C NMR (125 MHz, acetonitrile-d3, 23° C., δ): 153.8, 153.2, 151.1, 151.0, 150.1, 148.0, 147.3, 143.5, 141.7, 138.9, 136.1, 135.2, 134.7, 134.3, 132.7, 132.0, 131.8, 131.6, 131.5, 130.6, 129.4, 128.7, 127.2, 126.9, 126.6, 126.1, 126.0, 125.9, 125.0, 124.3. ¹⁹F NMR (375 MHz, acetonitrile-d₃, 23° C., δ): −152.0 (s, 4F), −278.0 (br, 1F).

### Bis(pyridinium)palladium(II)tetrafluoroborate complex 12

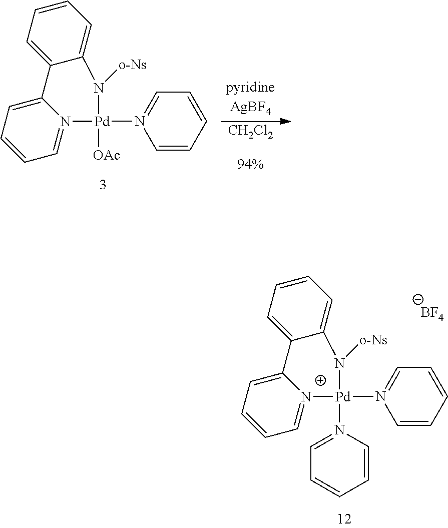

To acetato palladium complex 3 (30.0 mg, 0.501 mmol, 1.00 equiv) in CH₂Cl₂ (1.0 mL) at 23° C. is added pyridine (4.1 μL, 0.050 mmol, 1.0 equiv) and silver tetrafluoroborate (19.5 mg, 0.100 mmol, 2.00 equiv). After stirring for 30 min at 23° C., the reaction mixture is filtered through a pad of celite. The filtrate is concentrated in vacuo to afford 33 mg of the title compound as yellow oil (94% yield)

NMR Spectroscopy: ¹H NMR (500 MHz, acetonitrile-d3, 23° C., δ): 8.84-8.78 (m, 4H), 7.98-7.93 (m, 2H), 7.76 (dd, J=6.0 Hz, 1.0 Hz, 1H), 7.74-7.68 (m, 2H), 7.60 (ddd, J=7.5 Hz, 7.5 Hz, 1.5 Hz, 1H), 7.54-7.48 (m, 6H), 7.45 (dd, J=8.0 Hz, 1.0 Hz, 1H), 7.32 (ddd, J=8.0 Hz, 7.0 Hz, 2.0 Hz, 1H), 7.26 (dd, J=7.5 Hz, 1.0 Hz, 1H), 7.18-7.12 (m, 2H), 7.32 (ddd, J=7.5 Hz, 5.5 Hz, 1.5 Hz, 1H). ¹³C NMR (125 MHz, acetonitrile-d₃, 23° C., δ): 154.8, 151.9, 151.6, 151.0, 147.1, 141.1, 140.9, 140.5, 139.0, 137.0, 133.4, 132.9, 132.1, 131.7, 130.9, 130.5, 129.1, 127.5, 126.8, 126.5, 125.4, 124.9, 123.3. ¹⁹F NMR (375 MHz, acetonitrile-d3, 23° C., δ): −152.0 (s). Mass Spectrometry: HRMS-FIA (m/z): Calc'd for [C₂₇H₂₂BF₄N₅O₄PdS—BF₄], 618.0427. Found, 618.0434.

### X-ray Crystallographic Analysis

### Difluoro palladium(IV) complex 11 (CCDC 686490)

### Experimental

The compound was crystallized from an acetonitrile solution as orange prisms. A crystal 0.025 mm×0.050 mm×0.075 mm in size was selected, mounted on a nylon loop with Paratone-N oil, and transferred to a Bruker SMART APEX II diffractometer equipped with an Oxford Cryosystems 700 Series Cryostream Cooler and Mo Kα radiation (λ=0.71073 Å). A total of 2147 frames were collected at 193 (2) K to θₘₐₓ=22.490 with an oscillation range of 0.5°/frame, and an exposure time of 15 s/frame using the APEX2 suite of software. (Bruker AXS, 2006a) Data were collected to θₘₐₓ=22.490 rather than the routine value of θₘₐₓ=27.50° because the crystal examined did not exhibit usable diffraction beyond 22.49°. Unit cell refinement on all observed reflections, and data reduction with corrections for Lp and decay were performed using SAINT. (Bruker AXS, 2006b) Scaling and a multi-scan absorption correction were done using SADABS. (Bruker AXS, 2004) The minimum and maximum transmission factors were 0.9421 and 0.9802, respectively. A total of 34170 reflections were collected, 3643 were unique (Rᵢₙₜ=0.147), and 2584 had I>2σ(I). Systematic absences were consistent with the compound having crystallized in the monoclinic space group P2₁/n. The observed mean |E²-1| value was 0.912 (versus the expectation values of 0.968 and 0.736 for centric and noncentric data, respectively).

The structure was solved by direct methods and refined by full-matrix least-squares on F² using SHELXTL. (Bruker AXS, 2001) The asymmetric unit was found to contain one molecule of (Benzo[h]quinolinato){(2-nitrophenyl-sulfonyl)[(2-(pyridin-2-yl)phenyl)amido]difluoro-palladium(IV) and one molecule of acetonitrile. All of the nonhydrogen atoms were refined with anisotropic displacement coefficients. The hydrogen atoms were assigned isotropic displacement coefficients U(H)=1.2U(C) or 1.5U(C_{methyl}), and their coordinates were allowed to ride on their respective carbons. The acetonitrile was treated with a two-site disorder model consisting of partial atoms with fixed site occupancy factors of a half. The atoms associated with one of the two sites were specified with an asterisk, e.g., N1S and N1S*, and included in the least-squares refinement with 1,2-distance, rigid-bond and similar Uᵢⱼ restraints. The refinement converged to R(F)=0.0383, wR(F²)=0.0703, and S=1.042 for 2584 reflections with I>2σ(I), and R(F)=0.0728, wR(F²)=0.0829, and S=1.042 for 3643 unique reflections, 424 parameters, and 58 restraints. The maximum |Δ/σ| in the final cycle of least-squares was 0.001, and the residual peaks on the final difference-Fourier map ranged from −0.505 to 0.392 eÅ⁻³. Scattering factors were taken from the International Tables for Crystallography, Volume C. (Maslen et al., 1992, and Creagh & McAuley, 1992)

### REFERENCES

Bruker AXS (2001). SHELXTL v6.12. Bruker Analytical X-ray Systems Inc., Madison, Wis., USA.
Bruker AXS (2004). SADABS. Bruker Analytical X-ray Systems Inc., Madison, Wis., USA.
Bruker AXS (2006a). APEX2 v2.1-0. Bruker Analytical X-ray Systems Inc., Madison, Wis., USA.
Bruker AXS (2006b). SAINT V7.34A. Bruker Analytical X-ray Systems Inc., Madison, Wis., USA.
Creagh, D. C. & McAuley, W. J. (1992). International Tables for Crystallography: Mathematical, Physical and Chemical Tables, Vol C, edited by A. J. C. Wilson, pp. 206-222. Dordrecht, The Netherlands: Kluwer.
Maslen, E. N., Fox, A. G. & O'Keefe, M. A. (1992). International Tables for Crystallography: Mathematical, Physical and Chemical Tables, Vol C, edited by A. J. C. Wilson, pp. 476-516. Dordrecht, The Netherlands: Kluwer.

R(F)=R1=Σ∥Fₒ|−|F_{c}∥/Σ|Fₒ|, wR(F²)=wR2=[Σw(Fₒ²−F_{c}²)²/Σw(Fₒ²)²]^{1/2}, and S=Goodness-of-fit on F²=[w(Fₒ²−Fₑ²)²/(n−p)]^{1/2}, where n is the number of reflections and p is the number of parameters refined.

TABLE 10-1
Crystal data and structure refinement for the
difluoro palladium(IV) complex 11.

| Identification code | difluoro palladium(IV) complex 11 | |
|---|---|---|
| Empirical formula | C32H23F2N5O4PdS | |
| Formula weight | 718.01 | |
| Temperature | 193(2) K | |
| Wavelength | 0.71073≈ | |
| Crystal system | Monoclinic | |
| Space group | P 21/n | |
| Unit cell dimensions | a = 10.0089(3)≈ | α = 90∞. |
| | b = 13.3937(4)≈ | β = 99.197(3)∞. |
| | c = 21.0503(7)≈ | γ = 90∞. |

| | |
|---|---|
| Volume | 2785.65(15)≈³ |
| Z | 4 |
| Density (calculated) | 1.712 Mg/m³ |
| Absorption coefficient | 0.805 mm⁻¹ |
| F(000) | 1448 |
| Crystal size | 0.075 × 0.050 × 0.025 mm³ |
| Theta range for data collection | 1.81 to 22.49∞. |
| Index ranges | −10 <= h <= 10, −14 <= k <= 14, |
| | −22 <= l <= 22 |
| Reflections collected | 34170 |
| Independent reflections | 3643 [R(int) = 0.1469] |
| Completeness to theta = 22.49∞ | 100.0% |
| Absorption correction | Semi-empirical from equivalents |
| Max. and min. transmission | 0.9802 and 0.9421 |
| Refinement method | Full-matrix least-squares on F² |
| Data/restraints/parameters | 3643/58/424 |
| Goodness-of-fit on F² | 1.042 |
| Final R indices [I > 2sigma(I)] | R1 = 0.0383, wR2 = 0.0703 |
| R indices (all data) | R1 = 0.0728, wR2 = 0.0829 |
| Largest diff. peak and hole | 0.392 and −0.505 e · ≈⁻³ |

TABLE 10-2
Atomic coordinates (× 10⁴) and equivalent isotropic displacement
parameters (Å² × 10³) for the difluoro palladium(IV) complex 11.
U(eq) is defined as one third of the trace of the orthogonalized Uᵢⱼ tensor.

| | x | y | z | U(eq) |
|---|---|---|---|---|
| Pd | 3838(1) | 1424(1) | 1183(1) | 22(1) |
| F(1) | 5817(3) | 1199(2) | 1097(1) | 32(1) |
| F(2) | 3569(3) | 2017(2) | 324(1) | 34(1) |
| N(1) | 4481(4) | 2737(3) | 1604(2) | 26(1) |
| C(2) | 5333(6) | 3254(4) | 1286(3) | 33(2) |
| C(3) | 6042(6) | 4070(5) | 1567(4) | 41(2) |
| C(4) | 5884(6) | 4335(5) | 2178(3) | 41(2) |
| C(5) | 5021(6) | 3826(4) | 2496(3) | 33(2) |
| C(6) | 4298(5) | 3020(4) | 2202(3) | 27(2) |
| C(7) | 3327(5) | 2461(4) | 2521(2) | 20(1) |
| C(8) | 2497(5) | 3008(4) | 2879(3) | 27(2) |
| C(9) | 1528(6) | 2546(5) | 3150(3) | 33(2) |
| C(10) | 1354(6) | 1517(5) | 3100(3) | 35(2) |
| C(11) | 2160(5) | 960(5) | 2757(3) | 26(2) |
| C(12) | 3140(5) | 1421(5) | 2464(2) | 21(1) |
| N(13) | 3895(4) | 854(3) | 2075(2) | 21(1) |
| S(14) | 5103(1) | 186(1) | 2466(1) | 25(1) |
| O(15) | 5754(4) | −350(3) | 2017(2) | 31(1) |
| O(16) | 4573(4) | −359(3) | 2957(2) | 29(1) |
| C(17) | 6294(5) | 1061(4) | 2875(3) | 23(1) |
| C(18) | 7074(5) | 1609(4) | 2510(3) | 26(2) |
| C(19) | 7959(6) | 2328(5) | 2790(3) | 34(2) |
| C(20) | 8057(6) | 2534(5) | 3443(3) | 36(2) |
| C(21) | 7315(6) | 1998(5) | 3822(3) | 34(2) |
| C(22) | 6435(5) | 1268(4) | 3528(3) | 27(2) |
| N(23) | 5678(6) | 732(5) | 3970(2) | 39(1) |
| O(24) | 6112(5) | −78(4) | 4178(2) | 54(1) |
| O(25) | 4687(5) | 1156(4) | 4113(2) | 63(2) |
| N(26) | 3319(4) | 74(3) | 802(2) | 24(1) |
| C(27) | 4128(6) | −563(5) | 580(3) | 30(2) |
| C(28) | 3656(6) | −1483(5) | 337(3) | 34(2) |
| C(29) | 2333(7) | −1735(5) | 339(3) | 38(2) |
| C(29A) | 1433(6) | −1060(5) | 560(3) | 34(2) |
| C(30) | 12(7) | −1190(5) | 562(3) | 41(2) |
| C(31) | −753(7) | −461(5) | 763(3) | 38(2) |
| C(31A) | −199(6) | 495(5) | 976(3) | 32(2) |
| C(32) | −953(6) | 1317(6) | 1133(3) | 39(2) |
| C(33) | −330(6) | 2224(6) | 1277(3) | 40(2) |
| C(34) | 1070(6) | 2354(5) | 1295(3) | 28(2) |
| C(35) | 1834(5) | 1549(5) | 1167(2) | 25(1) |
| C(35A) | 1193(5) | 636(5) | 991(2) | 23(1) |
| C(35B) | 1984(6) | −138(4) | 791(3) | 23(1) |
| N(1S) | 4160(20) | 5940(20) | 230(20) | 65(8) |
| C(2S) | 3430(50) | 5390(30) | 400(30) | 47(8) |
| C(3S) | 2607(7) | 4559(5) | 577(3) | 52(2) |
| N(1S*) | 3720(20) | 6080(20) | 110(20) | 55(7) |
| C(2S*) | 3270(60) | 5370(30) | 290(30) | 48(8) |

TABLE 10-3
Bond lengths [Å] and angles [°] for the difluoro palladium(IV) complex 11.

| | | | |
|---|---|---|---|
| Pd—F(2) | 1.955(3) | C(18)—C(19) | 1.376(8) |
| Pd—C(35) | 2.008(5) | C(18)—H(18) | 0.9500 |
| Pd—N(26) | 2.012(5) | C(19)—C(20) | 1.389(8) |
| Pd—N(13) | 2.019(4) | C(19)—H(19) | 0.9500 |
| Pd—N(1) | 2.027(5) | C(20)—C(21) | 1.376(8) |
| Pd—F(1) | 2.040(3) | C(20)—H(20) | 0.9500 |
| N(1)—C(6) | 1.354(7) | C(21)—C(22) | 1.393(8) |
| N(1)—C(2) | 1.355(7) | C(21)—H(21) | 0.9500 |
| C(2)—C(3) | 1.385(8) | C(22)—N(23) | 1.477(7) |
| C(2)—H(2) | 0.9500 | N(23)—O(25) | 1.222(6) |
| C(3)—C(4) | 1.367(9) | N(23)—O(24) | 1.224(7) |
| C(3)—H(3) | 0.9500 | N(26)—C(27) | 1.314(7) |
| C(4)—C(5) | 1.357(8) | N(26)—C(35B) | 1.363(7) |
| C(4)—H(4) | 0.9500 | C(27)—C(28) | 1.388(8) |
| C(5)—C(6) | 1.389(8) | C(27)—H(27) | 0.9500 |
| C(5)—H(5) | 0.9500 | C(28)—C(29) | 1.367(8) |
| C(6)—C(7) | 1.471(8) | C(28)—H(28) | 0.9500 |
| C(7)—C(12) | 1.408(8) | C(29)—C(29A) | 1.408(8) |
| C(7)—C(8) | 1.412(7) | C(29)—H(29) | 0.9500 |
| C(8)—C(9) | 1.351(8) | C(29A)—C(35B) | 1.407(8) |
| C(8)—H(8) | 0.9500 | C(29A)—C(30) | 1.433(8) |
| C(9)—C(10) | 1.390(8) | C(30)—C(31) | 1.350(9) |
| C(9)—H(9) | 0.9500 | C(30)—H(30) | 0.9500 |
| C(10)—C(11) | 1.383(8) | C(31)—C(31A) | 1.438(8) |
| C(10)—H(10) | 0.9500 | C(31)—H(31) | 0.9500 |
| C(11)—C(12) | 1.385(7) | C(31A)—C(35A) | 1.401(8) |
| C(11)—H(11) | 0.9500 | C(31A)—C(32) | 1.403(8) |
| C(12)—N(13) | 1.421(7) | C(32)—C(33) | 1.378(9) |
| N(13)—S(14) | 1.619(4) | C(32)—H(32) | 0.9500 |
| S(14)—O(15) | 1.426(4) | C(33)—C(34) | 1.406(8) |
| S(14)—O(16) | 1.435(4) | C(33)—H(33) | 0.9500 |
| S(14)—C(17) | 1.791(6) | C(34)—C(35) | 1.374(8) |
| C(17)—C(22) | 1.386(8) | C(34)—H(34) | 0.9500 |
| C(17)—C(18) | 1.389(7) | C(35)—C(35A) | 1.403(8) |
| C(35A)—C(35B) | 1.408(8) | C(3S)—H(3SC) | 0.9800 |
| N(1S)—C(2S) | 1.139(10) | C(3S)—H(3SD) | 0.9800 |
| C(2S)—C(3S) | 1.462(10) | C(3S)—H(3SE) | 0.9800 |
| C(3S)—C(2S*) | 1.458(10) | C(3S)—H(3SF) | 0.9800 |
| C(3S)—H(3SA) | 0.9800 | N(1S*)—C(2S*) | 1.136(10) |
| C(3S)—H(3SB) | 0.9800 | C(6)—C(5)—H(5) | 120.1 |
| F(2)—Pd—C(35) | 87.81(18) | N(1)—C(6)—C(5) | 119.5(5) |
| F(2)—Pd—N(26) | 90.47(15) | N(1)—C(6)—C(7) | 118.7(5) |
| C(35)—Pd—N(26) | 82.8(2) | C(5)—C(6)—C(7) | 121.8(5) |
| F(2)—Pd—N(13) | 173.48(15) | C(12)—C(7)—C(8) | 118.5(5) |
| C(35)—Pd—N(13) | 85.77(19) | C(12)—C(7)—C(6) | 123.5(5) |
| N(26)—Pd—N(13) | 89.85(18) | C(8)—C(7)—C(6) | 117.9(5) |
| F(2)—Pd—N(1) | 92.23(17) | C(9)—C(8)—C(7) | 120.7(6) |
| C(35)—Pd—N(1) | 100.5(2) | C(9)—C(8)—H(8) | 119.6 |
| N(26)—Pd—N(1) | 175.84(18) | C(7)—C(8)—H(8) | 119.6 |
| N(13)—Pd—N(1) | 87.82(18) | C(8)—C(9)—C(10) | 120.8(6) |
| F(2)—Pd—F(1) | 88.27(13) | C(8)—C(9)—H(9) | 119.6 |
| C(35)—Pd—F(1) | 172.95(18) | C(10)—C(9)—H(9) | 119.6 |
| N(26)—Pd—F(1) | 91.38(16) | C(11)—C(10)—C(9) | 119.8(6) |
| N(13)—Pd—F(1) | 98.24(14) | C(11)—C(10)—H(10) | 120.1 |
| N(1)—Pd—F(1) | 85.54(15) | C(9)—C(10)—H(10) | 120.1 |
| C(6)—N(1)—C(2) | 120.3(5) | C(10)—C(11)—C(12) | 120.4(6) |
| C(6)—N(1)—Pd | 124.6(4) | C(10)—C(11)—H(11) | 119.8 |
| C(2)—N(1)—Pd | 114.1(4) | C(12)—C(11)—H(11) | 119.8 |
| N(1)—C(2)—C(3) | 120.8(6) | C(11)—C(12)—C(7) | 119.8(5) |
| N(1)—C(2)—H(2) | 119.6 | C(11)—C(12)—N(13) | 119.9(5) |
| C(3)—C(2)—H(2) | 119.6 | C(7)—C(12)—N(13) | 120.2(5) |
| C(4)—C(3)—C(2) | 118.5(6) | C(12)—N(13)—S(14) | 115.2(3) |
| C(4)—C(3)—H(3) | 120.7 | C(12)—N(13)—Pd | 113.3(3) |
| C(2)—C(3)—H(3) | 120.7 | S(14)—N(13)—Pd | 126.1(2) |
| C(5)—C(4)—C(3) | 120.8(6) | O(15)—S(14)—O(16) | 118.9(2) |
| C(5)—C(4)—H(4) | 119.6 | O(15)—S(14)—N(13) | 108.9(2) |
| C(3)—C(4)—H(4) | 119.6 | O(16)—S(14)—N(13) | 108.4(2) |
| C(4)—C(5)—C(6) | 119.9(6) | O(15)—S(14)—C(17) | 108.0(2) |
| C(4)—C(5)—H(5) | 120.1 | O(16)—S(14)—C(17) | 106.3(3) |
| N(13)—S(14)—C(17) | 105.6(2) | C(29)—C(29A)—C(30) | 127.6(6) |
| C(22)—C(17)—C(18) | 117.7(5) | C(31)—C(30)—C(29A) | 121.7(6) |
| C(22)—C(17)—S(14) | 124.2(4) | C(31)—C(30)—H(30) | 119.2 |
| C(18)—C(17)—S(14) | 118.0(4) | C(29A)—C(30)—H(30) | 119.2 |
| C(19)—C(18)—C(17) | 120.8(5) | C(30)—C(31)—C(31A) | 122.2(6) |
| C(19)—C(18)—H(18) | 119.6 | C(30)—C(31)—H(31) | 118.9 |
| C(17)—C(18)—H(18) | 119.6 | C(31A)—C(31)—H(31) | 118.9 |
| C(18)—C(19)—C(20) | 120.1(6) | C(35A)—C(31A)—C(32) | 117.4(6) |
| C(18)—C(19)—H(19) | 119.9 | C(35A)—C(31A)—C(31) | 117.3(6) |
| C(20)—C(19)—H(19) | 119.9 | C(32)—C(31A)—C(31) | 125.2(6) |
| C(21)—C(20)—C(19) | 120.7(6) | C(33)—C(32)—C(31A) | 120.1(6) |
| C(21)—C(20)—H(20) | 119.7 | C(33)—C(32)—H(32) | 119.9 |
| C(19)—C(20)—H(20) | 119.7 | C(31A)—C(32)—H(32) | 119.9 |
| C(20)—C(21)—C(22) | 118.0(6) | C(32)—C(33)—C(34) | 121.9(6) |
| C(20)—C(21)—H(21) | 121.0 | C(32)—C(33)—H(33) | 119.0 |
| C(22)—C(21)—H(21) | 121.0 | C(34)—C(33)—H(33) | 119.0 |
| C(17)—C(22)—C(21) | 122.5(6) | C(35)—C(34)—C(33) | 118.8(6) |
| C(17)—C(22)—N(23) | 123.1(5) | C(35)—C(34)—H(34) | 120.6 |
| C(21)—C(22)—N(23) | 114.3(5) | C(33)—C(34)—H(34) | 120.6 |
| O(25)—N(23)—O(24) | 125.4(6) | C(34)—C(35)—C(35A) | 119.4(5) |
| O(25)—N(23)—C(22) | 116.6(6) | C(34)—C(35)—Pd | 130.4(5) |
| O(24)—N(23)—C(22) | 117.9(6) | C(35A)—C(35)—Pd | 110.2(4) |
| C(27)—N(26)—C(35B) | 121.2(5) | C(31A)—C(35A)—C(35) | 122.2(6) |
| C(27)—N(26)—Pd | 126.2(4) | C(31A)—C(35A)—C(35B) | 119.9(6) |
| C(35B)—N(26)—Pd | 112.6(4) | C(35)—C(35A)—C(35B) | 117.7(5) |
| N(26)—C(27)—C(28) | 121.0(6) | N(26)—C(35B)—C(29A) | 121.2(5) |
| N(26)—C(27)—H(27) | 119.5 | N(26)—C(35B)—C(35A) | 116.0(5) |
| C(28)—C(27)—H(27) | 119.5 | C(29A)—C(35B)—C(35A) | 122.7(5) |
| C(29)—C(28)—C(27) | 119.4(6) | N(1S)—C(2S)—C(3S) | 171(6) |
| C(29)—C(28)—H(28) | 120.3 | C(2S*)—C(3S)—H(3SA) | 99.0 |
| C(27)—C(28)—H(28) | 120.3 | C(2S)—C(3S)—H(3SA) | 109.5 |
| C(28)—C(29)—C(29A) | 120.9(6) | C(2S*)—C(3S)—H(3SB) | 112.8 |
| C(28)—C(29)—H(29) | 119.5 | C(2S)—C(3S)—H(3SB) | 109.5 |
| C(29A)—C(29)—H(29) | 119.5 | H(3SA)—C(3S)—H(3SB) | 109.5 |
| C(35B)—C(29A)—C(29) | 116.2(6) | C(2S*)—C(3S)—H(3SC) | 116.1 |
| C(35B)—C(29A)—C(30) | 116.1(6) | C(2S)—C(3S)—H(3SC) | 109.5 |
| H(3SA)—C(3S)—H(3SC) | 109.5 | H(3SB)—C(3S)—H(3SE) | 137.3 |
| H(3SB)—C(3S)—H(3SC) | 109.5 | H(3SD)—C(3S)—H(3SE) | 109.5 |
| C(2S*)—C(3S)—H(3SD) | 109.5 | C(2S*)—C(3S)—H(3SF) | 109.5 |
| C(2S)—C(3S)—H(3SD) | 99.5 | C(2S)—C(3S)—H(3SF) | 117.5 |
| H(3SA)—C(3S)—H(3SD) | 149.6 | H(3SA)—C(3S)—H(3SF) | 48.7 |
| H(3SB)—C(3S)—H(3SD) | 49.8 | H(3SB)—C(3S)—H(3SF) | 61.5 |
| H(3SC)—C(3S)—H(3SD) | 67.5 | H(3SC)—C(3S)—H(3SF) | 132.5 |
| C(2S*)—C(3S)—H(3SE) | 109.5 | H(3SD)—C(3S)—H(3SF) | 109.5 |
| C(2S)—C(3S)—H(3SE) | 110.9 | H(3SE)—C(3S)—H(3SF) | 109.5 |
| H(3SA)—C(3S)—H(3SE) | 69.0 | N(1S*)—C(2S*)—C(3S) | 172(6) |

TABLE 10-4
Anisotropic displacement parameters (Å² × 10³) for the difluoro
palladium(IV) complex 11. The anisotropic displacement factor
exponent takes the form: −2π²[h²a*²U₁₁ + . . . + 2 h k a* b* U₁₂]

| | U₁₁ | U₂₂ | U₃₃ | U₂₃ | U₁₃ | U₁₂ |
|---|---|---|---|---|---|---|
| Pd | 19(1) | 24(1) | 22(1) | 0(1) | 4(1) | 0(1) |
| F(1) | 21(2) | 42(2) | 33(2) | 0(2) | 8(2) | 1(2) |
| F(2) | 36(2) | 40(2) | 26(2) | 6(2) | 8(2) | −1(2) |
| N(1) | 23(3) | 20(3) | 34(3) | −3(2) | 5(2) | −2(2) |
| C(2) | 33(4) | 31(4) | 36(4) | 7(3) | 13(3) | 0(3) |
| C(3) | 27(4) | 27(4) | 72(6) | 4(4) | 13(4) | −10(3) |
| C(4) | 38(4) | 23(4) | 61(5) | −13(4) | 10(4) | −2(3) |
| C(5) | 28(4) | 31(4) | 41(4) | −9(3) | 5(3) | −1(3) |
| C(6) | 17(3) | 25(4) | 38(4) | −2(3) | 1(3) | 5(3) |
| C(7) | 18(3) | 20(4) | 19(3) | −3(3) | −1(3) | 3(3) |
| C(8) | 23(3) | 24(4) | 32(4) | −6(3) | −2(3) | 3(3) |
| C(9) | 19(3) | 46(5) | 32(4) | −14(3) | 1(3) | 2(3) |
| C(10) | 25(3) | 52(5) | 28(4) | −3(4) | 7(3) | −8(4) |
| C(11) | 19(3) | 35(4) | 25(4) | 1(3) | 4(3) | −2(3) |
| C(12) | 14(3) | 27(3) | 19(3) | −1(3) | −5(2) | 6(3) |
| N(13) | 15(2) | 21(3) | 26(3) | 0(2) | 2(2) | 6(2) |
| S(14) | 24(1) | 20(1) | 31(1) | −1(1) | 0(1) | 1(1) |
| O(15) | 28(2) | 26(2) | 39(3) | −6(2) | 4(2) | 13(2) |
| O(16) | 30(2) | 23(2) | 31(2) | 10(2) | 0(2) | −5(2) |
| C(17) | 16(3) | 23(4) | 31(4) | 0(3) | 2(3) | 6(3) |
| C(18) | 18(3) | 35(4) | 27(3) | 8(3) | 5(3) | 8(3) |
| C(19) | 18(3) | 39(4) | 45(4) | 2(3) | 6(3) | −3(3) |
| C(20) | 24(4) | 43(5) | 39(4) | −8(4) | 1(3) | −7(3) |
| C(21) | 29(4) | 38(4) | 34(4) | −11(3) | 0(3) | 1(3) |
| C(22) | 24(3) | 27(4) | 31(4) | 3(3) | 7(3) | 8(3) |
| N(23) | 38(4) | 53(4) | 26(3) | −7(3) | 3(3) | −11(3) |
| O(24) | 59(3) | 54(4) | 44(3) | 18(3) | −6(2) | −6(3) |
| O(25) | 64(4) | 62(4) | 77(4) | −13(3) | 48(3) | −10(3) |
| N(26) | 25(3) | 29(3) | 19(3) | −1(2) | 5(2) | 0(2) |
| C(27) | 39(4) | 30(4) | 20(3) | 3(3) | 7(3) | 6(3) |
| C(28) | 44(4) | 33(4) | 22(3) | −7(3) | 0(3) | 5(4) |
| C(29) | 63(5) | 25(4) | 22(4) | 1(3) | −5(3) | −3(4) |
| C(29A) | 43(4) | 34(4) | 22(4) | 5(3) | −4(3) | −10(3) |
| C(30) | 43(4) | 44(5) | 29(4) | 0(3) | −11(3) | −20(4) |
| C(31) | 33(4) | 55(5) | 26(4) | 5(4) | 3(3) | −19(4) |
| C(31A) | 31(4) | 44(5) | 20(4) | 4(3) | 1(3) | −1(3) |
| C(32) | 21(3) | 74(5) | 21(3) | 8(4) | 1(3) | 2(4) |
| C(33) | 28(4) | 62(5) | 32(4) | −1(4) | 7(3) | 17(4) |
| C(34) | 23(3) | 37(4) | 23(3) | 2(3) | 1(3) | 0(3) |
| C(35) | 18(3) | 39(4) | 18(3) | −1(3) | 4(2) | −2(3) |
| C(35A) | 19(3) | 40(4) | 7(3) | 4(3) | −2(2) | 1(3) |
| C(35B) | 26(4) | 27(4) | 16(3) | 2(3) | 2(3) | 2(3) |
| N(1S) | 70(17) | 51(11) | 80(20) | 13(10) | 26(17) | 23(12) |
| C(2S) | 66(17) | 51(13) | 26(16) | −3(11) | 11(15) | 15(10) |
| C(3S) | 66(5) | 48(5) | 42(4) | 4(4) | 4(4) | 9(4) |
| N(1S*) | 55(14) | 42(12) | 69(16) | −2(9) | 10(14) | 6(11) |
| C(2S*) | 58(14) | 44(12) | 40(20) | −14(11) | −4(11) | −3(10) |

TABLE 10-5
Hydrogen coordinates (×10⁴) and isotropic displacement parameters
(Å² × 10³) for the difluoro palladium(IV) complex 11.

| | x | y | z | U(eq) |
|---|---|---|---|---|
| H(2) | 5444 | 3054 | 865 | 39 |
| H(3) | 6627 | 4439 | 1341 | 50 |
| H(4) | 6383 | 4881 | 2382 | 49 |
| H(5) | 4911 | 4021 | 2918 | 40 |
| H(8) | 2622 | 3708 | 2930 | 33 |
| H(9) | 957 | 2928 | 3376 | 39 |
| H(10) | 684 | 1198 | 3301 | 41 |
| H(11) | 2040 | 258 | 2723 | 32 |
| H(18) | 6995 | 1486 | 2061 | 32 |
| H(19) | 8505 | 2684 | 2537 | 41 |
| H(20) | 8643 | 3050 | 3630 | 43 |
| H(21) | 7400 | 2122 | 4271 | 41 |
| H(27) | 5051 | −392 | 585 | 36 |
| H(28) | 4247 | −1934 | 170 | 40 |
| H(29) | 2017 | −2376 | 190 | 45 |
| H(30) | −402 | −1805 | 418 | 49 |
| H(31) | −1687 | −583 | 764 | 46 |
| H(32) | −1896 | 1248 | 1139 | 47 |
| H(33) | −861 | 2779 | 1368 | 48 |
| H(34) | 1479 | 2987 | 1394 | 33 |
| H(3SA) | 2038 | 4302 | 189 | 78 |
| H(3SB) | 2030 | 4792 | 882 | 78 |
| H(3SC) | 3201 | 4026 | 776 | 78 |
| H(3SD) | 2798 | 4616 | 1047 | 78 |
| H(3SE) | 2951 | 3919 | 445 | 78 |
| H(3SF) | 1627 | 4593 | 433 | 78 |

TABLE 10-6
Torsion angles [°] for the difluoro palladium(IV) complex 11.

| | | | |
|---|---|---|---|
| F(2)—Pd—N(1)—C(6) | 149.0(4) | C(11)—C(12)—N(13)—S(14) | −78.2(5) |
| C(35)—Pd—N(1)—C(6) | 60.8(5) | C(7)—C(12)—N(13)—S(14) | 105.6(5) |
| N(13)—Pd—N(1)—C(6) | −24.5(4) | C(11)—C(12)—N(13)—Pd | 126.1(4) |
| F(1)—Pd—N(1)—C(6) | −122.9(4) | C(7)—C(12)—N(13)—Pd | −50.0(5) |
| F(2)—Pd—N(1)—C(2) | −42.1(4) | C(35)—Pd—N(13)—C(12) | −46.7(4) |
| C(35)—Pd—N(1)—C(2) | −130.3(4) | N(26)—Pd—N(13)—C(12) | −129.5(4) |
| N(13)—Pd—N(1)—C(2) | 144.4(4) | N(1)—Pd—N(13)—C(12) | 54.0(4) |
| F(1)—Pd—N(1)—C(2) | 46.0(4) | F(1)—Pd—N(13)—C(12) | 139.1(3) |
| C(6)—N(1)—C(2)—C(3) | 0.8(8) | C(35)—Pd—N(13)—S(14) | 160.8(3) |
| Pd—N(1)—C(2)—C(3) | −168.6(4) | N(26)—Pd—N(13)—S(14) | 78.0(3) |
| N(1)—C(2)—C(3)—C(4) | 1.1(9) | N(1)—Pd—N(13)—S(14) | −98.5(3) |
| C(2)—C(3)—C(4)—C(5) | −1.8(10) | F(1)—Pd—N(13)—S(14) | −13.4(3) |
| C(3)—C(4)—C(5)—C(6) | 0.7(9) | C(12)—N(13)—S(14)—O(15) | 179.1(4) |
| C(2)—N(1)—C(6)—C(5) | −2.0(8) | Pd—N(13)—S(14)—O(15) | −28.8(4) |
| Pd—N(1)—C(6)—C(5) | 166.2(4) | C(12)—N(13)—S(14)—O(16) | 48.5(5) |
| C(2)—N(1)—C(6)—C(7) | 177.8(5) | Pd—N(13)—S(14)—O(16) | −159.5(3) |
| Pd—N(1)—C(6)—C(7) | −14.0(7) | C(12)—N(13)—S(14)—C(17) | −65.1(4) |
| C(4)—C(5)—C(6)—N(1) | 1.2(9) | Pd—N(13)—S(14)—C(17) | 86.9(3) |
| C(4)—C(5)—C(6)—C(7) | −178.5(5) | O(15)—S(14)—C(17)—C(22) | −139.7(5) |
| N(1)—C(6)—C(7)—C(12) | 36.8(8) | O(16)—S(14)—C(17)—C(22) | −11.0(5) |
| C(5)—C(6)—C(7)—C(12) | −143.4(6) | N(13)—S(14)—C(17)—C(22) | 103.9(5) |
| N(1)—C(6)—C(7)—C(8) | −140.1(5) | O(15)—S(14)—C(17)—C(18) | 44.1(5) |
| C(5)—C(6)—C(7)—C(8) | 39.6(8) | O(16)—S(14)—C(17)—C(18) | 172.7(4) |
| C(12)—C(7)—C(8)—C(9) | −1.3(8) | N(13)—S(14)—C(17)—C(18) | −72.3(5) |
| C(6)—C(7)—C(8)—C(9) | 175.8(5) | C(22)—C(17)—C(18)—C(19) | 0.3(8) |
| C(7)—C(8)—C(9)—C(10) | 2.2(9) | S(14)—C(17)—C(18)—C(19) | 176.8(4) |
| C(8)—C(9)—C(10)—C(11) | −1.6(9) | C(17)—C(18)—C(19)—C(20) | −1.7(9) |
| C(9)—C(10)—C(11)—C(12) | 0.1(8) | C(18)—C(19)—C(20)—C(21) | 2.6(9) |
| C(10)—C(11)—C(12)—C(7) | 0.7(8) | C(19)—C(20)—C(21)—C(22) | −2.0(9) |
| C(10)—C(11)—C(12)—N(13) | −175.5(5) | C(18)—C(17)—C(22)—C(21) | 0.2(8) |
| C(8)—C(7)—C(12)—C(11) | −0.1(7) | S(14)—C(17)—C(22)—C(21) | −176.0(4) |
| C(6)—C(7)—C(12)—C(11) | −177.0(5) | C(18)—C(17)—C(22)—N(23) | −179.3(5) |
| C(8)—C(7)—C(12)—N(13) | 176.0(4) | S(14)—C(17)—C(22)—N(23) | 4.5(8) |
| C(6)—C(7)—C(12)—N(13) | −0.9(8) | C(20)—C(21)—C(22)—C(17) | 0.6(9) |
| C(20)—C(21)—C(22)—N(23) | −179.9(5) | N(13)—Pd—C(35)—C(35A) | −83.0(4) |
| C(17)—C(22)—N(23)—O(25) | −100.3(7) | N(1)—Pd—C(35)—C(35A) | −170.0(4) |
| C(21)—C(22)—N(23)—O(25) | 80.1(7) | C(32)—C(31A)—C(35A)—C(35) | 1.4(8) |
| C(17)—C(22)—N(23)—O(24) | 81.9(7) | C(31)—C(31A)—C(35A)—C(35) | 177.6(5) |
| C(21)—C(22)—N(23)—O(24) | −97.7(6) | C(32)—C(31A)—C(35A)—C(35B) | −175.1(5) |
| F(2)—Pd—N(26)—C(27) | 85.0(4) | C(31)—C(31A)—C(35A)—C(35B) | 1.0(8) |
| C(35)—Pd—N(26)—C(27) | 172.8(5) | C(34)—C(35)—C(35A)—C(31A) | −3.8(8) |
| N(13)—Pd—N(26)—C(27) | −101.5(5) | Pd—C(35)—C(35A)—C(31A) | 176.4(4) |
| F(1)—Pd—N(26)—C(27) | −3.2(5) | C(34)—C(35)—C(35A)—C(35B) | 172.8(5) |
| F(2)—Pd—N(26)—C(35B) | −94.7(4) | Pd—C(35)—C(35A)—C(35B) | −7.0(6) |
| C(35)—Pd—N(26)—C(35B) | −7.0(4) | C(27)—N(26)—C(35B)—C(29A) | 2.1(8) |
| N(13)—Pd—N(26)—C(35B) | 78.7(4) | Pd—N(26)—C(35B)—C(29A) | −178.1(4) |
| F(1)—Pd—N(26)—C(35B) | 177.0(4) | C(27)—N(26)—C(35B)—C(35A) | −174.7(5) |
| C(35B)—N(26)—C(27)—C(28) | −1.3(8) | Pd—N(26)—C(35B)—C(35A) | 5.1(6) |
| Pd—N(26)—C(27)—C(28) | 179.0(4) | C(29)—C(29A)—C(35B)—N(26) | −0.5(8) |
| N(26)—C(27)—C(28)—C(29) | −1.0(9) | C(30)—C(29A)—C(35B)—N(26) | −178.9(5) |
| C(27)—C(28)—C(29)—C(29A) | 2.6(9) | C(29)—C(29A)—C(35B)—C(35A) | 176.0(5) |
| C(28)—C(29)—C(29A)—C(35B) | −1.8(8) | C(30)—C(29A)—C(35B)—C(35A) | −2.4(8) |
| C(28)—C(29)—C(29A)—C(30) | 176.4(6) | C(31A)—C(35A)—C(35B)—N(26) | 178.0(5) |
| C(35B)—C(29A)—C(30)—C(31) | 1.2(9) | C(35)—C(35A)—C(35B)—N(26) | 1.3(7) |
| C(29)—C(29A)—C(30)—C(31) | −177.1(6) | C(31A)—C(35A)—C(35B)—C(29A) | 1.3(8) |
| C(29A)—C(30)—C(31)—C(31A) | 1.2(9) | C(35)—C(35A)—C(35B)—C(29A) | −175.3(5) |
| C(30)—C(31)—C(31A)—C(35A) | −2.3(9) | | |
| C(30)—C(31)—C(31A)—C(32) | 173.5(6) | | |
| C(35A)—C(31A)—C(32)—C(33) | 1.6(8) | | |
| C(31)—C(31A)—C(32)—C(33) | −174.3(6) | | |
| C(31A)—C(32)—C(33)—C(34) | −2.1(9) | | |
| C(32)—C(33)—C(34)—C(35) | −0.3(9) | | |
| C(33)—C(34)—C(35)—C(35A) | 3.2(8) | | |
| C(33)—C(34)—C(35)—Pd | −177.1(4) | | |
| F(2)—Pd—C(35)—C(34) | −81.6(5) | | |
| N(26)—Pd—C(35)—C(34) | −172.3(5) | | |
| N(13)—Pd—C(35)—C(34) | 97.3(5) | | |
| N(1)—Pd—C(35)—C(34) | 10.3(5) | | |
| F(2)—Pd—C(35)—C(35A) | 98.2(4) | | |
| N(26)—Pd—C(35)—C(35A) | 7.4(4) | | |

### Example 11

### Synthesis of 3-deoxy-3-fluoromorphine

### 3-trifluoromethanesulfonyl morphine

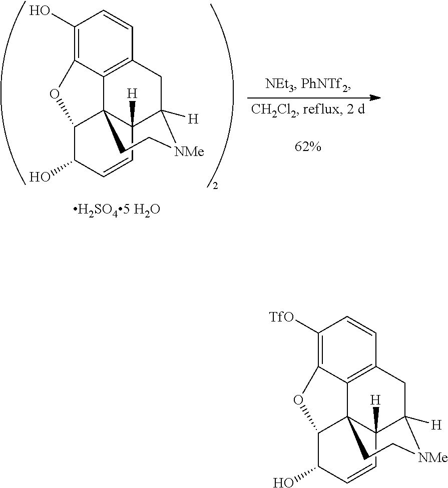

To morphine sulfate pentahydrate (1.03 g, 1.36 mmol, 1.00 equiv) in CH₂Cl₂ (23 mL) in a pressure tube was added N-phenyltriflamide (1.16 g, 3.26 mmol, 2.40 equiv) and triethylamine (560 μL, 4.07 mmol, 3.0 equiv). The reaction mixture was heated to 60° C. and stirred for 2 days. The reaction was allowed to cool to 23° C. and diluted with CH₂Cl₂ (15 mL). The organic phase was washed with NaHCO₃ (30 mL) and the aqueous layer was extracted with CH₂Cl₂ (3×10 mL). The combined organic phases were washed with brine (20 mL) and dried (Na₂SO₄). The filtrate as concentrated in vacuo and the resulting residue was purified by chromatography on silica gel eluting with CH₂Cl₂/MeOH 9:1 (v/v) to afford 703 mg of the title compound as a white solid (62% yield).

### 3-trifluoromethanesulfonyl morphine carbamate

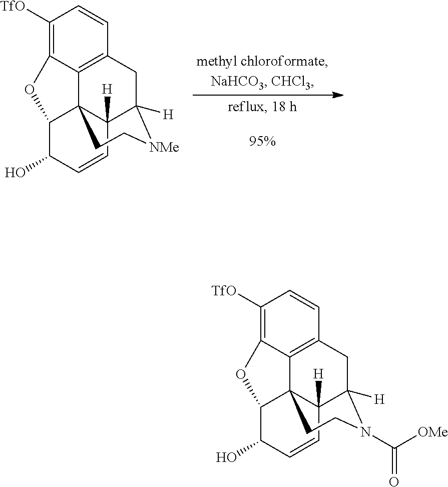 [SMILES: CN1CC[C@]23c4c5ccc(OS(=O)(=O)C(F)(F)F)c4O[C@H]2[C@@H](O)C=C[C@H]3C1C5 ; COC(=O)N1CC[C@]23c4c5ccc(OS(=O)(=O)C(F)(F)F)c4O[C@H]2[C@@H](O)C=C[C@H]3C1C5]

To 3-trifluoromethanesulfonyl morphine (754 mg, 1.80 mmol, 1.00 equiv) in CHCl₃ (2.4 mL) was added NaHCO₃ (2.30 g, 27.0 mmol, 15.0 equiv) and methyl chloroformate (2.40 mL, 30.6 mmol, 17.0 equiv). The reaction mixture was heated to 62° C. and stirred for 18 h. The reaction was allowed to cool to 23° C. and quenched with H₂O (3 mL). The aqueous layer was extracted with CH₂Cl₂ (3×5 mL). The combined organic phases were washed with brine (10 mL) and dried (Na₂SO₄). The filtrate was concentrated in vacuo and the resulting residue was purified by chromatography on silica gel eluting with hexane/EtOAc 3:2 (v/v) to afford 753 mg of the title compound as a pale yellow solid (95% yield). NMR Spectroscopy: ¹H NMR (500 MHz, CDCl₃, 23° C., δ): 6.94 (d, J=8.5 Hz, 1H), 6.66 (d, J=8.5 Hz, 1H), 5.78 (d, J=9.0 Hz, 1H), 5.31-5.30 (m, 1H), 5.03 (d, J=6.5 Hz, 1H), 4.22-4.19 (m, 1H), 3.76 (s (rotamers), 1H), 3.01-2.88 (m, 3H), 2.81 (d, J=19.5 Hz, 1H), 2.58 (s, 1H), 2.05-1.92 (m, 2H).

### Morphine carbamate 3-pinacolboronic ester

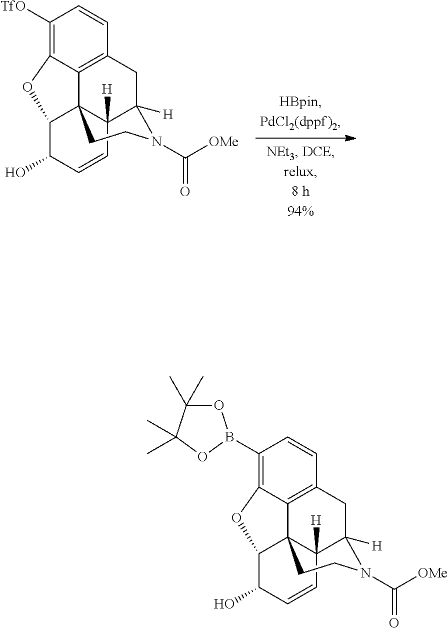 [SMILES: COC(=O)N1CC[C@]23c4c5ccc(OS(=O)(=O)C(F)(F)F)c4O[C@H]2[C@@H](O)C=C[C@H]3C1C5 ; COC(=O)N1CC[C@]23c4c5ccc(B6OC(C)(C)C(C)(C)O6)c4O[C@H]2[C@@H](O)C=C[C@H]3C1C5]

To 3-trifluoromethanesulfonyl morphine carbamate (204 mg, 0.440 mmol, 1.00 equiv) in DCE (4.7 mL) in a schlenck was added triethylamine (100 μL, 0.700 mmol, 1.50 equiv) and pinacol borane (200 μL, 1.40 mmol, 3.00 equiv). The reaction mixture was degassed and PdCl₂ dppf was added under N₂. The reaction mixture was sealed, heated to 83° C., and stirred for 8.0 h. The reaction was allowed to cool to 23° C. and quenched with H₂O (5 mL). The aqueous layer was extracted with CHCl₃ (3×5 mL). The combined organic phases were washed with brine (20 mL) and dried (Na₂SO₄). The filtrate was concentrated in vacuo and the resulting residue was purified by chromatography on silica gel eluting with hexane/EtOAc 1:1 (v/v) to afford 182 mg of the title compound as a pale yellow solid (94% yield). NMR Spectroscopy: ¹H NMR (500 MHz, CDCl₃, 23° C., δ): 7.45 (d, J=7.5 Hz, 1H), 6.66 (d, J=8.0 Hz, 1H), 5.78 (d, J=9.0 Hz, 1H), 5.31-5.30 (m, 1H), 5.03 (d, J=6.5 Hz, 1H), 4.22-4.19 (m, 1H), 3.76 (s (rotamers), 1H), 3.01-2.88 (m, 3H), 2.81 (d, J=19.5 Hz, 1H), 2.58 (s, 1H), 2.05-1.92 (m, 2H).

### Morphine carbamate 6-tertbutyldimethylsilyoxy 3-pinacolboronic ester

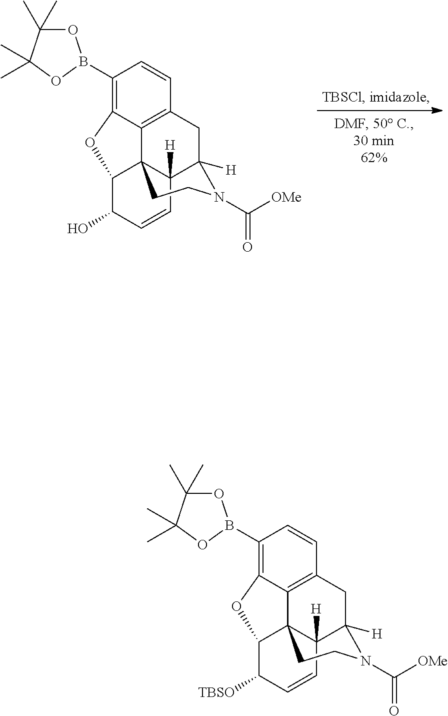 [SMILES: COC(=O)N1CC[C@]23c4c5ccc(B6OC(C)(C)C(C)(C)O6)c4O[C@H]2[C@@H](O)C=C[C@H]3C1C5 ; COC(=O)N1CC[C@]23c4c5ccc(B6OC(C)(C)C(C)(C)O6)c4O[C@H]2[C@@H](O[Si](C)(C)C(C)(C)C)C=C[C@H]3C1C5]

To morphine carbamate 3-pinacolboronic ester (23.5 mg, 0.0530 mmol, 1.00 equiv) in DMF (250 μL) was added TBSCl (39.9 mg, 0.265 mmol, 5.00 equiv) and imidazole (36.1 mg, 0.530 mmol, 10.0 equiv). The reaction mixture was heated to 50° C., and stirred for 30 min. The reaction was allowed to cool to 23° C. and washed with H₂O (3 mL). The aqueous layer was extracted with Et₂O (3×5 mL). The combined organic phases were dried (Na₂SO₄). The filtrate as concentrated in vacuo and the resulting residue affords 18.3 mg of the title compound as a pale yellow solid (94% yield).

### Aryl Pd complex

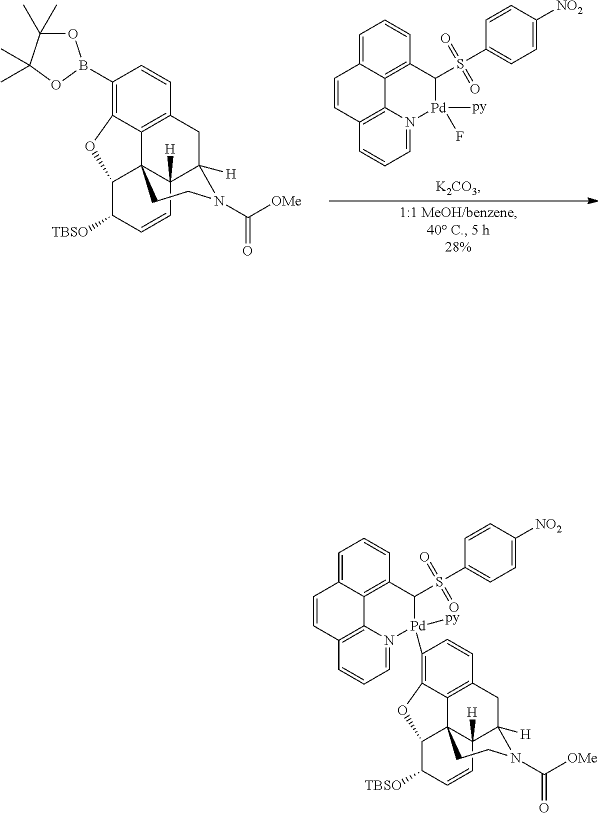

To morphine carbamate 6-tertbutyldimethylsilyoxy 3-pinacolboronic ester (8.2 mg, 0.015 mmol, 1.0 equiv) in MeOH/benzene 1:1 (v/v) (0.25 mL) was added K₂CO₃ (6.2 mg, 0.045 mmol, 3.0 equiv) and Pd(II) fluoride (8.7 mg, 0.015 mmol, 1.0 equiv). The reaction mixture was stirred for 1.5 h at 23° C. and heated to 40° C. and stirred for an additional 5 h. The reaction was allowed to cool and concentrated in vacuo. The resulting solid was triturated with CHCl₃ and filtered through a pad of celite. The filtrate was concentrated in vacuo and the resulting residue was purified by chromatography on silica gel eluting with hexane/EtOAc 3:2 (v/v) to afford 4.2 mg of the title compound as a pale yellow solid (28% yield).

### 3-fluoro-6-tertbutyldimethylsilyoxymorphine carbamate

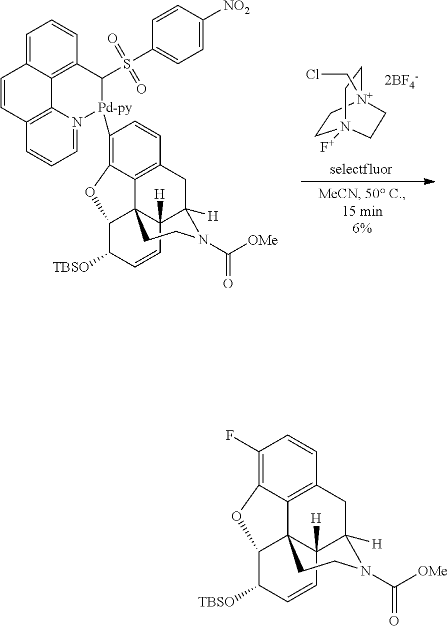

To SELECTFLUOR® (3.7 mg, 0.010 mmol, 1.2 equiv) in CD₃CN (0.25 mL) was added a solution of aryl Pd complex (8.6 mg, 0.0087 mmol, 1.0 equiv) in CD₃CN (0.50 mL) dropwise over 10 min. The reaction mixture was for an additional 5 mins. The reaction was allowed to cool to 23° C. and was concentrated in vacuo and the resulting residue was purified by chromatography on silica gel eluting with hexane/EtOAc 3:1 (v/v) to afford 0.2 mg of the title compound as a white solid (6% yield).

### 3-fluoromorphine carbamate

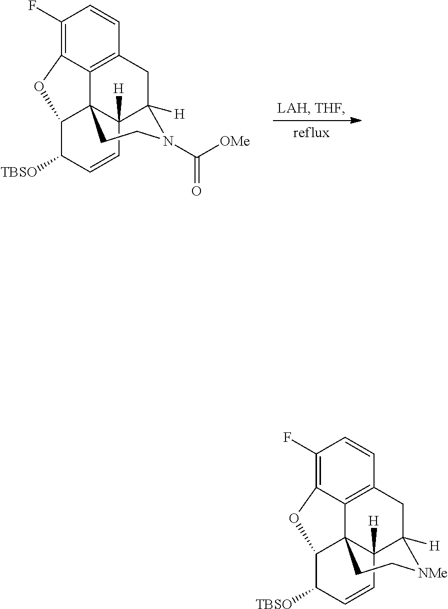 [SMILES: COC(=O)N1CC[C@]23c4c5ccc(F)c4O[C@H]2[C@@H](O[Si](C)(C)C(C)(C)C)C=C[C@H]3C1C5 ; CN1CC[C@]23c4c5ccc(F)c4O[C@H]2[C@@H](O[Si](C)(C)C(C)(C)C)C=C[C@H]3C1C5]

To 3-fluoro-6-tertbutyldimethylsilyoxymorphine carbamate (69.6 mg, 0.156 mmol, 1.00 equiv) in THF (3.0 mL) is added TBAF (240 μL, 0.234 mmol, 1.50 equiv). The reaction mixture is stirred for 30 min at 23° C. and is concentrated in vacuo. The residue is diluted with CH₂Cl₂ (2 mL) and washed with NH₄Cl (1 mL). The aqueous layer is extracted with CH₂Cl₂ (3×2 mL) and dried (Na₂SO₄). The resulting filtrate is concentrated in vacuo and the residue is purified by chromatography on silica gel eluting with hexane/EtOAc 2:3 (v/v) to afford 37.2 mg of the title compound as a white solid (72% yield).

### 3-fluoromorphine

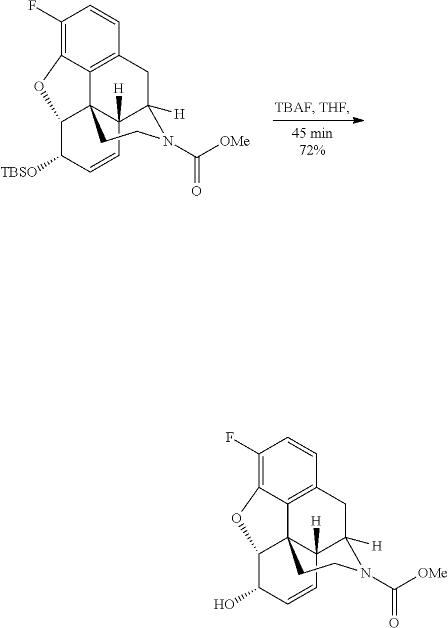 [SMILES: COC(=O)N1CC[C@]23c4c5ccc(F)c4O[C@H]2[C@@H](O[Si](C)(C)C(C)(C)C)C=C[C@H]3C1C5 ; COC(=O)N1CC[C@]23c4c5ccc(F)c4O[C@H]2[C@@H](O)C=C[C@H]3C1C5]

To 3-fluoromorphine carbamate (34.5 mg, 0.104 mmol, 1.00 equiv) in THF (0.5 mL) was added lithium aluminum hydride (1.0 M solution in THF) (520 μL, 0.521 mmol, 5.00 equiv). The reaction mixture was stirred for 30 min at 23° C. The reaction was quenched with 1.0 M solution of Rochelle's salt. The resulting solution was diluted with Et₂O (2 mL) and stirred vigorously overnight. The aqueous layer was extracted with Et₂O (10×1 mL), washed with brine (5 mL), dried (Na₂SO₄), and the filtrate as concentrated in vacuo. The resulting residue was purified by chromatography on silica gel eluting with CH₂Cl₂/MeOH 9:1 (v/v) to afford 23.4 mg of the title compound as a white solid (78% yield). R_{f}=0.05 (CH₂Cl₂/MeOH 9:1 (v/v)). NMR Spectroscopy: ¹H NMR (500 MHz, CDCl₃, 23° C., δ): 6.81 (dd, J=8.5 Hz, 5.35 Hz, 1H), 6.55 (dd, J=3.5 Hz, 3.8 Hz, 1H), 5.71 (dd, J=1.5 Hz, 5.0 Hz, 1H), 5.30-5.28 (m, 1H), 4.95 (d, J=6.0 Hz, 1H), 4.21-4.20 (m, 1H), 3.37 (dd, J=3.0 Hz, 2.8 Hz, 1H), 3.07 (d, J=18.5 Hz, 1H), 2.68 (s, 1H), 2.62 (dd, J=4.5 Hz, 6.0 Hz, 1H), 2.43 (s, 3H), 2.40 (dt, J=3.5 Hz, 12.3 Hz, 6.1 Hz, 1H), 2.31 (dd, J=5.5 Hz, 9.3 Hz, 1H), 2.11 (dt, J=5.0 Hz, 12.4 Hz, 6.1 Hz, 1H), 1.89-1.87 (m, 1H). ¹³C NMR (125 MHz, CDCl₃, 23° C., δ): 146.36 (d, J=244 Hz), 144.28 (d, J=10.1 Hz), 133.26, 133.06 (d, J=2.8 Hz), 130.31, 128.35, 119.81 (d, J=4.6 Hz), 115.97 (d, J=17.4 Hz), 92.37, 66.42, 58.69, 46.24, 43.21 (d, J=84 Hz), 40.65, 35.60, 20.59. ¹⁹F NMR (280 MHz, CDCl₃, 23° C., δ): −139.8. Mass Spectrometry: HRMS-FIA (m/z): Calcd for [M+H]⁺, 288.13943. Found, 288.13962.

### Example 12

### Synthesis of 3-deoxy-3-fluoromorphine

### 3-trifluoromethanesulfonyl morphine

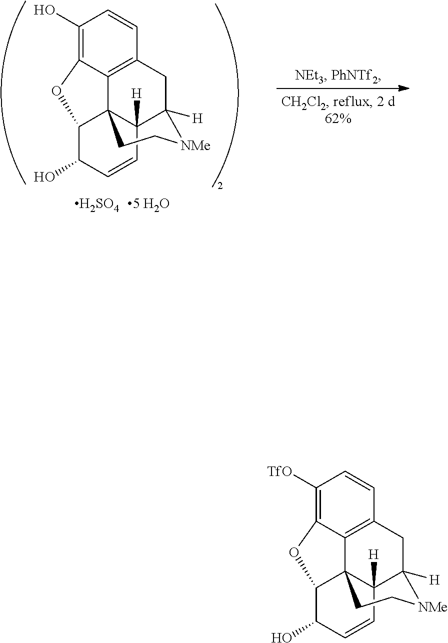

To morphine sulfate pentahydrate (1.03 g, 1.36 mmol, 1.00 equiv) in CH₂Cl₂ (23 mL) in a pressure tube was added N-phenyltriflamide (1.16 g, 3.26 mmol, 2.40 equiv) and triethylamine (560 μL, 4.07 mmol, 3.0 equiv). The reaction mixture was heated to 60° C. and stirred for 2 days. The reaction as allowed to cool to 23° C. and diluted with CH₂Cl₂ (15 mL). The organic phase as washed with NaHCO₃ (30 mL) and the aqueous layer is extracted with CH₂Cl₂ (3×10 mL). The combined organic phases were washed with brine (20 mL) and dried (Na₂SO₄). The filtrate was concentrated in vacuo and the resulting residue the residue was purified by chromatography on silica gel eluting with CH₂Cl₂/MeOH 9:1 (v/v) to afford 703 mg of the title compound as a white solid (62% yield).

### 3-trifluoromethanesulfonyl morphine carbamate

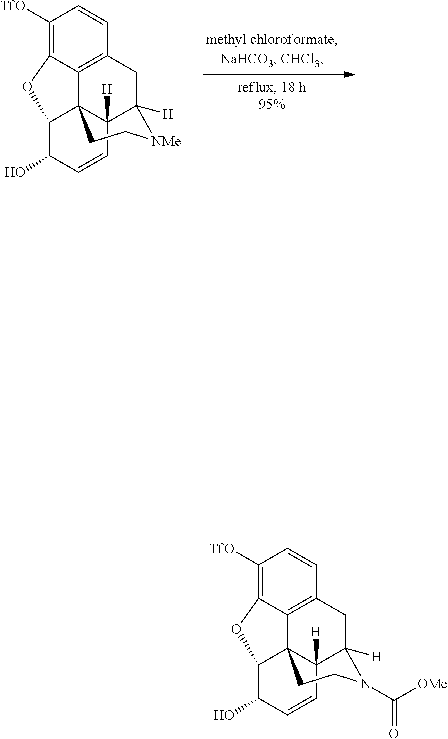 [SMILES: CN1CC[C@]23c4c5ccc(OS(=O)(=O)C(F)(F)F)c4O[C@H]2[C@@H](O)C=C[C@H]3C1C5 ; COC(=O)N1CC[C@]23c4c5ccc(OS(=O)(=O)C(F)(F)F)c4O[C@H]2[C@@H](O)C=C[C@H]3C1C5]

To 3-trifluoromethanesulfonyl morphine (754 mg, 1.80 mmol, 1.00 equiv) in CHCl₃ (2.4 mL) was added NaHCO₃ (2.30 g, 27.0 mmol, 15.0 equiv) and methyl chloroformate (2.40 mL, 30.6 mmol, 17.0 equiv). The reaction mixture as heated to 62° C. and stirred for 18 h. The reaction was allowed to cool to 23° C. and quenched with H₂O (3 mL). The aqueous layer as extracted with CH₂Cl₂ (3×5 mL). The combined organic phases were washed with brine (10 mL) and dried (Na₂SO₄). The filtrate was concentrated in vacuo and the resulting residue was purified by chromatography on silica gel eluting with hexane/EtOAc 3:2 (v/v) to afford 753 mg of the title compound as a pale yellow solid (95% yield). NMR Spectroscopy: ¹H NMR (500 MHz, CDCl₃, 23° C., δ): 6.94 (d, J=8.5 Hz, 1H), 6.66 (d, J=8.5 Hz, 1H), 5.78 (d, J=9.0 Hz, 1H), 5.31-5.30 (m, 1H), 5.03 (d, J=6.5 Hz, 1H), 4.22-4.19 (m, 1H), 3.76 (s (rotamers), 1H), 3.01-2.88 (m, 3H), 2.81 (d, J=19.5 Hz, 1H), 2.58 (s, 1H), 2.05-1.92 (m, 2H).

### Morphine carbamate 3-pinacolboronic ester

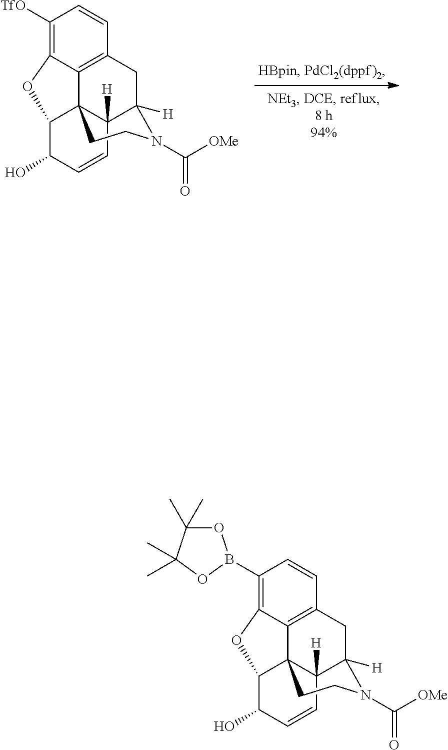 [SMILES: COC(=O)N1CC[C@]23c4c5ccc(OS(=O)(=O)C(F)(F)F)c4O[C@H]2[C@@H](O)C=C[C@H]3C1C5 ; COC(=O)N1CC[C@]23c4c5ccc(B6OC(C)(C)C(C)(C)O6)c4O[C@H]2[C@@H](O)C=C[C@H]3C1C5]

To 3-trifluoromethanesulfonyl morphine carbamate (204 mg, 0.440 mmol, 1.00 equiv) in DCE (4.7 mL) in a schlenck was added triethylamine (100 μL, 0.700 mmol, 1.50 equiv) and pinacol borane (200 μL, 1.40 mmol, 3.00 equiv). The reaction mixture was degassed and PdCl₂dppf was added under N₂. The reaction mixture was sealed, heated to 83° C., and stirred for 8.0 h. The reaction was allowed to cool to 23° C. and quenched with H₂O (5 mL). The aqueous layer was extracted with CHCl₃ (3×5 mL). The combined organic phases were washed with brine (20 mL) and dried (Na₂SO₄). The filtrate was concentrated in vacuo and the resulting residue was purified by chromatography on silica gel eluting with hexane/EtOAc 1:1 (v/v) to afford 182 mg of the title compound as a pale yellow solid (94% yield). NMR Spectroscopy: ¹H NMR (500 MHz, CDCl₃, 23° C., δ): 7.45 (d, J=7.5 Hz, 1H), 6.66 (d, J=8.0 Hz, 1H), 5.78 (d, J=9.0 Hz, 1H), 5.31-5.30 (m, 1H), 5.03 (d, J=6.5 Hz, 1H), 4.22-4.19 (m, 1H), 3.76 (s (rotamers), 1H), 3.01-2.88 (m, 3H), 2.81 (d, J=19.5 Hz, 1H), 2.58 (s, 1H), 2.05-1.92 (m, 2H).

### 3-bromo-morphine carbamate

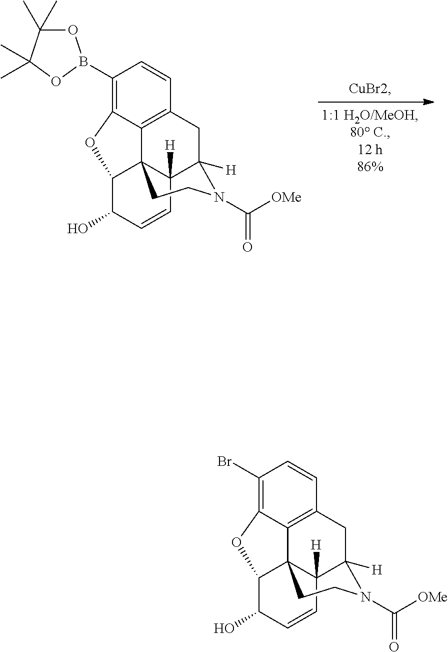 [SMILES: COC(=O)N1CC[C@]23c4c5ccc(B6OC(C)(C)C(C)(C)O6)c4O[C@H]2[C@@H](O)C=C[C@H]3C1C5 ; COC(=O)N1CC[C@]23c4c5ccc(Br)c4O[C@H]2[C@@H](O)C=C[C@H]3C1C5]

To morphine carbamate 3-pinacolboronic ester (54.5 mg, 0.124 mmol, 1.00 equiv) in MeOH/H₂O 1:1 (v/v) (1.0 mL) was added CuBr₂ (83.1 mg, 0.372 mmol, 3.00 equiv). The reaction mixture was heated to 80° C., and stirred for 12 h. The reaction was allowed to cool to 23° C. and washed with Na₂S (1.0 mL). The aqueous layer was extracted with EtOAc (10×2 mL). The combined organic phases were filtered through a pad of celite and dried (Na₂SO₄). The filtrate was concentrated in vacuo and the resulting residue afforded 41.7 mg of the title compound as a white solid (86% yield).

### 3-bromo-6-tertbutyldimethylsilyoxymorphine carbamate

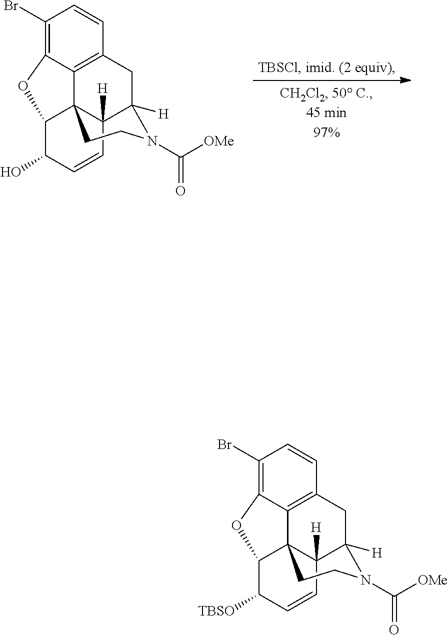 [SMILES: COC(=O)N1CC[C@]23c4c5ccc(Br)c4O[C@H]2[C@@H](O)C=C[C@H]3C1C5 ; COC(=O)N1CC[C@]23c4c5ccc(Br)c4O[C@H]2[C@@H](O[Si](C)(C)C(C)(C)C)C=C[C@H]3C1C5]

To 3-bromo-morphine carbamate (41.7 mg, 0.106 mmol, 1.00 equiv) in CH₂Cl₂ (500 μL) was added TBSCl (40.1 mg, 0.266 mmol, 2.50 equiv) and imidazole (36.2 mg, 0.532 mmol, 5.00 equiv). The reaction mixture was heated to 50° C., and stirred for 45 min. The reaction was allowed to cool to 23° C. and washed with H₂O (1 mL). The aqueous layer was extracted with CH₂Cl₂ (3×1 mL). The combined organic phases were dried (Na₂SO₄). The filtrate was concentrated in vacuo and the resulting residue was purified by chromatography on silica gel eluting with hexane/EtOAc 3:2 (v/v) to afford 51.9 mg of the title compound as a pale yellow solid (97% yield).

### 3-fluoro-6-tertbutyldimethylsilyoxymorphine carbamate

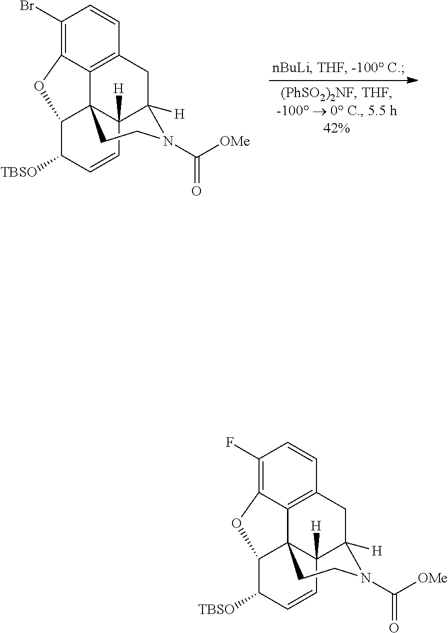 [SMILES: COC(=O)N1CC[C@]23c4c5ccc(Br)c4O[C@H]2[C@@H](O[Si](C)(C)C(C)(C)C)C=C[C@H]3C1C5 ; COC(=O)N1CC[C@]23c4c5ccc(F)c4O[C@H]2[C@@H](O[Si](C)(C)C(C)(C)C)C=C[C@H]3C1C5]

To 3-bromo-6-tertbutyldimethylsilyoxymorphine carbamate (186.5 mg, 0.368 mmol, 1.0 equiv) in anhydrous THF (3.00 mL) at −100° C. was added nBuLi dropwise (2.1 M in hexanes) (176 μL, 1.0 equiv), followed by N-fluorobenzenesulfonimide in anhydrous THF dropwise (15 mL) (146.0 mg, 0.463 mmol, 1.25 equiv). The reaction mixture was stirred for 5.5 h, allowing to reaction to warm to 0° C. The reaction was quenched with NH₄Cl (5 mL) and concentrated in vacuo. The aqueous layer was extracted with CH₂Cl₂ (3×10 mL). The combined organic phases were dried (Na₂SO₄). The filtrate was concentrated in vacuo and the resulting residue was purified by chromatography on silica gel eluting with hexane/EtOAc 3:1 (v/v) to afford 69.6 mg of the title compound as a white solid (42% yield).

### 3-fluoromorphine carbamate

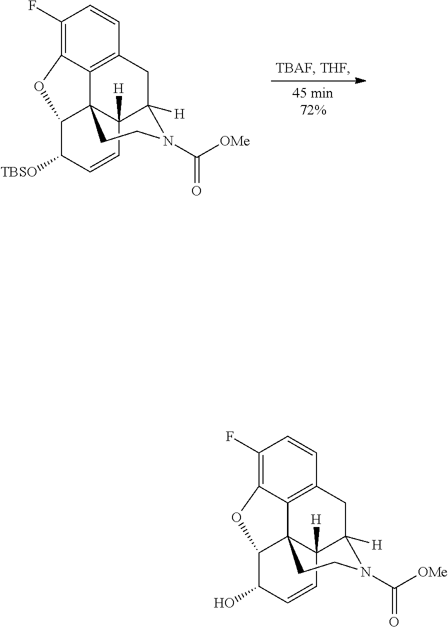 [SMILES: COC(=O)N1CC[C@]23c4c5ccc(F)c4O[C@H]2[C@@H](O[Si](C)(C)C(C)(C)C)C=C[C@H]3C1C5 ; COC(=O)N1CC[C@]23c4c5ccc(F)c4O[C@H]2[C@@H](O)C=C[C@H]3C1C5]

To 3-fluoro-6-tertbutyldimethylsilyoxymorphine carbamate (69.6 mg, 0.156 mmol, 1.00 equiv) in THF (3.0 mL) was added TBAF (240 μL, 0.234 mmol, 1.50 equiv). The reaction mixture was stirred for 30 min at 23° C. and is concentrated in vacuo. The residue was diluted with CH₂Cl₂ (2 mL) and washed with NH₄Cl (1 mL). The aqueous layer was extracted with CH₂Cl₂ (3×2 mL) and dried (Na₂SO₄). The resulting filtrate was concentrated in vacuo and the residue was purified by chromatography on silica gel eluting with hexane/EtOAc 2:3 (v/v) to afford 37.2 mg of the title compound as a white solid (72% yield).

### 3-fluoromorphine

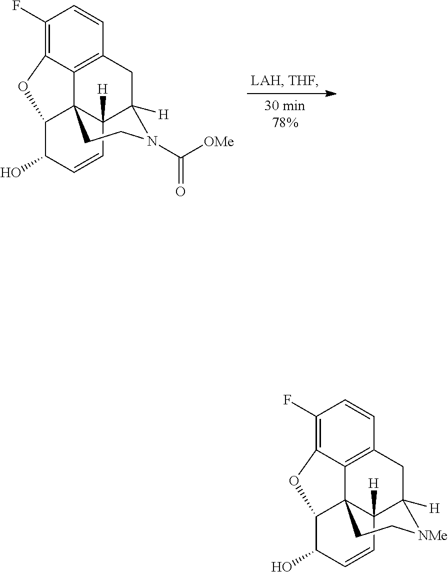 [SMILES: COC(=O)N1CC[C@]23c4c5ccc(F)c4O[C@H]2[C@@H](O)C=C[C@H]3C1C5 ; CN1CC[C@]23c4c5ccc(F)c4O[C@H]2[C@@H](O)C=C[C@H]3C1C5]

To 3-fluoromorphine carbamate (34.5 mg, 0.104 mmol, 1.00 equiv) in THF (0.5 mL) was added lithium aluminum hydride (1.0 M solution in THF) (520 μL, 0.521 mmol, 5.00 equiv). The reaction mixture was stirred for 30 min at 23° C. The reaction was quenched with 1.0 M solution of Rochelle's salt. The resulting solution was diluted with Et₂O (2 mL) and stirred vigorously overnight. The aqueous layer was extracted with Et₂O (10×1 mL), washed with brine (5 mL), dried (Na₂SO₄), and the filtrate was concentrated in vacuo. The resulting residue was purified by chromatography on silica gel eluting with CH₂Cl₂/MeOH 9:1 (v/v) to afford 23.4 mg of the title compound as a white solid (78% yield). R_{f}=0.05 (CH₂Cl₂/MeOH 9:1 (v/v)). NMR Spectroscopy: ¹H NMR (500 MHz, CDCl₃, 23° C., δ): 6.81 (dd, J=8.5 Hz, 5.35 Hz, 1H), 6.55 (dd, J=3.5 Hz, 3.8 Hz, 1H), 5.71 (dd, J=1.5 Hz, 5.0 Hz, 1H), 5.30-5.28 (m, 1H), 4.95 (d, J=6.0 Hz, 1H), 4.21-4.20 (m, 1H), 3.37 (dd, J=3.0 Hz, 2.8 Hz, 1H), 3.07 (d, J=18.5 Hz, 1H), 2.68 (s, 1H), 2.62 (dd, J=4.5 Hz, 6.0 Hz, 1H), 2.43 (s, 3H), 2.40 (dt, J=3.5 Hz, 12.3 Hz, 6.1 Hz, 1H), 2.31 (dd, J=5.5 Hz, 9.3 Hz, 1H), 2.11 (dt, J=5.0 Hz, 12.4 Hz, 6.1 Hz, 1H), 1.89-1.87 (m, 1H). ¹³C NMR (125 MHz, CDCl₃, 23° C., ): 146.36 (d, J=244 Hz), 144.28 (d, J=10.1 Hz), 133.26, 133.06 (d, J=2.75 Hz), 130.31, 128.35, 119.81 (d, J=4.58 Hz), 115.97 (d, J=17.4 Hz), 92.37, 66.42, 58.69, 46.24, 43.21 (d, J=84 Hz), 40.65, 35.60, 20.59. ¹⁹F NMR (280 MHz, CDCl₃, 23° C., δ): −139.8. Mass Spectrometry: HRMS-FIA (m/z): Calcd for [M+H]⁺, 288.13943. Found, 288.13962.

### Example 13

### Synthesis of Palladium(II) fluoride complex 13

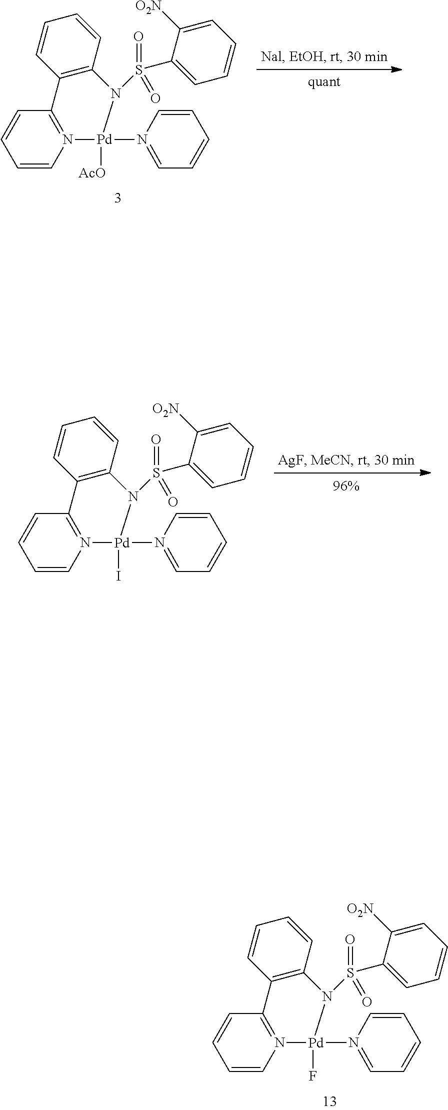

To the acetato palladium complex (500 mg, 0.840 mmol, 1.00 equiv) in EtOH (10 mL) at 23° C. was added NaI (1.26 g, 8.40 mmol, 10.0 equiv). The reaction mixture was stirred at 23° C. for 30 min. The reaction mixture was filtered and washed with water (3×5 mL), EtOH (3×5 mL) and Et₂O (10 mL) to afford 556 mg iodo palladium compound as orange solid (quant).

To the iodo palladium complex (300 mg, 0.45 mmol, 1.00 equiv) in MeCN (5 mL) at 23° C. was added AgF (283 mg, 2.25 mmol, 5.00 equiv). The reaction mixture was stirred at 23° C. for 30 min, the solvent was removed in vacuo. The solid was dissolved in CH₂Cl₂ and filtered through a pad of celite. The filtrate was concentrated in vacuo to afford 241 mg of the palladium fluoride compound as a yellow solid (96% yield).

¹H NMR (400 MHz, CDCl₃): δ 8.83-8.79 (m, 3H), 7.83 (t, J=7.5 Hz, 1H), 7.65 (dd, J=8.0, 1.2 Hz, 1H), 7.57 (dt, J=7.6, 1.2 Hz, 1H), 7.52 (dt, J=7.6, 1.2 Hz, 1H), 7.47-7.35 (m, 5H), 7.27 (d, J=7.6 Hz, 1H), 7.19 (dt, J=7.6, 1.2 Hz, 1H), 7.13 (dt, J=7.6, 1.2 Hz, 2H), 7.05 (dd, J=7.6, 1.2 Hz, 1H). ¹⁹F NMR (375 Hz, CDCl₃): −324.0 (s).

### Example 14

### Crystal Structure of Palladium(II) fluoride complex 13

The compound was crystallized from a dichloromethane/diethyl ether solution as colorless prisms. One of the prisms was cut to 0.120 mm×0.180 mm×0.230 mm in size, mounted on a nylon loop with Paratone-N oil, and transferred to a Bruker SMART APEX II diffractometer equipped with an Oxford Cryosystems 700 Series Cryostream Cooler and Mo Kα radiation (λ=0.71073 Å). A total of 3064 frames were collected at 193 (2) K to θₘₐₓ=27.500 with an oscillation range of 0.5°/frame, and an exposure time of 20 s/frame using the APEX2 suite of software. (Bruker AXS, 2006a) Unit cell refinement on all observed reflections, and data reduction with corrections for Lp and decay were performed using SAINT. (Bruker AXS, 2006b) Scaling was done using SADABS. (Bruker AXS, 2004) The minimum and maximum transmission factors were 0.7875 and 0.8802, respectively. A total of 95236 reflections were collected, 9929 were unique (Rᵢₙₜ=0.0382), and 8417 had I>2σ(I). Systematic absences were consistent with the compound having crystallized in the monoclinic space group P2₁/c (No. 14). The observed mean |E²−1| value was 0.875 (versus the expectation values of 0.968 and 0.736 for centric and noncentric data, respectively).

The structure was solved by direct methods and refined by full-matrix least-squares on F² using SHELXTL. (Bruker AXS, 2001) The asymmetric unit was found to contain one molecule of [(2-Nitrophenylsulfonyl)(2-(pyridin-2-yl)phenyl)amido](pyridine)palladium(II) fluoride and one molecule of dichloromethane. The pyridine ligand was found to be mildly disordered; this disorder was not treated since treatment of the disorder would not significantly improve the R(F) and wR(F²) values. All of the nonhydrogen atoms were refined with anisotropic displacement coefficients. The hydrogen atoms were assigned isotropic displacement coefficients U(H)=1.2U(C) and their coordinates were allowed to ride on their respective carbons. The refinement converged to R(F)=0.0257, wR(F²)=0.0625, and S=1.048 for 8417 reflections with I>2σ(I), and R(F)=0.0347, wR(F²)=0.0674, and S=1.048 for 9929 unique reflections, 325 parameters, and 0 restraints. The maximum |Δ/σ| in the final cycle of least-squares was 0.003, and the residual peaks on the final difference-Fourier map ranged from −0.730 to 0.819 eÅ⁻³. Scattering factors were taken from the International Tables for Crystallography, Volume C. (Maslen et al., 1992, and Creagh & McAuley, 1992). R(F)=R1=Σ∥Fₒ|−|F_{c}∥/Σ|Fₒ|, wR(F²)=wR2=[Σw(Fₒ²−F_{c}²)²/Σw(Fₒ²)²]^{1/2}, and S=Goodness-of-fit on F²=[Σw(Fₒ²−F_{c}²)²/(n−p)]^{1/2}, where n is the number of reflections and p is the number of parameters refined.

### REFERENCES

Bruker AXS (2001). SHELXTL v6.12. Bruker Analytical X-ray Systems Inc., Madison, Wis., USA.; Bruker AXS (2004). SADABS. Bruker Analytical X-ray Systems Inc., Madison, Wis., USA.; Bruker AXS (2006a). APEX2 v2.1-0. Bruker Analytical X-ray Systems Inc., Madison, Wis., USA.; Bruker AXS (2006b). SAINT V7.34A. Bruker Analytical X-ray Systems Inc., Madison, Wis., USA.; Creagh, D. C. & McAuley, W. J. (1992). International Tables for Crystallography: Mathematical, Physical and Chemical Tables, Vol C, edited by A. J. C. Wilson, pp. 206-222. Dordrecht, The Netherlands: Kluwer.; Maslen, E. N., Fox, A. G. & O'Keefe, M. A. (1992). International Tables for Crystallography: Mathematical, Physical and Chemical Tables, Vol C, edited by A. J. C. Wilson, pp. 476-516. Dordrecht, The Netherlands: Kluwer.

TABLE 19
Crystal data and structure refinement for complex 13.

| Identification code | tr050 | |
|---|---|---|
| Empirical formula | C23H19Cl2FN4O4PdS | |
| Formula weight | 643.78 | |
| Temperature | 193(2) K | |
| Wavelength | 0.71073 Å | |
| Crystal system | Monoclinic | |
| Space group | P 2₁/c | |
| Unit cell dimensions | a = 15.2325(5) Å | α = 90° |
| | b = 11.9436(4) Å | β = 101.9750(10)° |
| | c = 13.9760(5) Å | γ = 90° |

| | |
|---|---|
| Volume | 2487.33(15) Å³ |
| Z | 4 |
| Density (calculated) | 1.719 mg/m³ |
| Absorption coefficient | 1.091 mm⁻¹ |
| F(000) | 1288 |
| Crystal size | 0.230 × 0.180 × 0.120 mm³ |
| Theta range for data collection | 2.19 to 33.73°. |
| Index ranges | −23 <= h <= 23, −18 <= k <= 18, |
| | −21 <= l <= 21 |
| Reflections collected | 95236 |
| Independent reflections | 9929 [R(int) = 0.0382] |
| Completeness to theta = 33.73° | 99.9% |
| Absorption correction | Semi-empirical from equivalents |
| Max. and min. transmission | 0.8802 and 0.7875 |
| Refinement method | Full-matrix least-squares on F² |
| Data/restraints/parameters | 9929/0/325 |
| Goodness-of-fit on F² | 1.048 |
| Final R indices [I > 2sigma(I)] | R1 = 0.0257, wR2 = 0.0625 |
| R indices (all data) | R1 = 0.0347, wR2 = 0.0674 |
| Largest diff. peak and hole | 0.819 and −0.730 e · Å⁻³ |

TABLE 20
Atomic coordinates (×10⁴) and equivalent isotropic displacement
parameters (Å² × 10³) for complex 13. U(eq) is defined as
one third of the trace of the orthogonalized Uᵢⱼ tensor.

| | x | y | z | U(eq) |
|---|---|---|---|---|
| Pd(1) | 2696(1) | 9893(1) | 2172(1) | 20(1) |
| F(1) | 3641(1) | 10458(1) | 1513(1) | 31(1) |
| N(1) | 3388(1) | 8472(1) | 2560(1) | 22(1) |
| C(2) | 4289(1) | 8544(1) | 2808(1) | 27(1) |
| C(3) | 4823(1) | 7616(1) | 3072(1) | 33(1) |
| C(4) | 4421(1) | 6582(1) | 3091(1) | 35(1) |
| C(5) | 3497(1) | 6510(1) | 2847(1) | 31(1) |
| C(6) | 2978(1) | 7465(1) | 2567(1) | 23(1) |
| C(7) | 1985(1) | 7380(1) | 2277(1) | 24(1) |
| C(8) | 1608(1) | 6379(1) | 1856(1) | 34(1) |
| C(9) | 690(1) | 6220(2) | 1624(1) | 41(1) |
| C(10) | 122(1) | 7058(2) | 1799(1) | 39(1) |
| C(11) | 472(1) | 8071(1) | 2194(1) | 31(1) |
| C(12) | 1398(1) | 8238(1) | 2435(1) | 23(1) |
| N(13) | 1735(1) | 9300(1) | 2805(1) | 21(1) |
| S(14) | 1739(1) | 9579(1) | 3927(1) | 23(1) |
| O(14) | 2081(1) | 10694(1) | 4124(1) | 32(1) |
| O(15) | 887(1) | 9304(1) | 4155(1) | 33(1) |
| C(17) | 2515(1) | 8622(1) | 4617(1) | 23(1) |
| C(18) | 2227(1) | 7517(1) | 4679(1) | 28(1) |
| C(19) | 2828(1) | 6687(1) | 5072(1) | 32(1) |
| C(20) | 3726(1) | 6939(1) | 5406(1) | 33(1) |
| C(21) | 4024(1) | 8033(1) | 5386(1) | 32(1) |
| C(22) | 3413(1) | 8856(1) | 4996(1) | 25(1) |
| N(23) | 3762(1) | 10011(1) | 5030(1) | 34(1) |
| O(24) | 3747(1) | 10546(1) | 5768(1) | 55(1) |
| O(25) | 4061(1) | 10351(1) | 4342(1) | 47(1) |
| N(26) | 1956(1) | 11264(1) | 1692(1) | 28(1) |
| C(27) | 1082(1) | 11173(2) | 1287(1) | 43(1) |
| C(28) | 593(1) | 12071(2) | 826(2) | 57(1) |
| C(29) | 1009(2) | 13085(2) | 790(2) | 55(1) |
| C(30) | 1895(2) | 13183(2) | 1220(2) | 51(1) |
| C(31) | 2356(1) | 12255(1) | 1662(1) | 37(1) |
| C(1S) | 3028(1) | 3293(2) | 4461(2) | 46(1) |
| Cl(1S) | 2004(1) | 3921(1) | 3904(1) | 62(1) |
| Cl(2S) | 3866(1) | 3582(1) | 3793(1) | 59(1) |

TABLE 21
Bond lengths [Å] and angles [°] for complex 13.

| | | | |
|---|---|---|---|
| Pd(1)—F(1) | 1.9806(9) | S(14)—C(17) | 1.7780(13) |
| Pd(1)—N(13) | 1.9917(11) | C(17)—C(22) | 1.3888(19) |
| Pd(1)—N(1) | 2.0112(11) | C(17)—C(18) | 1.3984(19) |
| Pd(1)—N(26) | 2.0218(12) | C(18)—C(19) | 1.383(2) |
| N(1)—C(2) | 1.3476(18) | C(18)—H(18) | 0.9500 |
| N(1)—C(6) | 1.3561(16) | C(19)—C(20) | 1.383(2) |
| C(2)—C(3) | 1.380(2) | C(19)—H(19) | 0.9500 |
| C(2)—H(2) | 0.9500 | C(20)—C(21) | 1.385(2) |
| C(3)—C(4) | 1.380(2) | C(20)—H(20) | 0.9500 |
| C(3)—H(3) | 0.9500 | C(21)—C(22) | 1.386(2) |
| C(4)—C(5) | 1.382(2) | C(21)—H(21) | 0.9500 |
| C(4)—H(4) | 0.9500 | C(22)—N(23) | 1.4756(18) |
| C(5)—C(6) | 1.3961(19) | N(23)—O(24) | 1.217(2) |
| C(5)—H(5) | 0.9500 | N(23)—O(25) | 1.217(2) |
| C(6)—C(7) | 1.4862(19) | N(26)—C(31) | 1.337(2) |
| C(7)—C(8) | 1.4008(19) | N(26)—C(27) | 1.339(2) |
| C(7)—C(12) | 1.4077(19) | C(27)—C(28) | 1.386(2) |
| C(8)—C(9) | 1.381(2) | C(27)—H(27) | 0.9500 |
| C(8)—H(8) | 0.9500 | C(28)—C(29) | 1.372(3) |
| C(9)—C(10) | 1.378(3) | C(28)—H(28) | 0.9500 |
| C(9)—H(9) | 0.9500 | C(29)—C(30) | 1.363(3) |
| C(10)—C(11) | 1.390(2) | C(29)—H(29) | 0.9500 |
| C(10)—H(10) | 0.9500 | C(30)—C(31) | 1.386(2) |
| C(11)—C(12) | 1.3937(19) | C(30)—H(30) | 0.9500 |
| C(11)—H(11) | 0.9500 | C(31)—H(31) | 0.9500 |
| C(12)—N(13) | 1.4249(16) | C(1S)—Cl(1S) | 1.760(2) |
| N(13)—S(14) | 1.6023(11) | C(1S)—Cl(2S) | 1.764(2) |
| S(14)—O(14) | 1.4356(11) | C(1S)—H(2S) | 0.9900 |
| S(14)—O(15) | 1.4377(11) | C(1S)—H(1S) | 0.9900 |
| F(1)—Pd(1)—N(13) | 178.59(4) | N(13)—Pd(1)—N(26) | 91.41(5) |
| F(1)—Pd(1)—N(1) | 91.32(4) | N(1)—Pd(1)—N(26) | 175.88(5) |
| N(13)—Pd(1)—N(1) | 88.40(4) | C(2)—N(1)—C(6) | 120.07(12) |
| F(1)—Pd(1)—N(26) | 88.78(4) | C(2)—N(1)—Pd(1) | 117.64(9) |
| C(6)—N(1)—Pd(1) | 122.28(9) | S(14)—N(13)—Pd(1) | 120.48(6) |
| N(1)—C(2)—C(3) | 122.00(14) | O(14)—S(14)—O(15) | 118.61(7) |
| N(1)—C(2)—H(2) | 119.0 | O(14)—S(14)—N(13) | 107.94(6) |
| C(3)—C(2)—H(2) | 119.0 | O(15)—S(14)—N(13) | 110.43(6) |
| C(2)—C(3)—C(4) | 119.01(14) | O(14)—S(14)—C(17) | 108.60(7) |
| C(2)—C(3)—H(3) | 120.5 | O(15)—S(14)—C(17) | 105.18(7) |
| C(4)—C(3)—H(3) | 120.5 | N(13)—S(14)—C(17) | 105.27(6) |
| C(3)—C(4)—C(5) | 118.96(14) | C(22)—C(17)—C(18) | 117.57(12) |
| C(3)—C(4)—H(4) | 120.5 | C(22)—C(17)—S(14) | 124.64(10) |
| C(5)—C(4)—H(4) | 120.5 | C(18)—C(17)—S(14) | 117.32(10) |
| C(4)—C(5)—C(6) | 120.47(14) | C(19)—C(18)—C(17) | 120.71(14) |
| C(4)—C(5)—H(5) | 119.8 | C(19)—C(18)—H(18) | 119.6 |
| C(6)—C(5)—H(5) | 119.8 | C(17)—C(18)—H(18) | 119.6 |
| N(1)—C(6)—C(5) | 119.47(13) | C(18)—C(19)—C(20) | 120.29(14) |
| N(1)—C(6)—C(7) | 120.20(11) | C(18)—C(19)—H(19) | 119.9 |
| C(5)—C(6)—C(7) | 120.34(12) | C(20)—C(19)—H(19) | 119.9 |
| C(8)—C(7)—C(12) | 117.94(13) | C(19)—C(20)—C(21) | 120.27(14) |
| C(8)—C(7)—C(6) | 118.57(13) | C(19)—C(20)—H(20) | 119.9 |
| C(12)—C(7)—C(6) | 123.45(11) | C(21)—C(20)—H(20) | 119.9 |
| C(9)—C(8)—C(7) | 121.50(15) | C(20)—C(21)—C(22) | 118.69(14) |
| C(9)—C(8)—H(8) | 119.3 | C(20)—C(21)—H(21) | 120.7 |
| C(7)—C(8)—H(8) | 119.3 | C(22)—C(21)—H(21) | 120.7 |
| C(10)—C(9)—C(8) | 120.08(15) | C(21)—C(22)—C(17) | 122.38(13) |
| C(10)—C(9)—H(9) | 120.0 | C(21)—C(22)—N(23) | 116.25(13) |
| C(8)—C(9)—H(9) | 120.0 | C(17)—C(22)—N(23) | 121.35(12) |
| C(9)—C(10)—C(11) | 119.93(15) | O(24)—N(23)—O(25) | 124.74(15) |
| C(9)—C(10)—H(10) | 120.0 | O(24)—N(23)—C(22) | 116.70(15) |
| C(11)—C(10)—H(10) | 120.0 | O(25)—N(23)—C(22) | 118.53(14) |
| C(10)—C(11)—C(12) | 120.38(15) | C(31)—N(26)—C(27) | 118.56(14) |
| C(10)—C(11)—H(11) | 119.8 | C(31)—N(26)—Pd(1) | 120.11(11) |
| C(12)—C(11)—H(11) | 119.8 | C(27)—N(26)—Pd(1) | 120.79(11) |
| C(11)—C(12)—C(7) | 120.15(12) | N(26)—C(27)—C(28) | 121.68(19) |
| C(11)—C(12)—N(13) | 119.03(12) | N(26)—C(27)—H(27) | 119.2 |
| C(7)—C(12)—N(13) | 120.78(12) | C(28)—C(27)—H(27) | 119.2 |
| C(12)—N(13)—S(14) | 117.94(9) | C(29)—C(28)—C(27) | 119.5(2) |
| C(12)—N(13)—Pd(1) | 113.48(8) | C(29)—C(28)—H(28) | 120.3 |
| C(27)—C(28)—H(28) | 120.3 | N(26)—C(31)—H(31) | 119.0 |
| C(30)—C(29)—C(28) | 118.81(17) | C(30)—C(31)—H(31) | 119.0 |
| C(30)—C(29)—H(29) | 120.6 | Cl(1S)—C(1S)—Cl(2S) | 110.76(11) |
| C(28)—C(29)—H(29) | 120.6 | Cl(1S)—C(1S)—H(2S) | 109.5 |
| C(29)—C(30)—C(31) | 119.44(19) | Cl(2S)—C(1S)—H(2S) | 109.5 |
| C(29)—C(30)—H(30) | 120.3 | Cl(1S)—C(1S)—H(1S) | 109.5 |
| C(31)—C(30)—H(30) | 120.3 | Cl(2S)—C(1S)—H(1S) | 109.5 |
| N(26)—C(31)—C(30) | 121.99(18) | H(2S)—C(1S)—H(1S) | 108.1 |

TABLE 22
Anisotropic displacement parameters (Å² × 10³) for complex 13.
The anisotropic displacement factor exponent takes the form:
−2π²[h²a*²U₁₁ + . . . + 2 h k a* b* U₁₂]

| | U₁₁ | U₂₂ | U₃₃ | U₂₃ | U₁₃ | U₁₂ |
|---|---|---|---|---|---|---|
| Pd(1) | 18(1) | 19(1) | 24(1) | 1(1) | 4(1) | −1(1) |
| F(1) | 26(1) | 29(1) | 40(1) | 4(1) | 13(1) | −5(1) |
| N(1) | 20(1) | 23(1) | 24(1) | 0(1) | 5(1) | 0(1) |
| C(2) | 20(1) | 32(1) | 30(1) | −2(1) | 6(1) | −1(1) |
| C(3) | 22(1) | 42(1) | 36(1) | −1(1) | 6(1) | 6(1) |
| C(4) | 32(1) | 35(1) | 39(1) | 2(1) | 8(1) | 13(1) |
| C(5) | 32(1) | 24(1) | 38(1) | 2(1) | 10(1) | 5(1) |
| C(6) | 23(1) | 22(1) | 24(1) | −1(1) | 6(1) | 1(1) |
| C(7) | 24(1) | 22(1) | 25(1) | −1(1) | 5(1) | −4(1) |
| C(8) | 36(1) | 26(1) | 39(1) | −7(1) | 7(1) | −7(1) |
| C(9) | 40(1) | 37(1) | 44(1) | −10(1) | 3(1) | −18(1) |
| C(10) | 27(1) | 45(1) | 41(1) | −3(1) | 0(1) | −14(1) |
| C(11) | 20(1) | 35(1) | 35(1) | 1(1) | 2(1) | −5(1) |
| C(12) | 21(1) | 24(1) | 23(1) | 1(1) | 3(1) | −3(1) |
| N(13) | 18(1) | 21(1) | 24(1) | 1(1) | 4(1) | −1(1) |
| S(14) | 20(1) | 23(1) | 26(1) | 0(1) | 5(1) | 3(1) |
| O(14) | 36(1) | 22(1) | 38(1) | −6(1) | 6(1) | 3(1) |
| O(15) | 22(1) | 44(1) | 35(1) | 4(1) | 11(1) | 5(1) |
| C(17) | 21(1) | 25(1) | 22(1) | 1(1) | 5(1) | 1(1) |
| C(18) | 28(1) | 28(1) | 27(1) | 4(1) | 7(1) | −3(1) |
| C(19) | 40(1) | 27(1) | 29(1) | 6(1) | 8(1) | 0(1) |
| C(20) | 35(1) | 34(1) | 30(1) | 8(1) | 5(1) | 10(1) |
| C(21) | 25(1) | 39(1) | 30(1) | 2(1) | 1(1) | 4(1) |
| C(22) | 24(1) | 26(1) | 25(1) | −2(1) | 3(1) | 0(1) |
| N(23) | 25(1) | 31(1) | 41(1) | −5(1) | −3(1) | −4(1) |
| O(24) | 62(1) | 47(1) | 52(1) | −24(1) | 0(1) | −9(1) |
| O(25) | 43(1) | 39(1) | 61(1) | 5(1) | 13(1) | −13(1) |
| N(26) | 28(1) | 26(1) | 31(1) | 6(1) | 11(1) | 4(1) |
| C(27) | 28(1) | 48(1) | 53(1) | 20(1) | 10(1) | 9(1) |
| C(28) | 39(1) | 73(1) | 63(1) | 30(1) | 18(1) | 27(1) |
| C(29) | 72(2) | 50(1) | 53(1) | 24(1) | 34(1) | 37(1) |
| C(30) | 80(2) | 26(1) | 54(1) | 9(1) | 31(1) | 14(1) |
| C(31) | 49(1) | 25(1) | 41(1) | 2(1) | 16(1) | 1(1) |
| C(1S) | 56(1) | 38(1) | 45(1) | 1(1) | 15(1) | −5(1) |
| Cl(1S) | 40(1) | 73(1) | 73(1) | −19(1) | 11(1) | −6(1) |
| Cl(2S) | 44(1) | 66(1) | 70(1) | 12(1) | 18(1) | 6(1) |

TABLE 23
Hydrogen coordinates (× 10⁴) and isotropic displacement
parameters (Å² × 10³) for complex 13.

| | x | y | z | U(eq) |
|---|---|---|---|---|
| H(2) | 4566 | 9257 | 2801 | 33 |
| H(3) | 5458 | 7686 | 3237 | 40 |
| H(4) | 4775 | 5930 | 3269 | 42 |
| H(5) | 3211 | 5806 | 2869 | 37 |
| H(8) | 1993 | 5797 | 1727 | 40 |
| H(9) | 451 | 5532 | 1343 | 49 |
| H(10) | −509 | 6944 | 1650 | 46 |
| H(11) | 79 | 8654 | 2301 | 37 |
| H(18) | 1612 | 7335 | 4449 | 33 |
| H(19) | 2623 | 5941 | 5112 | 38 |
| H(20) | 4139 | 6360 | 5650 | 40 |
| H(21) | 4636 | 8215 | 5635 | 38 |
| H(27) | 790 | 10475 | 1315 | 51 |
| H(28) | −25 | 11986 | 538 | 68 |
| H(29) | 686 | 13707 | 471 | 66 |
| H(30) | 2193 | 13881 | 1218 | 61 |
| H(31) | 2976 | 12327 | 1952 | 45 |
| H(2S) | 2946 | 2473 | 4497 | 55 |
| H(1S) | 3220 | 3580 | 5137 | 55 |

TABLE 24
Torsion angles [°] for complex 13.

| | | | |
|---|---|---|---|
| F(1)—Pd(1)—N(1)—C(2) | 36.39(10) | C(10)—C(11)—C(12)—C(7) | 0.1(2) |
| N(13)—Pd(1)—N(1)—C(2) | −145.00(10) | C(10)—C(11)—C(12)—N(13) | 177.69(14) |
| F(1)—Pd(1)—N(1)—C(6) | −142.81(10) | C(8)—C(7)—C(12)—C(11) | 1.3(2) |
| N(13)—Pd(1)—N(1)—C(6) | 35.81(11) | C(6)—C(7)—C(12)—C(11) | −176.26(13) |
| C(6)—N(1)—C(2)—C(3) | 0.2(2) | C(8)—C(7)—C(12)—N(13) | −176.17(13) |
| Pd(1)—N(1)—C(2)—C(3) | −179.04(11) | C(6)—C(7)—C(12)—N(13) | 6.2(2) |
| N(1)—C(2)—C(3)—C(4) | −0.6(2) | C(11)—C(12)—N(13)—S(14) | 77.79(14) |
| C(2)—C(3)—C(4)—C(5) | −0.1(2) | C(7)—C(12)—N(13)—S(14) | −104.68(13) |
| C(3)—C(4)—C(5)—C(6) | 1.2(2) | C(11)—C(12)—N(13)—Pd(1) | −133.30(11) |
| C(2)—N(1)—C(6)—C(5) | 1.0(2) | C(7)—C(12)—N(13)—Pd(1) | 44.23(14) |
| Pd(1)—N(1)—C(6)—C(5) | −179.84(10) | N(1)—Pd(1)—N(13)—C(12) | −56.02(9) |
| C(2)—N(1)—C(6)—C(7) | −178.91(12) | N(26)—Pd(1)—N(13)—C(12) | 119.86(9) |
| Pd(1)—N(1)—C(6)—C(7) | 0.26(17) | N(1)—Pd(1)—N(13)—S(14) | 92.02(7) |
| C(4)—C(5)—C(6)—N(1) | −1.7(2) | N(26)—Pd(1)—N(13)—S(14) | −92.10(7) |
| C(4)—C(5)—C(6)—C(7) | 178.20(14) | C(12)—N(13)—S(14)—O(14) | −178.41(10) |
| N(1)—C(6)—C(7)—C(8) | 150.72(14) | Pd(1)—N(13)—S(14)—O(14) | 34.92(9) |
| C(5)—C(6)—C(7)—C(8) | −29.2(2) | C(12)—N(13)—S(14)—O(15) | −47.29(11) |
| N(1)—C(6)—C(7)—C(12) | −31.7(2) | Pd(1)—N(13)—S(14)—O(15) | 166.04(7) |
| C(5)—C(6)—C(7)—C(12) | 148.40(14) | C(12)—N(13)—S(14)—C(17) | 65.75(11) |
| C(12)—C(7)—C(8)—C(9) | −1.6(2) | Pd(1)—N(13)—S(14)—C(17) | −80.92(8) |
| C(6)—C(7)—C(8)—C(9) | 176.07(15) | O(14)—S(14)—C(17)—C(22) | −20.39(14) |
| C(7)—C(8)—C(9)—C(10) | 0.4(3) | O(15)—S(14)—C(17)—C(22) | −148.32(12) |
| C(8)—C(9)—C(10)—C(11) | 1.1(3) | N(13)—S(14)—C(17)—C(22) | 95.00(13) |
| C(9)—C(10)—C(11)—C(12) | −1.4(3) | O(14)—S(14)—C(17)—C(18) | 167.70(11) |
| O(15)—S(14)—C(17)—C(18) | 39.76(13) | | |
| N(13)—S(14)—C(17)—C(18) | −76.91(12) | | |
| C(22)—C(17)—C(18)—C(19) | −2.4(2) | | |
| S(14)—C(17)—C(18)—C(19) | 170.11(11) | | |
| C(17)—C(18)—C(19)—C(20) | −0.3(2) | | |
| C(18)—C(19)—C(20)—C(21) | 2.6(2) | | |
| C(19)—C(20)—C(21)—C(22) | −2.2(2) | | |
| C(20)—C(21)—C(22)—C(17) | −0.6(2) | | |
| C(20)—C(21)—C(22)—N(23) | 177.62(14) | | |
| C(18)—C(17)—C(22)—C(21) | 2.8(2) | | |
| S(14)—C(17)—C(22)—C(21) | −169.05(12) | | |
| C(18)—C(17)—C(22)—N(23) | −175.30(13) | | |
| S(14)—C(17)—C(22)—N(23) | 12.8(2) | | |
| C(21)—C(22)—N(23)—O(24) | −87.84(18) | | |
| C(17)—C(22)—N(23)—O(24) | 90.41(18) | | |
| C(21)—C(22)—N(23)—O(25) | 90.40(18) | | |
| C(17)—C(22)—N(23)—O(25) | −91.35(19) | | |
| F(1)—Pd(1)—N(26)—C(31) | −37.49(12) | | |
| N(13)—Pd(1)—N(26)—C(31) | 143.91(12) | | |
| F(1)—Pd(1)—N(26)—C(27) | 133.99(13) | | |
| N(13)—Pd(1)—N(26)—C(27) | −44.60(13) | | |
| C(31)—N(26)—C(27)—C(28) | 1.1(3) | | |
| Pd(1)—N(26)—C(27)—C(28) | −170.47(16) | | |
| N(26)—C(27)—C(28)—C(29) | −0.6(3) | | |
| C(27)—C(28)—C(29)—C(30) | −0.8(3) | | |
| C(28)—C(29)—C(30)—C(31) | 1.5(3) | | |
| C(27)—N(26)—C(31)—C(30) | −0.4(3) | | |
| Pd(1)—N(26)—C(31)—C(30) | 171.30(14) | | |
| C(29)—C(30)—C(31)—N(26) | −1.0(3) | | |

### OTHER EMBODIMENTS

The foregoing has been a description of certain embodiments of the invention. Those of ordinary skill in the art will appreciate that various changes and modifications to this description may be made without departing from the spirit or scope of the present invention, as defined in the following claims.

What is claimed is:

1. A palladium complex of formula: 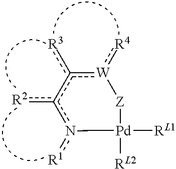
wherein:
Pd has a valency of +2;
R^{L1} is —N(R^{c})₂ wherein two R^{c} groups are joined to form an optionally substituted 6-membered heteroaryl ring;
R^{L2} is optionally substituted heteroaryl, halogen, —OR^{a}, —SR^{b}, —N(R^{c})₂, —N(R^{c})₃, or —P(R^{x})₃;
wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;
wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{b3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;
wherein each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group [symbol] C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;
wherein each instance of R^{x} is, independently, hydrogen, an optionally substituted aliphatic, optionally substituted heteroaliphatic, optionally substituted aryl, or optionally substituted heteroaryl group;
when W is —C— or —C(R^{d})— then
Z is —O—, —S—, —C(R^{d})₂—, —C(R^{d})═C(R^{d})—, —C(R^{d})═N—, or —N(R^{e})—;
or
when W is —N— or —N(R^{e})— then Z is —C(R^{d})₂—, —C(R^{d})═C(R^{d})—, or —C(R^{d})═N—,
wherein each instance of R^{d} is, independently, hydrogen, or an optionally substituted aliphatic, optionally substituted heteroaliphatic, optionally substituted aryl, or optionally substituted heteroaryl group; and
each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, or a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;
R¹ and R² are joined to form an optionally substituted 5- to 7-membered heteroaryl, aryl, heterocyclic or carbocyclic ring;
R² and R³ are joined to form an optionally substituted 5- to 7-membered heteroaryl, aryl, heterocyclic or carbocyclic ring;
R³ and R⁴ are joined to form an optionally substituted 5- to 7-membered heteroaryl, aryl, heterocyclic or carbocyclic ring;
wherein each of the curved dotted lines [symbol] independently represents joining of an optionally substituted 5- to 7-membered ring;
wherein [symbol] represents a single or double bond;
provided that when each of the curved dotted lines [symbol] represents joining of a 6-membered ring, and R^{L2} is —Cl, then R^{e1} is not 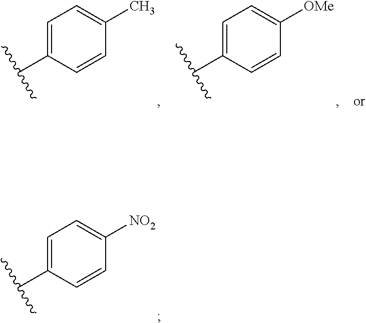
and
further provided that when each of the curved dotted lines [symbol] represents joining of a 6-membered ring, and R^{L2} is —OC(═O)CH₃, then R^{e1} is not

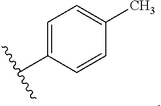 [SMILES: *c1ccc(C)cc1]

2. The palladium complex of claim 1, wherein the palladium complex further comprises a negatively charged counterion X⁻, wherein X⁻ is selected from BF₄⁻, BPh₄⁻, PF₆⁻, [BArF₄]⁻, B(C₆F₅)₄⁻, SbF₆⁻, and CF₃SO₃⁻.

3. The palladium complex of claim 1, wherein Z is —N(R^{e})—.

4. The palladium complex of claim 3, wherein R^{e} is —S(O)₂R^{e1}.

5. The palladium complex of claim 4, wherein R^{e1} is optionally substituted aryl.

6. The palladium complex of claim 5, wherein R^{e} is:

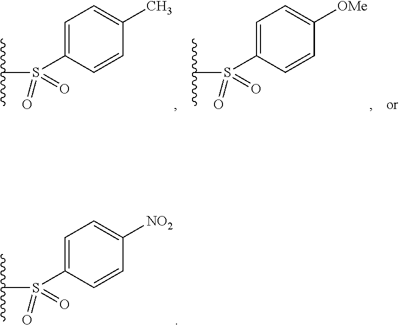 [SMILES: *S(=O)(=O)c1ccc(C)cc1 ; *S(=O)(=O)c1ccc(OC)cc1 ; *S(=O)(=O)c1ccc([N+](=O)[O-])cc1]

7. The palladium complex of claim 1, wherein R^{L1} is pyridyl.

8. The palladium complex of claim 1, wherein R^{L2} is halogen, an optionally substituted heteroaryl, or —OR^{a}.

9. The palladium complex of claim 8, wherein R^{L2} is —Cl or pyridyl.

10. The palladium complex of claim 1, wherein R^{a} is —C(═O)R^{a1} or —S(O)₂R^{a1}.

11. The palladium complex of claim 10, wherein R^{a1} is an optionally substituted aliphatic.

12. The palladium complex of claim 11, wherein R^{a} is —C(═O)CH₃ or —S(O)₂CF₃.

13. The palladium complex of claim 1, wherein the palladium complex is:

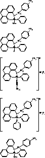

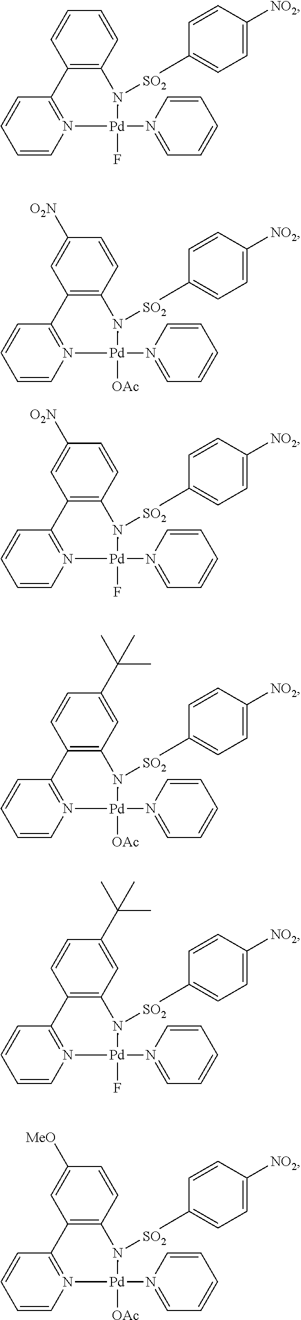

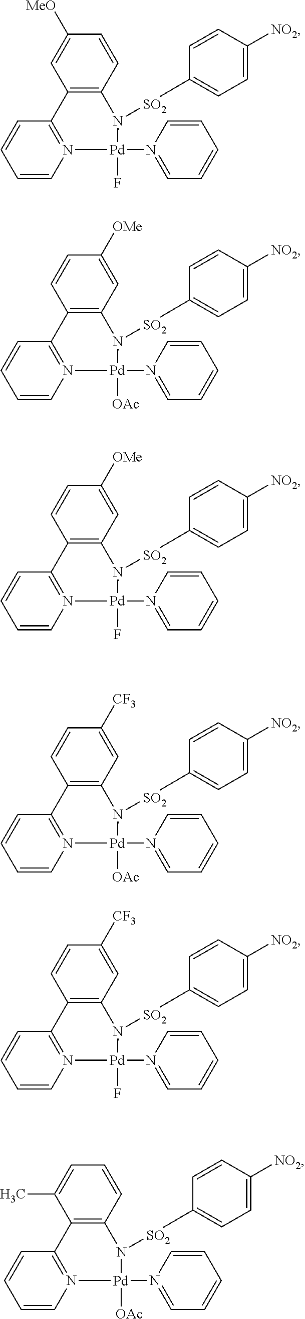

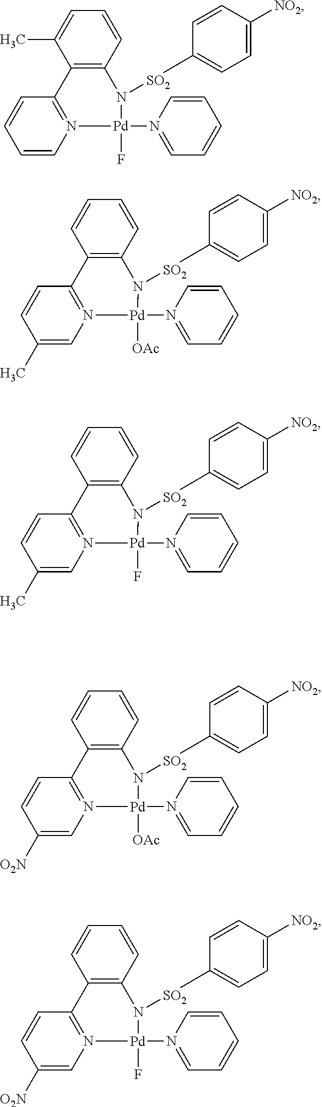

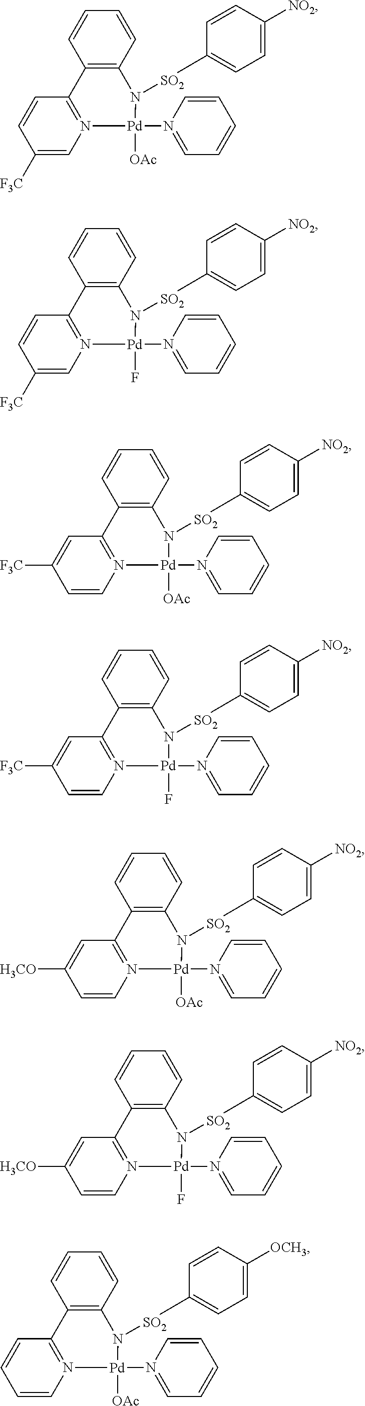

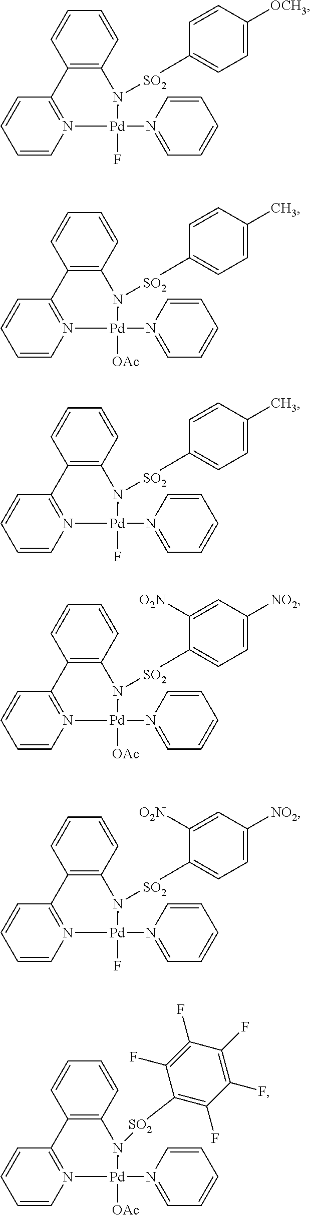

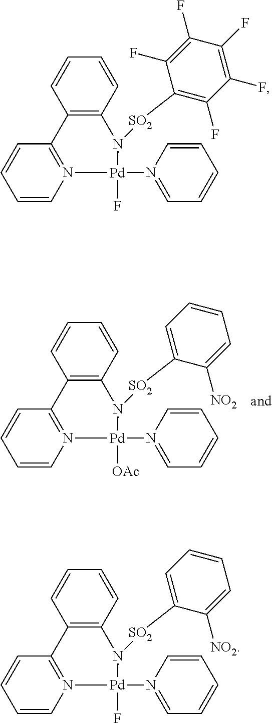

14. A method of fluorinating an organic compound, the method comprising mixing a palladium complex of formula: 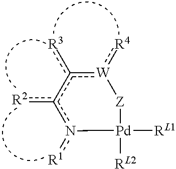
wherein:
Pd has a valency of +2;
R^{L1} is —N(R^{c})₂ wherein two R^{c} groups are joined to form an optionally substituted 6-membered heteroaryl ring;
R^{L2} is optionally substituted heteroaryl, halogen, —OR^{a}, —SR^{b}, —N(R^{c})₂, —N(R^{c})₃, or —P(R^{x})₃;
wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;
wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b}, C(═NR^{b3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;
wherein each instance of R^{c} is, independently, hydrogen, an optionally substituted aliphatic, optionally substituted heteroaliphatic, optionally substituted aryl, optionally substituted heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group [symbol] C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;
wherein each instance of R^{x} is, independently, hydrogen, an optionally substituted aliphatic, optionally substituted heteroaliphatic, optionally substituted aryl, or optionally substituted heteroaryl group;
when W is —C— or —C(R^{d})— then
Z is —O—, —S—, —C(R^{d})₂—, —C(R^{d})═C(R^{d})—, —C(R^{d})═N—, or —N(R^{e})—;
or
when W is —N— or —N(R^{e})— then Z is —C(R^{d})₂—, —C(R^{d})═C(R^{d})—, or —C(R^{d})═N—,
wherein each instance of R^{d} is, independently, hydrogen, or an optionally substituted aliphatic, optionally substituted heteroaliphatic, optionally substituted aryl, or optionally substituted heteroaryl group; and
each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, or a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;
R¹ and R² are joined to form an optionally substituted 5- to 7-membered heteroaryl, aryl, heterocyclic or carbocyclic ring;
R² and R³ are joined to form an optionally substituted 5- to 7-membered heteroaryl, aryl, heterocyclic or carbocyclic ring;
R³ and R⁴ are joined to form an optionally substituted 5- to 7-membered heteroaryl, aryl, heterocyclic or carbocyclic ring;
wherein each of the curved dotted lines [symbol] independently represents joining of an optionally substituted 5- to 7-membered ring; and
wherein [symbol] represents a single or double bond;
with a fluorinating agent and an organic compound, wherein the organic compound comprises a boron, organostannane or silane substituent, under conditions sufficient to fluorinate the organic compound, thereby providing a fluorinated organic compound.

15. The method of claim 14, wherein the organic compound comprises an aryl group.

16. The method of claim 14, wherein the organic compound comprises a boron substituent.

17. The method of claim 16, wherein the boron substituent is a group of the formulae: 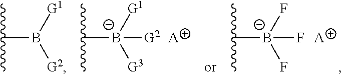
wherein G¹, G² and G³ are, independently, —OH, —OR^{G}, or —R^{G};
each R^{G} is, independently, optionally substituted aliphatic, optionally substituted heteroaliphatic, optionally substituted aryl, or optionally substituted heteroaryl, or G¹ and G² are joined to form a 5- to 8-membered ring having at least one O atom directly attached to B, wherein the ring is comprised of carbon atoms and optionally one or more additional heteroatoms independently selected from the group consisting of N, S, and O; and
wherein A^{⊕} is a metal cation or ammonium.

18. The method of claim 14, wherein the fluorinating agent provides a source of F⁺.

19. The palladium complex of claim 1, wherein the palladium complex is of the formula:

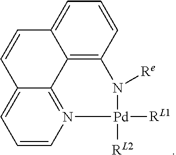

20. The palladium complex of claim 19, wherein R^{L1} is pyridyl.

21. The palladium complex of claim 19, wherein R^{L2} is halogen, an optionally substituted heteroaryl, or —OR^{a}.

22. The palladium complex of claim 21, wherein R^{L2} is —Cl or pyridyl.

23. The palladium complex of claim 21, wherein R^{a} is —C(═O)R^{a1} or —S(O)₂R^{a1}.

24. The palladium complex of claim 23, wherein R^{a1} is an optionally substituted aliphatic.

25. The palladium complex of claim 24, wherein R^{a} is —C(═O)CH₃ or —S(O)₂CF₃.

26. The palladium complex of claim 19, wherein R^{e} is: 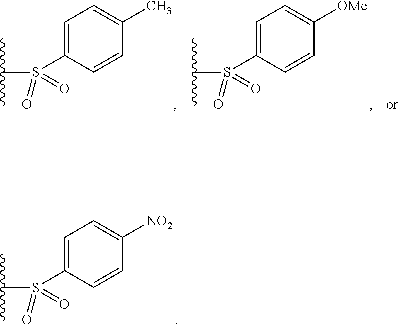 [SMILES: *S(=O)(=O)c1ccc(C)cc1 ; *S(=O)(=O)c1ccc(OC)cc1 ; *S(=O)(=O)c1ccc([N+](=O)[O-])cc1]
